SEQUENCE LISTING (434 sequences)

SEQ ID NO: 1 - protein, 214 aa, synthetic construct
  REGION 1..214: note = synthetic sequence
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 2 - protein, 450 aa, synthetic construct
  REGION 1..450: note = synthetic sequence
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 107 aa, synthetic construct [sequence omitted]

SEQ ID NO: 4 - protein, 120 aa, Homo sapiens [sequence omitted]

SEQ ID NO: 5 - protein, 50 aa, Homo sapiens
  VARIANT 1..50: note = Each copy of Gly Gly Gly Gly Ser at positions 1 to 50 is either present or absent such that the number of copies of Gly Gly Gly Gly Ser ranges from 1 to 10.
GGGGSGGGGSGGGGSGGGGSGGGGSGGGGSGGGGSGGGGSGGGGSGGGGS

SEQ ID NO: 6 - protein, 11 aa, Homo sapiens
RASQDVNTAVA

SEQ ID NO: 7 - protein, 6 aa, Homo sapiens
SASFLY

SEQ ID NO: 8 - protein, 8 aa, synthetic construct
  REGION 1..8: note = synthetic sequence
QQHYTTPP

SEQ ID NO: 9 - protein, 8 aa, synthetic construct
  REGION 1..8: note = synthetic sequence
GFNIKDTY

SEQ ID NO: 10 - protein, 8 aa, synthetic construct
  REGION 1..8: note = synthetic sequence
IYPTNGYT

SEQ ID NO: 11 - protein, 13 aa, synthetic construct
  REGION 1..13: note = synthetic sequence
SRWGGDGFYAMDY

SEQ ID NO: 12 - protein, 242 aa, synthetic construct
  REGION 1..242: note = synthetic sequence
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGGGGSGGGGSGGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIK

SEQ ID NO: 13 - protein, 214 aa, synthetic construct
  REGION 1..214: note = synthetic sequence
DIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 14 - protein, 448 aa, synthetic construct
  REGION 1..448: note = synthetic sequence
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 15 - protein, 107 aa, synthetic construct [sequence omitted]

SEQ ID NO: 16 - protein, 119 aa, synthetic construct [sequence omitted]

SEQ ID NO: 17 - protein, 11 aa, synthetic construct
  REGION 1..11: note = synthetic sequence
KASQDVSIGVA

SEQ ID NO: 18 - protein, 6 aa, synthetic construct
  REGION 1..6: note = synthetic sequence
SASYRY

SEQ ID NO: 19 - protein, 8 aa, synthetic construct
  REGION 1..8: note = synthetic sequence
QQYYIYPY

SEQ ID NO: 20 - protein, 10 aa, synthetic construct
  REGION 1..10: note = synthetic sequence
GFTFTDYTMD

SEQ ID NO: 21 - protein, 18 aa, synthetic construct
  REGION 1..18: note = synthetic sequence
ADVNPNSGGSIYNQRFKG

SEQ ID NO: 22 - protein, 12 aa, synthetic construct
  REGION 1..12: note = synthetic sequence
ARNLGPSFYFDY

SEQ ID NO: 23 - protein, 15 aa, synthetic construct
  REGION 1..15: note = synthetic sequence
KASQSVDYDGDSYLN

SEQ ID NO: 24 - protein, 7 aa, synthetic construct
  REGION 1..7: note = synthetic sequence
DASNLVS

SEQ ID NO: 25 - protein, 9 aa, synthetic construct
  REGION 1..9: note = synthetic sequence
QQSTEDPWT

SEQ ID NO: 26 - protein, 5 aa, synthetic construct
  REGION 1..5: note = synthetic sequence
SYWMN

SEQ ID NO: 27 - protein, 17 aa, synthetic construct
  REGION 1..17: note = synthetic sequence
QIWPGDGDTNYNGKFKG

SEQ ID NO: 28 - protein, 15 aa, synthetic construct
  REGION 1..15: note = synthetic sequence
RETTTVGRYYYAMDY

SEQ ID NO: 29 - protein, 249 aa, synthetic construct
  REGION 1..249: note = synthetic sequence
DIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKGGGGSGGGGSGGGGSQVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVS

SEQ ID NO: 30 - protein, 217 aa, synthetic construct
  REGION 1..217: note = synthetic sequence
DIALTQPASVSGSPGQSITISCTGTSSDIGGYNSVSWYQQHPGKAPKLMIYGVNNRPSGVSNRFSGSKSGNTASLTISGLQAEDEADYYCSSYDIESATPVFGGGTKLTVLGQPKAAPSVTLFPPSSEELQANKATLVCLISDFYPGAVTVAWKGDSSPVKAGVETTTPSKQSNNKYAASSYLSLTPEQWKSHRSYSCQVTHEGSTVEKTVAPTESS

SEQ ID NO: 31 - protein, 450 aa, synthetic construct
  REGION 1..450: note = synthetic sequence
QVELVQSGAEVKKPGESLKISCKGSGYSFTSYWIGWVRQAPGKGLEWMGIIDPGDSRTRYSPSFQGQVTISADKSISTAYLQWSSLKASDTAMYYCARGQLYGGTYMDGWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 32 - protein, 107 aa, synthetic construct
  REGION 1..107: note = synthetic sequence
DIALTQPASVSGSPGQSITISCTGTSSDIGGYNSVSWYQQHPGKAPKLMIYGVNNRPSGVSNRFSGSKSGNTASLTISGLQAEDEADYYCSSYDIESATPVFGGGTK

SEQ ID NO: 33 - protein, 120 aa, synthetic construct
  REGION 1..120: note = synthetic sequence
QVELVQSGAEVKKPGESLKISCKGSGYSFTSYWIGWVRQAPGKGLEWMGIIDPGDSRTRYSPSFQGQVTISADKSISTAYLQWSSLKASDTAMYYCARGQLYGGTYMDGWGQGTLVTVSS

SEQ ID NO: 34 - protein, 14 aa, synthetic construct
  REGION 1..14: note = synthetic sequence
TGTSSDIGGYNSVS

SEQ ID NO: 35 - protein, 11 aa, synthetic construct
  REGION 1..11: note = synthetic sequence
LMIYGVNNRPS

SEQ ID NO: 36 - protein, 10 aa, synthetic construct
  REGION 1..10: note = synthetic sequence
SSYDIESATP

SEQ ID NO: 37 - protein, 10 aa, synthetic construct
  REGION 1..10: note = synthetic sequence
GYSFTSYWIG

SEQ ID NO: 38 - protein, 16 aa, synthetic construct
  REGION 1..16: note = synthetic sequence
WMGIIDPGDSRTRYSP

SEQ ID NO: 39 - protein, 11 aa, synthetic construct
  REGION 1..11: note = synthetic sequence
GQLYGGTYMDG

SEQ ID NO: 40 - protein, 244 aa, synthetic construct
  REGION 1..244: note = synthetic sequence
QVQLQQSGAEVKKPGASVKVSCKASGYTFTGYYMHWVRQAPGQGLEWMGRINPNSGGTNYAQKFQGRVTMTRDTSISTAYMELSRLRSEDTAVYYCARGRYYGMDVWGQGTMVTVSSGGGGSGGGGSGGGGSGGGGSEIVLTQSPATLSLSPGERATISCRASQSVSSNFAWYQQRPGQAPRLLIYDASNRATGIPPRFSGSGSGTDFTLTISSLEPEDFAAYYCHQRSNWLYTFGQGTKVDIK

SEQ ID NO: 41 - protein, 253 aa, synthetic construct
  REGION 1..253: note = synthetic sequence
QVQLVQSGAEVKKPGASVKVSCKASGYTFTGYYMHWVRQAPGQGLEWMGWINPNSGGTNYAQKFQGRVTMTRDTSISTAYMELSRLRSDDTAVYYCARDLRRTVVTPRAYYGMDVWGQGTTVTVSSGGGGSGGGGSGGGGSGGGGSDIQLTQSPSTLSASVGDRVTITCQASQDISNSLNWYQQKAGKAPKLLIYDASTLETGVPSRFSGSGSGTDFSFTISSLQPEDIATYYCQQHDNLPLTFGQGTKVEIK

SEQ ID NO: 42 - protein, 217 aa, synthetic construct
  REGION 1..217: note = synthetic sequence
QSVLTQPPSASGTPGQRVTISCSGSSSNIGSNTVNWYQQLPGTAPKLLIFNYHQRPSGVPDRFSGSKSGSSASLAISGLQSEDEADYYCAAWDDSLNGWVFGGGTKLTVLGQPKAAPSVTLFPPSSEELQANKATLVCLISDFYPGAVTVAWKADSSPVKAGVETTTPDSKQSNNKYAASSYLSLTPEQWKSHRSYSCQVTHEGSTVEKTVAPTECS

SEQ ID NO: 43 - protein, 453 aa, synthetic construct
  REGION 1..453: note = synthetic sequence
EVQLVESGGGLVKPGGSLRLSCAASGFTFGDYALSWFRQAPGKGLEWVGVSRSKAYGGTTDYAASVKGRFTISRDDSKSTAYLQMNSLKTEDTAVYYCASSGYSSGWTPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 44 - protein, 111 aa, synthetic construct
  REGION 1..111: note = synthetic sequence
QSVLTQPPSASGTPGQRVTISCSGSSSNIGSNTVNWYQQLPGTAPKLLIFNYHQRPSGVPDRFSGSKSGSSASLAISGLQSEDEADYYCAAWDDSLNGWVFGGGTKLTVLG

SEQ ID NO: 45 - protein, 131 aa, synthetic construct
  REGION 1..131: note = synthetic sequence
EVQLVESGGGLVKPGGSLRLSCAASGFTFGDYALSWFRQAPGKGLEWVGVSRSKAYGGTTDYAASVKGRFTISRDDSKSTAYLQMNSLKTEDTAVYYCASSGYSSGWTPFDYWGQGTLVTVSSASTKGPSV

SEQ ID NO: 46 - protein, 8 aa, synthetic construct
  REGION 1..8: note = synthetic sequence
SSNIGSNT

SEQ ID NO: 47 - protein, 11 aa, synthetic construct
  REGION 1..11: note = synthetic sequence
AAWDDSLNGWV

SEQ ID NO: 48 - protein, 8 aa, synthetic construct
  REGION 1..8: note = synthetic sequence
GFTFGDYA

SEQ ID NO: 49 - protein, 10 aa, synthetic construct
  REGION 1..10: note = synthetic sequence
SRSKAYGGTT

SEQ ID NO: 50 - protein, 14 aa, synthetic construct
  REGION 1..14: note = synthetic sequence
ASSGYSSGWTPFDY

SEQ ID NO: 51 - protein, 249 aa, synthetic construct
  REGION 1..249: note = synthetic sequence
QVQLVQSGAEVKKPGSSVKVSCKASGGTFSNYWMHWVRQAPGQGLEWMGATYRGHSDTYYNQKFKGRVTITADKSTSTAYMELSSLRSEDTAVYYCARGAIYNGYDVLDNWGQGTLVTVSSGGGGSGGGGSGGGGSGGGGSDIQMTQSPSSLSASVGDRVTITCSASQDISNYLNWYQQKPGKAPKLLIYYTSNLHSGVPSRFSGSGSGTDFTLTISSLQPEDFATYYCQQYRKLPWTFGQGTKLEIKR

SEQ ID NO: 52 - protein, 249 aa, synthetic construct
  REGION 1..249: note = synthetic sequence
DIQMTQSPSSLSASVGDRVTITCSASQDISNYLNWYQQKPGKAPKLLIYYTSNLHSGVPSRFSGSGSGTDFTLTISSLQPEDFATYYCQQYRKLPWTFGQGTKLEIKRGGGGSGGGGSGGGGSGGGGSQVQLVQSGAEVKKPGSSVKVSCKASGGTFSNYWMHWVRQAPGQGLEWMGATYRGHSDTYYNQKFKGRVTITADKSTSTAYMELSSLRSEDTAVYYCARGAIYNGYDVLDNWGQGTLVTVSS

SEQ ID NO: 53 - protein, 11 aa, synthetic construct
  REGION 1..11: note = synthetic sequence
SASQDISNYLN

SEQ ID NO: 54 - protein, 7 aa, synthetic construct
  REGION 1..7: note = synthetic sequence
YTSNLHS

SEQ ID NO: 55 - protein, 9 aa, synthetic construct
  REGION 1..9: note = synthetic sequence
QQYRKLPWT

SEQ ID NO: 56 - protein, 5 aa, synthetic construct
  REGION 1..5: note = synthetic sequence
NYWMH

SEQ ID NO: 57 - protein, 17 aa, synthetic construct
  REGION 1..17: note = synthetic sequence
ATYRGHSDTYYNQKFKG

SEQ ID NO: 58 - protein, 12 aa, synthetic construct
  REGION 1..12: note = synthetic sequence
GAIYNGYDVLDN

SEQ ID NO: 59 - protein, 108 aa, synthetic construct
  REGION 1..108: note = synthetic sequence
DIQMTQSPSSLSASVGDRVTITCSASQDISNYLNWYQQKPGKAPKLLIYYTSNLHSGVPSRFSGSGSGTDFTLTISSLQPEDFATYYCQQYRKLPWTFGQGTKLEIKR

SEQ ID NO: 60 - protein, 121 aa, synthetic construct
  REGION 1..121: note = synthetic sequence
QVQLVQSGAEVKKPGSSVKVSCKASGGTFSNYWMHWVRQAPGQGLEWMGATYRGHSDTYYNQKFKGRVTITADKSTSTAYMELSSLRSEDTAVYYCARGAIYDGYDVLDNWGQGTLVTVSS

SEQ ID NO: 61 - protein, 20 aa, synthetic construct
  REGION 1..20: note = synthetic sequence
VTSAPDTRPAPGSTAPPAHG

SEQ ID NO: 62 - protein, 43 aa, synthetic construct
  REGION 1..43: note = synthetic sequence
SNIKFRPGSVVVQLTLAFREGTINVHDVETQFNQYKTEAASRY

SEQ ID NO: 63 - protein, 35 aa, synthetic construct
  REGION 1..35: note = synthetic sequence
SVVVQLTLAFREGTINVHDVETQFNQYKTEAASRY

SEQ ID NO: 64 - protein, 20 aa, synthetic construct
  REGION 1..20: note = synthetic sequence
LAFREGTINVHDVETQFNQY

SEQ ID NO: 65 - protein, 25 aa, synthetic construct
  REGION 1..25: note = synthetic sequence
SNIKFRPGSVVVQLTLAAFREGTIN

SEQ ID NO: 66 - protein, 5 aa, synthetic construct
  REGION 1..5: note = synthetic sequence
RYGMS

SEQ ID NO: 67 - protein, 17 aa, synthetic construct
  REGION 1..17: note = synthetic sequence
TISGGGTYIYYPDSVKG

SEQ ID NO: 68 - protein, 13 aa, synthetic construct
  REGION 1..13: note = synthetic sequence
DNYGRNYDYGMDY

SEQ ID NO: 69 - protein, 10 aa, synthetic construct
  REGION 1..10: note = synthetic sequence
SATSSVSYIH

SEQ ID NO: 70 - protein, 7 aa, synthetic construct
  REGION 1..7: note = synthetic sequence
STSNLAS

SEQ ID NO: 71 - protein, 9 aa, synthetic construct
  REGION 1..9: note = synthetic sequence
QQRSSSPFT

SEQ ID NO: 72 - protein, 5 aa, synthetic construct
  REGION 1..5: note = synthetic sequence
GYAMS

SEQ ID NO: 73 - protein, 17 aa, synthetic construct
  REGION 1..17: note = synthetic sequence
TISSGGTYIYYPDSVKG

SEQ ID NO: 74 - protein, 12 aa, synthetic construct
  REGION 1..12: note = synthetic sequence
LGGDNYYEYFDV

SEQ ID NO: 75 - protein, 15 aa, synthetic construct
  REGION 1..15: note = synthetic sequence
RASKSVSTSGYSYMH

SEQ ID NO: 76 - protein, 7 aa, synthetic construct
  REGION 1..7: note = synthetic sequence
LASNLES

SEQ ID NO: 77 - protein, 9 aa, synthetic construct
  REGION 1..9: note = synthetic sequence
QHSRELPFT

SEQ ID NO: 78 - protein, 5 aa, synthetic construct
  REGION 1..5: note = synthetic sequence
DYAMN

SEQ ID NO: 79 - protein, 17 aa, synthetic construct
  REGION 1..17: note = synthetic sequence
VISTFSGNINFNQKFKG

SEQ ID NO: 80 - protein, 10 aa, synthetic construct
  REGION 1..10: note = synthetic sequence
SDYYGPYFDY

SEQ ID NO: 81 - protein, 16 aa, synthetic construct
  REGION 1..16: note = synthetic sequence
RSSQTIVHSNGNTYLE

SEQ ID NO: 82 - protein, 7 aa, synthetic construct
  REGION 1..7: note = synthetic sequence
KVSNRFS

SEQ ID NO: 83 - protein, 9 aa, synthetic construct
  REGION 1..9: note = synthetic sequence
FQGSHVPFT

SEQ ID NO: 84 - protein, 9 aa, synthetic construct
  REGION 1..9: note = synthetic sequence
LGGDNYYEY

SEQ ID NO: 85 - protein, 15 aa, synthetic construct
  REGION 1..15: note = synthetic sequence
TASKSVSTSGYSYMH

SEQ ID NO: 86 - protein, 7 aa, synthetic construct
  REGION 1..7: note = synthetic sequence
LVSNLES

SEQ ID NO: 87 - protein, 10 aa, synthetic construct
  REGION 1..10: note = synthetic sequence
QHIRELTRSE

SEQ ID NO: 88 - protein, 8 aa, synthetic construct
  REGION 1..8: note = synthetic sequence
GFTFSNYY

SEQ ID NO: 89 - protein, 8 aa, synthetic construct
  REGION 1..8: note = synthetic sequence
ISGRGSTI

SEQ ID NO: 90 - protein, 10 aa, synthetic construct
  REGION 1..10: note = synthetic sequence
VKDRGGYSPY

SEQ ID NO: 91 - protein, 6 aa, synthetic construct
  REGION 1..6: note = synthetic sequence
QSISTY

SEQ ID NO: 92 - protein, 10 aa, synthetic construct
  REGION 1..10: note = synthetic sequence
QQSYSTPPIT

SEQ ID NO: 93 - protein, 5 aa, synthetic construct
  REGION 1..5: note = synthetic sequence
DKTHT

SEQ ID NO: 94 - protein, 4 aa, synthetic construct
  REGION 1..4: note = synthetic sequence
CPPC

SEQ ID NO: 95 - protein, 15 aa, synthetic construct
  REGION 1..15: note = synthetic sequence
CPEPKSCDTPPPCPR

SEQ ID NO: 96 - protein, 12 aa, synthetic construct
  REGION 1..12: note = synthetic sequence
ELKTPLGDTTHT

SEQ ID NO: 97 - protein, 10 aa, synthetic construct
  REGION 1..10: note = synthetic sequence
KSCDKTHTCP

SEQ ID NO: 98 - protein, 7 aa, synthetic construct
  REGION 1..7: note = synthetic sequence
KCCVDCP

SEQ ID NO: 99 - protein, 7 aa, synthetic construct
  REGION 1..7: note = synthetic sequence
KYGPPCP

SEQ ID NO: 100 - protein, 15 aa, synthetic construct
  REGION 1..15: note = synthetic sequence
EPKSCDKTHTCPPCP

SEQ ID NO: 101 - protein, 12 aa, synthetic construct
  REGION 1..12: note = synthetic sequence
ERKCCVECPPCP

SEQ ID NO: 102 - protein, 17 aa, synthetic construct
  REGION 1..17: note = synthetic sequence
ELKTPLGDTTHTCPRCP

SEQ ID NO: 103 - protein, 12 aa, synthetic construct
  REGION 1..12: note = synthetic sequence
SPNMVPHAHHAQ

SEQ ID NO: 104 - protein, 45 aa, synthetic construct
  REGION 1..45: note = synthetic sequence
TTTPAPRPPTPAPTIASQPLSLRPEACRPAAGGAVHTRGLDFACD

SEQ ID NO: 105 - protein, 24 aa, synthetic construct
  REGION 1..24: note = synthetic sequence
IYIWAPLAGTCGVLLLSLVITLYC

SEQ ID NO: 106 - protein, 23 aa, synthetic construct
  REGION 1..23: note = synthetic sequence
LGLLVAGVLVLLVSLGVAIHLCC

SEQ ID NO: 107 - protein, 25 aa, synthetic construct
  REGION 1..25: note = synthetic sequence
ALIVLGGVAGLLLFIGLGIFFCVRC

SEQ ID NO: 108 - protein, 23 aa, synthetic construct
  REGION 1..23: note = synthetic sequence
LCYLLDGILFIYGVILTALFLRV

SEQ ID NO: 109 - protein, 26 aa, synthetic construct
  REGION 1..26: note = synthetic sequence
WVLVVVGGVLACYSLLVTVAFIIFWV

SEQ ID NO: 110 - protein, 26 aa, synthetic construct
  REGION 1..26: note = synthetic sequence
VAAILGLGLVLGLLGPLAILLALYLL

SEQ ID NO: 111 - protein, 24 aa, synthetic construct
  REGION 1..24: note = synthetic sequence
ALPAALAVISFLLGLGLGVACVLA

SEQ ID NO: 112 - protein, 42 aa, synthetic construct
  REGION 1..42: note = synthetic sequence
KRGRKKLLYIFKQPFMRPVQTTQEEDGCSCRFPEEEEGGCEL

SEQ ID NO: 113 - protein, 44 aa, synthetic construct
  REGION 1..44: note = synthetic sequence
FWVRSKRSRLLHSDYMNMTPRRPGPTRKHYQPYAPPRDFAAYRS

SEQ ID NO: 114 - protein, 35 aa, synthetic construct
  REGION 1..35: note = synthetic sequence
TKKKYSSSVHDPNGEYMFMRAVNTAKKSRLTDVTL

SEQ ID NO: 115 - protein, 37 aa, synthetic construct
  REGION 1..37: note = synthetic sequence
RRDQRLPPDAHKPPGGGSFRTPIQEEQADAHSTLAKI

SEQ ID NO: 116 - protein, 114 aa, synthetic construct
  REGION 1..114: note = synthetic sequence
CCLRRHQGKQNELSDTAGREINLVDAHLKSEQTEASTRQNSQVLLSETGIYDNDPDLCFRMQEGSEVYSNPCLEENKPGIVYASLNHSVIGPNSRLARNVKEAPTEYASICVRS

SEQ ID NO: 117 - protein, 49 aa, synthetic construct
  REGION 1..49: note = synthetic sequence
HQRRKYRSNKGESPVEPAEPCRYSCPREEEGSTIPIQEDYRKPEPACSP

SEQ ID NO: 118 - protein, 187 aa, synthetic construct
  REGION 1..187: note = synthetic sequence
RRACRKRIRQKLHLCYPVQTSQPKLELVDSRPRRSSTQLRSGASVTEPVAEERGLMSQPLMETCHSVGAAYLESLPLQDASPAGGPSSPRDLPEPRVSTEHTNNKIEKIYIMKADTVIVGTVKAELPEGRGLAGPAEPELEEELEADHTPHYPEQETEPPLGSCSDVMLSVEEEGKEDPLPTAASGK

SEQ ID NO: 119 - protein, 54 aa, synthetic construct
  REGION 1..54: note = synthetic sequence
HIWQLRSQCMWPRETQLLLEVPPSTEDARSCQFPEEERGERSAEEKGRLGDLWV

SEQ ID NO: 120 - protein, 60 aa, synthetic construct
  REGION 1..60: note = synthetic sequence
CVKRRKPRGDVVKVIVSVQRKRQEAEGEATVIEALQAPPDVTTVAVEETIPSFTGRSPNH

SEQ ID NO: 121 - skipped

SEQ ID NO: 122 - protein, 16 aa, synthetic construct
  REGION 1..16: note = synthetic sequence
  VARIANT 2..3: note = The amino acids at positions 2 and 3 are independently any amino acid
  VARIANT 4: note = The amino acid at position 4 may also be Ile
  VARIANT 5..12: note = Where Xaa is independently any amino acid and each copy of Xaa at positions 5 to 12 is either present or absent such that the number of Xaa ranges from 6 to 8.
  VARIANT 14..15: note = The amino acids at positions 14 and 15 are independently any amino acid
  VARIANT 16: note = The amino acid at position 16 may also be Ile
YXXLXXXXXXXXYXXL

SEQ ID NO: 123 - protein, 561 aa, Cytomegalovirus
MESRGRRCPEMISVLGPISGHVLKAVFSRGDTPVLPHETRLLQTGIHVRVSQPSLILVSQYTPDSTPCHRGDNQLQVQHTYFTGSEVENVSVNVHNPTGRSICPSQEPMSIYVYALPLKMLNIPSINVHHYPSAAERKHRHLPVADAVIHASGKQMWQARLTVSGLAWTRQQNQWKEPDVYYTSAFVFPTKDVALRHVVCAHELVCSMENTRATKMQVIGDQYVKVYLESFCEDVPSGKLFMHVTLGSDVEEDLTMTRNPQPFMRPHERNGFTVLCPKNMIIKPGKISHIMLDVAFTSHEHFGLLCPKSIPGLSISGNLLMNGQQIFLEVQAIRETVELRQYDPVAALFFFDIDLLLQRGPQYSEHPTFTSQYRIQGKLEYRHTWDRHDEGAAQGDDDVWTSGSDSDEELVTTERKTPRVTGGGAMAGASTSAGRKRKSASSATACTSGVMTRGRLKAESTVAPEEDTDEDSDNEIHNPAVFTWPPWQAGILARNLVPMVATVQGQNLKYQEFFWDANDIYRIFAELEGVWQPAAQPKRRRHRQDALPGPCIASTPKKHRG

SEQ ID NO: 124 - protein, 9 aa, synthetic construct
  REGION 1..9: note = synthetic sequence
KLPQLCTEL

SEQ ID NO: 125 - protein, 9 aa, Human papillomavirus
LQTTIHDII

SEQ ID NO: 126 - protein, 9 aa, Human papillomavirus
VYDFAFRDL

SEQ ID NO: 127 - protein, 9 aa, Human papillomavirus
FAFRDLCIV

SEQ ID NO: 128 - protein, 9 aa, Human papillomavirus
KFYSKISEY

SEQ ID NO: 129 - protein, 9 aa, Human papillomavirus
ISEYRHYCY

SEQ ID NO: 130 - protein, 9 aa, Human papillomavirus
TLHEYMLDL

SEQ ID NO: 131 - protein, 9 aa, Human papillomavirus
YMLDLQPET

SEQ ID NO: 132 - protein, 9 aa, Human papillomavirus
QAEPDRAHY

SEQ ID NO: 133 - protein, 9 aa, Human papillomavirus
RAHYNIVTF

SEQ ID NO: 134 - protein, 9 aa, Human papillomavirus
CDSTLRLCV

SEQ ID NO: 135 - protein, 10 aa, Human papillomavirus
LCVQSTHVDI

SEQ ID NO: 136 - protein, 9 aa, Human papillomavirus
LLMGTLGIV

SEQ ID NO: 137 - protein, 8 aa, Human papillomavirus
TLGIVCPI

SEQ ID NO: 138 - protein, 12 aa, Human papillomavirus
LLMGTLGIVCPI

SEQ ID NO: 139 - protein, 9 aa, Human papillomavirus
CYSLYGTTL

SEQ ID NO: 140 - protein, 9 aa, Human papillomavirus
EYRHYCYSL

SEQ ID NO: 141 - protein, 9 aa, Human papillomavirus
DPQERPRKL

SEQ ID NO: 142 - protein, 9 aa, Human papillomavirus
HYCYSLYGT

SEQ ID NO: 143 - protein, 9 aa, Human papillomavirus
DFAFRDLCI

SEQ ID NO: 144 - protein, 10 aa, Human papillomavirus
LYGTTLEQQY

SEQ ID NO: 145 - protein, 10 aa, Human papillomavirus
HYCYSLYGTT

SEQ ID NO: 146 - protein, 10 aa, Human papillomavirus
EVYDFAFRDL

SEQ ID NO: 147 - protein, 10 aa, Human papillomavirus
EYRHYCYSLY

SEQ ID NO: 148 - protein, 10 aa, Human papillomavirus
VYDFAFRDLC

SEQ ID NO: 149 - protein, 10 aa, Human papillomavirus
YCYSIYGTTL

SEQ ID NO: 150 - protein, 9 aa, Human papillomavirus
VYCKTVLEL

SEQ ID NO: 151 - protein, 9 aa, Human papillomavirus
VYGDTLEKL

SEQ ID NO: 152 - protein, 10 aa, Human papillomavirus
LTNTGLYNLL

SEQ ID NO: 153 - protein, 9 aa, Human papillomavirus
DLQPETTDL

SEQ ID NO: 154 - protein, 9 aa, Human papillomavirus
TPTLHEYML

SEQ ID NO: 155 - protein, 9 aa, Human papillomavirus
GTLGIVCPI

SEQ ID NO: 156 - protein, 9 aa, Human papillomavirus
EPDRAHYNI

SEQ ID NO: 157 - protein, 9 aa, Human papillomavirus
QLFLNTLSF

SEQ ID NO: 158 - protein, 9 aa, Human papillomavirus
FQQLFLNTL

SEQ ID NO: 159 - protein, 10 aa, Human papillomavirus
AFQQLFLNTL

SEQ ID NO: 160 - protein, 9 aa, Influenza virus
GILGFVFTL

SEQ ID NO: 161 - protein, 14 aa, Clostridium tetani
QYIKANSKFIGIFE

SEQ ID NO: 162 - protein, 14 aa, Clostridium tetani
QYIKANSKFIGITE

SEQ ID NO: 163 - protein, 15 aa, Clostridium tetani
ILMQYIKANSKFIGI

SEQ ID NO: 164 - protein, 7 aa, Clostridium tetani
VNNESSE

SEQ ID NO: 165 - protein, 17 aa, Clostridium tetani
PGINGKAIHLVNNESSE

SEQ ID NO: 166 - protein, 6 aa, Clostridium tetani
PNRDIL

SEQ ID NO: 167 - protein, 7 aa, Clostridium tetani
FIGITEL

SEQ ID NO: 168 - protein, 6 aa, Clostridium tetani
SYFPSV

SEQ ID NO: 169 - protein, 20 aa, Clostridium tetani
NSVDDALINSTKIYSYFPSV

SEQ ID NO: 170 - protein, 20 aa, Clostridium tetani
IDKISDVSTIVPYIGPALNI

SEQ ID NO: 171 - protein, 9 aa, synthetic construct
  REGION 1..9: note = synthetic sequence
NLMPMVATV

SEQ ID NO: 172 - protein, 9 aa, Cytomegalovirus
NLVPMVATV

SEQ ID NO: 173 - protein, 906 aa, Cytomegalovirus
MESRIWCLVVCVNLCIVCLGAAVSSSSTSHATSSTHNGSHTSRTTSAQTRSVYSQHVTSSEAVSHRANETIYNTTLKYGDVVGVNTTKYPYRVCSMAQGTDLIRFERNIICTSMKPINEDLDEGIMVVYKRNIVAHTFKVRVYQKVLTFRRSYAYIYTTYLLGSNTEYVAPPMWEIHHINKFAQCYSSYSRVIGGTVFVAYHRDSYENKTMQLIPDDYSNTHSTRYVTVKDQWHSRGSTWLYRETCNLNCMLTITTARSKYPYHFFATSTGDVVYISPFYNGTNRNASYFGENADKFFIFPNYTIVSDFGRPNAAPETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETSGGLVVFWQGIKQKSLVELERLANRSSLNITHRTRRSTSDNNTTHLSSMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLENTDFRVLELYSQKELRSSNVFDLEEIMREFNSYKQRVKYVEDKVVDPLPPYLKGLDDLMSGLGAAGKAVGVAIGAVGGAVASVVEGVATFLKNPFGAFTIILVAIAVVIITYLIYTRQRRLCTQPLQNLFPYLVSADGTTVTSGSTKDTSLQAPPSYEESVYNSGRKGPGPPSSDASTAAPPYTNEQAYQMLLALARLDAEQRAQQNGTDSLDGQTGTQDKGQKPNLLDRLRHRKNGYRHLKDSDEEENV

SEQ ID NO: 174 - protein, 5 aa, synthetic construct
  REGION 1..5: note = synthetic sequence
GTLRG

SEQ ID NO: 175 - protein, 5 aa, synthetic construct
  REGION 1..5: note = synthetic sequence
YNQSE

SEQ ID NO: 176 - protein, 5 aa, synthetic construct
  REGION 1..5: note = synthetic sequence
TAADM

SEQ ID NO: 177 - protein, 5 aa, synthetic construct
  REGION 1..5: note = synthetic sequence
AQTTK

SEQ ID NO: 178 - protein, 5 aa, synthetic construct
  REGION 1..5: note = synthetic sequence
VETRP

SEQ ID NO: 179 - protein, 5 aa, synthetic construct
  REGION 1..5: note = synthetic sequence
GDGTF

SEQ ID NO: 180 - protein, 5 aa, synthetic construct
  REGION 1..5: note = synthetic sequence
RNLRG

SEQ ID NO: 181 - protein, 5 aa, synthetic construct
  REGION 1..5: note = synthetic sequence
TAADT

SEQ ID NO: 182 - protein, 5 aa, synthetic construct
  REGION 1..5: note = synthetic sequence
AQITQ

SEQ ID NO: 183 - protein, 5 aa, synthetic construct
  REGION 1..5: note = synthetic sequence
GDRTF

SEQ ID NO: 184 - protein, 276 aa, synthetic construct
  REGION 1..276: note = synthetic sequence
  VARIANT 9: note = Xaa is Phe, Tyr, Ser, or Thr
  VARIANT 44: note = Xaa is Lys or Arg
  VARIANT 62: note = Xaa is Gln, Gly, Glu, or Arg
  VARIANT 63: note = Xaa is Asn or Glu
  VARIANT 65: note = Xaa is Arg or Gly
  VARIANT 66: note = Xaa is Asn or Lys
  VARIANT 67: note = Xaa is Met or Val
  VARIANT 70: note = Xaa is His or Gln
  VARIANT 73: note = Xaa is His or Gln
  VARIANT 74: note = Xaa is Asp or His
  VARIANT 76: note = Xaa is Ala, Val, or Glu
  VARIANT 77: note = Xaa is Asn or Asp
  VARIANT 79: note = Xaa is Gly or Arg
  VARIANT 80: note = Xaa is Thr or Ile
  VARIANT 81: note = Xaa is Leu or Ala
  VARIANT 82: note = Xaa is Arg or Leu
  VARIANT 83: note = Xaa is Gly or Arg
  VARIANT 90: note = Xaa is Ala or Asp
  VARIANT 95: note = Xaa is Ile, Leu, or Val
  VARIANT 97: note = Xaa is Ile, Arg or Met
  VARIANT 99: note = Xaa is Phe or Tyr
  VARIANT 105: note = Xaa is Ser or Pro
  VARIANT 107: note = Xaa is Try or Gly
  VARIANT 114: note = Xaa is Arg, His, or Gln
  VARIANT 116: note = Xaa is Asp or Tyr
  VARIANT 127: note = Xaa is Asn or Lys
  VARIANT 142: note = Xaa is Thr or Ile
  VARIANT 144: note = Xaa is Lys or Gln
  VARIANT 145: note = Xaa is Arg or His
  VARIANT 149: note = Xaa is Ala or Thr
  VARIANT 150: note = Xaa is Ala or Val
  VARIANT 151: note = Xaa is His or Arg
  VARIANT 156: note = Xaa is Arg, Leu, Gln, or Try
  VARIANT 158: note = Xaa is Val or Ala
  VARIANT 161: note = Xaa is Asp or Glu
  VARIANT 163: note = Xaa is Arg or Thr
  VARIANT 166: note = Xaa is Asp or Glu
  VARIANT 167: note = Xaa is Try or Gly
  VARIANT 184: note = Xaa is Pro or Ala
  VARIANT 193: note = Xaa is Pro or Ala
  VARIANT 194: note = Xaa is Pro or Ala
  VARIANT 207: note = Xaa is Ser or Gly
  VARIANT 246: note = Xaa is Ala or Ser
  VARIANT 253: note = Xaa is Gln or Glu
  VARIANT 276: note = Xaa is Pro or Leu
GSHSMRYFXTSVSRPGRGEPRFIAVGYVDDTQFVRFDSDAASQXMEPRAPWIEQEGPEYWDXXTXXXKAXSQXXRXXLXXXXXYYNQSEXGSHTXQXMXGCDVGXDXRFLRGYXQXAYDGKDYIALXEDLRSWTAADMAAQXTXXKWEXXXEAEQXRXYLXGXCVXXLRRYLENGKETLQRTDXPKTHMTHHXXSDHEATLRCWALXFYPAEITLTWQRDGEDQTQDTELVETRPAGDGTFQKWAXVVVPSGXEQRYTCHVQHEGLPKPLTLRWEX

SEQ ID NO: 185 - protein, 276 aa, synthetic construct [sequence omitted]

SEQ ID NO: 186 - protein, 275 aa, synthetic construct [sequence omitted]

SEQ ID NO: 187 - protein, 276 aa, synthetic construct
  REGION 1..276: note = synthetic sequence
GSHSMRYFFTSVSRPGRGEPRFIAVGYVDDTQFVRFDSDAASQRMEPRAPWIEQEGPEYWDGETRKVKAHSQTHRVDLGTLRGAYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPCGDGTFQKWAAVVVPSGQEQRYTCHVQHEGLPKPLTLRWEP

SEQ ID NO: 188 - protein, 275 aa, synthetic construct
  REGION 1..275: note = synthetic sequence
GSHSMRYFFTSVSRPGRGEPRFIAVGYVDDTQFVRFDSDAASQRMEPRAPWIEQEGPEYWDGETRKVKAHSQTHRVDLGTLRGAYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPCGDGTFQKWAAVVVPSGQEQRYTCHVQHEGLPKPLTLRWE

SEQ ID NO: 189 - protein, 276 aa, synthetic construct
  REGION 1..276: note = synthetic sequence
GSHSMRYFFTSVSRPGRGEPRFIAVGYVDDTQFVRFDSDAASQRMEPRAPWIEQEGPEYWDGETRKVKAHSQTHRVDLGTLRGCYNQSEAGSHTVQRMYGCDVGSDWRFLRGYHQYAYDGKDYIALKEDLRSWTAADMCAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRWEP

SEQ ID NO: 190 - protein, 275 aa, synthetic construct
  REGION 1..275: note = synthetic sequence
GSHSMRYFYTSVSRPGRGEPRFIAVGYVDDTQFVRFDSDAASQRMEPRAPWIEQEGPEYWDQETRNVKAQSQTDRVDLGTLRGYYNQSEDGSHTIQIMYGCDVGPDGRFLRGYRQDAYDGKDYIALNEDLRSWTAADMAAQITKRKWEAAHAAEQQRAYLEGTCVEWLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWE

SEQ ID NO: 191 - protein, 275 aa, synthetic construct
  REGION 1..275: note = synthetic sequence
GSHSMRYFYTSVSRPGRGEPRFIAVGYVDDTQFVRFDSDAASQRMEPRAPWIEQEGPEYWDQETRNVKAQSQTDRVDLGTLRGAYNQSEDGSHTIQIMYGCDVGPDGRFLRGYRQDAYDGKDYIALNEDLRSWTAADMAAQITKRKWEAAHAAEQQRAYLEGTCVEWLRRYLENGKETLQRTDPPKTHMTHHPISDHEATLRCWALGFYPAEITLTWQRDGEDQTQDTELVETRPCGDGTFQKWAAVVVPSGEEQRYTCHVQHEGLPKPLTLRWE

SEQ ID NO: 192 - protein, 341 aa, synthetic construct
  REGION 1..341: note = synthetic sequence
GSHSMRYFSTSVSRPGRGEPRFIAVGYVDDTQFVRFDSDAASQRMEPRAPWIEQEGPEYWDEETGKVKAHSQTDRENLRIALRYYNQSEAGSHTLQMMFGCDVGSDGRFLRGYHQYAYDGKDYIALKEDLRSWTAADMAAQITKRKWEAAHVAEQQRAYLEGTCVDGLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWEPSSQPTVPIVGIIAGLVLLGAVITGAVVAAVMWRRNSSDRKGGSYSQAASSDSAQGSDVSLTACKV

SEQ ID NO: 193 - protein, 341 aa, synthetic construct
  REGION 1..341: note = synthetic sequence
GSHSMRYFTTSVSRPGRGEPRFIAVGYVDDTQFVRFDSDAASQRMEPRAPWIEQEGPEYWDRNTRNVKAHSQIDRVDLGTLRGYYNQSEAGSHTIQMMYGCDVGSDGRFLRGYQQDAYDGKDYIALNEDLRSWTAADMAAQITQRKWEAARVAEQLRAYLEGTCVEWLRRYLENGKETLQRTDPPKTHMTHHAVSDHEATLRCWALSFYPAEITLTWQRDGEDQTQDTELVETRPAGDGTFQKWASVVVPSGQEQRYTCHVQHEGLPKPLTLRWEPSSQPTIPIVGIIAGLVLFGAVFAGAVVAAVRWRRKSSDRKGGSYSQAASSDSAQGSDMSLTACKV

SEQ ID NO: 194 - protein, 276 aa, synthetic construct
  REGION 1..276: note = synthetic sequence
  VARIANT 9: note = Xaa is His, Tyr or Asp
  VARIANT 11: note = Xaa is Ala or Ser
  VARIANT 12: note = Xaa is Met or Val
  VARIANT 24: note = Xaa is Ala, Ser, or Thr
  VARIANT 32: note = Xaa is Gln or Leu
  VARIANT 41: note = Xaa is Ala or Thr
  VARIANT 45: note = Xaa is Glu, Met, Lys, or Thr
  VARIANT 46: note = Xaa is Ala or Thr
  VARIANT 63: note = Xaa is Glu or Asn
  VARIANT 66: note = Xaa is Ile or Lys
  VARIANT 67: note = Xaa is Tyr, Phe, Ser, or Cys
  VARIANT 70: note = Xaa is Asn or Gln
  VARIANT 71: note = Xaa is Ala or Thr
  VARIANT 74: note = Xaa is Asp or Tyr
  VARIANT 76: note = Xaa is Glu or Val
  VARIANT 77: note = Xaa is Ser or Asn
  VARIANT 80: note = Xaa is Thr, Asn or Ile
  VARIANT 81: note = Xaa is Ala or Leu
  VARIANT 82: note = Xaa is Leu or Arg
  VARIANT 83: note = Xaa is Arg or Gly
  VARIANT 94: note = Xaa is Thr or Ile
  VARIANT 95: note = Xaa is Leu or Ile
  VARIANT 97: note = Xaa is Arg or Ser
  VARIANT 131: note = Xaa is Arg or Ser
  VARIANT 143: note = Xaa is Ser or Thr
  VARIANT 147: note = Xaa is Leu or Try
  VARIANT 152: note = Xaa is Glu or Val
  VARIANT 156: note = Xaa is Arg, Asp, Leu or Try
  VARIANT 158: note = Xaa is Ala or Thr
  VARIANT 163: note = Xaa is Leu, Glu or Thr
  VARIANT 177: note = Xaa is Glu or Asp
  VARIANT 178: note = Xaa is Lys or Thr
  VARIANT 180: note = Xaa is Glu or Gln
  VARIANT 194: note = Xaa is Ile or Val
GSHSMRYFXTXXSRPGRGEPRFIXVGYVDDTXFVRFDSDAXSPRXXPRAPWIEQEGPEYWDRXTQXXKTXXTQXYXXNLXXXXYYNQSEAGSHXXQXMYGCDLGPDGRLLRGHDQSAYDGKDYIALNEDLXSWTAADTAAQIXQRKXEAARXAEQXRXYLEGXCVEWLRRYLENGKXXLXRADPPKTHVTHHPXSDHEATLRCWALGFYPAEITLTWQRDGEDQTQDTELVETRPAGDRTFQKWAAVVVPSGEEQRYTCHVQHEGLPKPLTLRWEP

SEQ ID NO: 195 - protein, 276 aa, synthetic construct [sequence omitted]

SEQ ID NO: 196 - protein, 276 aa, synthetic construct
  REGION 1..276: note = synthetic sequence
GSHSMRYFYTSVSRPGRGEPRFISVGYVDDTQFVRFDSDAASPREEPRAPWIEQEGPEYWDRNTQIYKAQAQTDRESLRNLRGAYNQSEAGSHTLQSMYGCDVGPDGRLLRGHDQYAYDGKDYIALNEDLRSWTAADTAAQITQRKWEAAREAEQRRAYLEGECVEWLRRYLENGKDKLERADPPKTHVTHHPISDHEATLRCWALGFYPAEITLTWQRDGEDQTQDTELVETRPCGDRTFQKWAAVVVPSGEEQRYTCHVQHEGLPKPLTLRWEP

SEQ ID NO: 197 - protein, 276 aa, synthetic construct
  REGION 1..276: note = synthetic sequence
GSHSMRYFYTSVSRPGRGEPRFISVGYVDDTQFVRFDSDAASPREEPRAPWIEQEGPEYWDRNTQIYKAQAQTDRESLRNLRGCYNQSEAGSHTLQSMYGCDVGPDGRLLRGHDQYAYDGKDYIALNEDLRSWTAADTCAQITQRKWEAAREAEQRRAYLEGECVEWLRRYLENGKDKLERADPPKTHVTHHPISDHEATLRCWALGFYPAEITLTWQRDGEDQTQDTELVETRPAGDRTFQKWAAVVVPSGEEQRYTCHVQHEGLPKPLTLRWEP

SEQ ID NO: 198 - protein, 276 aa, synthetic construct
  REGION 1..276: note = synthetic sequence
  VARIANT 1: note = Xaa is Cys or Gly
  VARIANT 6: note = Xaa is Arg or Lys
  VARIANT 9: note = Xaa is Phe, Tyr, Ser, or Asp
  VARIANT 14: note = Xaa is Arg or Try
  VARIANT 21: note = Xaa is His or Arg
  VARIANT 24: note = Xaa is Ala or Ser
  VARIANT 35: note = Xaa is Gln or Arg
  VARIANT 49: note = Xaa is Ala or Glu
  VARIANT 66: note = Xaa is Asn or Lys
  VARIANT 73: note = Xaa is Thr or Ala
  VARIANT 77: note = Xaa is Ser or Asn
  VARIANT 80: note = Xaa is Asn or Lys
  VARIANT 90: note = Xaa is Ala or Asp
  VARIANT 91: note = Xaa is Gly or Arg
  VARIANT 94: note = Xaa is Thr or Ile
  VARIANT 95: note = Xaa is Leu or Ile
  VARIANT 97: note = Xaa is Try or Arg
  VARIANT 99: note = Xaa is Cys, Tyr, Phe, or Ser
  VARIANT 103: note = Xaa is Leu or Val
  VARIANT 113: note = Xaa is Tyr or His
  VARIANT 114: note = Xaa is Asp or Asn
  VARIANT 116: note = Xaa is Tyr, Phe, Ser, or Leu
  VARIANT 147: note = Xaa is Leu or Try
  VARIANT 152: note = Xaa is Glu, Ala or Thr
  VARIANT 156: note = Xaa is Arg, Leu or Try
  VARIANT 163: note = Xaa is Leu or Thr
  VARIANT 173: note = Xaa is Glu or Lys
  VARIANT 177: note = Xaa is Glu or Lys
  VARIANT 184: note = Xaa is His or Pro
  VARIANT 194: note = Xaa is Arg or Val
  VARIANT 219: note = Xaa is Try or Arg
  VARIANT 248: note = Xaa is Val or Met
  VARIANT 253: note = Xaa is Glu or Gln
  VARIANT 261: note = Xaa is Met or Val
  VARIANT 267: note = Xaa is Pro or Gln
  VARIANT 273: note = Xaa is Arg or Ser
  VARIANT 275: note = Xaa is Pro or Gly
XSHSMXYFXTAVSXPGRGEPXFIXVGYVDDTQFVXFDSDAASPRGEPRXPWVEQEGPEYWDRETQXYKRQAQXDRVXLRXLRGYYNQSEXXSHXXQXMXGCDXGPDGRLLRGXXQXAYDGKDYIALNEDLRSWTAADTAAQITQRKXEAARXAEQXRAYLEGXCVEWLRRYLXNGKXTLQRAEXPKTHVTHHPXSDHEATLRCWALGFYPAEITLTWQXDGEDQTQDTELVETRPAGDGTFQKWAAVXVPSGXEQRYTCHXQHEGLXEPLTLXWXP

SEQ ID NO: 199 - protein, 276 aa, synthetic construct
  REGION 1..276: note = synthetic sequence
CSHSMRYFDTAVSRPGRGEPRFISVGYVDDTQFVRFDSDAASPRGEPRAPWVEQEGPEYWDRETQNYKRQAQADRVSLRNLRGYYNQSEDGSHTLQRMYGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTQDTELVETRPAGDGTFQKWAAVVVPSGQEQRYTCHMQHEGLQEPLTLSWEP

SEQ ID NO: 200 - protein, 276 aa, synthetic construct
  REGION 1..276: note = synthetic sequence
CSHSMRYFDTAVSRPGRGEPRFISVGYVDDTQFVRFDSDAASPRGEPRAPWVEQEGPEYWDRETQNYKRQAQADRVSLRNLRGAYNQSEDGSHTLQRMYGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTQDTELVETRPCGDGTFQKWAAVVVPSGQEQRYTCHMQHEGLQEPLTLSWEP

SEQ ID NO: 201 - protein, 276 aa, synthetic construct
  REGION 1..276: note = synthetic sequence
CSHSMRYFDTAVSRPGRGEPRFISVGYVDDTQFVRFDSDAASPRGEPRAPWVEQEGPEYWDRETQNYKRQAQADRVSLRNLRGCYNQSEDGSHTLQRMYGCDLGPDGRLLRGYDQSAYDGKDYIALNEDLRSWTAADTCAQITQRKLEAARAAEQLRAYLEGTCVEWLRRYLENGKETLQRAEPPKTHVTHHPLSDHEATLRCWALGFYPAEITLTWQRDGEDQTQDTELVETRPAGDGTFQKWAAVVVPSGQEQRYTCHMQHEGLQEPLTLSWEP

SEQ ID NO: 202 - protein, 99 aa, synthetic construct
  REGION 1..99: note = synthetic sequence
IQRTPKIQVYSCHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 203 - protein, 276 aa, synthetic construct
  REGION 1..276: note = synthetic sequence
  VARIANT 79..83: note = Xaa can be any naturally occurring amino acid
  VARIANT 85..88: note = Xaa can be any naturally occurring amino acid
  VARIANT 89..93: note = Where Xaa is independently any naturally occurring amino acid except proline or glycine and each copy of Xaa at positions 89 to 93 is either present or absent such that the number of Xaa ranges from 1 to 5.
  VARIANT 95..99: note = Where Xaa is independently any naturally occurring amino acid except proline or glycine and each copy of Xaa at positions 95 to 99 is either present or absent such that the number of Xaa ranges from 1 to 5.
  VARIANT 134..138: note = Xaa can be any naturally occurring amino acid
  VARIANT 140..143: note = Xaa can be any naturally occurring amino acid
  VARIANT 144..148: note = Where Xaa is independently any naturally occurring amino acid except proline or glycine and each copy of Xaa at positions 144 to 148 is either present or absent such that the number of Xaa ranges from 1 to 5.
  VARIANT 150..154: note = Where Xaa is independently any naturally occurring amino acid except proline or glycine and each copy of Xaa at positions 150 to 154 is either present or absent such that the number of Xaa ranges from 1 to 5.
  VARIANT 231..235: note = Where Xaa is independently any naturally occurring amino acid except proline or glycine and each copy of Xaa at positions 231 to 235 is either present or absent such that the number of Xaa ranges from 1 to 5.
  VARIANT 237..241: note = Where Xaa is independently any naturally occurring amino acid except proline or glycine and each copy of Xaa at positions 237 to 241 is either present or absent such that the number of Xaa ranges from 1 to 5.
GSHSMRYFFTSVSRPGRGEPRFIAVGYVDDTQFVRFDSDAASQRMEPRAPWIEQEGPEYWDGETRKVKAHSQTHRVDLXXXXXCXXXXXAGSHTVQRMYGCDVGSDWRFLRGYHQYAYDGKDYIALKEDLRSWXXXXXCXXXXXHKWEAAHVAEQLRAYLEGTCVEWLRRYLENGKETLQRTDAPKTHMTHHAVSDHEATLRCWALSFYPAEITLTWQRDGEDQTQDTELXXXXXIXXXXXQKWAAVVVPSGQEQRYTCHVQHEGLPKPLTLRWEP

SEQ ID NO: 204 - protein, 99 aa, synthetic construct [sequence omitted]

SEQ ID NO: 205 - protein, 276 aa, synthetic construct
  REGION 1..276: note = synthetic sequence
GSHSMRYFFTSVSRPGRGEPRFIAVGYVDDTQFVRFDSDAASQRMEPRAPWIEQEGPEYWDGETRKVKAHSQTHRVDLGTLRGYYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPCGDGTFQKWAAVVVPSGQEQRYTCHVQHEGLPKPLTLRWEP

SEQ ID NO: 206 - protein, 55 aa, synthetic construct
  REGION 1..55: note = synthetic sequence
  VARIANT 6..55: note = Each copy of Gly Gly Gly Gly Ser at positions 6 to 55 is either present or absent such that the number of copies of Gly Gly Gly Gly Ser ranges from 1 to 10.
GCGGSGGGGSGGGGSGGGGSGGGGSGGGGSGGGGSGGGGSGGGGSGGGGSGGGGS

SEQ ID NO: 207 - protein, 20 aa, synthetic construct
  REGION 1..20: note = synthetic sequence
GCGGSGGGGSGGGGSGGGGS

SEQ ID NO: 208 - protein, 15 aa, synthetic construct
  REGION 1..15: note = synthetic sequence
GCGGSGGGGSGGGGS

SEQ ID NO: 209 - protein, 208 aa, synthetic construct
  REGION 1..208: note = synthetic sequence
  VARIANT 19: note = Xaa is any amino acid other than Asn. In some cases, Xaa is Ala
VIHVTKEVKEVATLSCGHXVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDN

SEQ ID NO: 210 - protein, 208 aa, synthetic construct
  REGION 1..208: note = synthetic sequence
  VARIANT 63: note = Xaa is any amino acid other than Asn. In some cases, Xaa is Ala
VIHVTKEVKEVATLSCGHNVSVEELAQTRIYWQKEKKMVLTMMSGDMNIWPEYKNRTIFDITXNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDN

SEQ ID NO: 211 - protein, 208 aa, synthetic construct
  REGION 1..208: note = synthetic sequence
  VARIANT 67: note = Xaa is any amino acid other than Ile. In some cases, Xaa is Ala
VIHVTKEVKEVATLSCGHNVSVEELAQTRIYWQKEKKMVLTMMSGDMNIWPEYKNRTIFDITNNLSXVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDN

SEQ ID NO: 212 - protein, 208 aa, synthetic construct
  REGION 1..208: note = synthetic sequence
  VARIANT 86: note = Xaa is any amino acid other than Lys. In some cases, Xaa is Ala
VIHVTKEVKEVATLSCGHNVSVEELAQTRIYWQKEKKMVLTMMSGDMNIWPEYKNRTIFDITNNLSIVILALRPSDEGTYECVVLXYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDN

SEQ ID NO: 213 - protein, 208 aa, synthetic construct
  REGION 1..208: note = synthetic sequence
  VARIANT 157: note = Xaa is any amino acid other than Gln. In some cases, Xaa is Ala
VIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSXDPETELYAVSSKLDFNMTTNHSFMCLIKYGHLRVNQTFNWNTTKQEHFPDN

SEQ ID NO: 214 - protein, 208 aa, synthetic construct
  REGION 1..208: note = synthetic sequence
  VARIANT 158: note = Xaa is any amino acid other than Asp. In some cases, Xaa is Ala
VIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQXPETELYAVSSKLDFNMTTNHSFMCLIKYGHLRVNQTFNWNTTKQEHFPDN

SEQ ID NO: 215 - protein, 208 aa, synthetic construct
  REGION 1..208: note = synthetic sequence
  VARIANT 25: note = Xaa is any amino acid other than Leu. In some cases, Xaa is Ala
VIHVTKEVKEVATLSCGHNVSVEEXAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDN

SEQ ID NO: 216 - protein, 208 aa, synthetic construct
  REGION 1..208: note = synthetic sequence
  VARIANT 31: note = Xaa is any amino acid other than Tyr. In some cases, Xaa is Ala
VIHVTKEVKEVATLSCGHNVSVEELAQTRIXWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDN

SEQ ID NO: 217 - protein, 208 aa, synthetic construct
  REGION 1..208: note = synthetic sequence
  VARIANT 33: note = Xaa is any amino acid other than Gln. In some cases, Xaa is Ala
VIHVTKEVKEVATLSCGHNVSVEELAQTRIYWXKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDN

SEQ ID NO: 218 - protein, 208 aa, synthetic construct
  REGION 1..208: note = synthetic sequence
  VARIANT 38: note = Xaa is any amino acid other than Met. In some cases, Xaa is Ala
VIHVTKEVKEVATLSCGHNVSVEELAQTRIYWQKEKKXVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDN

SEQ ID NO: 219 - protein, 208 aa, synthetic construct
  REGION 1..208: note = synthetic sequence
  VARIANT 39: note = Xaa is any amino acid other than Val. In some cases, Xaa is Ala
VIHVTKEVKEVATLSCGHNVSVEELAQTRIYWQKEKKMXLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDN

SEQ ID NO: 220 - protein, 208 aa, synthetic construct
  REGION 1..208: note = synthetic sequence
  VARIANT 49: note = Xaa is any amino acid other than Ile. In some cases, Xaa is Ala
VIHVTKEVKEVATLSCGHNVSVEELAQTRIYWQKEKKMVLTMMSGDMNXWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDN

SEQ ID NO: 221 - protein, 208 aa, synthetic construct
  REGION 1..208: note = synthetic sequence
  VARIANT 53: note = Xaa is any amino acid other than Tyr. In some cases, Xaa is Ala
VIHVTKEVKEVATLSCGHNVSVEELAQTRIYWQKEKKMVLTMMSGDMNIWPEXKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDN

SEQ ID NO: 222 - protein, 208 aa, synthetic construct
  REGION 1..208: note = synthetic sequence
  VARIANT 60: note = Xaa is any amino acid other than Asp. In some cases, Xaa is Ala
VIHVTKEVKEVATLSCGHNVSVEELAQTRIYWQKEKKMVLTMMSGDMNIWPEYKNRTIFXITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDN

SEQ ID NO: 223 - protein, 208 aa, synthetic construct
  REGION 1..208: note = synthetic sequence
  VARIANT 108: note = Xaa is any amino acid other than Phe. In some cases, Xaa is Ala
VIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADXPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDN

SEQ ID NO: 224 - protein, 208 aa, synthetic construct
  REGION 1..208: note = synthetic sequence
  VARIANT 156: note = Xaa is any amino acid other than Ser. In some cases, Xaa is Ala
VIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVXQDPETELYAVSSKLDFNMTTNHSFMCLIKYGHLRVNQTFNWNTTKQEHFPDN

SEQ ID NO: 225 - protein, 208 aa, synthetic construct
  REGION 1..208: note = synthetic sequence
  VARIANT 111: note = Xaa is any amino acid other than Pro. In some cases, Xaa is Ala
VIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTXSISDFEIPTSNIRRIICSTSGGFPEPHLSWLENGEELNAINTTVSQDPETELYAVSSKLDFNMTTNHSFMCLIKYGHLRVNQTFNWNTTKQEHFPDN

SEQ ID NO: 226 - protein, 224 aa, Homo sapiens
APLKIQAYFNETADLPCQFANSQNQSLSELVVFWQDQENLVLNEVYLGKEKFDSVHSKYMNRTSFDSDSWTLRLHNLQIKDKGLYQCIIHHKKPTGMIRIHQMNSELSVLANFSQPEIVPISNITENVYINLTCSSIHGYPEPKKMSVLLRTKNSTIEYDGIMQKSQDNVTELYDVSISLSVSFPDVTSNMTIFCILETDKTRLLSSPFSIELEDPQPPPDHIP

SEQ ID NO: 227 - protein, 110 aa, Homo sapiens
APLKIQAYFNETADLPCQFANSQNQSLSELVVFWQDQENLVLNEVYLGKEKFDSVHSKYMNRTSFDSDSWTLRLHNLQIKDKGLYQCIIHHKKPTGMIRIHQMNSELSVL

SEQ ID NO: 228 - protein, 224 aa, synthetic construct
  REGION 1..224: note = synthetic sequence
  VARIANT 61: note = Xaa is any amino acid other than Asn. In some cases, Xaa is Ala
APLKIQAYFNETADLPCQFANSQNQSLSELVVFWQDQENLVLNEVYLGKEKFDSVHSKYMXRTSFDSDSWTLRLHNLQIKDKGLYQCIIHHKKPTGMIRIHQMNSELSVLANFSQPEIVPISNITENVYINLTCSSIHGYPEPKKMSVLLRTKNSTIEYDGIMQKSQDNVTELYDVSISLSVSFPDVTSNMTIFCILETDKTRLLSSPFSIELEDPQPPPDHIP

SEQ ID NO: 229 - protein, 224 aa, synthetic construct
  REGION 1..224: note = synthetic sequence
  VARIANT 66: note = Xaa is any amino acid other than Asp. In some cases, Xaa is Ala
APLKIQAYFNETADLPCQFANSQNQSLSELVVFWQDQENLVLNEVYLGKEKFDSVHSKYMNRTSFXSDSWTLRLHNLQIKDKGLYQCIIHHKKPTGMIRIHQMNSELSVLANFSQPEIVPISNITENVYINLTCSSIHGYPEPKKMSVLLRTKNSTIEYDGIMQKSQDNVTELYDVSISLSVSFPDVTSNMTIFCILETDKTRLLSSPFSIELEDPQPPPDHIP

SEQ ID NO: 230 - protein, 224 aa, synthetic construct
  REGION 1..224: note = synthetic sequence
  VARIANT 70: note = Xaa is any amino acid other than Trp. In some cases, Xaa is Ala
APLKIQAYFNETADLPCQFANSQNQSLSELVVFWQDQENLVLNEVYLGKEKFDSVHSKYMNRTSFDSDSXTLRLHNLQIKDKGLYQCIIHHKKPTGMIRIHQMNSELSVLANFSQPEIVPISNITENVYINLTCSSIHGYPEPKKMSVLLRTKNSTIEYDGIMQKSQDNVTELYDVSISLSVSFPDVTSNMTIFCILETDKTRLLSSPFSIELEDPQPPPDHIP

SEQ ID NO: 231 - protein, 224 aa, synthetic construct
  REGION 1..224: note = synthetic sequence
  VARIANT 91: note = Xaa is any amino acid other than His. In some cases, Xaa is Ala
APLKIQAYFNETADLPCQFANSQNQSLSELVVFWQDQENLVLNEVYLGKEKFDSVHSKYMNRTSFDSDSWTLRLHNLQIKDKGLYQCIIHXKKPTGMIRIHQMNSELSVLANFSQPEIVPISNITENVYINLTCSSIHGYPEPKKMSVLLRTKNSTIEYDGIMQKSQDNVTELYDVSISLSVSFPDVTSNMTIFCILETDKTRLLSSPFSIELEDPQPPPDHIP

SEQ ID NO: 232 - protein, 110 aa, synthetic construct
  REGION 1..110: note = synthetic sequence
  VARIANT 61: note = where Xaa is any amino acid other than Asn. In some cases, Xaa is Ala
APLKIQAYFNETADLPCQFANSQNQSLSELVVFWQDQENLVLNEVYLGKEKFDSVHSKYMXRTSFDSDSWTLRLHNLQIKDKGLYQCIIHHKKPTGMIRIHQMNSELSVL

SEQ ID NO: 233 - protein, 110 aa, synthetic construct
  REGION 1..110: note = synthetic sequence
  VARIANT 66: note = Xaa is any amino acid other than Asp. In some cases, Xaa is Ala
APLKIQAYFNETADLPCQFANSQNQSLSELVVFWQDQENLVLNEVYLGKEKFDSVHSKYMNRTSFXSDSWTLRLHNLQIKDKGLYQCIIHHKKPTGMIRIHQMNSELSVL

SEQ ID NO: 234 - protein, 110 aa, synthetic construct
  REGION 1..110: note = synthetic sequence
  VARIANT 70: note = Xaa is any amino acid other than Trp. In some cases, Xaa is Ala
APLKIQAYFNETADLPCQFANSQNQSLSELVVFWQDQENLVLNEVYLGKEKFDSVHSKYMNRTSFDSDSXTLRLHNLQIKDKGLYQCIIHHKKPTGMIRIHQMNSELSVL

SEQ ID NO: 235 - protein, 110 aa, synthetic construct
  REGION 1..110: note = synthetic sequence
  VARIANT 91: note = Xaa is any amino acid other than His. In some cases, Xaa is Ala
APLKIQAYFNETADLPCQFANSQNQSLSELVVFWQDQENLVLNEVYLGKEKFDSVHSKYMNRTSFDSDSWTLRLHNLQIKDKGLYQCIIHXKKPTGMIRIHQMNSELSVL

SEQ ID NO: 236 - protein, 224 aa, synthetic construct
  REGION 1..224: note = synthetic sequence
  VARIANT 41: note = Xaa is any amino acid other than Val. In some cases, Xaa is Ala
APLKIQAYFNETADLPCQFANSQNQSLSELVVFWQDQENLXLNEVYLGKEKFDSVHSKYMNRTSFDSDSWTLRLHNLQIKDKGLYQCIIHHKKPTGMIRIHQMNSELSVLANFSQPEIVPISNITENVYINLTCSSIHGYPEPKKMSVLLRTKNSTIEYDGIMQKSQDNVTELYDVSISLSVSFPDVTSNMTIFCILETDKTRLLSSPFSIELEDPQPPPDHIP

SEQ ID NO: 237 - protein, 110 aa, synthetic construct
  REGION 1..110: note = synthetic sequence
  VARIANT 41: note = Xaa is any amino acid other than Val. In some cases, Xaa is Ala
APLKIQAYFNETADLPCQFANSQNQSLSELVVFWQDQENLXLNEVYLGKEKFDSVHSKYMNRTSFDSDSWTLRLHNLQIKDKGLYQCIIHHKKPTGMIRIHQMNSELSVL

SEQ ID NO: 238 - protein, 224 aa, synthetic construct
  REGION 1..224: note = synthetic sequence
  VARIANT 35: note = Xaa is any amino acid other than Gln. In some cases, Xaa is Ala
APLKIQAYFNETADLPCQFANSQNQSLSELVVFWXDQENLVLNEVYLGKEKFDSVHSKYMNRTSFDSDSWTLRLHNLQIKDKGLYQCIIHHKKPTGMIRIHQMNSELSVLANFSQPEIVPISNITENVYINLTCSSIHGYPEPKKMSVLLRTKNSTIEYDGIMQKSQDNVTELYDVSISLSVSFPDVTSNMTIFCILETDKTRLLSSPFSIELEDPQPPPDHIP

SEQ ID NO: 239 - protein, 110 aa, synthetic construct
  REGION 1..110: note = synthetic sequence
  VARIANT 35: note = Xaa is any amino acid other than Gln. In some cases, Xaa is Ala
APLKIQAYFNETADLPCQFANSQNQSLSELVVFWXDQENLVLNEVYLGKEKFDSVHSKYMNRTSFDSDSWTLRLHNLQIKDKGLYQCIIHHKKPTGMIRIHQMNSELSVL

SEQ ID NO: 240 - protein, 224 aa, synthetic construct
  REGION 1..224: note = synthetic sequence
  VARIANT 33: note = Xaa is any amino acid other than Phe. In some cases, Xaa is Ala
APLKIQAYFNETADLPCQFANSQNQSLSELVVXWQDQENLVLNEVYLGKEKFDSVHSKYMNRTSFDSDSWTLRLHNLQIKDKGLYQCIIHHKKPTGMIRIHQMNSELSVLANFSQPEIVPISNITENVYINLTCSSIHGYPEPKKMSVLLRTKNSTIEYDGIMQKSQDNVTELYDVSISLSVSFPDVTSNMTIFCILETDKTRLLSSPFSIELEDPQPPPDHIP

SEQ ID NO: 241 - protein, 110 aa, synthetic construct
  REGION 1..110: note = synthetic sequence
  VARIANT 33: note = Xaa is any amino acid other than Phe. In some cases, Xaa is Ala
APLKIQAYFNETADLPCQFANSQNQSLSELVVXWQDQENLVLNEVYLGKEKFDSVHSKYMNRTSFDSDSWTLRLHNLQIKDKGLYQCIIHHKKPTGMIRIHQMNSELSVL

SEQ ID NO: 242 - protein, 224 aa, synthetic construct
  REGION 1..224: note = synthetic sequence
  VARIANT 72: note = Xaa is any amino acid other than Leu. In some cases, Xaa is Ala
APLKIQAYFNETADLPCQFANSQNQSLSELVVFWQDQENLVLNEVYLGKEKFDSVHSKYMNRTSFDSDSWTXRLHNLQIKDKGLYQCIIHHKKPTGMIRIHQMNSELSVLANFSQPEIVPISNITENVYINLTCSSIHGYPEPKKMSVLLRTKNSTIEYDGIMQKSQDNVTELYDVSISLSVSFPDVTSNMTIFCILETDKTRLLSSPFSIELEDPQPPPDHIP

SEQ ID NO: 243 - protein, 110 aa, synthetic construct
  REGION 1..110: note = synthetic sequence
  VARIANT 72: note = Xaa is any amino acid other than Leu. In some cases, Xaa is Ala
APLKIQAYFNETADLPCQFANSQNQSLSELVVFWQDQENLVLNEVYLGKEKFDSVHSKYMNRTSFDSDSWTXRLHNLQIKDKGLYQCIIHHKKPTGMIRIHQMNSELSVL

SEQ ID NO: 244 - protein, 224 aa, synthetic construct
  REGION 1..224: note = synthetic sequence
  VARIANT 59: note = Xaa is any amino acid other than Tyr. In some cases, Xaa is Ala
APLKIQAYFNETADLPCQFANSQNQSLSELVVFWQDQENLVLNEVYLGKEKFDSVHSKXMNRTSFDSDSWTLRLHNLQIKDKGLYQCIIHHKKPTGMIRIHQMNSELSVLANFSQPEIVPISNITENVYINLTCSSIHGYPEPKKMSVLLRTKNSTIEYDGIMQKSQDNVTELYDVSISLSVSFPDVTSNMTIFCILETDKTRLLSSPFSIELEDPQPPPDHIP

SEQ ID NO: 245 - protein, 110 aa, synthetic construct
  REGION 1..110: note = synthetic sequence
  VARIANT 59: note = Xaa is any amino acid other than Tyr. In some cases, Xaa is Ala
APLKIQAYFNETADLPCQFANSQNQSLSELVVFWQDQENLVLNEVYLGKEKFDSVHSKXMNRTSFDSDSWTLRLHNLQIKDKGLYQCIIHHKKPTGMIRIHQMNSELSVL

SEQ ID NO: 246 - protein, 224 aa, synthetic construct
  REGION 1..224: note = synthetic sequence
  VARIANT 61: note = Xaa is any amino acid other than Asn, In some cases, Xaa is Ala
  VARIANT 91: note = Xaa is any amino acid other than His, In some cases, Xaa is Ala
APLKIQAYFNETADLPCQFANSQNQSLSELVVFWQDQENLVLNEVYLGKEKFDSVHSKYMXRTSFDSDSWTLRLHNLQIKDKGLYQCIIHXKKPTGMIRIHQMNSELSVLANFSQPEIVPISNITENVYINLTCSSIHGYPEPKKMSVLLRTKNSTIEYDGIMQKSQDNVTELYDVSISLSVSFPDVTSNMTIFCILETDKTRLLSSPFSIELEDPQPPPDHIP

SEQ ID NO: 247 - protein, 110 aa, synthetic construct
  REGION 1..110: note = synthetic sequence
  VARIANT 61: note = Xaa is any amino acid other than Asn. In some cases, Xaa is Ala.
  VARIANT 91: note = Xaa is any amino acid other than His. In some cases, Xaa is Ala.
APLKIQAYFNETADLPCQFANSQNQSLSELVVFWQDQENLVLNEVYLGKEKFDSVHSKYMXRTSFDSDSWTLRLHNLQIKDKGLYQCIIHXKKPTGMIRIHQMNSELSVL

SEQ ID NO: 248 - protein, 224 aa, synthetic construct
  REGION 1..224: note = synthetic sequence
  VARIANT 66: note = Xaa is any amino acid other than Asp. In some cases, Xaa is Ala.
  VARIANT 91: note = Xaa is any amino acid other than His. In some cases, Xaa is Ala.
APLKIQAYFNETADLPCQFANSQNQSLSELVVFWQDQENLVLNEVYLGKEKFDSVHSKYMNRTSFXSDSWTLRLHNLQIKDKGLYQCIIHXKKPTGMIRIHQMNSELSVLANFSQPEIVPISNITENVYINLTCSSIHGYPEPKKMSVLLRTKNSTIEYDGIMQKSQDNVTELYDVSISLSVSFPDVTSNMTIFCILETDKTRLLSSPFSIELEDPQPPPDHIP

SEQ ID NO: 249 - protein, 110 aa, synthetic construct
  REGION 1..110: note = synthetic sequence
  VARIANT 66: note = Xaa is any amino acid other than Asn. In some cases, Xaa is Ala
  VARIANT 91: note = Xaa is any amino acid other than His. In some cases, Xaa is Ala
APLKIQAYFNETADLPCQFANSQNQSLSELVVFWQDQENLVLNEVYLGKEKFDSVHSKYMNRTSFXSDSWTLRLHNLQIKDKGLYQCIIHXKKPTGMIRIHQMNSELSVL

SEQ ID NO: 250 - protein, 224 aa, synthetic construct
  REGION 1..224: note = synthetic sequence
  VARIANT 61: note = Xaa is any amino acid other than Asn. In some cases, Xaa is Ala.
  VARIANT 66: note = Xaa is any amino acid other than Asp. In some cases, Xaa is Ala.
  VARIANT 91: note = Xaa is any amino acid other than His. In some cases, Xaa is Ala.
APLKIQAYFNETADLPCQFANSQNQSLSELVVFWQDQENLVLNEVYLGKEKFDSVHSKYMXRTSFXSDSWTLRLHNLQIKDKGLYQCIIHXKKPTGMIRIHQMNSELSVLANFSQPEIVPISNITENVYINLTCSSIHGYPEPKKMSVLLRTKNSTIEYDGIMQKSQDNVTELYDVSISLSVSFPDVTSNMTIFCILETDKTRLLSSPFSIELEDPQPPPDHIP

SEQ ID NO: 251 - protein, 110 aa, synthetic construct
  REGION 1..110: note = synthetic sequence
  VARIANT 61: note = Xaa is any amino acid other than Asn. In some cases, Xaa is Ala
  VARIANT 66: note = Xaa is any amino acid other than Asp. In some cases, Xaa is Ala
  VARIANT 91: note = Xaa is any amino acid other than His. In some cases, Xaa is Ala
APLKIQAYFNETADLPCQFANSQNQSLSELVVFWQDQENLVLNEVYLGKEKFDSVHSKYMXRTSFXSDSWTLRLHNLQIKDKGLYQCIIHXKKPTGMIRIHQMNSELSVL

SEQ ID NO: 252 - protein, 254 aa, synthetic construct
  REGION 1..254: note = synthetic sequence
MEYASDASLDPEAPWPPAPRARACRVLPWALVAGLLLLLLLAAACAVFLACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 253 - protein, 174 aa, Homo sapiens
PAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 254 - protein, 175 aa, Homo sapiens
DPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 255 - protein, 167 aa, Homo sapiens
DPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPA

SEQ ID NO: 256 - protein, 174 aa, Homo sapiens
  VARIANT 47: note = Xaa is any amino acid other than Lys. In some cases, Xaa is Ala
PAGLLDLRQGMFAQLVAQNVLLIDGPLSWYSDPGLAGVSLTGGLSYXEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 257 - protein, 174 aa, synthetic construct
  REGION 1..174: note = synthetic sequence
  VARIANT 147: note = Xaa is any amino acid other than Gln. In some cases, Xaa is Ala
PAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWXLTQGATVLGLFRVTPEIPAGLPSPRSE

SEQ ID NO: 258 - protein, 174 aa, synthetic construct
  REGION 1..174: note = synthetic sequence
  VARIANT 11: note = Xaa is any amino acid other than Met. In some cases, Xaa is Ala
PAGLLDLRQGXFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 259 - protein, 174 aa, synthetic construct
  REGION 1..174: note = synthetic sequence
  VARIANT 12: note = Xaa is any amino acid other than Phe. In some cases, Xaa is Ala
PAGLLDLRQGMXAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 260 - protein, 174 aa, synthetic construct
  REGION 1..174: note = synthetic sequence
  VARIANT 14: note = Xaa is any amino acid other than Gln. In some cases, Xaa is Ala
PAGLLDLRQGMFAXLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 261 - protein, 174 aa, synthetic construct
  REGION 1..174: note = synthetic sequence
  VARIANT 15: note = Xaa is any amino acid other than Leu. In some cases, Xaa is Ala
PAGLLDLRQGMFAQXVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 262 - protein, 174 aa, synthetic construct
  REGION 1..174: note = synthetic sequence
  VARIANT 16: note = Xaa is any amino acid other than Val. In some cases, Xaa is Ala
PAGLLDLRQGMFAQLXAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 263 - protein, 174 aa, synthetic construct
  REGION 1..174: note = synthetic sequence
  VARIANT 18: note = Xaa is any amino acid other than Gln. In some cases, Xaa is Ala
PAGLLDLRQGMFAQLVAXNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 264 - protein, 174 aa, synthetic construct
  REGION 1..174: note = synthetic sequence
  VARIANT 19: note = Xaa is any amino acid other than Asn. In some cases, Xaa is Ala
PAGLLDLRQGMFAQLVAQXVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 265 - protein, 174 aa, synthetic construct
  REGION 1..174: note = synthetic sequence
  VARIANT 20: note = Xaa is any amino acid other than Val. In some cases, Xaa is Ala
PAGLLDLRQGMFAQLVAQNXLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 266 - protein, 174 aa, synthetic construct
  REGION 1..174: note = synthetic sequence
  VARIANT 21: note = Xaa is any amino acid other than Leu. In some cases, Xaa is Ala
PAGLLDLRQGMFAQLVAQNVXLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 267 - protein, 174 aa, synthetic construct
  REGION 1..174: note = synthetic sequence
  VARIANT 22: note = Xaa is any amino acid other than Leu. In some cases, Xaa is Ala
PAGLLDLRQGMFAQLVAQNVLXIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 268 - protein, 174 aa, synthetic construct
  REGION 1..174: note = synthetic sequence
  VARIANT 23: note = Xaa is any amino acid other than Ile. In some cases, Xaa is Ala
PAGLLDLRQGMFAQLVAQNVLLXDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 269 - protein, 174 aa, synthetic construct
  REGION 1..174: note = synthetic sequence
  VARIANT 24: note = Xaa is any amino acid other than Asp. In some cases, Xaa is Ala
PAGLLDLRQGMFAQLVAQNVLLIXGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 270 - protein, 174 aa, synthetic construct
  REGION 1..174: note = synthetic sequence
  VARIANT 25: note = Xaa is any amino acid other than Gly. In some cases, Xaa is Ala
PAGLLDLRQGMFAQLVAQNVLLIDXPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 271 - protein, 174 aa, synthetic construct
  REGION 1..174: note = synthetic sequence
  VARIANT 26: note = Xaa is any amino acid other than Pro. In some cases, Xaa is Ala
PAGLLDLRQGMFAQLVAQNVLLIDGXLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 272 - protein, 174 aa, synthetic construct
  REGION 1..174: note = synthetic sequence
  VARIANT 27: note = Xaa is any amino acid other than Leu. In some cases, Xaa is Ala
PAGLLDLRQGMFAQLVAQNVLLIDGPXSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 273 - protein, 174 aa, synthetic construct
  REGION 1..174: note = synthetic sequence
  VARIANT 28: note = Xaa is any amino acid other than Ser. In some cases, Xaa is Ala
PAGLLDLRQGMFAQLVAQNVLLIDGPLXWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 274 - protein, 174 aa, synthetic construct
  REGION 1..174: note = synthetic sequence
  VARIANT 29: note = Xaa is any amino acid other than Trp. In some cases, Xaa is Ala
PAGLLDLRQGMFAQLVAQNVLLIDGPLSXYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 275 - protein, 174 aa, synthetic construct
  REGION 1..174: note = synthetic sequence
  VARIANT 30: note = Xaa is any amino acid other than Tyr. In some cases, Xaa is Ala
PAGLLDLRQGMFAQLVAQNVLLIDGPLSWXSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 276 - protein, 174 aa, synthetic construct
  REGION 1..174: note = synthetic sequence
  VARIANT 31: note = Xaa is any amino acid other than Ser. In some cases, Xaa is Ala
PAGLLDLRQGMFAQLVAQNVLLIDGPLSWYXDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 277 - protein, 174 aa, synthetic construct
  REGION 1..174: note = synthetic sequence
  VARIANT 32: note = Xaa is any amino acid other than Asp. In some cases, Xaa is Ala
PAGLLDLRQGMFAQLVAQNVLLIDGPLSWYSXPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 278 - protein, 174 aa, synthetic construct
  REGION 1..174: note = synthetic sequence
  VARIANT 33: note = Xaa is any amino acid other than Pro. In some cases, Xaa is Ala
PAGLLDLRQGMFAQLVAQNVLLIDGPLSWYSDXGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 279 - protein, 174 aa, synthetic construct
  REGION 1..174: note = synthetic sequence
  VARIANT 34: note = Xaa is any amino acid other than Gly. In some cases, Xaa is Ala
PAGLLDLRQGMFAQLVAQNVLLIDGPLSWYSDPXLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 280 - protein, 174 aa, synthetic construct
  REGION 1..174: note = synthetic sequence
  VARIANT 35: note = Xaa is any amino acid other than Leu. In some cases, Xaa is Ala
PAGLLDLRQGMFAQLVAQNVLLIDGPLSWYSDPGXAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 281 - protein, 174 aa, synthetic construct
  REGION 1..174: note = synthetic sequence
  VARIANT 37: note = Xaa is any amino acid other than Gly. In some cases, Xaa is Ala
PAGLLDLRQGMFAQLVAQNVLLIDGPLSWYSDPGLAXVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 282 - protein, 174 aa, synthetic construct
  REGION 1..174: note = synthetic sequence
  VARIANT 38: note = Xaa is any amino acid other than Val. In some cases, Xaa is Ala
PAGLLDLRQGMFAQLVAQNVLLIDGPLSWYSDPGLAGXSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 283 - protein, 174 aa, synthetic construct
  REGION 1..174: note = synthetic sequence
  VARIANT 39: note = Xaa is any amino acid other than Ser. In some cases, Xaa is Ala
PAGLLDLRQGMFAQLVAQNVLLIDGPLSWYSDPGLAGVXLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 284 - protein, 174 aa, synthetic construct
  REGION 1..174: note = synthetic sequence
  VARIANT 40: note = Xaa is any amino acid other than Leu. In some cases, Xaa is Ala
PAGLLDLRQGMFAQLVAQNVLLIDGPLSWYSDPGLAGVSXTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 285 - protein, 174 aa, synthetic construct
  REGION 1..174: note = synthetic sequence
  VARIANT 41: note = Xaa is any amino acid other than Thr. In some cases, Xaa is Ala
PAGLLDLRQGMFAQLVAQNVLLIDGPLSWYSDPGLAGVSLXGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 286 - protein, 174 aa, synthetic construct
  REGION 1..174: note = synthetic sequence
  VARIANT 42: note = Xaa is any amino acid other than Gly. In some cases, Xaa is Ala
PAGLLDLRQGMFAQLVAQNVLLIDGPLSWYSDPGLAGVSLTXGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 287 - protein, 174 aa, synthetic construct
  REGION 1..174: note = synthetic sequence
  VARIANT 43: note = Xaa is any amino acid other than Gly. In some cases, Xaa is Ala
PAGLLDLRQGMFAQLVAQNVLLIDGPLSWYSDPGLAGVSLTGXLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 288 - protein, 174 aa, synthetic construct
  REGION 1..174: note = synthetic sequence
  VARIANT 44: note = Xaa is any amino acid other than Leu. In some cases, Xaa is Ala
PAGLLDLRQGMFAQLVAQNVLLIDGPLSWYSDPGLAGVSLTGGXSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 289 - protein, 174 aa, synthetic construct
  REGION 1..174: note = synthetic sequence
  VARIANT 45: note = Xaa is any amino acid other than Ser. In some cases, Xaa is Ala
PAGLLDLRQGMFAQLVAQNVLLIDGPLSWYSDPGLAGVSLTGGLXYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 290 - protein, 174 aa, synthetic construct
  REGION 1..174: note = synthetic sequence
  VARIANT 46: note = Xaa is any amino acid other than Tyr. In some cases, Xaa is Ala
PAGLLDLRQGMFAQLVAQNVLLIDGPLSWYSDPGLAGVSLTGGLSXKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 291 - protein, 174 aa, synthetic construct
  REGION 1..174: note = synthetic sequence
  VARIANT 48: note = Xaa is any amino acid other than Glu. In some cases, Xaa is Ala
PAGLLDLRQGMFAQLVAQNVLLIDGPLSWYSDPGLAGVSLTGGLSYKXDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 292 - protein, 174 aa, synthetic construct
  REGION 1..174: note = synthetic sequence
  VARIANT 49: note = Xaa is any amino acid other than Asp. In some cases, Xaa is Ala
PAGLLDLRQGMFAQLVAQNVLLIDGPLSWYSDPGLAGVSLTGGLSYKEXTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 293 - protein, 174 aa, synthetic construct
  REGION 1..174: note = synthetic sequence
  VARIANT 50: note = Xaa is any amino acid other than Thr. In some cases, Xaa is Ala
PAGLLDLRQGMFAQLVAQNVLLIDGPLSWYSDPGLAGVSLTGGLSYKEDXKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 294 - protein, 174 aa, synthetic construct
  REGION 1..174: note = synthetic sequence
  VARIANT 51: note = Xaa is any amino acid other than Lys. In some cases, Xaa is Ala
PAGLLDLRQGMFAQLVAQNVLLIDGPLSWYSDPGLAGVSLTGGLSYKEDTXELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 295 - protein, 174 aa, synthetic construct
  REGION 1..174: note = synthetic sequence
  VARIANT 52: note = Xaa is any amino acid other than Glu. In some cases, Xaa is Ala
PAGLLDLRQGMFAQLVAQNVLLIDGPLSWYSDPGLAGVSLTGGLSYKEDTKXLVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 296 - protein, 174 aa, synthetic construct
  REGION 1..174: note = synthetic sequence
  VARIANT 64: note = Xaa is any amino acid other than Phe. In some cases, Xaa is Ala
PAGLLDLRQGMFAQLVAQNVLLIDGPLSWYSDPGLAGVSLTGGLSYKEDTKELVVAKAGVYYVXFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 297 - protein, 174 aa, synthetic construct
  REGION 1..174: note = synthetic sequence
  VARIANT 65: note = Xaa is any amino acid other than Phe. In some cases, Xaa is Ala
PAGLLDLRQGMFAQLVAQNVLLIDGPLSWYSDPGLAGVSLTGGLSYKEDTKELVVAKAGVYYVFXQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 298 - protein, 174 aa, synthetic construct
  REGION 1..174: note = synthetic sequence
  VARIANT 66: note = Xaa is any amino acid other than Gln. In some cases, Xaa is Ala
PAGLLDLRQGMFAQLVAQNVLLIDGPLSWYSDPGLAGVSLTGGLSYKEDTKELVVAKAGVYYVFFXLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 299 - protein, 174 aa, synthetic construct
  REGION 1..174: note = synthetic sequence
  VARIANT 67: note = Xaa is any amino acid other than Leu. In some cases, Xaa is Ala
PAGLLDLRQGMFAQLVAQNVLLIDGPLSWYSDPGLAGVSLTGGLSYKEDTKELVVAKAGVYYVFFQXELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 300 - protein, 174 aa, synthetic construct
  REGION 1..174: note = synthetic sequence
  VARIANT 68: note = Xaa is any amino acid other than Glu. In some cases, Xaa is Ala
PAGLLDLRQGMFAQLVAQNVLLIDGPLSWYSDPGLAGVSLTGGLSYKEDTKELVVAKAGVYYVFFQLXLRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 301 - protein, 174 aa, synthetic construct
  REGION 1..174: note = synthetic sequence
  VARIANT 69: note = Xaa is any amino acid other than Leu. In some cases, Xaa is Ala
PAGLLDLRQGMFAQLVAQNVLLIDGPLSWYSDPGLAGVSLTGGLSYKEDTKELVVAKAGVYYVFFQLEXRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 302 - protein, 174 aa, synthetic construct
  REGION 1..174: note = synthetic sequence
  VARIANT 70: note = Xaa is any amino acid other than Arg. In some cases, Xaa is Ala
PAGLLDLRQGMFAQLVAQNVLLIDGPLSWYSDPGLAGVSLTGGLSYKEDTKELVVAKAGVYYVFFQLELXRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 303 - protein, 174 aa, synthetic construct
  REGION 1..174: note = synthetic sequence
  VARIANT 71: note = Xaa is any amino acid other than Arg. In some cases, Xaa is Ala
PAGLLDLRQGMFAQLVAQNVLLIDGPLSWYSDPGLAGVSLTGGLSYKEDTKELVVAKAGVYYVFFQLELRXVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 304 - protein, 174 aa, synthetic construct
  REGION 1..174: note = synthetic sequence
  VARIANT 72: note = Xaa is any amino acid other than Val. In some cases, Xaa is Ala
PAGLLDLRQGMFAQLVAQNVLLIDGPLSWYSDPGLAGVSLTGGLSYKEDTKELVVAKAGVYYVFFQLELRRXVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 305 - protein, 174 aa, synthetic construct
  REGION 1..174: note = synthetic sequence
  VARIANT 73: note = Xaa is any amino acid other than Val. In some cases, Xaa is Ala
PAGLLDLRQGMFAQLVAQNVLLIDGPLSWYSDPGLAGVSLTGGLSYKEDTKELVVAKAGVYYVFFQLELRRVXAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 306 - protein, 174 aa, synthetic construct
  REGION 1..174: note = synthetic sequence
  VARIANT 75: note = Xaa is any amino acid other than Gly. In some cases, Xaa is Ala
PAGLLDLRQGMFAQLVAQNVLLIDGPLSWYSDPGLAGVSLTGGLSYKEDTKELVVAKAGVYYVFFQLELRRVVAXEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 307 - protein, 174 aa, synthetic construct
  REGION 1..174: note = synthetic sequence
  VARIANT 76: note = Xaa is any amino acid other than Glu. In some cases, Xaa is Ala
PAGLLDLRQGMFAQLVAQNVLLIDGPLSWYSDPGLAGVSLTGGLSYKEDTKELVVAKAGVYYVFFQLELRRVVAGXGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 308 - protein, 174 aa, synthetic construct
  REGION 1..174: note = synthetic sequence
  VARIANT 77: note = Xaa is any amino acid other than Gly. In some cases, Xaa is Ala
PAGLLDLRQGMFAQLVAQNVLLIDGPLSWYSDPGLAGVSLTGGLSYKEDTKELVVAKAGVYYVFFQLELRRVVAGEXSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 309 - protein, 174 aa, synthetic construct
  REGION 1..174: note = synthetic sequence
  VARIANT 78: note = Xaa is any amino acid other than Ser. In some cases, Xaa is Ala
PAGLLDLRQGMFAQLVAQNVLLIDGPLSWYSDPGLAGVSLTGGLSYKEDTKELVVAKAGVYYVFFQLELRRVVAGEGXGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 310 - protein, 174 aa, synthetic construct
  REGION 1..174: note = synthetic sequence
  VARIANT 104: note = Xaa is any amino acid other than Asp. In some cases, Xaa is Ala
PAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVXLPPASSEARNSAFGFQGRLLHLSAGQRLGVHLHTEARARHAWQLTQGATVLGLFRVTPEIPAGLPSPRSE

SEQ ID NO: 311 - protein, 174 aa, synthetic construct
  REGION 1..174: note = synthetic sequence
  VARIANT 105: note = Xaa is any amino acid other than Leu. In some cases, Xaa is Ala
PAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDXPPASSEARNSAFGFQGRLLHLSAGQRLGVHLHTEARARHAWQLTQGATVLGLFRVTPEIPAGLPSPRSE

SEQ ID NO: 312 - protein, 174 aa, synthetic construct
  REGION 1..174: note = synthetic sequence
  VARIANT 106: note = Xaa is any amino acid other than Pro. In some cases, Xaa is Ala
PAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLXPASSEARNSAFGFQGRLLHLSAGQRLGVHLHTEARARHAWQLTQGATVLGLFRVTPEIPAGLPSPRSE

SEQ ID NO: 313 - protein, 174 aa, synthetic construct
  REGION 1..174: note = synthetic sequence
  VARIANT 109: note = Xaa is any amino acid other than Ser. In some cases, Xaa is Ala
PAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPAXSEARNSAFGFQGRLLHLSAGQRLGVHLHTEARARHAWQLTQGATVLGLFRVTPEIPAGLPSPRSE

SEQ ID NO: 314 - protein, 174 aa, synthetic construct
  REGION 1..174: note = synthetic sequence
  VARIANT 110: note = Xaa is any amino acid other than Ser. In some cases, Xaa is Ala
PAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASXEARNSAFGFQGRLLHLSAGQRLGVHLHTEARARHAWQLTQGATVLGLFRVTPEIPAGLPSPRSE

SEQ ID NO: 315 - protein, 174 aa, synthetic construct
  REGION 1..174: note = synthetic sequence
  VARIANT 111: note = Xaa is any amino acid other than Glu. In some cases, Xaa is Ala
PAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSXARNSAFGFQGRLLHLSAGQRLGVHLHTEARARHAWQLTQGATVLGLFRVTPEIPAGLPSPRSE

SEQ ID NO: 316 - protein, 174 aa, synthetic construct
  REGION 1..174: note = synthetic sequence
  VARIANT 113: note = Xaa is any amino acid other than Arg. In some cases, Xaa is Ala
PAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEAXNSAFGFQGRLLHLSAGQRLGVHLHTEARARHAWQLTQGATVLGLFRVTPEIPAGLPSPRSE

SEQ ID NO: 317 - protein, 174 aa, synthetic construct
  REGION 1..174: note = synthetic sequence
  VARIANT 114: note = Xaa is any amino acid other than Asn. In some cases, Xaa is Ala
PAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARXSAFGFQGRLLHLSAGQRLGVHLHTEARARHAWQLTQGATVLGLFRVTPEIPAGLPSPRSE

SEQ ID NO: 318 - protein, 174 aa, synthetic construct
  REGION 1..174: note = synthetic sequence
  VARIANT 115: note = Xaa is any amino acid other than Ser. In some cases, Xaa is Ala
PAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNXAFGFQGRLLHLSAGQRLGVHLHTEARARHAWQLTQGATVLGLFRVTPEIPAGLPSPRSE

SEQ ID NO: 319 - protein, 174 aa, synthetic construct
  REGION 1..174: note = synthetic sequence
  VARIANT 117: note = Xaa is any amino acid other than Phe. In some cases, Xaa is Ala
PAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAXGFQGRLLHLSAGQRLGVHLHTEARARHAWQLTQGATVLGLFRVTPEIPAGLPSPRSE

SEQ ID NO: 320 - protein, 174 aa, synthetic construct
  REGION 1..174: note = synthetic sequence
  VARIANT 130: note = Xaa is any amino acid other than Gln. In some cases, Xaa is Ala
PAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGXRLGVHLHTEARARHAWQLTQGATVLGLFRVTPEIPAGLPSPRSE

SEQ ID NO: 321 - protein, 174 aa, synthetic construct
  REGION 1..174: note = synthetic sequence
  VARIANT 131: note = Xaa is any amino acid other than Arg. In some cases, Xaa is Ala
PAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQXLGVHLHTEARARHAWQLTQGATVLGLFRVTPEIPAGLPSPRSE

SEQ ID NO: 322 - protein, 174 aa, synthetic construct
  REGION 1..174: note = synthetic sequence
  VARIANT 132: note = Xaa is any amino acid other than Leu. In some cases, Xaa is Ala
PAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRXGVHLHTEARARHAWQLTQGATVLGLFRVTPEIPAGLPSPRSE

SEQ ID NO: 323 - protein, 174 aa, synthetic construct
  REGION 1..174: note = synthetic sequence
  VARIANT 133: note = Xaa is any amino acid other than Gly. In some cases, Xaa is Ala
PAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLXVHLHTEARARHAWQLTQGATVLGLFRVTPEIPAGLPSPRSE

SEQ ID NO: 324 - protein, 174 aa, synthetic construct
  REGION 1..174: note = synthetic sequence
  VARIANT 134: note = Xaa is any amino acid other than Val. In some cases, Xaa is Ala
PAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGXHLHTEARARHAWQLTQGATVLGLFRVTPEIPAGLPSPRSE

SEQ ID NO: 325 - protein, 174 aa, synthetic construct
  REGION 1..174: note = synthetic sequence
  VARIANT 135: note = Xaa is any amino acid other than His. In some cases, Xaa is Ala
PAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVXLHTEARARHAWQLTQGATVLGLFRVTPEIPAGLPSPRSE

SEQ ID NO: 326 - protein, 174 aa, synthetic construct
  REGION 1..174: note = synthetic sequence
  VARIANT 136: note = Xaa is any amino acid other than Leu. In some cases, Xaa is Ala
PAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHXHTEARARHAWQLTQGATVLGLFRVTPEIPAGLPSPRSE

SEQ ID NO: 327 - protein, 174 aa, synthetic construct
  REGION 1..174: note = synthetic sequence
  VARIANT 137: note = Xaa is any amino acid other than His. In some cases, Xaa is Ala
PAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLXTEARARHAWQLTQGATVLGLFRVTPEIPAGLPSPRSE

SEQ ID NO: 328 - protein, 174 aa, synthetic construct
  REGION 1..174: note = synthetic sequence
  VARIANT 138: note = Xaa is any amino acid other than Thr. In some cases, Xaa is Ala
PAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHXEARARHAWQLTQGATVLGLFRVTPEIPAGLPSPRSE

SEQ ID NO: 329 - protein, 174 aa, synthetic construct
  REGION 1..174: note = synthetic sequence
  VARIANT 139: note = Xaa is any amino acid other than Glu. In some cases, Xaa is Ala
PAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTXARARHAWQLTQGATVLGLFRVTPEIPAGLPSPRSE

SEQ ID NO: 330 - protein, 174 aa, synthetic construct
  REGION 1..174: note = synthetic sequence
  VARIANT 141: note = Xaa is any amino acid other than Arg. In some cases, Xaa is Ala
PAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEAXARHAWQLTQGATVLGLFRVTPEIPAGLPSPRSE

SEQ ID NO: 331 - protein, 174 aa, synthetic construct
  REGION 1..174: note = synthetic sequence
  VARIANT 143: note = Xaa is any amino acid other than Arg. In some cases, Xaa is Ala
PAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARAXHAWQLTQGATVLGLFRVTPEIPAGLPSPRSE

SEQ ID NO: 332 - protein, 174 aa, synthetic construct
  REGION 1..174: note = synthetic sequence
  VARIANT 144: note = Xaa is any amino acid other than His. In some cases, Xaa is Ala
PAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARXAWQLTQGATVLGLFRVTPEIPAGLPSPRSE

SEQ ID NO: 333 - protein, 174 aa, synthetic construct
  REGION 1..174: note = synthetic sequence
  VARIANT 146: note = Xaa is any amino acid other than Trp. In some cases, Xaa is Ala
PAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAXQLTQGATVLGLFRVTPEIPAGLPSPRSE

SEQ ID NO: 334 - protein, 174 aa, synthetic construct
  REGION 1..174: note = synthetic sequence
  VARIANT 148: note = Xaa is any amino acid other than Leu. In some cases, Xaa is Ala
PAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQXTQGATVLGLFRVTPEIPAGLPSPRSE

SEQ ID NO: 335 - protein, 174 aa, synthetic construct
  REGION 1..174: note = synthetic sequence
  VARIANT 149: note = Xaa is any amino acid other than Thr. In some cases, Xaa is Ala
PAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLXQGATVLGLFRVTPEIPAGLPSPRSE

SEQ ID NO: 336 - protein, 174 aa, synthetic construct
  REGION 1..174: note = synthetic sequence
  VARIANT 150: note = Xaa is any amino acid other than Gln. In some cases, Xaa is Ala
PAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTXGATVLGLFRVTPEIPAGLPSPRSE

SEQ ID NO: 337 - protein, 174 aa, synthetic construct
  REGION 1..174: note = synthetic sequence
  VARIANT 151: note = Xaa is any amino acid other than Gly. In some cases, Xaa is Ala
PAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQXATVLGLFRVTPEIPAGLPSPRSE

SEQ ID NO: 338 - protein, 174 aa, synthetic construct
  REGION 1..174: note = synthetic sequence
  VARIANT 153: note = Xaa is any amino acid other than Thr. In some cases, Xaa is Ala
PAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGAXVLGLFRVTPEIPAGLPSPRSE

SEQ ID NO: 339 - protein, 174 aa, synthetic construct
  REGION 1..174: note = synthetic sequence
  VARIANT 154: note = Xaa is any amino acid other than Val. In some cases, Xaa is Ala
PAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATXLGLFRVTPEIPAGLPSPRSE

SEQ ID NO: 340 - protein, 133 aa, Homo sapiens [sequence omitted]

SEQ ID NO: 341 - protein, 251 aa, Homo sapiens
ELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQVAVAGCVFLLISVLLLSGLTWQRRQRKSRRTI

SEQ ID NO: 342 - protein, 524 aa, Homo sapiens
VNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 343 - protein, 347 aa, Homo sapiens
LNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNPLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 344 - protein, 133 aa, synthetic construct
  REGION 1..133: note = synthetic sequence
  VARIANT 42: note = Xaa is any amino acid other than Phe. In some cases, Xaa is Ala, Met, Pro, Ser, Thr, Trp, Tyr, Val or His.
APTSSSTKKTQLQLEHLLLDLQMILNGINNYKNPKLTRMLTXKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 345 - protein, 133 aa, synthetic construct
  REGION 1..133: note = synthetic sequence
  VARIANT 20: note = Xaa is any amino acid other than Asp. In some cases, Xaa is Ala
APTSSSTKKTQLQLEHLLLXLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 346 - protein, 133 aa, synthetic construct
  REGION 1..133: note = synthetic sequence
  VARIANT 15: note = Xaa is any amino acid other than Glu. In some cases, Xaa is Ala
APTSSSTKKTQLQLXHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 347 - protein, 133 aa, synthetic construct
  REGION 1..133: note = synthetic sequence
  VARIANT 16: note = Xaa is any amino acid other than His. In some cases, Xaa is Ala. In some cases, Xaa is Thr. In some cases, Xaa is Asn. In some cases, Xaa is Cys. In some cases, Xaa is Gln. In some cases, Xaa is Met. In some cases, Xaa is Val. In some cases, Xaa is Trp
APTSSSTKKTQLQLEXLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 348 - protein, 133 aa, synthetic construct; identical to SEQ ID NO: 347
  REGION 1..133: note = synthetic sequence
  VARIANT 16: note = Xaa is any amino acid other than His. In some cases, Xaa is Ala. In some cases, Xaa is Arg. In some cases, Xaa is Asn. In some cases, Xaa is Asp. In some cases, Xaa is Cys. In some cases, Xaa is Glu, Gln, Gly, Ile, Lys, Met, Phe, Pro, Ser, Thr, Tyr, Trp or

SEQ ID NO: 349 - protein, 133 aa, synthetic construct
  REGION 1..133: note = synthetic sequence
  VARIANT 45: note = Xaa is any amino acid other than Tyr. In some cases, Xaa is Ala
APTSSSTKKTQLQLEHLLLDLQMILNGINNYKNPKLTRMLTFKFXMPKKATELKHLQCLEEELKPLEEVLNLAQSKNFHLRPRDLISNINVIVLELKGSETTFMCEYADETATIVEFLNRWITFCQSIISTLT

SEQ ID NO: 350 - protein, 133 aa, synthetic construct
  REGION 1..133: note = synthetic sequence
  VARIANT 126: note = Xaa is any amino acid other than Gln. In some cases, Xaa is Ala
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCXSIISTLT

SEQ ID NO: 351 - protein, 133 aa, synthetic construct
  REGION 1..133: note = synthetic sequence
  VARIANT 16: note = Xaa is any amino acid other than His. In some cases, Xaa is Ala. In some cases, Xaa is Thr.
  VARIANT 42: note = Xaa is any amino acid other than Phe. In some cases, Xaa is Ala
APTSSSTKKTQLQLEXLLLDLQMILNGINNYKNPKLTRMLTXKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 352 - protein, 133 aa, synthetic construct
  REGION 1..133: note = synthetic sequence
  VARIANT 20: note = Xaa is any amino acid other than Asp. In some cases, Xaa is Ala.
  VARIANT 42: note = Xaa is any amino acid other than Phe. In some cases, Xaa is Ala.
APTSSSTKKTQLQLEHLLLXLQMILNGINNYKNPKLTRMLTXKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 353 - protein, 133 aa, synthetic construct
  REGION 1..133: note = synthetic sequence
  VARIANT 15: note = Xaa is any amino acid other than Glu. In some cases, Xaa is Ala
  VARIANT 20: note = Xaa is any amino acid other than Asp. In some cases, Xaa is Ala
  VARIANT 42: note = Xaa is any amino acid other than Phe. In some cases, Xaa is Ala
APTSSSTKKTQLQLXHLLLXLQMILNGINNYKNPKLTRMLTXKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 354 - protein, 133 aa, synthetic construct
  REGION 1..133: note = synthetic sequence
  VARIANT 16: note = Xaa is any amino acid other than His. In some cases, Xaa is Ala
  VARIANT 20: note = Xaa is any amino acid other than Asp. In some cases, Xaa is Ala
  VARIANT 42: note = Xaa is any amino acid other than Phe. In some cases, Xaa is Ala
APTSSSTKKTQLQLEXLLLXLQMILNGINNYKNPKLTRMLTXKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 355 - protein, 133 aa, synthetic construct
  REGION 1..133: note = synthetic sequence
  VARIANT 20: note = Xaa is any amino acid other than Asp. In some cases, Xaa is Ala
  VARIANT 42: note = Xaa is any amino acid other than Phe. In some cases, Xaa is Ala
  VARIANT 126: note = Xaa is any amino acid other than Gln. In some cases, Xaa is Ala
APTSSSTKKTQLQLEHLLLXLQMILNGINNYKNPKLTRMLTXKFYMPKKATELKHLQCLEEELKPLEEVLNLAQSKNFHLRPRDLISNINVIVLELKGSETTFMCEYADETATIVEFLNRWITFCXSIISTLT

SEQ ID NO: 356 - protein, 133 aa, synthetic construct
  REGION 1..133: note = synthetic sequence
  VARIANT 20: note = Xaa is any amino acid other than Asp. In some cases, Xaa is Ala
  VARIANT 42: note = Xaa is any amino acid other than Phe. In some cases, Xaa is Ala
  VARIANT 45: note = Xaa is any amino acid other than Tyr. In some cases, Xaa is Ala
APTSSSTKKTQLQLEHLLLXLQMILNGINNYKNPKLTRMLTXKFXMPKKATELKHLQCLEEELKPLEEVLNLAQSKNFHLRPRDLISNINVIVLELKGSETTFMCEYADETATIVEFLNRWITFCQSIISTLT

SEQ ID NO: 357 - protein, 133 aa, synthetic construct
  REGION 1..133: note = synthetic sequence
  VARIANT 16: note = Xaa is any amino acid other than His. In some cases, Xaa is Ala
  VARIANT 20: note = Xaa is any amino acid other than Asp. In some cases, Xaa is Ala
  VARIANT 42: note = Xaa is any amino acid other than Phe. In some cases, Xaa is Ala
  VARIANT 45: note = Xaa is any amino acid other than Tyr. In some cases, Xaa is Ala
APTSSSTKKTQLQLEXLLLXLQMILNGINNYKNPKLTRMLTXKFXMPKKATELKHLQCLEEELKPLEEVLNLAQSKNFHLRPRDLISNINVIVLELKGSETTFMCEYADETATIVEFLNRWITFCQSIISTLT

SEQ ID NO: 358 - protein, 133 aa, synthetic construct
  REGION 1..133: note = synthetic sequence
  VARIANT 20: note = Xaa is any amino acid other than Asp. In some cases, Xaa is Ala
  VARIANT 42: note = Xaa is any amino acid other than Phe. In some cases, Xaa is Ala
  VARIANT 45: note = Xaa is any amino acid other than Tyr. In some cases, Xaa is Ala
  VARIANT 126: note = Xaa is any amino acid other than Gln. In some cases, Xaa is Ala
APTSSSTKKTQLQLEHLLLXLQMILNGINNYKNPKLTRMLTXKFXMPKKATELKHLQCLEEELKPLEEVLNLAQSKNFHLRPRDLISNINVIVLELKGSETTFMCEYADETATIVEFLNRWITFCXSIISTLT

SEQ ID NO: 359 - protein, 133 aa, synthetic construct
  REGION 1..133: note = synthetic sequence
  VARIANT 16: note = Xaa is any amino acid other than His. In some cases, Xaa is Ala
  VARIANT 20: note = Xaa is any amino acid other than Asp. In some cases, Xaa is Ala
  VARIANT 42: note = Xaa is any amino acid other than Phe. In some cases, Xaa is Ala
  VARIANT 45: note = Xaa is any amino acid other than Tyr. In some cases, Xaa is Ala
  VARIANT 126: note = Xaa is any amino acid other than Gln. In some cases, Xaa is Ala
APTSSSTKKTQLQLEXLLLXLQMILNGINNYKNPKLTRMLTXKFXMPKKATELKHLQCLEEELKPLEEVLNLAQSKNFHLRPRDLISNINVIVLELKGSETTFMCEYADETATIVEFLNRWITFCXSIISTLT

SEQ ID NO: 360 - protein, 133 aa, synthetic construct
  REGION 1..133: note = synthetic sequence
  VARIANT 16: note = Xaa is any amino acid other than His. In some cases, Xaa is Ala
  VARIANT 42: note = Xaa is any amino acid other than Phe. In some cases, Xaa is Ala
  VARIANT 126: note = Xaa is any amino acid other than Gln. In some cases, Xaa is Ala
APTSSSTKKTQLQLEXLLLDLQMILNGINNYKNPKLTRMLTXKFYMPKKATELKHLQCLEEELKPLEEVLNLAQSKNFHLRPRDLISNINVIVLELKGSETTFMCEYADETATIVEFLNRWITFCXSIISTLT

SEQ ID NO: 361 - protein, 5 aa, synthetic construct
  REGION 1..5: note = synthetic sequence
  VARIANT 4: note = Xaa is any amino acid other than Pro
VPGXG

SEQ ID NO: 362 - protein, 223 aa, synthetic construct
  REGION 1..223: note = synthetic sequence
PPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPG

SEQ ID NO: 363 - protein, 5 aa, synthetic construct
  REGION 1..5: note = synthetic sequence
GSGGS

SEQ ID NO: 364 - protein, 4 aa, synthetic construct
  REGION 1..4: note = synthetic sequence
GGGS

SEQ ID NO: 365 - protein, 4 aa, synthetic construct
  REGION 1..4: note = synthetic sequence
GGSG

SEQ ID NO: 366 - protein, 5 aa, synthetic construct
  REGION 1..5: note = synthetic sequence
GGSGG

SEQ ID NO: 367 - protein, 5 aa, synthetic construct
  REGION 1..5: note = synthetic sequence
GSGSG

SEQ ID NO: 368 - protein, 5 aa, synthetic construct
  REGION 1..5: note = synthetic sequence
GSGGG

SEQ ID NO: 369 - protein, 5 aa, synthetic construct
  REGION 1..5: note = synthetic sequence
GGGSG

SEQ ID NO: 370 - protein, 5 aa, synthetic construct
  REGION 1..5: note = synthetic sequence
GSSSG

SEQ ID NO: 371 - protein, 50 aa, synthetic construct
  REGION 1..50: note = synthetic sequence
  VARIANT 1..50: note = Each copy of Ser Gly Gly Gly Gly at positions 1 to 50 is either present or absent such that the number of copies of Ser Gly Gly Gly Gly ranges from 1 to 10.
GSSSSGSSSSGSSSSGSSSSGSSSSGSSSSGSSSSGSSSSGSSSSGSSSS

SEQ ID NO: 372 - protein, 5 aa, synthetic construct
  REGION 1..5: note = synthetic sequence
AAAGG

SEQ ID NO: 373 - protein, 5 aa, synthetic construct
  REGION 1..5: note = synthetic sequence
GCGGS

SEQ ID NO: 374 - protein, 5 aa, synthetic construct
  REGION 1..5: note = synthetic sequence
CGGGS

SEQ ID NO: 375 - protein, 55 aa, synthetic construct
  REGION 1..55: note = synthetic sequence
  VARIANT 6..55: note = Each copy of Gly Gly Gly Gly Ser at positions 6 to 55 is either present or absent such that the number of copies of Gly Gly Gly Gly Ser ranges from 1 to 10.
CGGGSGGGGSGGGGSGGGGSGGGGSGGGGSGGGGSGGGGSGGGGSGGGGSGGGGS

SEQ ID NO: 376 - protein, 227 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 377 - protein, 325 aa, Homo sapiens
STKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 378 - protein, 246 aa, Homo sapiens
HKPSNTKVDKRVELKTPLGDTTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 379 - protein, 383 aa, Homo sapiens
PTKAPDVFPIISGCRHPKDNSPVVLACLITGYHPTSVTVTWYMGTQSQPQRTFPEIQRRDSYYMTSSQLSTPLQQWRQGEYKCVVQHTASKSKKEIFRWPESPKAQASSVPTAQPQAEGSLAKATTAPATTRNTGRGGEEKKKEKEKEEQEERETKTPECPSHTQPLGVYLLTPAVQDLWLRDKATFTCFVVGSDLKDAHLTWEVAGKVPTGGVEEGLLERHSNGSQSQHSRLTLPRSLWNAGTSVTCTLNHPSLPPQRLMALREPAAQAPVKLSLNLLASSDPPEAASWLLCEVSGFSPPNILLMWLEDQREVNTSGFAPARPPPQPRSTTFWAWSVLRVPAPPSPQPATYTCVVSHEDSRTLLNASRSLEVSYVTDHGPMK

SEQ ID NO: 380 - protein, 276 aa, Homo sapiens
VTSTLTIKZSDWLGESMFTCRVDHRGLTFQQNASSMCVPDQDTAIRVFAIPPSFASIFLTKSTKLTCLVTDLTTYBSVTISWTREENGAVKTHTNISESHPNATFSAVGEASICEDBDWSGERFTCTVTHTDLPSPLKQTISRPKGVALHRPBVYLLPPARZZLNLRESATITCLVTGFSPADVFVEWMQRGEPLSPQKYVTSAPMPEPQAPGRYFAHSILTVSEEEWNTGGTYTCVVAHEALPNRVTERTVDKSTGKPTLYNVSLVMSDTAGTCY

SEQ ID NO: 381 - protein, 353 aa, Homo sapiens
ASPTSPKVFPLSLCSTQPDGNVVIACLVQGFFPQEPLSVTWSESGQGVTARNFPPSQDASGDLYTTSSQLTLPATQCLAGKSVTCHVKHYTNPSQDVTVPCPVPSTPPTPSPSTPPTPSPSCCHPRLSLHRPALEDLLLGSEANLTCTLTGLRDASGVTFTWTPSSGKSAVQGPPERDLCGCYSVSSVLPGCAEPWNHGKTFTCTAAYPESKTPLTATLSKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 382 - protein, 222 aa, Homo sapiens
ADPCDSNPRGVSAYLSRPSPFDLFIRKSPTITCLVVDLAPSKGTVNLTWSRASGKPVNHSTRKEEKQRNGTLTVTSTLPVGTRDWIEGETYQCRVTHPHLPRALMRSTTKTSGPRAAPEVYAFATPEWPGSRDKRTLACLIQNFMPEDISVQWLHNEVQLPDARHSTTQPRKTKGSGFFVFSRLEVTRAEWEQKDEFICRAVHEAASPSQTVQRAVSVNPGK

SEQ ID NO: 383 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 384 - protein, 227 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 385 - protein, 227 aa, Homo sapiens
DKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 386 - protein, 227 aa, Homo sapiens
DKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 387 - protein, 227 aa, Homo sapiens
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 388 - protein, 119 aa, unidentified
  REGION 1..119: note = Originating from from either Homo sapiens or Pan troglodytes.
MSRSVALAVLALLSLSGLEAIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 389 - protein, 119 aa, Macaca mulatta
MSRSVALAVLALLSLSGLEAIQRTPKIQVYSRHPPENGKPNFLNCYVSGFHPSDIEVDLLKNGEKMGKVEHSDLSFSKDWSFYLLYYTEFTPNEKDEYACRVNHVTLSGPRTVKWDRDM

SEQ ID NO: 390 - protein, 118 aa, Bos taurus
MARFVALVLLGLLSLSGLDAIQRPPKIQVYSRHPPEDGKPNYLNCYVYGFHPPQIEIDLLKNGEKIKSEQSDLSFSKDWSFYLLSHAEFTPNSKDQYSCRVKHVTLEQPRIVKWDRDL

SEQ ID NO: 391 - protein, 119 aa, Mus musculus
MARSVTLVFLVLVSLTGLYAIQKTPQIQVYSRHPPENGKPNILNCYVTQFHPPHIEIQMLKNGKKIPKVEMSDMSFSKDWSFYILAHTEFTPTETDTYACRVKHASMAEPKTVYWDRDM

SEQ ID NO: 392 - protein, 365 aa, Homo sapiens
MAVMAPRTLLLLLSGALALTQTWAGSHSMRYFFTSVSRPGRGEPRFIAVGYVDDTQFVRFDSDAASQKMEPRAPWIEQEGPEYWDQETRNMKAHSQTDRANLGTLRGYYNQSEDGSHTIQIMYGCDVGPDGRFLRGYRQDAYDGKDYIALNEDLRSWTAADMAAQITKRKWEAVHAAEQRRVYLEGRCVDGLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWELSSQPTIPIVGIIAGLVLLGAVITGAVVAAVMWRRKSSDRKGGSYTQAASSDSAQGSDVSLTACKV

SEQ ID NO: 393 - protein, 365 aa, Homo sapiens
MAVMAPRTLLLLLSGALALTQTWAGSHSMRYFYTSVSRPGRGEPRFIAVGYVDDTQFVRFDSDAASQRMEPRAPWIEQEGPEYWDQETRNVKAQSQTDRVDLGTLRGYYNQSEDGSHTIQIMYGCDVGPDGRFLRGYRQDAYDGKDYIALNEDLRSWTAADMAAQITKRKWEAAHAAEQQRAYLEGRCVEWLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWELSSQPTIPIVGIIAGLVLLGAVITGAVVAAVMWRRKSSDRKGGSYTQAASSDSAQGSDVSLTACKV

SEQ ID NO: 394 - protein, 365 aa, Homo sapiens [sequence omitted]

SEQ ID NO: 395 - protein, 365 aa, Homo sapiens [sequence omitted]

SEQ ID NO: 396 - protein, 362 aa, Homo sapiens
MLVMAPRTVLLLLSAALALTETWAGSHSMRYFYTSVSRPGRGEPRFISVGYVDDTQFVRFDSDAASPREEPRAPWIEQEGPEYWDRNTQIYKAQAQTDRESLRNLRGYYNQSEAGSHTLQSMYGCDVGPDGRLLRGHDQYAYDGKDYIALNEDLRSWTAADTAAQITQRKWEAAREAEQRRAYLEGECVEWLRRYLENGKDKLERADPPKTHVTHHPISDHEATLRCWALGFYPAEITLTWQRDGEDQTQDTELVETRPAGDRTFQKWAAVVVPSGEEQRYTCHVQHEGLPKPLTLRWEPSSQSTVPIVGIVAGLAVLAVVVIGAVVAAVMCRRKSSGGKGGSYSQAACSDSAQGSDVSLTA

SEQ ID NO: 397 - protein, 366 aa, Homo sapiens
MRVMAPRALLLLLSGGLALTETWACSHSMRYFDTAVSRPGRGEPRFISVGYVDDTQFVRFDSDAASPRGEPRAPWVEQEGPEYWDRETQNYKRQAQADRVSLRNLRGYYNQSEDGSHTLQRMYGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTQDTELVETRPAGDGTFQKWAAVVVPSGQEQRYTCHMQHEGLQEPLTLSWEPSSQPTIPIMGIVAGLAVLVVLAVLGAVVTAMMCRRKSSGGKGGSCSQAACSNSAQGSDESLITCKA

SEQ ID NO: 398 - protein, 275 aa, synthetic construct
  REGION 1..275: note = synthetic sequence
GSHSMRYFFTSVSRPGRGEPRFIAVGYVDDTQFVRFDSDAASQKMEPRAPWIEQEGPEYWDQETRNMKAHSQTDRANLGTLRGYYNQSEDGSHTIQIMYGCDVGPDGRFLRGYRQDAYDGKDYIALNEDLRSWTAADMAAQITKRKWEAVHAAEQRRVYLEGRCVDGLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWE

SEQ ID NO: 399 - protein, 275 aa, synthetic construct
  REGION 1..275: note = synthetic sequence
GSHSMRYFYTSVSRPGRGEPRFISVGYVDDTQFVRFDSDAASPREEPRAPWIEQEGPEYWDRNTQIYKAQAQTDRESLRNLRGYYNQSEAGSHTLQSMYGCDVGPDGRLLRGHDQYAYDGKDYIALNEDLRSWTAADTAAQITQRKWEAAREAEQRRAYLEGECVEWLRRYLENGKDKLERADPPKTHVTHHPISDHEATLRCWALGFYPAEITLTWQRDGEDQTQDTELVETRPAGDRTFQKWAAVVVPSGEEQRYTCHVQHEGLPKPLTLRWE

SEQ ID NO: 400 - protein, 275 aa, synthetic construct
  REGION 1..275: note = synthetic sequence
CSHSMRYFDTAVSRPGRGEPRFISVGYVDDTQFVRFDSDAASPRGEPRAPWVEQEGPEYWDRETQNYKRQAQADRVSLRNLRGYYNQSEDGSHTLQRMYGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTQDTELVETRPAGDGTFQKWAAVVVPSGQEQRYTCHMQHEGLQEPLTLSWE

SEQ ID NO: 401 - protein, 274 aa, synthetic construct
  REGION 1..274: note = synthetic sequence
GPHSLRYFVTAVSRPGLGEPRFIAVGYVDDTQFVRFDSDADNPRFEPRAPWMEQEGPEYWEEQTQRAKSDEQWFRVSLRTAQRYYNQSKGGSHTFQRMFGCDVGSDWRLLRGYQQFAYDGRDYIALNEDLKTWTAADTAALITRRKWEQAGDAEYYRAYLEGECVEWLRRYLELGNETLLRTDSPKAHVTYHPRSQVDVTLRCWALGFYPADITLTWQLNGEDLTQDMELVETRPAGDGTFQKWAAVVVPLGKEQNYTCHVHHKGLPEPLTLRW

SEQ ID NO: 402 - protein, 275 aa, synthetic construct
  REGION 1..275: note = synthetic sequence
GSHSMRYFFTSVSRPGRGEPRFIAVGYVDDTQFVRFDSDAASQRMEPRAPWIEQEGPEYWDGETRKVKAHSQTHRVDLGTLRGCYNQSEAGSHTVQRMYGCDVGSDWRFLRGYHQYAYDGKDYIALKEDLRSWTAADMCAQTTKHKWEAAHVAEQLRAYLEGTCVEWLRRYLENGKETLQRTDAPKTHMTHHAVSDHEATLRCWALSFYPAEITLTWQRDGEDQTQDTELVETRPCGDGTFQKWAAVVVPSGQEQRYTCHVQHEGLPKPLTLRWE

SEQ ID NO: 403 - protein, 276 aa, synthetic construct
  REGION 1..276: note = synthetic sequence
GSHSMRYFYTSVSRPGRGEPRFIAVGYVDDTQFVRFDSDAASQRMEPRAPWIEQEGPEYWDQETRNVKAQSQTDRVDLGTLRGYYNQSEDGSHTIQIMYGCDVGPDGRFLRGYRQDAYDGKDYIALNEDLRSWTAADMAAQITKRKWEAAHAAEQQRAYLEGRCVEWLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWEL

SEQ ID NO: 404 - protein, 276 aa, synthetic construct
  REGION 1..276: note = synthetic sequence
GSHSMRYFSTSVSRPGRGEPRFIAVGYVDDTQFVRFDSDAASQRMEPRAPWIEQEGPEYWDEETGKVKAHSQTDRENLRIALRYYNQSEAGSHTLQMMFGCDVGSDGRFLRGYHQYAYDGKDYIALKEDLRSWTAADMAAQITKRKWEAAHVAEQQRAYLEGTCVDGLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWEP

SEQ ID NO: 405 - protein, 276 aa, synthetic construct
  REGION 1..276: note = synthetic sequence
GSHSMRYFTTSVSRPGRGEPRFIAVGYVDDTQFVRFDSDAASQRMEPRAPWIEQEGPEYWDRNTRNVKAHSQIDRVDLGTLRGYYNQSEAGSHTIQMMYGCDVGSDGRFLRGYQQDAYDGKDYIALNEDLRSWTAADMAAQITQRKWEAARVAEQLRAYLEGTCVEWLRRYLENGKETLQRTDPPKTHMTHHAVSDHEATLRCWALSFYPAEITLTWQRDGEDQTQDTELVETRPAGDGTFQKWASVVVPSGQEQRYTCHVQHEGLPKPLTLRWEP

SEQ ID NO: 406 - protein, 276 aa, synthetic construct
  REGION 1..276: note = synthetic sequence
GSHSMRYFFTSVSRPGRGEPRFIAVGYVDDTQFVRFDSDAASQKMEPRAPWIEQEGPEYWDQETRNMKAHSQTDRANLGTLRGYYNQSEDGSHTIQIMYGCDVGPDGRFLRGYRQDAYDGKDYIALNEDLRSWTAADMAAQITKRKWEAVHAAEQRRVYLEGRCVDGLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWEL

SEQ ID NO: 407 - protein, 276 aa, synthetic construct
  REGION 1..276: note = synthetic sequence
GSHSMRYFFTSVSRPGRGEPRFIAVGYVDDTQFVRFDSDAASQRMEPRAPWIEQEGPEYWDQETRNVKAQSQTDRVDLGTLRGYYNQSEAGSHTIQIMYGCDVGSDGRFLRGYRQDAYDGKDYIALNEDLRSWTAADMAAQITHHKWEAAHEAEQLRAYLDGTCVEWLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWEL

SEQ ID NO: 408 - protein, 311 aa, synthetic construct
  REGION 1..311: note = synthetic sequence
GSHSMRYFYTSVSRPGRGEPRFIAVGYVDDTQFVRFDSDAASQRMEPRAPWIEQEGPEYWDQETRNVKAQSQTDRVDLGTLRGYYNQSEDGSHTIQIMYGCDVGPDGRFLRGYRQDAYDGKDYIALNEDLRSWTAADMAAQITKRKWEAAHAAEQQRAYLEGRCVEWLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWELQKWAAVVVPSGEEQRYTCHVQHEGLPKPLTLRWEP

SEQ ID NO: 409 - protein, 381 aa, synthetic construct
  REGION 1..381: note = synthetic sequence
GSHSMRYFSTSVSRPGRGEPRFIAVGYVDDTQFVRFDSDAASQRMEPRAPWIEQEGPEYWDEETGKVKAHSQTDRENLRIALRYYNQSEAGSHTLQMMFGCDVGSDGRFLRGYHQYAYDGKDYIALKEDLRSWTAADMAAQITQRKWEAARVAEQLRAYLEGTCVDGLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWEPQKWAAVVVPSGEEQRYTCHVQHEGLPKPLTLRWEPQKWASVVVPSGQEQRYTCHVQHEGLPKPLTLRWEPQKWASVVVPSGQEQRYTCHVQHEGLPKPLTLRWEP

SEQ ID NO: 410 - protein, 241 aa, synthetic construct
  REGION 1..241: note = synthetic sequence
GSHSMRYFSTSVSRPGRGEPRFIAVGYVDDTQFVRFDSDAASQRMEPRAPWIEQEGPEYWDEETGKVKAHSQTDRENLRIALRYYNQSEAGSHTLQMMFGCDVGSDGRFLRGYHQYAYDGKDYIALKEDLRSWTAADMAAQITKRKWEAAHVAEQQRAYLEGTCVDGLRRYLENGKETLQRTDPPKTHMTHHPISDHEATLRCWALGFYPAEITLTWQRDGEDQTQDTELVETRPAGDGTF

SEQ ID NO: 411 - protein, 241 aa, synthetic construct
  REGION 1..241: note = synthetic sequence
GSHSMRYFSTSVSRPGRGEPRFIAVGYVDDTQFVRFDSDAASQRMEPRAPWIEQEGPEYWDEETGKVKAQSQTDRENLRIALRYYNQSEAGSHTLQMMFGCDVGSDGRFLRGYHQYAYDGKDYIALKEDLRSWTAADMAAQITKRKWEAAHVAEQQRAYLEGTCVDGLRRYLENGKETLQRTDPPKTHMTHHPISDHEATLRCWALGFYPAEITLTWQRDGEDQTQDTELVETRPAGDGTF

SEQ ID NO: 412 - protein, 241 aa, synthetic construct
  REGION 1..241: note = synthetic sequence
GSHSMRYFTTSVSRPGRGEPRFIAVGYVDDTQFVRFDSDAASQRMEPRAPWIEQEGPEYWDRNTRNVKAHSQIDRVDLGTLRGYYNQSEAGSHTIQMMYGCDVGSDGRFLRGYQQDAYDGKDYIALNEDLRSWTAADMAAQITQRKWEAARVAEQLRAYLEGTCVEWLRRYLENGKETLQRTDPPKTHMTHHAVSDHEATLRCWALSFYPAEITLTWQRDGEDQTQDTELVETRPAGDGTF

SEQ ID NO: 413 - protein, 241 aa, synthetic construct
  REGION 1..241: note = synthetic sequence
GSHSMRYFYTSVSRPGRGEPRFIAVGYVDDTQFVRFDSDAASQRMEPRAPWIEQEGPEYWDRNTRKVKAQSQTDRVDLGTLRGYYNQSEAGSHTIQRMYGCDVGPDGRFLRGYQQDAYDGKDYIALNEDLRSWTAADMAAQITQRKWETAHEAEQWRAYLEGTCVEWLRRYLENGKETLQRTDAPKTHMTHHAVSDHEATLRCWALSFYPAEITLTWQRDGEDQTQDTELVETRPAGDGTF

SEQ ID NO: 414 - protein, 276 aa, synthetic construct
  REGION 1..276: note = synthetic sequence
GSHSMRYFDTAMSRPGRGEPRFISVGYVDDTQFVRFDSDAASPREEPRAPWIEQEGPEYWDRNTQIFKTNTQTDRESLRNLRGYYNQSEAGSHTLQSMYGCDVGPDGRLLRGHNQYAYDGKDYIALNEDLRSWTAADTAAQITQRKWEAARVAEQDRAYLEGTCVEWLRRYLENGKDTLERADPPKTHVTHHPISDHEATLRCWALGFYPAEITLTWQRDGEDQTQDTELVETRPAGDRTFQKWAAVVVPSGEEQRYTCHVQHEGLPKPLTLRWEP

SEQ ID NO: 415 - protein, 276 aa, synthetic construct
  REGION 1..276: note = synthetic sequence
GSHSMRYFYTAMSRPGRGEPRFIAVGYVDDTQFVRFDSDAASPRMAPRAPWIEQEGPEYWDRNTQISKTNTQTYRESLRNLRGYYNQSEAGSHIIQRMYGCDVGPDGRLLRGYDQSAYDGKDYIALNEDLSSWTAADTAAQITQRKWEAAREAEQLRAYLEGLCVEWLRRYLENGKETLQRADPPKTHVTHHPISDHEATLRCWALGFYPAEITLTWQRDGEDQTQDTELVETRPAGDRTFQKWAAVVVPSGEEQRYTCHVQHEGLPKPLTLRWEP

SEQ ID NO: 416 - protein, 275 aa, synthetic construct
  REGION 1..275: note = synthetic sequence
GSHSMRYFYTSVSRPGRGEPRFISVGVDDTQFVRFDSDAASPREEPRAPWIEQEGPEYWDRNTQICKTNTQTYRENLRTALRYYNQSEAGSHTLQRMYGCDVGPDGRLLRGHNQFAYDGKDYIALNEDLSSWTAADTAAQITQRKWEAARVAEQLRTYLEGTCVEWLRRYLENGKETLQRADPPKTHVTHHPISDHEATLRCWALGFYPAEITLTWQRDGEDQTQDTELVETRPAGDRTFQKWAAVVVPSGEEQRYTCHVQHEGLPKPLTLRWEP

SEQ ID NO: 417 - protein, 276 aa, synthetic construct
  REGION 1..276: note = synthetic sequence
GSHSMRYFHTAMSRPGRGEPRFITVGYVDDTLFVRFDSDATSPRKEPRAPWIEQEGPEYWDRETQISKTNTQTYRESLRNLRGYYNQSEAGSHTLQRMYGCDVGPDGRLLRGHNQYAYDGKDYIALNEDLRSWTAADTAAQISQRKLEAARVAEQLRAYLEGECVEWLRRYLENGKDKLERADPPKTHVTHHPISDHEATLRCWALGFYPAEITLTWQRDGEDQTQDTELVETRPAGDRTFQKWAAVVVPSGEEQRYTCHVQHEGLPKPLTLRWEP

SEQ ID NO: 418 - protein, 276 aa, synthetic construct
  REGION 1..276: note = synthetic sequence
GSHSMRYFYTAMSRPGRGEPRFIAVGYVDDTQFVRFDSDAASPRMAPRAPWIEQEGPEYWDRETQKYKRQAQTDRVSLRNLRGYYNQSEAGSHTLQRMYGCDVGPDGRLLRGHDQSAYDGKDYIALNEDLSSWTAADTAAQITQRKWEAAREAEQWRAYLEGLCVEWLRRYLENGKETLQRADPPKTHVTHHPISDHEATLRCWALGFYPAEITLTWQRDGEDQTQDTELVETRPAGDRTFQKWAAVVVPSGEEQRYTCHVQHEGLPKPLTLRWEP

SEQ ID NO: 419 - protein, 276 aa, synthetic construct
  REGION 1..276: note = synthetic sequence
GSHSMRYFYTAMSRPGRGEPRFIAVGYVDDTQFVRFDSDAASPRTEPRAPWIEQEGPEYWDRNTQIFKTNTQTYRENLRIALRYYNQSEAGSHIIQRMYGCDLGPDGRLLRGHDQSAYDGKDYIALNEDLSSWTAADTAAQITQRKWEAARVAEQLRAYLEGLCVEWLRRYLENGKETLQRADPPKTHVTHHPVSDHEATLRCWALGFYPAEITLTWQRDGEDQTQDTELVETRPAGDRTFQKWAAVVVPSGEEQRYTCHVQHEGLPKPLTLRWEP

SEQ ID NO: 420 - protein, 276 aa, synthetic construct
  REGION 1..276: note = synthetic sequence
CSHSMKYFFTSVSRPGRGEPRFISVGYVDDTQFVRFDSDAASPRGEPRAPWVEQEGPEYWDRETQKYKRQAQTDRVSLRNLRGYYNQSEAGSHTLQWMCGCDLGPDGRLLRGYDQYAYDGKDYIALNEDLRSWTAADTAAQITQRKWEAAREAEQRRAYLEGTCVEWLRRYLENGKETLQRAEHPKTHVTHHPVSDHEATLRCWALGFYPAEITLTWQWDGEDQTQDTELVETRPAGDGTFQKWAAVMVPSGEEQRYTCHVQHEGLPEPLTLRWEP

SEQ ID NO: 421 - protein, 276 aa, synthetic construct
  REGION 1..276: note = synthetic sequence
GSHSMRYFYTAVSRPGRGEPHFIAVGYVDDTQFVRFDSDAASPRGEPRAPWVEQEGPEYWDRETQKYKRQAQTDRVSLRNLRGYYNQSEARSHIIQRMYGCDVGPDGRLLRGYDQYAYDGKDYIALNEDLRSWTAADTAAQITQRKWEAAREAEQLRAYLEGLCVEWLRRYLKNGKETLQRAEHPKTHVTHHPVSDHEATLRCWALGFYPAEITLTWQWDGEDQTQDTELVETRPAGDGTFQKWAAVVVPSGEEQRYTCHVQHEGLPEPLTLRWEP

SEQ ID NO: 422 - protein, 276 aa, synthetic construct
  REGION 1..276: note = synthetic sequence
GSHSMRYFYTAVSRPGRGEPHFIAVGYVDDTQFVRFDSDAASPRGEPRAPWVEQEGPEYWDRETQKYKRQAQTDRVSLRNLRGYYNQSEAGSHIIQRMYGCDVGPDGRLLRGYDQYAYDGKDYIALNEDLRSWTAADTAAQITQRKWEAAREAEQLRAYLEGLCVEWLRRYLKNGKETLQRAEHPKTHVTHHPVSDHEATLRCWALGFYPAEITLTWQWDGEDQTQDTELVETRPAGDGTFQKWAAVVVPSGEEQRYTCHVQHEGLPEPLTLRWEP

SEQ ID NO: 423 - protein, 276 aa, synthetic construct
  REGION 1..276: note = synthetic sequence
GSHSMRYFSTSVSWPGRGEPRFIAVGYVDDTQFVRFDSDAASPRGEPREPWVEQEGPEYWDRETQKYKRQAQADRVNLRKLRGYYNQSEDGSHTLQRMFGCDLGPDGRLLRGYNQFAYDGKDYIALNEDLRSWTAADTAAQITQRKWEAAREAEQRRAYLEGTCVEWLRRYLENGKETLQRAEHPKTHVTHHPVSDHEATLRCWALGFYPAEITLTWQWDGEDQTQDTELVETRPAGDGTFQKWAAVVVPSGEEQRYTCHVQHEGLPEPLTLRWKP

SEQ ID NO: 424 - protein, 276 aa, synthetic construct
  REGION 1..276: note = synthetic sequence
CSHSMRYFDTAVSRPGRGEPRFISVGYVDDTQFVRFDSDAASPRGEPRAPWVEQEGPEYWDRETQKYKRQAQADRVNLRKLRGYYNQSEDGSHTLQWMYGCDLGPDGRLLRGYDQSAYDGKDYIALNEDLRSWTAADTAAQITQRKWEAAREAEQWRAYLECTCVEWLRRYLENCKETLQRAEHPKTHVTHHPVSDHEATLRCWALGFYPAEITLTWQRDGEDQTQDTELVETRPAGDGTFQKWAAVVVPSGEEQRYTCHVQHEGLPEPLTLRWEP

SEQ ID NO: 425 - protein, 276 aa, synthetic construct
  REGION 1..276: note = synthetic sequence
CSHSMRYFDTAVSRPGRGEPRFISVGYVDDTQFVRFDSDAASPRGEPRAPWVEQEGPEYWDRETQKYKRQAQADRVSLRNLRGYYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTQDTELVETRPAGDGTFQKWAAVVVPSGQEQRYTCHMQHEGLQEPLTLSWEP

SEQ ID NO: 426 - protein, 276 aa, synthetic construct
  REGION 1..276: note = synthetic sequence
CSHSMRYFYTAVSRPGRGEPRFIAVGYVDDTQFVQFDSDAASPRGEPRAPWVEQEGPEYWDRETQKYKRQAQTDRVSLRNLRGYYNQSEAGSHTLQRMYGCDLGPDGRLLRGYNQFAYDGKDYIALNEDLRSWTAADTAAQITQRKWEAARTAEQLRAYLEGTCVEWLRRYLENGKKTLQRAEHPKTHVTHHPVSDHEATLRCWALGFYPAEITLTWQRDGEDQTQDTELVETRPAGDGTFQKWAAVVVPSGEEQRYTCHVQHEGLPEPLTLRWGP

SEQ ID NO: 427 - protein, 276 aa, synthetic construct
  REGION 1..276: note = synthetic sequence
CSHSMRYFYTAVSRPGRGEPHFIAVGYVDDTQFVRFDSDAASPRGEPRAPWVEQEGPEYWDRETQNYKRQAQTDRVNLRKLRGYYNQSEAGSHIIQRMYGCDLGPDGRLLRGHDQLAYDGKDYIALNEDLRSWTAADTAAQITQRKWEAAREAEQLRAYLEGTCVEWLRRYLENGKETLQRAEHPKTHVTHHPVSDHEATLRCWALGFYPAEITLTWQRDGEDQTQDTELVETRPAGDGTFQKWAAVVVPSGEEQRYTCHVQHEGLPEPLTLRWEP

SEQ ID NO: 428 - protein, 286 aa, synthetic construct
  REGION 1..286: note = synthetic sequence
  VARIANT 89: note = Xaa is Lys or Glu
  VARIANT 108: note = Xaa is Arg or Gly
  VARIANT 158: note = Xaa is Arg or Gly
  VARIANT 159: note = Xaa is Ala or Val
  VARIANT 256: note = Xaa is Gln or Pro
  VARIANT 269: note = Xaa is Pro or Ser
GSHSLKYFHTSVSRPGRGEPRFISVGYVDDTQFVRFDNDAASPRMVPRAPWMEQEGSEYWDRETRSARDTAQIFRVNLRTLRGYYNQSXLAGSHTLQWMHGCELGPDXRFLRGYEQFAYDGKDYLTLNEDLRSWTAVDTAAQISEQKSNDASEAEHQXXYLEDTCVEWLHKYLEKGKETLLHLEPPKTHVTHHPISDHEATLRCWALGFYPAEITLTWQQDGEGHTQDTELVETRPAGDGTFQKWAAVVVPSGEEXSRYTCHVQHEGLXEPVTLRWKPASQPTIPI

SEQ ID NO: 429 - protein, 285 aa, synthetic construct
  REGION 1..285: note = synthetic sequence
  VARIANT 7: note = Xaa is Tyr or Phe
  VARIANT 51: note = Xaa is Pro or Gln
  VARIANT 252: note = Xaa is Ser or Pro
  VARIANT 279: note = Xaa is Pro or Leu
GSHSLRXLFSTAVSRPGRGEPRYIAVEYVDDTQFLRFDSDAAIPRMEPREXWVEQEGPQYWEWTTGYAKANAQTDRVALRNLLRRYNQSEAGSHTLQGMNGCDMGPDGRLLRGYHQHAYDGKDYISLNEDLRSWTAADTVAQITQRFYEAEEYAEEFRTYLEGECLELLRRYLENGKETLQRADPPKAHVAHHPISDHEATLRCWALGFYPAEITLTWQRDGEEQTQDTELVETRPAGDGTFQKWAAVVVPXGEEQRYTCHVQHEGLPQPLILRWEQSXQPTIPI

SEQ ID NO: 430 - protein, 286 aa, synthetic construct
  REGION 1..286: note = synthetic sequence
  VARIANT 13: note = Xaa is Ser or Phe
  VARIANT 27: note = Xaa is Tyr or His
  VARIANT 31: note = Xaa is Thr, Ser or Met
  VARIANT 34: note = Xaa is Leu or Val
  VARIANT 54: note = Xaa is Gln or Arg
  VARIANT 82: note = Xaa is Pro or Leu
  VARIANT 101: note = Xaa is Gly or Asp
  VARIANT 105: note = Xaa is Gly or Val
  VARIANT 106: note = Xaa is Ser or Cys
  VARIANT 111: note = Xaa is Leu or Ile
  VARIANT 160: note = Xaa is Tyr or His
  VARIANT 170: note = Xaa is His or Arg
  VARIANT 172: note = Xaa is Tyr or His
  VARIANT 179: note = Xaa is Met or Thr
  VARIANT 186: note = Xaa is Pro or Ala
  VARIANT 221: note = Xaa is Arg, Try or Gln
  VARIANT 260: note = Xaa is Thr or Met
  VARIANT 277: note = Xaa is Lys or Glu
GSHSMRYFSAAVXRPGRGEPRFIAMGXVDDXQFXRFDSDSACPRMEPRAPWVEXSEGPEYWEEETRNTKAHAQTDRMNLQTXRGYYNQSEASSHTLQWMIXCDLXXDGRLXRGYEQYAYDGKDYLALNEDLRSWTAADTAAQISKRKCEAANVAEQRRAXLEGTCVEWLXRXLENGKEXLQRADPXSKTHVTHHPVFDYEATLRCWALGFYPAEIILTWQXDGEDQTQDVELVETRPAGDGTFQKWAAVVVPSGEEQRYXCHVQHEGLPEPLMLRWXQSSLPTIPI

SEQ ID NO: 431 - protein, 284 aa, synthetic construct
  REGION 1..284: note = synthetic sequence
  VARIANT 89: note = Xaa can be any naturally occurring amino acid
  VARIANT 107: note = Xaa can be any naturally occurring amino acid
  VARIANT 157..158: note = Xaa can be any naturally occurring amino acid
  VARIANT 255: note = Xaa can be any naturally occurring amino acid
  VARIANT 267: note = Xaa can be any naturally occurring amino acid
GSHSLKYFHTSVSRPGRGEPRFISVGYVDDTQFVRFDNDAASPRMVPRAPWMEQEGSEYWDRETRSARDTAQIFRVNLRTLRGYYNQSXAGSHTLQWMHGCELGPDXRFLRGYEQFAYDGKDYLTLNEDLRSWTAVDTAAQISEQKSNDASEAEHQXXYLEDTCVEWLHKYLEKGKETLLHLEPPKTHVTHHPISDHEATLRCWALGFYPAEITLTWQQDGEGHTQDTELVETRPAGDGTFQKWAAVVVPSGEEXRYTCHVQHEGLXEPVTLRWKPASQPTIPI

SEQ ID NO: 432 - protein, 284 aa, synthetic construct
  REGION 1..284: note = synthetic sequence
  VARIANT 7: note = Xaa can be any naturally occurring amino acid
  VARIANT 50: note = Xaa can be any naturally occurring amino acid
  VARIANT 251: note = Xaa can be any naturally occurring amino acid
  VARIANT 278: note = Xaa can be any naturally occurring amino acid
GSHSLRXFSTAVSRPGRGEPRYIAVEYVDDTQFLRFDSDAAIPRMEPREXWVEQEGPQYWEWTTGYAKANAQTDRVALRNLLRRYNQSEAGSHTLQGMNGCDMGPDGRLLRGYHQHAYDGKDYISLNEDLRSWTAADTVAQITQRFYEAEEYAEEFRTYLEGECLELLRRYLENGKETLQRADPPKAHVAHHPISDHEATLRCWALGFYPAEITLTWQRDGEEQTQDTELVETRPAGDGTFQKWAAVVVPXGEEQRYTCHVQHEGLPQPLILRWEQSXQPTIPI

SEQ ID NO: 433 - protein, 284 aa, synthetic construct
  REGION 1..284: note = synthetic sequence
  VARIANT 13: note = Xaa can be any naturally occurring amino acid
  VARIANT 27: note = Xaa can be any naturally occurring amino acid
  VARIANT 31: note = Xaa can be any naturally occurring amino acid
  VARIANT 34: note = Xaa can be any naturally occurring amino acid
  VARIANT 54: note = Xaa can be any naturally occurring amino acid
  VARIANT 81: note = Xaa can be any naturally occurring amino acid
  VARIANT 100: note = Xaa can be any naturally occurring amino acid
  VARIANT 104..105: note = Xaa can be any naturally occurring amino acid
  VARIANT 110: note = Xaa can be any naturally occurring amino acid
  VARIANT 159: note = Xaa can be any naturally occurring amino acid
  VARIANT 169: note = Xaa can be any naturally occurring amino acid
  VARIANT 171: note = Xaa can be any naturally occurring amino acid
  VARIANT 178: note = Xaa can be any naturally occurring amino acid
  VARIANT 185: note = Xaa can be any naturally occurring amino acid
  VARIANT 219: note = Xaa can be any naturally occurring amino acid
  VARIANT 258: note = Xaa can be any naturally occurring amino acid
  VARIANT 275: note = Xaa can be any naturally occurring amino acid
GSHSMRYFSAAVXRPGRGEPRFIAMGXVDDXQFXRFDSDSACPRMEPRAPWVEXEGPEYWEEETRNTKAHAQTDRMNLQTXRGYYNQSEASSHTLQWMIXCDLXXDGRLXRGYEQYAYDGKDYLALNEDLRSWTAADTAAQISKRKCEAANVAEQRRAXLEGTCVEWLXRXLENGKEXLQRADPXKTHVTHHPVFDYEATLRCWALGFYPAEIILTWQXDGEDQTQDVELVETRPAGDGTFQKWAAVVVPSGEEQRYXCHVQHEGLPEPLMLRWXQSSLPTIPI

SEQ ID NO: 434 - protein, 255 aa, Homo sapiens
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCEL

(19) United States
(12) Patent Application Publication
(10) Pub. No.: US 2026/0053855 A1
(43) Pub. Date: Feb. 26, 2026

(54) Modified Cytotoxic T Cells and Methods of Use Thereof

(71) Applicant: Cue Biopharma, Inc., Boston, MA (US)

(72) Inventor: Anish Suri, Boston, MA (US)

(21) Appl. No.: 19/314,609

(22) Filed: Aug. 29, 2025

Related U.S. Application Data
(63) Continuation of application No. 17/678,735, filed on Feb. 23, 2022.
Application No. 17/678,735 is a continuation of application No. PCT/US2020/056901, filed on Oct. 22, 2020.
(60) Provisional application No. 62/925,111, filed on Oct. 23, 2019.

(51) Int. Cl. A61K 35/17; A61K 38/17; A61K 39/00; A61K 40/11; A61K 40/31; A61K 40/32; A61K 40/42; C12N 5/0783; C12N 15/62

(52) U.S. Cl. CPC A61K 35/17; A61K 38/1774; A61K 39/0011; A61K 40/11; A61K 40/31; A61K 40/32; A61K 40/42; C12N 5/0638; C12N 15/625

(57) ABSTRACT

The present disclosure provides in vitro modified cytotoxic T cells (CTLs) that comprise: a) a T-cell receptor (TCR) specific for a preselected antigen in a human; and b) a nucleic acid(s) encoding a chimeric antigen receptor (CAR) specific for a cancer-associated antigen. The present disclosure provides methods of producing the modified CTLs. The present disclosure provides methods of treating cancer, comprising administering the modified CTLs to an individual in need thereof.

### CROSS-REFERENCE

This application is a continuation of U.S. patent application Ser. No. 17/678,735, filed Feb. 23, 2022, which is a continuation of PCT Application No. PCT/US2020/056901, filed Oct. 22, 2020, which claims the benefit of U.S. Provisional Patent Application No. 62/925,111, filed Oct. 23, 2019, which applications are incorporated herein by reference in their entirety.

### INCORPORATION BY REFERENCE OF SEQUENCE LISTING

A Sequence Listing is provided herewith as a Sequence Listing XML, “CUEB-127CON2_SEQLIST_11-10-25.xml” created on Nov. 10, 2025 created and having a size of 710,331 bytes. The contents of the Sequence Listing XML are incorporated herein by reference in their entirety.

### INTRODUCTION

An adaptive immune response involves the engagement of the T cell receptor (TCR), present on the surface of a T cell, with a small peptide antigen non-covalently presented on the surface of an antigen presenting cell (APC) by a major histocompatibility complex (MHC; also referred to in humans as a human leukocyte antigen (HLA) complex). This engagement represents the immune system's targeting mechanism and is a requisite molecular interaction for T cell modulation (activation or inhibition) and effector function. Following epitope-specific cell targeting, the targeted T cells are activated through engagement of costimulatory proteins found on the APC with counterpart costimulatory proteins the T cells. Both signals—epitope/TCR binding and engagement of APC costimulatory proteins with T cell costimulatory proteins—are required to drive T cell specificity and activation or inhibition. The TCR is specific for a given epitope; however, the costimulatory protein not epitope specific and instead is generally expressed on all T cells or on large T cell subsets.

### SUMMARY

The present disclosure provides in vitro modified cytotoxic T cells (CTLs) that comprise: a) a T-cell receptor (TCR) specific for a preselected antigen in a human; and b) a nucleic acid(s) encoding a chimeric antigen receptor (CAR) specific for a cancer-associated antigen. The present disclosure provides methods of producing the modified CTLs. The present disclosure provides methods of treating cancer, comprising administering the modified CTLs to an individual in need thereof.

### BRIEF DESCRIPTION OF THE DRAWINGS

FIG. 1 is a schematic depiction of generation and use of modified CTLs according to the present disclosure.

FIG. 2A-2D provide schematic depictions of exemplary embodiments of TMMPs.

FIGS. 3A-3G provide amino acid sequences (from top to bottom SEQ ID NOs: 376-387) of immunoglobulin Fc polypeptides.

FIG. 4 provides a multiple amino acid sequence alignment of beta-2 microglobulin (β2M) precursors (i.e., including the leader sequence) from Homo sapiens (NP_004039.1; SEQ ID NO:388), Pan troglodytes (NP_001009066.1; SEQ ID NO:388), Macaca mulatta (NP_001040602.1; SEQ ID NO:389), Bos taurus (NP_776318.1; SEQ ID NO:390) and Mus musculus (NP_033865.2; SEQ ID NO:391). Amino acids 1-20 are a signal peptide.

FIGS. 5A-5C provide amino acid sequences of full-length human HLA heavy chains of alleles A*0101 (SEQ ID NO:392), A*1101 (SEQ ID NO:393), A*2402 (SEQ ID NO:394), and A*3303 (SEQ ID NO:395) (FIG. 5A); full-length human HLA heavy chain of allele B*0702 (FIG. 5B; SEQ ID NO:396); and a full-length human HLA-C heavy chain (FIG. 5C; SEQ ID NO:397).

FIG. 6 provides an alignment of eleven mature MHC class I heavy chain amino acid sequences without their leader sequences, transmembrane domains, and intracellular domains.

FIGS. 7A-7B provide an alignment of HLA-A heavy chain amino acid sequences (FIG. 7A; from top to bottom SEQ ID NOs: 406, 185, 407-413) and a consensus sequence (FIG. 7B; SEQ ID NO: 184).

FIGS. 8A-8B provide an alignment of HLA-B heavy chain amino acid sequences (FIG. 8A; from top to bottom SEQ ID NOs: 195, 414-419) and a consensus sequence (FIG. 8B; SEQ ID NO: 194).

FIGS. 9A-9B provide an alignment of HLA-C heavy chain amino acid sequences (FIG. 9A; from top to bottom SEQ ID NOs: 420-424, 199, 425-427) and a consensus sequence (FIG. 9B; SEQ ID NO: 198).

FIG. 10 provides a consensus amino acid sequence for each of HLA-E (SEQ ID NO:428), -F (SEQ ID NO:429), and -G (SEQ ID NO:430) heavy chains. Variable amino acid (aa) positions are indicated as “X” residues sequentially numbered; the locations of amino acids 84, 139, and 236 are double underlined.

FIG. 11 provides an alignment of consensus amino acid sequences for HLA-A (SEQ ID NO: 184), -B (SEQ ID NO: 194), -C(SEQ ID NO: 198), -E (SEQ ID NO:431), -F (SEQ ID NO:432), and -G (SEQ ID NO:433).

### DEFINITIONS

The terms “polynucleotide” and “nucleic acid,” used interchangeably herein, refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases.

The terms “peptide,” “polypeptide,” and “protein” are used interchangeably herein, and refer to a polymeric form of amino acids of any length, which can include coded and non-coded amino acids, chemically or biochemically modified or derivatized amino acids, and polypeptides having modified peptide backbones.

A polynucleotide or polypeptide has a certain percent “sequence identity” to another polynucleotide or polypeptide, meaning that, when aligned, that percentage of bases or amino acids are the same, and in the same relative position, when comparing the two sequences. Sequence identity can be determined in a number of different ways. To determine sequence identity, sequences can be aligned using various convenient methods and computer programs (e.g., BLAST, T-COFFEE, MUSCLE, MAFFT, etc.), available over the world wide web at sites including ncbi.nlm.nili.gov/BLAST, ebi.ac.uk/Tools/msa/tcoffee/, ebi.ac.uk/Tools/msa/muscle/, mafft.cbrc.jp/alignment/software/. See, e.g., Altschul et al. (1990), J. Mol. Biol. 215:403-10.

The term “conservative amino acid substitution” refers to the interchangeability in proteins of amino acid residues having similar side chains. For example, a group of amino acids having aliphatic side chains consists of glycine, alanine, valine, leucine, and isoleucine; a group of amino acids having aliphatic-hydroxyl side chains consists of serine and threonine; a group of amino acids having amide containing side chains consisting of asparagine and glutamine; a group of amino acids having aromatic side chains consists of phenylalanine, tyrosine, and tryptophan; a group of amino acids having basic side chains consists of lysine, arginine, and histidine; a group of amino acids having acidic side chains consists of glutamate and aspartate; and a group of amino acids having sulfur containing side chains consists of cysteine and methionine. Exemplary conservative amino acid substitution groups are: valine-leucine-isoleucine, phenylalanine-tyrosine, lysine-arginine, alanine-valine-glycine, and asparagine-glutamine.

The term “immunological synapse” or “immune synapse” as used herein generally refers to the natural interface between two interacting immune cells of an adaptive immune response including, e.g., the interface between an antigen-presenting cell (APC) or target cell and an effector cell, e.g., a lymphocyte, an effector T cell, a natural killer cell, and the like. An immunological synapse between an APC and a T cell is generally initiated by the interaction of a T cell antigen receptor and major histocompatibility complex molecules, e.g., as described in Bromley et al., Annu Rev Immunol. 2001; 19:375-96; the disclosure of which is incorporated herein by reference in its entirety.

“T cell” includes all types of immune cells expressing CD3, including T-helper cells (CD4⁺ cells), cytotoxic T-cells (CD8⁺ cells), T-regulatory cells (Treg), and NK-T cells.

The term “immunomodulatory polypeptide” (also referred to as a “co-stimulatory polypeptide”), as used herein, includes a polypeptide on an antigen presenting cell (APC) (e.g., a dendritic cell, a B cell, and the like) that specifically binds a cognate co-immunomodulatory polypeptide on a T cell, thereby providing a signal which, in addition to the primary signal provided by, for instance, binding of a TCR/CD3 complex with a major histocompatibility complex (MHC) polypeptide loaded with peptide, mediates a T cell response, including, but not limited to, proliferation, activation, differentiation, and the like. An immunomodulatory polypeptide can include, but is not limited to, CD7, B7-1 (CD80), B7-2 (CD86), PD-L1, PD-L2, 4-1BBL, OX40L, Fas ligand (FasL), inducible costimulatory ligand (ICOS-L), intercellular adhesion molecule (ICAM), CD30L, CD40, CD70, CD83, HLA-G, MICA, MICB, HVEM, lymphotoxin beta receptor, 3/TR6, ILT3, ILT4, HVEM, an agonist or antibody that binds Toll ligand receptor and a ligand that specifically binds with B7-H3.

As noted above, an “immunomodulatory polypeptide” (also referred to herein as a “MOD”) specifically binds a cognate co-immunomodulatory polypeptide on a T cell.

An “immunomodulatory domain” (“MOD”) of a TMMP binds a cognate co-immunomodulatory polypeptide, which may be present on a target T cell.

In general, a T-cell modulatory polypeptide (TMP) comprises a polypeptide that preferentially binds to and activates target T cells bearing a T cell receptor (TCR) specific for an antigen of interest. Likewise, a T-cell modulatory multimeric polypeptide (TMMP) comprises a multimeric T-cell modulatory polypeptide that preferentially binds to and activates target T cells bearing a T cell receptor (TCR) specific for an antigen of interest. For example, a TMMP can comprise at least one heterodimer comprising 2 polypeptide chains: a) a first polypeptide comprising: i) a peptide epitope (e.g., a peptide that is at least 4 amino acids in length (e.g., from 4 amino acids to about 25 amino acids in length); and ii) first MHC polypeptide; b) a second polypeptide comprising a second MHC polypeptide, and c) at least one immunomodulatory polypeptide, where the first and/or the second polypeptide comprises the immunomodulatory polypeptide. A TMP or a TMMP also may be referred to as a “synTac” or an “Immuno-STAT™.”

“Heterologous,” as used herein, means a nucleotide or polypeptide that is not found in the native nucleic acid or protein, respectively.

The terms “expression construct,” or “DNA construct” are used interchangeably herein to refer to a DNA molecule comprising a vector and at least one insert.

As used herein, the term “affinity” refers to the equilibrium constant for the reversible binding of two agents (e.g., an antibody and an antigen) and is expressed as a dissociation constant (K_{D}). Affinity can be at least 1-fold greater, at least 2-fold greater, at least 3-fold greater, at least 4-fold greater, at least 5-fold greater, at least 6-fold greater, at least 7-fold greater, at least 8-fold greater, at least 9-fold greater, at least 10-fold greater, at least 20-fold greater, at least 30-fold greater, at least 40-fold greater, at least 50-fold greater, at least 60-fold greater, at least 70-fold greater, at least 80-fold greater, at least 90-fold greater, at least 100-fold greater, or at least 1,000-fold greater, or more, than the affinity of an antibody for unrelated amino acid sequences. Affinity of an antibody to a target protein can be, for example, from about 100 nanomolar (nM) to about 0.1 nM, from about 100 nM to about 1 picomolar (pM), or from about 100 nM to about 1 femtomolar (fM) or more. As used herein, the term “avidity” refers to the resistance of a complex of two or more agents to dissociation after dilution. The terms “immunoreactive” and “preferentially binds” are used interchangeably herein with respect to antibodies and/or antigen-binding fragments. Unless otherwise indicated herein, when the word “about” is used in reference to a numeric value, it means a range of ±10% of the stated numeric value, e.g., “about 10” means a value from 9 to 11.

The term “binding,” as used herein (e.g. with reference to binding of a TMMP to a polypeptide (e.g., a T-cell receptor) on a T cell; or with reference to binding of an antigen-binding polypeptide present in a CAR to an antigen such as a cancer-associated antigen), refers to a non-covalent interaction between two molecules. Non-covalent binding refers to a direct association between two molecules, due to, for example, electrostatic, hydrophobic, ionic, and/or hydrogen-bond interactions, including interactions such as salt bridges and water bridges. Non-covalent binding interactions are generally characterized by a dissociation constant (K_{D}) of less than 10⁻⁶ M, less than 10⁻⁷ M, less than 10⁻⁸ M, less than 10⁻⁹ M, less than 10⁻¹⁰ M, less than 10⁻¹¹ M, less than 10⁻¹² M, less than 10⁻¹³ M, less than 10⁻¹⁴ M, or less than 10⁻¹⁵ M. “Affinity” refers to the strength of non-covalent binding, increased binding affinity being correlated with a lower K_{D}. “Specific binding” generally refers to binding with an affinity of at least about 10⁻⁷ M or greater, e.g., 5×10⁻⁷ M, 10⁻⁸ M, 5×10⁻⁸ M, 10⁻⁹ M, and greater. “Non-specific binding” generally refers to binding (e.g., the binding of a ligand to a moiety other than its designated binding site or receptor) with an affinity of less than about 10⁻⁷ M (e.g., binding with an affinity of 10⁻⁶ M, 10⁻⁵ M, 10⁻⁴ M). However, in some contexts, e.g., binding between a TCR and a peptide/MHC complex, “specific binding” can be in the range of from 1 μM to 100 μM, or from 100 μM to 1 mM. “Covalent binding” or “covalent bond,” as used herein, refers to the formation of one or more covalent chemical binds between two different molecules.

The terms “treatment”, “treating” and the like are used herein to generally mean obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment” as used herein covers any treatment of a disease or symptom in a mammal, and includes: (a) preventing the disease or symptom from occurring in a subject which may be predisposed to acquiring the disease or symptom but has not yet been diagnosed as having it; (b) inhibiting the disease or symptom, i.e., arresting its development; and/or (c) relieving the disease, i.e., causing regression of the disease. The therapeutic agent may be administered before, during or after the onset of disease or injury. The treatment of ongoing disease, where the treatment stabilizes or reduces the undesirable clinical symptoms of the patient, is of particular interest. Such treatment is desirably performed prior to complete loss of function in the affected tissues. The subject therapy will desirably be administered during the symptomatic stage of the disease, and in some cases after the symptomatic stage of the disease.

The terms “individual,” “subject,” “host,” and “patient,” are used interchangeably herein and refer to any mammalian subject for whom diagnosis, treatment, or therapy is desired. Mammals include, e.g., humans, non-human primates, rodents (e.g., rats; mice), lagomorphs (e.g., rabbits), ungulates (e.g., cows, sheep, pigs, horses, goats, and the like), etc.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “a modified T cell” includes a plurality of such T cells and reference to “the T-cell modulatory multimeric polypeptide” includes reference to one or more T-cell modulatory multimeric polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION

The present disclosure provides in vitro modified cytotoxic T cells (CTLs) that comprise: a) a T-cell receptor (TCR) specific for a preselected antigen in a human; and b) a nucleic acid(s) encoding a chimeric antigen receptor (CAR) specific for a cancer-associated antigen. The present disclosure provides methods of producing the modified CTLs. The present disclosure provides methods of treating cancer, comprising administering the modified CTLs to an individual in need thereof.

### Modified Cytotoxic T Cells

The present disclosure provides in vitro modified T cells that comprise: a) a TCR specific for a preselected antigen present in a human; and b) one or more nucleic acids comprising nucleotide sequences encoding a CAR, where the CAR comprises an antigen-binding domain that binds to a cancer-associated antigen. The present disclosure provides an in vitro composition comprising a quantity of (a population of) target modified T cells that comprise: a) a TCR specific for a preselected antigen present in a human; and b) one or more nucleic acids comprising nucleotide sequences encoding a CAR, where the CAR comprises an antigen-binding domain that binds to a cancer-associated antigen. The present disclosure provides in vitro modified CTLs that comprise: a) a TCR specific for a preselected antigen present in a human; and b) one or more nucleic acids comprising nucleotide sequences encoding a CAR, where the CAR comprises an antigen-binding domain that binds to a cancer-associated antigen. The present disclosure provides an in vitro composition comprising a quantity of (a population of) target modified CTLs that comprise: a) a TCR specific for a preselected antigen present in a human; and b) one or more nucleic acids comprising nucleotide sequences encoding a CAR, where the CAR comprises an antigen-binding domain that binds to a cancer-associated antigen.

An in vitro composition of the present disclosure can comprise a population of modified T cells that may contain modified cells (e.g., T cells, such as CTLs) other than the target modified T cells (e.g., target modified CTLs). Such cells are referred to as “non-target modified T cells,” where non-target T cells can include non-target CTLs. Non-target modified T cells comprise a TCR that is not specific for the preselected antigen. Thus, an in vitro composition of the present disclosure can be a heterogeneous population comprising target modified T cells (e.g., target modified CTLs) and non-target modified T cells (e.g., non-target modified CTLs). In some cases, from 1% to 20% of the total number of T cells in the composition are target modified T cells (e.g., target modified CTLs). In some cases, from 1% to 5%, from 5% to 10%, from 10% to 15%, or from 15% to 20% of the total number of T cells in the composition are target modified T cells (e.g., target modified CTLs). In some cases, at least 20%, at least 25%, at least 30%, at least 35%, at least 40%, at least 50%, at least 60%, at least 70%, at least 75%, at least 80%, at least 85%, at least 90%, at least 95%, at least 98%, at least 99%, or more than 99%, of the total number of T cells in the composition are target modified T cells (e.g., target modified CTLs). In some cases, from 20% to 30%, from 30% to 40%, from 40% to 50%, from 50% to 60%, from 60% to 70%, from 70% to 80%, from 80% to 90%, or from 90% to 100%, of the total number of T cells in the composition are target modified T cells (e.g., target modified CTLs). In some cases, therefore, the population of T cells in the composition is a substantially homogeneous population of target modified T cells (e.g., target modified CTLs). Unless otherwise indicated, when used herein the term “substantially” means “wholly or largely but not wholly”. Hence, for example, a “substantially homogeneous population” means a population that is wholly homogeneous or largely but not wholly homogeneous.

As noted above, target modified T cells (e.g., target modified CTLs) comprise (e.g., express on their cell surface) a TCR specific for a preselected antigen present in a human. Such antigens can be antigens of pathogens that infect humans. Such antigens can be antigens present in vaccines administered to humans. In some cases, the antigen is a viral antigen. In some cases, a viral antigen is encoded by a virus that infects a majority of the human population, where such viruses include, e.g., cytomegalovirus (CMV), Epstein-Barr virus (EBV), human papilloma virus, influenza virus, adenovirus, and the like. In some cases, the antigen is a bacterial epitope, e.g., a bacterial epitope that is included in a vaccine and to which a majority of the human population has immunity. For example, in some cases, the antigen is a tetanus antigen.

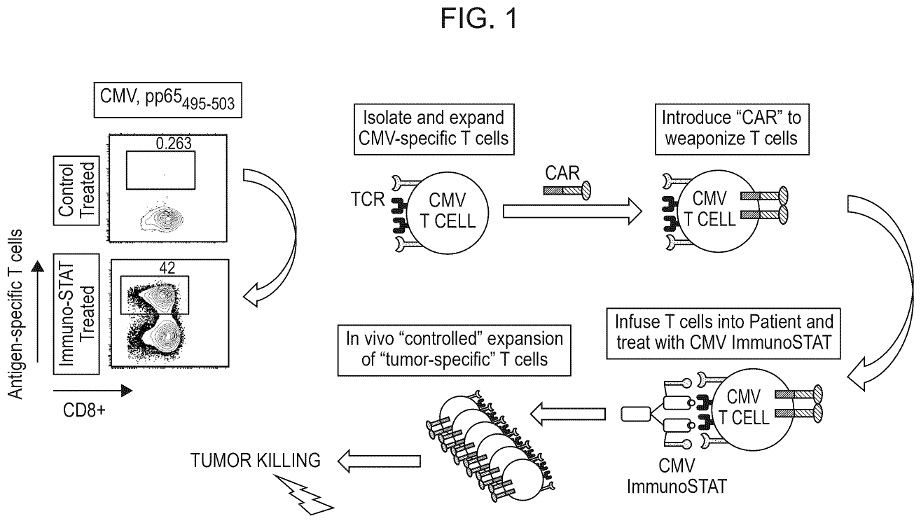

Use of an in vitro composition comprising modified target T cells is depicted schematically in FIG. 1. The in vitro cell population is modified to express a chimeric antigen receptor (CAR) specific for a cancer-associated antigen. Target modified T cells in the population comprise TCRs specific for preselected antigens present in humans. The in vitro composition comprising target modified T cells can be administered to an individual in need thereof, e.g., an individual having a cancer. A T-cell modulatory multimeric polypeptide (TMMP) that comprises a peptide epitope that is bound by the TCR present on the modified target T cells can also be administered to the individual. The TMMP comprises an immunomodulatory polypeptide that provides for activation of T cells comprising TCRs that bind the peptide epitope present in the TMMP. The modified target T cells (e.g., modified CTLs) will target cancer cells expressing the cancer-associated antigen to which the CAR present. The TMMP will activate the modified target T cells by binding to TCRs present on the modified target T cells. Such a method takes advantage of the presence in human populations of T cells (e.g., CTLs) specific for antigens such as those associated with common human pathogens and/or commonly administered human vaccines.

### Chimeric Antigen Receptor

As noted above, a modified T cell is modified to express a CAR specific for a cancer-associated antigen. A CAR generally comprises: a) an extracellular domain comprising an antigen-binding domain (antigen-binding polypeptide); b) a transmembrane region; and c) a cytoplasmic domain comprising an intracellular signaling domain (intracellular signaling polypeptide). In some cases, a CAR comprises: a) an extracellular domain comprising the antigen-binding domain; b) a transmembrane region; and c) a cytoplasmic domain comprising: i) one or more co-stimulatory polypeptides; and ii) an intracellular signaling domain. In some cases, a CAR comprises hinge region between the extracellular antigen-binding domain and the transmembrane domain. Thus, in some cases, a CAR comprises: a) an extracellular domain comprising the antigen-binding domain; b) a hinge region; c) a transmembrane region; and d) a cytoplasmic domain comprising an intracellular signaling domain. In some cases, a CAR comprises: a) an extracellular domain comprising the antigen-binding domain; b) a hinge region; c) a transmembrane region; and d) a cytoplasmic domain comprising: i) one or more co-stimulatory polypeptides; and ii) an intracellular signaling domain.

Exemplary CAR structures are known in the art (See e.g., WO 2009/091826; US 20130287748; WO 2015/142675; WO 2014/055657; WO 2015/090229; and U.S. Pat. No. 9,587,020.

In some cases, a CAR is a single polypeptide chain. In some cases, a CAR comprises two polypeptide chains. Generally, any CAR structure known to those skilled in the art may be used to modify T cells in order to prepare compositions as disclosed herein.

CARs specific for a variety of tumor antigens are known in the art; for example CD171-specific CARs (Park et al., Mol Ther (2007) 15(4):825-833), EGFRvIII-specific CARs (Morgan et al., Hum Gene Ther (2012) 23(10):1043-1053), EGF-R-specific CARs (Kobold et al., J. Natl Cancer Inst (2014) 107(1):364), carbonic anhydrase IX-specific CARs (Lamers et al., Biochem Soc Trans (2016) 44(3):951-959), folate receptor-α (FR-α)-specific CARs (Kershaw et al., Clin Cancer Res (2006) 12(20):6106-6015), HER2-specific CARs (Ahmed et al., J Clin Oncol (2015) 33(15)1688-1696; Nakazawa et al., Mol Ther (2011) 19(12):2133-2143; Ahmed et al., Mol Ther (2009) 17(10):1779-1787; Luo et al., Cell Res (2016) 26(7):850-853; Morgan et al., Mol Ther (2010) 18(4):843-851; Grada et al., Mol Ther Nucleic Acids (2013) 9(2):32), CEA-specific CARs (Katz et al., Clin Cancer Res (2015) 21(14):3149-3159), IL-13Rα2-specific CARs (Brown et al., Clin Cancer Res (2015) 21(18):4062-4072), ganglioside GD2-specific CARs (Louis et al., Blood (2011) 118(23):6050-6056; Caruana et al., Nat Med (2015) 21(5):524-529; Yu et al. (2018) J. Hematol. Oncol. 11:1), ErbB2-specific CARS (Wilkie et al., J Clin Immunol (2012) 32(5):1059-1070), VEGF-R-specific CARs (Chinnasamy et al., Cancer Res (2016) 22(2):436-447), FAP-specific CARs (Wang et al., Cancer Immunol Res (2014) 2(2): 154-166), mesothelin (MSLN)-specific CARs (Moon et al, Clin Cancer Res (2011) 17(14):4719-30), NKG2D-specific CARs (VanSeggelen et al., Mol Ther (2015) 23(10):1600-1610), CD19-specific CARs (Axicabtagene ciloleucel (Yescarta™) and Tisagenlecleucel (Kymriah™). See also, Li et al., J Hematol and Oncol (2018) 11:22, reviewing clinical trials of tumor-specific CARs; Heyman and Yan (2019) Cancers 11:pii:E191; Baybutt et al. (2019) Clin. Pharmacol. Ther. 105:71.

### Antigen-Binding Domain

As noted above, a CAR comprises an extracellular domain comprising an antigen-binding domain. The antigen-binding domain present in a CAR can be any antigen-binding polypeptide, a wide variety of which are known in the art. In some instances, the antigen-binding domain is a single chain Fv (scFv). Other antibody-based recognition domains (cAb VHH (camelid antibody variable domains) and humanized versions, IgNAR VH (shark antibody variable domains) and humanized versions, sdAb VH (single domain antibody variable domains) and “camelized” antibody variable domains are suitable. In some cases, the antigen-binding domain is a nanobody.

In some cases, the antigen bound by the antigen-binding domain of a CAR is selected from: a MUC1 polypeptide, an LMP2 polypeptide, an epidermal growth factor receptor (EGFR) vIII polypeptide, a HER-2/neu polypeptide, a melanoma antigen family A, 3 (MAGE A3) polypeptide, a p53 polypeptide, a mutant p53 polypeptide, an NY-ESO-1 polypeptide, a folate hydrolase (prostate-specific membrane antigen; PSMA) polypeptide, a carcinoembryonic antigen (CEA) polypeptide, a melanoma antigen recognized by T-cells (melanA/MART1) polypeptide, a Ras polypeptide, a gp100 polypeptide, a proteinase3 (PR1) polypeptide, a bcr-abl polypeptide, a tyrosinase polypeptide, a survivin polypeptide, a prostate specific antigen (PSA) polypeptide, an hTERT polypeptide, a sarcoma translocation breakpoints polypeptide, a synovial sarcoma X (SSX) breakpoint polypeptide, an EphA2 polypeptide, an acid phosphatase, prostate (PAP) polypeptide, a melanoma inhibitor of apoptosis (ML-IAP) polypeptide, an epithelial cell adhesion molecule (EpCAM) polypeptide, an ERG (TMPRSS2 ETS fusion) polypeptide, a NA17 polypeptide, a paired-box-3 (PAX3) polypeptide, an anaplastic lymphoma kinase (ALK) polypeptide, an androgen receptor polypeptide, a cyclin B1 polypeptide, an N-myc proto-oncogene (MYCN) polypeptide, a Ras homolog gene family member C (RhoC) polypeptide, a tyrosinase-related protein-2 (TRP-2) polypeptide, a mesothelin polypeptide, a prostate stem cell antigen (PSCA) polypeptide, a melanoma associated antigen-1 (MAGE A1) polypeptide, a cytochrome P450 1B1 (CYP1B1) polypeptide, a placenta-specific protein 1 (PLAC1) polypeptide, a BORIS polypeptide (also known as CCCTC-binding factor or CTCF), an ETV6-AML polypeptide, a breast cancer antigen NY-BR-1 polypeptide (also referred to as ankyrin repeat domain-containing protein 30A), a regulator of G-protein signaling (RGS5) polypeptide, a squamous cell carcinoma antigen recognized by T-cells (SART3) polypeptide, a carbonic anhydrase IX polypeptide, a paired box-5 (PAX5) polypeptide, an OY-TES1 (testis antigen; also known as acrosin binding protein) polypeptide, a sperm protein 17 polypeptide, a lymphocyte cell-specific protein-tyrosine kinase (LCK) polypeptide, a high molecular weight melanoma associated antigen (HMW-MAA), an A-kinase anchoring protein-4 (AKAP-4), a synovial sarcoma X breakpoint 2 (SSX2) polypeptide, an X antigen family member 1 (XAGE1) polypeptide, a B7 homolog 3 (B7H3; also known as CD276) polypeptide, a legumain polypeptide (LGMN1; also known as asparaginyl endopeptidase), atyrosine kinase with Ig and EGF homology domains-2 (Tie-2; also known as angiopoietin-1 receptor) polypeptide, a P antigen family member 4 (PAGE4) polypeptide, a vascular endothelial growth factor receptor 2 (VEGF2) polypeptide, a MAD-CT-1 polypeptide, a fibroblast activation protein (FAP) polypeptide, a platelet derived growth factor receptor beta (PDGFβ) polypeptide, a MAD-CT-2 polypeptide, or a Fos-related antigen-1 (FOSL) polypeptide. In some cases, the antigen is a human papilloma virus (HPV) antigen. In some cases, the antigen is an alpha-feto protein (AFP) antigen. In some cases, the antigen is a Wilms tumor-1 (WT1) antigen.

The antigen-binding polypeptide of a CAR can bind any of a variety of cancer-associated antigens, including, e.g., CD19, CD20, CD38, CD30, Her2/neu, ERBB2, CA125, MUC-1, prostate-specific membrane antigen (PSMA), CD44 surface adhesion molecule, mesothelin, carcinoembryonic antigen (CEA), epidermal growth factor receptor (EGFR), EGFRvIII, vascular endothelial growth factor receptor-2 (VEGFR2), B-cell maturation antigen (BCMA), high molecular weight-melanoma associated antigen (HMW-MAA), MAGE-A1, IL-13R-a2, GD2, and the like. Cancer-associated antigens also include, e.g., 4-1BB, 5T4, adenocarcinoma antigen, alpha-fetoprotein (AFP), BAFF, B-lymphoma cell, C242 antigen, CA-125, carbonic anhydrase 9 (CA-IX), C-MET, CCR4, CD152, CD19, CD20, CD200, CD22, CD221, CD23 (IgE receptor), CD28, CD30 (TNFRSF8), CD33, CD4, CD40, CD44 v6, CD51, CD52, CD56, CD74, CD80, CEA, CNTO888, CTLA-4, DRS, EGFR, EpCAM, CD3, FAP, fibronectin extra domain-B, folate receptor 1, GD2, GD3 ganglioside, glycoprotein 75, GPNMB, HER2/neu, HGF, human scatter factor receptor kinase, IGF-1 receptor, IGF-1, IgG1, L1-CAM, IL-13, IL-6, insulin-like growth factor I receptor, integrin α5β1, integrin αvβ, MORAb-009, MS4A1, MUC1, mucin CanAg, N-glycolylneuraminic acid, NPC-1C, PDGF-R α, PDL192, phosphatidylserine, prostatic carcinoma cells, RANKL, RON, ROR1, SCH 900105, SDC1, SLAMF7, TAG-72, tenascin C, TGF beta 2, TGF-β, TRAIL-R1, TRAIL-R2, tumor antigen CTAA16.88, VEGF-A, VEGFR-1, VEGFR2, and vimentin.

In some cases, the cancer-associated antigen bound by the antigen-binding polypeptide of a CAR is selected from AFP, BCMA, CD10, CD117, CD123, CD133, CD128, CD171, CD19, CD20, CD22, CD30, CD33, CD34, CD38, CD5, CD56, CD7, CD70, CD80, CD86, CEA, CLD18, CLL-1, cMet, EGFR, EGFRvIII, EpCAM, EphA2, GD-2, glypican-3, GPC3, HER-2, kappa immunoglobulin, LeY, LMP1, mesothlin, MG7, MUC1, NKG2D ligand, PD-L1, PSCA, PSMA, ROR1, ROR1R, TACI, and VEGFR2. In some cases, the cancer-associated antigen is BCMA. In some cases, the cancer-associated antigen is MUC1. In some cases, the cancer-associated antigen is CD19. In some cases, the cancer-associated antigen is AFP.

VH and VL amino acid sequences of various cancer-associated antigen-binding antibodies are known in the art, as are the light chain and heavy chain CDRs of such antibodies. See, e.g., Ling et al. (2018) Frontiers Immunol. 9:469; WO 2005/012493; US 2019/0119375; US 2013/0066055. The following are non-limiting examples of antibodies that bind cancer-associated antigens.

### 1) Anti-Her2

In some cases, an anti-Her2 antibody comprises: a) a light chain comprising an amino acid sequence having at least 90%, at least 95%, at least 98%, at least 99%, or 100%, amino acid sequence identity to the following amino acid sequence:

DIQMTQSPSSLSASVGDRVTITCRASQDVNTAVAWYQQKPGKAPKLLIYSASFLY SGVPSRFSGSRSGTDFTLTISSLQPEDFATYYCQQHYTTPPTFGQGTKVEIKRTVAAPSVFIFPPSD EQLKSGTASVVCLLNNFYPREAKVQWKVDNALQSGNSQESVTEQDSKDSTYSLSSTLTLSKAD YEKHKVYACEVTHQGLSSPVTKSFNRGEC (SEQ ID NO: 1); and b) a heavy chain comprising an amino acid sequence having at least 90%, at least 95%, at least 98%, at least 99%, or 100%, amino acid sequence identity to the following amino acid sequence:

(SEQ ID NO: 2)
EVQLVESGGGLVQPGGSLRLSCAASGFNIKDTYIHWVRQAPGKGLEWVA
RIYPTNGYTRYADSVKGRFTISADTSKNTAYLQMNSLRAEDTAVYYCSR
WGGDGFYAMDYWGQGTLVTVSSASTKGPSVFPLAPSSKSTSGGTAALGC
LVKDYFPEPVTVSWNSGALTSGVHTFPAVLQSSGLYSLSSVVTVPSSSL
GTQTYICNVNHKPSNTKVDKKVEPKSCDKTHTCPPCPAPELLGGPSVFL
FPPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKAK
GQPREPQVYTLPPSREEMTKNQVSLTCLVKGFYPSDIAVEWESNGQPEN
NYKTTPPVLDSDGSFFLYSKLTVDKSRWQQGNVFSCSVMHEALHNHYTQ
KSLSLSPGK.

In some cases, an anti-Her2 antibody comprises a light chain variable region (VL) present in the light chain amino acid sequence provided above; and a heavy chain variable region (VH) present in the heavy chain amino acid sequence provided above. For example, an anti-Her2 antibody can comprise: a) a VL comprising an amino acid sequence having at least 90%, at least 95%, at least 98%, at least 99%, or 100%, amino acid sequence identity to the amino acid sequence: DIQMTQSPSSLSASVGDRVTITCRASQDVNTAVAWYQQKPGKAPKLLIYSASFLYSGVPSRFSGS RSGTDFTLTISSLQPEDFATYYCQQHYTTPPTFGQGTKVEIK (SEQ ID NO:3); and b) a VH comprising an amino acid sequence having at least 90%, at least 95%, at least 98%, at least 99%, or 100%, amino acid sequence identity to the amino acid sequence: EVQLVESGGGLVQPGGSLRLSCAASGFNIKDTYIHWVRQAPGKGLEWVARIYPTNGYTRYADS VKGRFTISADTSKNTAYLQMNSLRAEDTAVYYCSRWGGDGFYAMDYWGQGTLVTVSS (SEQ ID NO:4). In some cases, an anti-Her2 antibody comprises, in order from N-terminus to C-terminus: a) a VH comprising an amino acid sequence having at least 90%, at least 95%, at least 98%, at least 99%, or 100%, amino acid sequence identity to the amino acid sequence: EVQLVESGGGLVQPGGSLRLSCAASGFNIKDTYIHWVRQAPGKGLEWVARIYPTNGYTRYADS VKGRFTISADTSKNTAYLQMNSLRAEDTAVYYCSRWGGDGFYAMDYWGQGTLVTVSS (SEQ ID NO:4); b) a linker; and c) a VL comprising an amino acid sequence having at least 90%, at least 95%, at least 98%, at least 99%, or 100%, amino acid sequence identity to the amino acid sequence: DIQMTQSPSSLSASVGDRVTITCRASQDVNTAVAWYQQKPGKAPKLLIYSASFLYSGVPSRFSGS RSGTDFTLTISSLQPEDFATYYCQQHYTTPPTFGQGTKVEIK (SEQ ID NO:3). Suitable linkers are described elsewhere herein and include, e.g., (GGGGS)n (SEQ ID NO:5), where n is an integer from 1 to 10 (e.g., 1, 2, 3, 4, 5, 6, 7, 8, 9, or 10).

In some cases, an anti-Her2 antibody comprises VL CDR1, VL CDR2, and VL CDR3 present in the light chain amino acid sequence provided above; and VH CDR1, CDR2, and CDR3 present in the heavy chain amino acid sequence provided above. In some cases, the V_{H} and V_{L} CDRs are as defined by Kabat (see, e.g., Table 1, above; and Kabat 1991). In some cases, the V_{H} and V_{L} CDRs are as defined by Chothia (see, e.g., Table 1, above; and Chothia 1987).

For example, an anti-Her2 antibody can comprise a VL CDR1 having the amino acid sequence RASQDVNTAVA (SEQ ID NO:6); a VL CDR2 having the amino acid sequence SASFLY (SEQ ID NO:7); a VL CDR3 having the amino acid sequence QQHYTTPP (SEQ ID NO:8); a VH CDR1 having the amino acid sequence GFNIKDTY (SEQ ID NO:9); a VH CDR2 having the amino acid sequence 1YPTNGYT (SEQ ID NO: 10); and a VH CDR3 having the amino acid sequence SRWGGDGFYAMDY (SEQ ID NO: 11).

In some cases, an anti-Her2 antibody is a scFv antibody. For example, an anti-Her2 scFv can comprise an amino acid sequence having at least 90%, at least 95%, at least 98%, at least 99%, or 100%, amino acid sequence identity to the following amino acid sequence:

(SEQ ID NO: 12)
EVQLVESGGGLVQPGGSLRLSCAASGFNIKDTYIHWVRQAPGKGLEWVA
RIYPTNGYTRYADSVKGRFTISADTSKNTAYLQMNSLRAEDTAVYYCSR
WGGDGFYAMDYWGQGTLVTVSSGGGGSGGGGSGGGGSDIQMTQSPSSLS
ASVGDRVTITCRASQDVNTAVAWYQQKPGKAPKLLIYSASFLYSGVPSR
FSGSRSGTDFTLTISSLQPEDFATYYCQQHYTTPPTFGQGTKVEIK.

As another example, in some cases, an anti-Her2 antibody comprises: a) a light chain variable region (VL) comprising an amino acid sequence having at least 90%, at least 95%, at least 98%, at least 99%, or 100%, amino acid sequence identity to the following amino acid sequence:
DIQMTQSPSSLSASVGDRVTITCKASQDVSIGVAWYQQKPGKAPKLLIYSASYRY TGVPSRFSGSGSGTDFTLTISSLQPEDFATYYCQQYYIYPYTFGQGTKVEIKRTVAAPSVFIFPPSD EQLKSGTASVVCLLNNFYPREAKVQWKVDNALQSGNSQESVTEQDSKDSTYSLSSTLTLSKAD YEKHKVYACEVTHQGLSSPVTKSFNRGEC (SEQ ID NO: 13); and b) a heavy chain variable region (VH) comprising an amino acid sequence having at least 90%, at least 95%, at least 98%, at least 99%, or 100%, amino acid sequence identity to the following amino acid sequence:

(SEQ ID NO: 14)
EVQLVESGGGLVQPGGSLRLSCAASGFTFTDYTMDWVRQAPGKGLEWVA
DVNPNSGGSIYNQRFKGRFTLSVDRSKNTLYLQMNSLRAEDTAVYYCAR
NLGPSFYFDYWGQGTLVTVSSASTKGPSVFPLAPSSKSTSGGTAALGCL
VKDYFPEPVTVSWNSGALTSGVHTFPAVLQSSGLYSLSSVVTVPSSSLG
TQTYICNVNHKPSNTKVDKKVEPKSCDKTHTCPPCPAPELLGGPSVFLF
PPKPKDTLMISRTPEVTCVVVDVSHEDPEVKFNWYVDGVEVHNAKTKPR
EEQYNSTYRVVSVLTVLHQDWLNGKEYKCKVSNKALPAPIEKTISKAKG
QPREPQVYTLPPSREEMTKNQVSLTCLVKGFYPSDIAVEWESNGQPENN
YKTTPPVLDSDGSFFLYSKLTVDKSRWQQGNVFSCSVMHEALHNHYTQK
SLSLSPG.

In some cases, an anti-Her2 antibody comprises a VL present in the light chain amino acid sequence provided above; and a VH present in the heavy chain amino acid sequence provided above. For example, an anti-Her2 antibody can comprise: a) a VL comprising an amino acid sequence having at least 90%, at least 95%, at least 98%, at least 99%, or 100%, amino acid sequence identity to the amino acid sequence: DIQMTQSPSSLSASVGDRVTITCKASQDVSIGVAWYQQKPGKAPKLLIYSASYRYTGVPSRFSGS GSGTDFTLTISSLQPEDFATYYCQQYYIYPYTFGQGTKVEIK (SEQ ID NO: 15); and b) a VH comprising an amino acid sequence having at least 90%, at least 95%, at least 98%, at least 99%, or 100%, amino acid sequence identity to the amino acid sequence:

(SEQ ID NO: 16)
EVQLVESGGGLVQPGGSLRLSCAASGFTFTDYTMDWVRQAPGKGLEWVA
DVNPNSGGSIYNQRFKGRFTLSVDRSKNTLYLQMNSLRAEDTAVYYCAR
NLGPSFYFDYWGQGTLVTVSS.

In some cases, an anti-Her2 antibody comprises VL CDR1, VL CDR2, and VL CDR3 present in the light chain amino acid sequence provided above; and VH CDR1, CDR2, and CDR3 present in the heavy chain amino acid sequence provided above. In some cases, the V_{H} and V_{L} CDRs are as defined by Kabat (see, e.g., Table 1, above; and Kabat 1991). In some cases, the V_{H} and V_{L} CDRs are as defined by Chothia (see, e.g., Table 1, above; and Chothia 1987).

For example, an anti-HER2 antibody can comprise a VL CDR1 having the amino acid sequence KASQDVSIGVA (SEQ ID NO: 17); a VL CDR2 having the amino acid sequence SASYRY (SEQ ID NO: 18); a VL CDR3 having the amino acid sequence QQYYIYPY (SEQ ID NO: 19); a VH CDR1 having the amino acid sequence GFTFTDYTMD (SEQ ID NO:20); a VH CDR2 having the amino acid sequence ADVNPNSGGSIYNQRFKG (SEQ ID NO:21); and a VH CDR3 having the amino acid sequence ARNLGPSFYFDY (SEQ ID NO:22).

In some cases, an anti-Her2 antibody is a scFv. For example, in some cases, an anti-Her2 scFv comprises an amino acid sequence having at least 90%, at least 95%, at least 98%, at least 99%, or 100%, amino acid sequence identity to the following amino acid sequence:

(SEQ ID NO: 12)
EVQLVESGGGLVQPGGSLRLSCAASGFNIKDTYIHWVRQAPGKGLEWVA
RIYPTNGYTRYADSVKGRFTISADTSKNTAYLQMNSLRAEDTAVYYCSR
WGGDGFYAMDYWGQGTLVTVSSGGGGSGGGGSGGGGSDIQMTQSPSSLS
ASVGDRVTITCRASQDVNTAVAWYQQKPGKAPKLLIYSASFLYSGVPSR
FSGSRSGTDFTLTISSLQPEDFATYYCQQHYTTPPTFGQGTKVEIK.

### 2) Anti-CD19

Anti-CD19 antibodies are known in the art; and the VH and VL, or the VH and VL CDRs, of any anti-CD19 antibody can be included in a CAR. See e.g., WO 2005/012493.

In some cases, an anti-CD19 antibody includes a VL CDR1 comprising the amino acid sequence KASQSVDYDGDSYLN (SEQ ID NO:23); a VL CDR2 comprising the amino acid sequence DASNLVS (SEQ ID NO:24); and a VL CDR3 comprising the amino acid sequence QQSTEDPWT (SEQ ID NO:25). In some cases, an anti-CD19 antibody includes a VH CDR1 comprising the amino acid sequence SYWMN (SEQ ID NO:26); a VH CDR2 comprising the amino acid sequence QIWPGDGDTNYNGKFKG (SEQ ID NO:27); and a VH CDR3 comprising the amino acid sequence RETTTVGRYYYAMDY (SEQ ID NO:28). In some cases, an anti-CD19 antibody includes a VL CDR1 comprising the amino acid sequence KASQSVDYDGDSYLN (SEQ ID NO:23); a VL CDR2 comprising the amino acid sequence DASNLVS (SEQ ID NO:24); a VL CDR3 comprising the amino acid sequence QQSTEDPWT (SEQ ID NO:25); a VH CDR1 comprising the amino acid sequence SYWMN (SEQ ID NO:26); a VH CDR2 comprising the amino acid sequence QIWPGDGDTNYNGKFKG (SEQ ID NO:27); and a VH CDR3 comprising the amino acid sequence RETTTVGRYYYAMDY (SEQ ID NO:28).

In some cases, an anti-CD19 antibody is a scFv. For example, in some cases, an anti-CD19 scFv comprises an amino acid sequence having at least 90%, at least 95%, at least 98%, at least 99%, or 100%, amino acid sequence identity to the following amino acid sequence:

(SEQ ID NO: 29)
DIQLTQSPASLAVSLGQRATISCKASQSVDYDGDSYLNWYQQIPGQPPKL
LIYDASNLVSGIPPRFSGSGSGTDFTLNIHPVEKVDAATYHCQQSTEDPW
TFGGGTKLEIKGGGGSGGGGSGGGGSQVQLQQSGAELVRPGSSVKISCKA
SGYAFSSYWMNWVKQRPGQGLEWIGQIWPGDGDTNYNGKFKGKATLTADE
SSSTAYMQLSSLASEDSAVYFCARRETTTVGRYYYAMDYWGQGTTVTVS.

### 3) Anti-Mesothelin

Anti-mesothelin antibodies are known in the art; and the VH and VL, or the VH and VL CDRs, of any anti-mesothelin antibody can be included in a CAR. See, e.g., U.S. 2019/0000944; WO 2009/045957; WO 2014/031476; U.S. Pat. No. 8,460,660; US 2013/0066055; and WO 2009/068204.

In some cases, an anti-mesothelin antibody comprises: a) a light chain comprising an amino acid sequence having at least 90%, at least 95%, at least 98%, at least 99%, or 100%, amino acid sequence identity to the following amino acid sequence:
DIALTQPASVSGSPGQSITISCTGTSSDIGGYNSVSWYQQHPGKAPKLMIYGVNNR PSGVSNRFSGSKSGNTASLTISGLQAEDEADYYCSSYDIESATPVFGGGTKLTVLGQPKAAPSVT LFPPSSEELQANKATLVCLISDFYPGAVTVAWKGDSSPVKAGVETTTPSKQSNNKYAASSYLSL TPEQWKSHRSYSCQVTHEGSTVEKTVAPTESS (SEQ ID NO:30); and
b) a heavy chain comprising an amino acid sequence having at least 90%, at least 95%, at least 98%, at least 99%, or 100%, amino acid sequence identity to the following amino acid sequence:

(SEQ ID NO: 31)
QVELVQSGAEVKKPGESLKISCKGSGYSFTSYWIGWVRQAPGKGLEWMGI
IDPGDSRTRYSPSFQGQVTISADKSISTAYLQWSSLKASDTAMYYCARGQ
LYGGTYMDGWGQGTLVTVSSASTKGPSVFPLAPSSKSTSGGTAALGCLVK
DYFPEPVTVSWNSGALTSGVHTFPAVLQSSGLYSLSSVVTVPSSSLGTQT
YICNVNHKPSNTKVDKKVEPKSCDKTHTCPPCPAPELLGGPSVFLFPPKP
KDTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREPQ
VYTLPPSRDELTKNQVSLTCLVKGFYPSDIAVEWESNGQPENNYKTTPPV
LDSDGSFFLYSKLTVDKSRWQQGNVFSCSVMHEALHNHYTQKSLSLSPG
K.

In some cases, an anti-mesothelin antibody comprises a VL present in the light chain amino acid sequence provided above; and a VH present in the heavy chain amino acid sequence provided above. For example, an anti-mesothelin antibody can comprise: a) a VL comprising an amino acid sequence having at least 90%, at least 95%, at least 98%, at least 99%, or 100%, amino acid sequence identity to the amino acid sequence: DIALTQPASVSGSPGQSITISCTGTSSDIGGYNSVSWYQQHPGKAPKLMIYGVNNRPSGVSNRFS GSKSGNTASLTISGLQAEDEADYYCSSYDIESATPVFGGGTK (SEQ ID NO:32); and b) a VH comprising an amino acid sequence having at least 90%, at least 95%, at least 98%, at least 99%, or 100%, amino acid sequence identity to the amino acid sequence:

(SEQ ID NO: 33)
QVELVQSGAEVKKPGESLKISCKGSGYSFTSYWIGWVRQAPGKGLEWMGI
IDPGDSRTRYSPSFQGQVTISADKSISTAYLQWSSLKASDTAMYYCARGQ
LYGGTYMDGWGQGTLVTVSS.

In some cases, an anti-mesothelin antibody comprises VL CDR1, VL CDR2, and VL CDR3 present in the light chain amino acid sequence provided above; and VH CDR1, CDR2, and CDR3 present in the heavy chain amino acid sequence provided above. In some cases, the V_{H} and V_{L} CDRs are as defined by Kabat (see, e.g., Table 1, above; and Kabat 1991). In some cases, the V_{H} and V_{L} CDRs are as defined by Chothia (see, e.g., Table 1, above; and Chothia 1987).

For example, an anti-mesothelin antibody can comprise a VL CDR1 having the amino acid sequence TGTSSDIGGYNSVS (SEQ ID NO:34); a VL CDR2 having the amino acid sequence LMIYGVNNRPS (SEQ ID NO:35); a VL CDR3 having the amino acid sequence SSYDIESATP (SEQ ID NO:36); a VH CDR1 having the amino acid sequence GYSFTSYWIG (SEQ ID NO:37); a VH CDR2 having the amino acid sequence WMGIIDPGDSRTRYSP (SEQ ID NO:38); and a VH CDR3 having the amino acid sequence GQLYGGTYMDG (SEQ ID NO:39).

An anti-mesothelin antibody can be a scFv. As one non-limiting example, an anti-mesothelin scFv can comprise the following amino acid sequence:

(SEQ ID NO: 40)
QVQLQQSGAEVKKPGASVKVSCKASGYTFTGYYMHWVRQAPGQGLEWMGR
INPNSGGTNYAQKFQGRVTMTRDTSISTAYMELSRLRSEDTAVYYCARGR
YYGMDVWGQGTMVTVSSGGGGSGGGGSGGGGSGGGGSEIVLTQSPATLSL
SPGERATISCRASQSVSSNFAWYQQRPGQAPRLLIYDASNRATGIPPRFS
GSGSGTDFTLTISSLEPED FAAYYCHQRSNWLYTFGQGTKVDIK,
where VH CDR1, CDR2, and CDR3 are underlined; and
VL CDR1, CDR2, and CDR3 are bolded and underlined.

As one non-limiting example, an anti-mesothelin scFv can comprise the following amino acid sequence:

(SEQ ID NO: 41)
QVQLVQSGAEVKKPGASVKVSCKASGYTFTGYYMHWVRQAPGQGLEWMGW
INPNSGGTNYAQKFQGRVTMTRDTSISTAYMELSRLRSDDTAVYYCARDL
RRTVVTPRAYYGMDVWGQGTTVTVSSGGGGSGGGGSGGGGSGGGGSDIQL
TQSPSTLSASVGDRVTITCQASQDISNSLNWYQQKAGKAPKLLIYDASTL
ETGVPSRFSGSGSGTDFSFTISSLQPEDIATYYCQQHDNLPLTFGQGTKV
EIK,
where VH CDR1, CDR2, and CDR3 are underlined; and
VL CDR1, CDR2, and CDR3 are bolded and underlined.

### 4) Anti-BCMA

Anti-BCMA (B-cell maturation antigen) antibodies are known in the art; and the VH and VL, or the VH and VL CDRs, of any anti-BCMA antibody can be included in a CAR. See, e.g., WO 2014/089335; US 2019/0153061; and WO 2017/093942.

In some cases, an anti-BCMA antibody comprises: a) a light chain comprising an amino acid sequence having at least 90%, at least 95%, at least 98%, at least 99%, or 100%, amino acid sequence identity to the following amino acid sequence:
QSVLTQPPSASGTPGQRVTISCSGSSSNIGSNTVNWYQQLPGTAPKLLIFNYHQRP SGVPDRFSGSKSGSSASLAISGLQSEDEADYYCAAWDDSLNGWVFGGGTKLTVLGQPKAAPSV TLFPPSSEELQANKATLVCLISDFYPGAVTVAWKADSSPVKAGVETTTPDSKQSNNKYAASSYL SLTPEQWKSHRSYSCQVTHEGSTVEKTVAPTECS (SEQ ID NO:42); and
b) a heavy chain comprising an amino acid sequence having at least 90%, at least 95%, at least 98%, at least 99%, or 100%, amino acid sequence identity to the following amino acid sequence:

(SEQ ID NO: 43)
EVQLVESGGGLVKPGGSLRLSCAASGFTFGDYALSWFRQAPGKGLEWVGV
SRSKAYGGTTDYAASVKGRFTISRDDSKSTAYLQMNSLKTEDTAVYYCAS
SGYSSGWTPFDYWGQGTLVTVSSASTKGPSVFPLAPSSKSTSGGTAALGC
LVKDYFPEPVTVSWNSGALTSGVHTFPAVLQSSGLYSLSSVVTVPSSSLG
TQTYICNVNHKPSNTKVDKKVEPKSCDKTHTCPPCPAPELLGGPSVFLFP
PKPKDTLMISRTPEVTCVVVDVSHEDPEVKFNWYVDGVEVHNAKTKPREE
QYNSTYRVVSVLTVLHQDWLNGKEYKCKVSNKALPAPIEKTISKAKGQPR
EPQVYTLPPSREEMTKNQVSLTCLVKGFYPSDIAVEWESNGQPENNYKTT
PPVLDSDGSFFLYSKLTVDKSRWQQGNVFSCSVMHEALHNHYTQKSLSLS
PGK.

In some cases, an anti-BCMA antibody comprises a VL present in the light chain amino acid sequence provided above; and a VH present in the heavy chain amino acid sequence provided above. For example, an anti-BCMA antibody can comprise: a) a VL comprising an amino acid sequence having at least 90%, at least 95%, at least 98%, at least 99%, or 100%, amino acid sequence identity to the amino acid sequence:
QSVLTQPPSASGTPGQRVTISCSGSSSNIGSNTVNWYQQLPGTAPKLLIFNYHQRP SGVPDRFSGSKSGSSASLAISGLQSEDEADYYCAAWDDSLNGWVFGGGTKLTVLG (SEQ ID NO:44); and b) a VH comprising an amino acid sequence having at least 90%, at least 95%, at least 98%, at least 99%, or 100%, amino acid sequence identity to the amino acid sequence:

(SEQ ID NO: 45)
EVQLVESGGGLVKPGGSLRLSCAASGFTFGDYALSWFRQAPGKGLEWVGV
SRSKAYGGTTDYAASVKGRFTISRDDSKSTAYLQMNSLKTEDTAVYYCAS
SGYSSGWTPFDYWGQGTLVTVSSASTKGPSV.

In some cases, an anti-BCMA antibody comprises VL CDR1, VL CDR2, and VL CDR3 present in the light chain amino acid sequence provided above; and VH CDR1, CDR2, and CDR3 present in the heavy chain amino acid sequence provided above. In some cases, the V_{H} and V_{L} CDRs are as defined by Kabat (see, e.g., Table 1, above; and Kabat 1991). In some cases, the V_{H} and V_{L} CDRs are as defined by Chothia (see, e.g., Table 1, above; and Chothia 1987).

For example, an anti-BCMA antibody can comprise a VL CDR1 having the amino acid sequence SSNIGSNT (SEQ ID NO:46), a VL CDR2 having the amino acid sequence NYH, a VL CDR3 having the amino acid sequence AAWDDSLNGWV (SEQ ID NO:47)), a VH CDR1 having the amino acid sequence GFTFGDYA (SEQ ID NO:48), a VH CDR2 having the amino acid sequence SRSKAYGGTT (SEQ ID NO:49), and a VH CDR3 having the amino acid sequence ASSGYSSGWTPFDY (SEQ ID NO:50).

An anti-BCMA antibody can be a scFv. As one non-limiting example, an anti-BCMA scFv can comprise the following amino acid sequence:

(SEQ ID NO: 51)
QVQLVQSGAEVKKPGSSVKVSCKASGGTFSNYWMHWVRQAPGQGLEWMGA
TYRGHSDTYYNQKFKGRVTITADKSTSTAYMELSSLRSEDTAVYYCARGA
IYNGYDVLDNWGQGTLVTVSSGGGGSGGGGSGGGGSGGGGSDIQMTQSPS
SLSASVGDRVTITCSASQDISNYLNWYQQKPGKAPKLLIYYTSNLHSGVP
SRFSGSGSGTDFTLTISSLQPEDFATYYCQQYRKLPWTFGQGTKLEIKR.

As another example, an anti-BCMA scFv can comprise the following amino acid sequence:

(SEQ ID NO: 52)
DIQMTQSPSSLSASVGDRVTITCSASQDISNYLNWYQQKPGKAPKLLIYY
TSNLHSGVPSRFSGSGSGTDFTLTISSLQPEDFATYYCQQYRKLPWTFGQ
GTKLEIKRGGGGSGGGGSGGGGSGGGGSQVQLVQSGAEVKKPGSSVKVSC
KASGGTFSNYWMHWVRQAPGQGLEWMGATYRGHSDTYYNQKFKGRVTITA
DKSTSTAYMELSSLRSEDTAVYYCARGAIYNGYDVLDNWGQGTLVTVSS.

In some cases, an anti-BCMA antibody can comprise a VL CDR1 having the amino acid sequence SASQDISNYLN (SEQ ID NO:53); a VL CDR2 having the amino acid sequence YTSNLHS (SEQ ID NO:54); a VL CDR3 having the amino acid sequence QQYRKLPWT (SEQ ID NO:55); a VH CDR1 having the amino acid sequence NYWMH (SEQ ID NO:56); a VH CDR2 having the amino acid sequence ATYRGHSDTYYNQKFKG (SEQ ID NO:57); and a VH CDR3 having the amino acid sequence GAIYNGYDVLDN (SEQ ID NO:58).

In some cases, an anti-BCMA antibody comprises: a) a light chain comprising an amino acid sequence having at least 90%, at least 95%, at least 98%, at least 99%, or 100%, amino acid sequence identity to the following amino acid sequence:

(SEQ ID NO: 59)
DIQMTQSPSSLSASVGDRVTITCSASQDISNYLNWYQQKPGKAPKLLIYY
TSNLHSGVPSRFSGSGSGTDFTLTISSLQPEDFATYYCQQYRKLPWTFGQ
GTKLEIKR.

In some cases, an anti-BCMA antibody comprises: a) a heavy chain comprising an amino acid sequence having at least 90%, at least 95%, at least 98%, at least 99%, or 100%, amino acid sequence identity to the following amino acid sequence:

(SEQ ID NO: 60)
QVQLVQSGAEVKKPGSSVKVSCKASGGTFSNYWMHWVRQAPGQGLEWMGA
TYRGHSDTYYNQKFKGRVTITADKSTSTAYMELSSLRSEDTAVYYCARGA
IYDGYDVLDNWGQGTLVTVSS.

In some cases, an anti-BCMA antibody (e.g., an antibody referred to in the literature as belantamab) comprises a light chain comprising the amino acid sequence: DIQMTQSPSSLSASVGDRVTITCSASQDISNYLNWYQQKPGKAPKLLIYYTSNLHSGVPSRFSGS GSGTDFTLTISSLQPEDFATYYCQQYRKLPWTFGQGTKLEIKR (SEQ ID NO:59); and a heavy chain comprising the amino acid sequence:

(SEQ ID NO: 60)
QVQLVQSGAEVKKPGSSVKVSCKASGGTFSNYWMHWVRQAPGQGLEWMGA
TYRGHSDTYYNQKFKGRVTITADKSTSTAYMELSSLRSEDTAVYYCARGA
IYDGYDVLDNWGQGTLVTVSS.

In some cases, the anti-BCMA antibody has a cancer chemotherapeutic agent linked to the antibody. For example, in some cases, the anti-BCMA antibody is GSK2857916 (belantamab-mafodotin), where monomethyl auristatin F (MMAF) is linked via a maleimidocaproyl linker to the anti-BCMA antibody belantamab.

### 5) Anti-MUC1

In some cases, an antigen-binding polypeptide present in a CAR is a single-chain Fv specific for MUC1. See, e.g., Singh et al. (2007) Mol. Cancer Ther. 6:562; Thie et al. (2011) PLoSOne 6:e15921; Imai et al. (2004) Leukemia 18:676; Posey et al. (2016) Immunity 44:1444; EP3130607; EP3164418; WO 2002/044217; and US 2018/0112007. In some cases, an antigen-binding polypeptide present in a CAR is a scFv specific for the MUC1 peptide VTSAPDTRPAPGSTAPPAHG (SEQ ID NO:61). In some cases, a TTP is a scFv specific for the MUC1 peptide SNIKFRPGSVVVQLTLAFREGTINVHDVETQFNQYKTEAASRY (SEQ ID NO:62). In some cases, an antigen-binding polypeptide present in a CAR is a scFv specific for the MUC1 peptide SVVVQLTLAFREGTINVHDVETQFNQYKTEAASRY (SEQ ID NO:63). In some cases, a TTP is a scFv specific for the MUC1 peptide LAFREGTINVHDVETQFNQY (SEQ ID NO:64). In some cases, an antigen-binding polypeptide present in a CAR is a scFv specific for the MUC1 peptide SNIKFRPGSVVVQLTLAAFREGTIN (SEQ ID NO:65).

As an example, an anti-MUC1 antibody can comprise: a VH CDR1 having the amino acid sequence RYGMS (SEQ ID NO:66); a VH CDR2 having the amino acid sequence TISGGGTYIYYPDSVKG (SEQ ID NO: 67); a VH CDR3 having the amino acid sequence DNYGRNYDYGMDY (SEQ ID NO:68); a VL CDR1 having the amino acid sequence SATSSVSYIH (SEQ ID NO:69); a VL CDR2 having the amino acid sequence STSNLAS (SEQ ID NO:70); and a VL CDR3 having the amino acid sequence QQRSSSPFT (SEQ ID NO:71). See, e.g., US 2018/0112007.

As another example, an anti-MUC1 antibody can comprise a VH CDR1 having the amino acid sequence GYAMS (SEQ ID NO:72); a VH CDR2 having the amino acid sequence TISSGGTYIYYPDSVKG (SEQ ID NO:73); a VH CDR3 having the amino acid sequence LGGDNYYEYFDV (SEQ ID NO: 74); a VL CDR1 having the amino acid sequence RASKSVSTSGYSYMH (SEQ ID NO:75); a VL CDR2 having the amino acid sequence LASNLES (SEQ ID NO:76); and a VL CDR3 having the amino acid sequence QHSRELPFT (SEQ ID NO:77). See, e.g., US 2018/0112007.

As another example, an anti-MUC1 antibody can comprise a VH CDR1 having the amino acid sequence DYAMN (SEQ ID NO:78); a VH CDR2 having the amino acid sequence VISTFSGNINFNQKFKG (SEQ ID NO: 79); a VH CDR3 having the amino acid sequence SDYYGPYFDY (SEQ ID NO: 80); a VL CDR1 having the amino acid sequence RSSQTIVHSNGNTYLE (SEQ ID NO:81); a VL CDR2 having the amino acid sequence KVSNRFS (SEQ ID NO:82); and a VL CDR3 having the amino acid sequence ((SEQ ID NO:83). See, e.g., US 2018/0112007.

As another example, an anti-MUC1 antibody can comprise a VH CDR1 having the amino acid sequence GYAMS (SEQ ID NO:72); a VH CDR2 having the amino acid sequence TISSGGTYIYYPDSVKG (SEQ ID NO:73); a VH CDR3 having the amino acid sequence LGGDNYYEY (SEQ ID NO:84); a VL CDR1 having the amino acid sequence TASKSVSTSGYSYMH (SEQ ID NO:85); a VL CDR2 having the amino acid sequence LVSNLES (SEQ ID NO:86); and a VL CDR3 having the amino acid sequence QHIRELTRSE (SEQ ID NO:87). See, e.g., US 2018/0112007.

### 6) Anti-MUC16

In some cases, an antigen-binding polypeptide present in a CAR is specific for a MUC16 polypeptide present on a cancer cell. See, e.g., US 2018/0118848; and US 2018/0112008. In some cases, a MUC16-specific antigen-binding polypeptide is a scFv. In some cases, a MUC16-specific antigen-binding polypeptide is a nanobody.

As one example, an anti-MUC16 antibody can comprise a VH CDR1 having the amino acid sequence GFTFSNYY (SEQ ID NO:88); a VH CDR2 having the amino acid sequence ISGRGSTI (SEQ ID NO:89); a VH CDR3 having the amino acid sequence VKDRGGYSPY (SEQ ID NO:90); a VL CDR1 having the amino acid sequence QSISTY (SEQ ID NO:91); a VL CDR2 having the amino acid sequence TAS; and a VL CDR3 having the amino acid sequence QQSYSTPPIT (SEQ ID NO:92). See, e.g., US 2018/0118848.

### 7) Examples of Antigen-Binding Domains

In some cases, a suitable CAR comprises a scFv specific for CD19. For example, in some cases, an anti-CD19 scFv comprises an amino acid sequence having at least 90%, at least 95%, at least 98%, at least 99%, or 100%, amino acid sequence identity to the following amino acid sequence:

(SEQ ID NO: 29)
DIQLTQSPASLAVSLGQRATISCKASQSVDYDGDSYLNWYQQIPGQPPKL
LIYDASNLVSGIPPRFSGSGSGTDFTLNIHPVEKVDAATYHCQQSTEDPW
TFGGGTKLEIKGGGGSGGGGSGGGGSQVQLQQSGAELVRPGSSVKISCKA
SGYAFSSYWMNWVKQRPGQGLEWIGQIWPGDGDTNYNGKFKGKATLTADE
SSSTAYMQLSSLASEDSAVYFCARRETTTVGRYYYAMDYWGQGTTVTVS.

In some cases, a suitable CAR comprises a scFv specific for mesothelin. For example, in some cases, an anti-mesothelin scFv comprises an amino acid sequence having at least 90%, at least 95%, at least 98%, at least 99%, or 100%, amino acid sequence identity to the following amino acid sequence:

(SEQ ID NO: 40)
QVQLQQSGAEVKKPGASVKVSCKASGYTFTGYYMHWVRQAPGQGLEWMG
RINPNSGGTNYAQKFQGRVTMTRDTSISTAYMELSRLRSEDTAVYYCAR
GRYYGMDVWGQGTMVTVSSGGGGSGGGGSGGGGSGGGGSEIVLTQSPAT
LSLSPGERATISCRASQSVSSNFAWYQQRPGQAPRLLIYDASNRATGIP
PRFSGSGSGTDFTLTISSLEPED FAAYYCHQRSNWLYTFGQGTKVDIK.

In some cases, an anti-mesothelin scFv comprises an amino acid sequence having at least 90%, at least 95%, at least 98%, at least 99%, or 100%, amino acid sequence identity to the following amino acid sequence:

(SEQ ID NO: 41)
QVQLVQSGAEVKKPGASVKVSCKASGYTFTGYYMHWVRQAPGQGLEWMG
WINPNSGGTNYAQKFQGRVTMTRDTSISTAYMELSRLRSDDTAVYYCAR
DLRRTVVTPRAYYGMDVWGQGTTVTVSSGGGGSGGGGSGGGGSGGGGSD
IQLTQSPSTLSASVGDRVTITCQASQDISNSLNWYQQKAGKAPKLLIYD
ASTLETGVPSRFSGSGSGTDFSF
TISSLQPEDIATYYCQQHDNLPLTFGQGTKVEIK.

In some cases, a suitable CAR comprises a scFv specific for B-cell maturation antigen (BCMA). For example, in some cases, an anti-BCMA scFv comprises an amino acid sequence having at least 90%, at least 95%, at least 98%, at least 99%, or 100%, amino acid sequence identity to the following amino acid sequence:

(SEQ ID NO: 51)
QVQLVQSGAEVKKPGSSVKVSCKASGGTFSNYWMHWVRQAPGQGLEWMG
ATYRGHSDTYYNQKFKGRVTITADKSTSTAYMELSSLRSEDTAVYYCAR
GAIYNGYDVLDNWGQGTLVTVSSGGGGSGGGGSGGGGSGGGGSDIQMTQ
SPSSLSASVGDRVTITCSASQDISNYLNWYQQKPGKAPKLLIYYTSNLH
SGVPSRFSGSGSGTDFTLTISSLQPEDFATYYCQQYRKLPWTFGQGTKL
EIKR.

In some cases, an anti-BCMA scFv comprises an amino acid sequence having at least 90%, at least 95%, at least 98%, at least 99%, or 100%, amino acid sequence identity to the following amino acid sequence:

(SEQ ID NO: 52)
DIQMTQSPSSLSASVGDRVTITCSASQDISNYLNWYQQKPGKAPKLLIY
YTSNLHSGVPSRFSGSGSGTDFTLTISSLQPEDFATYYCQQYRKLPWTF
GQGTKLEIKRGGGGSGGGGSGGGGSGGGGSQVQLVQSGAEVKKPGSSVK
VSCKASGGTFSNYWMHWVRQAPGQGLEWMGATYRGHSDTYYNQKFKGRV
TITADKSTSTAYMELSSLRSEDTAVYYCARGAIYNGYDVLDNWGQGTLV
TVSS.

### Hinge Region

As noted above, a CAR can include a hinge region between the extracellular domain and the transmembrane domain. As used herein, the term “hinge region” refers to a flexible polypeptide connector region (also referred to herein as “hinge” or “spacer”) providing structural flexibility and spacing to flanking polypeptide regions and can consist of natural or synthetic polypeptides. The hinge region can include complete hinge region derived from an antibody of a different class or subclass from that of the CH1 domain. The term “hinge region” can also include regions derived from CD8 and other receptors that provide a similar function in providing flexibility and spacing to flanking regions.

The hinge region can have a length of from about 4 amino acids to about 50 amino acids, e.g., from about 4 aa to about 10 aa, from about 10 aa to about 15 aa, from about 15 aa to about 20 aa, from about 20 aa to about 25 aa, from about 25 aa to about 30 aa, from about 30 aa to about 40 aa, or from about 40 aa to about 50 aa.

As non-limiting examples, an immunoglobulin hinge region can include one of the following amino acid sequences: DKTHT (SEQ ID NO:93); CPPC (SEQ ID NO:94); CPEPKSCDTPPPCPR (SEQ ID NO:95); ELKTPLGDTTHT (SEQ ID NO:96); KSCDKTHTCP (SEQ ID NO: 97); KCCVDCP (SEQ ID NO:98); KYGPPCP (SEQ ID NO:99); EPKSCDKTHTCPPCP (SEQ ID NO: 100) (human IgG1 hinge); ERKCCVECPPCP (SEQ ID NO:101) (human IgG2 hinge); ELKTPLGDTTHTCPRCP (SEQ ID NO: 102) (human IgG3 hinge); SPNMVPHAHHAQ (SEQ ID NO: 103) (human IgG4 hinge); and the like. The hinge region can comprise an amino acid sequence derived from human CD8; e.g., the hinge region can comprise the amino acid sequence: TTTPAPRPPTPAPTIASQPLSLRPEACRPAAGGAVHTRGLDFACD (SEQ ID NO: 104), or a variant thereof.

### Transmembrane Domain

Any transmembrane (TM) domain that provides for insertion of a polypeptide into the cell membrane of a eukaryotic (e.g., mammalian) cell is suitable for use. The transmembrane region of a CAR can be derived from (i.e. comprise at least the transmembrane region(s) of) the alpha, beta or zeta chain of the T-cell receptor, CD28, CD3 epsilon, CD45, CD4, CD5, CD8 (e.g., CD8 alpha, CD8 beta), CD9, CD16, CD22, CD33, CD37, CD64, CD80, CD86, CD134, CD137, or CD154, KIRDS2, OX40, CD2, CD27, LFA-1 (CD11a, CD18), ICOS (CD278), 4-1BB (CD137), GITR, CD40, BAFFR, HVEM (LIGHTR), SLAMF7, NKp80 (KLRF1), CD160, CD19, IL2Rbeta, IL2Rgamma, IL7R .alpha., ITGA1, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), SLAMF6 (NTB-A, Ly108), SLAM (SLAMF1, CD150, IPO-3), BLAME (SLAMF8), SELPLG (CD162), LTBR, and PAG/Cbp. The transmembrane domain can be synthetic, in which case it can comprise predominantly hydrophobic residues such as leucine and valine. In some cases, a triplet of phenylalanine, tryptophan and valine will be found at each end of a synthetic transmembrane domain.

As one non-limiting example, the TM sequence IYIWAPLAGTCGVLLLSLVITLYC (SEQ ID NO: 105) can be used. Additional non-limiting examples of suitable TM sequences include: a) CD8 beta derived TM: LGLLVAGVLVLLVSLGVAIHLCC (SEQ ID NO: 106); b) CD4 derived TM: ALIVLGGVAGLLLFIGLGIFFCVRC (SEQ ID NO: 107); c) CD3 zeta derived TM: LCYLLDGILFIYGVILTALFLRV (SEQ ID NO: 108); d) CD28 derived TM: WVLVVVGGVLACYSLLVTVAFIIFWV (SEQ ID NO: 109); e) CD134 (OX40) derived TM: VAAILGLGLVLGLLGPLAILLALYLL (SEQ ID NO: 110); and f) CD7 derived TM: ALPAALAVISFLLGLGLGVACVLA (SEQ ID NO:111).

### Intracellular Domain—Co-Stimulatory Polypeptide

The intracellular portion (cytoplasmic domain) of a CAR can comprise one or more co-stimulatory polypeptides. Non-limiting examples of suitable co-stimulatory polypeptides include, but are not limited to, 4-1BB (CD137), CD28, ICOS, OX-40, BTLA, CD27, CD30, GITR, and HVEM. Suitable co-stimulatory polypeptides include, e.g.: 1) a 4-1BB polypeptide having at least 90%, at least 95%, at least 98%, or 100%, amino acid sequence identity to the following amino acid sequence: KRGRKKLLYIFKQPFMRPVQTTQEEDGCSCRFPEEEEGGCEL (SEQ ID NO: 112); 2) a CD28 polypeptide having at least 90%, at least 95%, at least 98%, or 100%, amino acid sequence identity to the following amino acid sequence: FWVRSKRSRLLHSDYMNMTPRRPGPTRKHYQPYAPPRDFAAYRS (SEQ ID NO: 113); 3) an ICOS polypeptide having at least 90%, at least 95%, at least 98%, or 100%, amino acid sequence identity to the following amino acid sequence: TKKKYSSSVHDPNGEYMFMRAVNTAKKSRLTDVTL (SEQ ID NO: 114); 4) an OX40 polypeptide having at least 90%, at least 95%, at least 98%, or 100%, amino acid sequence identity to the following amino acid sequence: RRDQRLPPDAHKPPGGGSFRTPIQEEQADAHSTLAKI (SEQ ID NO: 115); 5) a BTLA polypeptide having at least 90%, at least 95%, at least 98%, or 100%, amino acid sequence identity to the following amino acid sequence: CCLRRHQGKQNELSDTAGREINLVDAHLKSEQTEASTRQNSQVLLSETGIYDNDPDLCFRMQEG SEVYSNPCLEENKPGIVYASLNHSVIGPNSRLARNVKEAPTEYASICVRS (SEQ ID NO: 116); 6) a CD27 polypeptide having at least 90%, at least 95%, at least 98%, or 100%, amino acid sequence identity to the following amino acid sequence: HQRRKYRSNKGESPVEPAEPCRYSCPREEEGSTIPIQEDYRKPEPACSP (SEQ ID NO: 117); 7) a CD30 polypeptide having at least 90%, at least 95%, at least 98%, or 100%, amino acid sequence identity to the following amino acid sequence: RRACRKRIRQKLHLCYPVQTSQPKLELVDSRPRRSSTQLRSGASVTEPVAEERGLMSQPLMETC HSVGAAYLESLPLQDASPAGGPSSPRDLPEPRVSTEHTNNKIEKIYIMKADTVIVGTVKAELPEG RGLAGPAEPELEEELEADHTPHYPEQETEPPLGSCSDVMLSVEEEGKEDPLPTAASGK (SEQ ID NO: 118); 8) a GITR polypeptide having at least 90%, at least 95%, at least 98%, or 100%, amino acid sequence identity to the following amino acid sequence: HIWQLRSQCMWPRETQLLLEVPPSTEDARSCQFPEEERGERSAEEKGRLGDLWV (SEQ ID NO: 119); and 9) an HVEM polypeptide having at least 90%, at least 95%, at least 98%, or 100%, amino acid sequence identity to the following amino acid sequence: CVKRRKPRGDVVKVIVSVQRKRQEAEGEATVIEALQAPPDVTTVAVEETIPSFTGRSPNH (SEQ ID NO: 120). The co-stimulatory polypeptide can have a length of from about 30 aa to about 35 aa, from about 35 aa to about 40 aa, from about 40 aa to about 45 aa, from about 45 aa to about 50 aa, from about 50 aa to about 55 aa, from about 55 aa to about 60 aa, from about 60 aa to about 65 aa, or from about 65 aa to about 70 aa.

### Intracellular Domain—Signaling Polypeptide

The intracellular portion of a CAR can comprise a signaling polypeptide. Suitable signaling polypeptides include, e.g., an immunoreceptor tyrosine-based activation motif (ITAM)-containing intracellular signaling polypeptide. An ITAM motif is YX₁X₂L/I, (SEQ ID NO: 121) where X₁ and X₂ are independently any amino acid. In some cases, the intracellular signaling domain of a subject CAR comprises 1, 2, 3, 4, or 5 ITAM motifs. In some cases, an ITAM motif is repeated twice in an intracellular signaling domain, where the first and second instances of the ITAM motif are separated from one another by 6 to 8 amino acids, e.g., (YX₁X₂L/I)(X₃)ₙ(YX₁X₂L/I) (SEQ ID NO: 122), where n is an integer from 6 to 8, and each of the 6-8 X₃ can be any amino acid. In some cases, the intracellular signaling domain of a CAR comprises 3 ITAM motifs.

A suitable intracellular signaling domain can be an ITAM motif-containing portion that is derived from a polypeptide that contains an ITAM motif. For example, a suitable intracellular signaling domain can be an ITAM motif-containing domain from any ITAM motif-containing protein. Thus, a suitable intracellular signaling domain need not contain the entire sequence of the entire protein from which it is derived. Examples of suitable ITAM motif-containing polypeptides include, but are not limited to: DAP12; FCER1G (Fc epsilon receptor I gamma chain); CD3D (CD3 delta); CD3E (CD3 epsilon); CD3G (CD3 gamma); CD3Z (CD3 zeta); and CD79A (antigen receptor complex-associated protein alpha chain).

### TCR

As noted above, target modified T cells (e.g., target modified CTLs) comprise (e.g., express on their cell surface) a TCR specific for a preselected antigen present in a human. Such antigens can be antigens of pathogens that infect humans. Such antigens can be antigens present in vaccines administered to humans. In some cases, the antigen is a viral antigen. In some cases, a viral antigen is encoded by a virus that infects a majority of the human population, where such viruses include, e.g., cytomegalovirus (CMV), Epstein-Barr virus (EBV), human papilloma virus, influenza virus, adenovirus, and the like. In some cases, the antigen is a bacterial epitope, e.g., a bacterial epitope that is included in a vaccine and to which a majority of the human population has immunity. For example, in some cases, the antigen is a tetanus antigen.

### CMV Peptides

In some cases, the TCR present on the surface of a target modified T cell binds a CMV peptide. In some cases, the TCR present on the surface of a target modified T cell binds a peptide from CMV pp65. In some cases, the TCR present on the surface of a target modified T cell binds a peptide from CMV gB (glycoprotein B).

For example, in some cases, the TCR present on the surface of a target modified T cell binds a CMV polypeptide having a length of at least 4 amino acids, e.g., from 4 amino acids to about 25 amino acids (e.g., 4 amino acids (aa), 5 aa, 6 aa, 7 aa, 8 aa, 9 aa, 10 aa, 11 aa, 12 aa, 13 aa, 14 aa, 15 aa, 16 aa, 17 aa, 18 aa, 19 aa, 20 aa, 21 aa, 22 aa, 23 aa, 24 aa, or 25 aa, including within a range of from 4 to 20 aa, from 6 to 18 aa, from 8 to 15 aa. from 8 to 12 aa, from 5 to 10 aa, from 10 to 15 aa, from 15 to 20 aa, from 10 to 20 aa, or from 15 to 25 aa. in length), and comprising an amino acid sequence having at least 80%, at least 85%, at least 90%, at least 95%, at least 98%, at least 99%, or 100%, amino acid sequence identity to the following CMV pp65 amino acid sequence:

(SEQ ID NO: 123)
MESRGRRCPE MISVLGPISG HVLKAVFSRG DTPVLPHETR
LLQTGIHVRV SQPSLILVSQ YTPDSTPCHR GDNQLQVQHT
YFTGSEVENV SVNVHNPTGR SICPSQEPMS IYVYALPLKM
LNIPSINVHH YPSAAERKHR HLPVADAVIH ASGKQMWQAR
LTVSGLAWTR QQNQWKEPDV YYTSAFVFPT KDVALRHVVC
AHELVCSMEN TRATKMQVIG DQYVKVYLES FCEDVPSGKL
FMHVTLGSDV EEDLTMTRNP QPFMRPHERN GFTVLCPKNM
IIKPGKISHI MLDVAFTSHE HFGLLCPKSI PGLSISGNLL
MNGQQIFLEV QAIRETVELR QYDPVAALFF FDIDLLLQRG
PQYSEHPTFT SQYRIQGKLE YRHTWDRHDE GAAQGDDDVW
TSGSDSDEEL VTTERKTPRV TGGGAMAGAS TSAGRKRKSA
SSATACTSGV MTRGRLKAES TVAPEEDTDE DSDNEIHNPA
VFTWPPWQAG ILARNLVPMV ATVQGQNLKY QEFFWDANDI
YRIFAELEGV WQPAAQPKRR RHRQDALPGP CIASTPKKHR G.

As one non-limiting example, the TCR present on the surface of a target modified T cell binds a peptide having the amino acid sequence NLVPMVATV (SEQ ID NO: 172) and having a length of 9 amino acids.

In some cases, the TCR present on the surface of a target modified T cell binds a peptide having a length of at least 4 amino acids, e.g., from 4 amino acids to about 25 amino acids (e.g., 4 amino acids (aa), 5 aa, 6 aa, 7 aa, 8 aa, 9 aa, 10 aa, 11 aa, 12 aa, 13 aa, 14 aa, 15 aa, 16 aa, 17 aa, 18 aa, 19 aa, 20 aa, 21 aa, 22 aa, 23 aa, 24 aa, or 25 aa, including within a range of from 4 to 20 aa, from 6 to 18 aa, from 8 to 15 aa. from 8 to 12 aa, from 5 to 10 aa, from 10 to 15 aa, from 15 to 20 aa, from 10 to 20 aa, or from 15 to 25 aa. in length) of a CMV polypeptide comprising an amino acid sequence having at least 80%, at least 85%, at least 90%, at least 95%, at least 98%, at least 99%, or 100%, amino acid sequence identity to the following CMV gB amino acid sequence:

(SEQ ID NO: 123)
MESRIWCLVVCVNLCIVCLGAAVSSSSTSHATSSTHNGSHTSRTTSAQ
TRSVYSQHVTSSEAVSHRANETIYNTTLKYGDVVGVNTTKYPYRVCSM
AQGTDLIRFERNIICTSMKPINEDLDEGIMVVYKRNIVAHTFKVRVYQ
KVLTFRRSYAYIYTTYLLGSNTEYVAPPMWEIHHINKFAQCYSSYSRV
IGGTVFVAYHRDSYENKTMQLIPDDYSNTHSTRYVTVKDQWHSRGSTW
LYRETCNLNCMLTITTARSKYPYHFFATSTGDVVYISPFYNGTNRNAS
YFGENADKFFIFPNYTIVSDFGRPNAAPETHRLVAFLERADSVISWDI
QDEKNVTCQLTFWEASERTIRSEAEDSYHFSSAKMTATFLSKKQEVNM
SDSALDCVRDEAINKLQQIFNTSYNQTYEKYGNVSVFETSGGLVVFWQ
GIKQKSLVELERLANRSSLNITHRTRRSTSDNNTTHLSSMESVHNLVY
AQLQFTYDTLRGYINRALAQIAEAWCVDQRRTLEVFKELSKINPSAIL
SAIYNKPIAARFMGDVLGLASCVTINQTSVKVLRDMNVKESPGRCYSR
PVVIFNFANSSYVQYGQLGEDNEILLGNHRTEECQLPSLKIFIAGNSA
YEYVDYLFKRMIDLSSISTVDSMIALDIDPLENTDFRVLELYSQKELR
SSNVFDLEEIMREFNSYKQRVKYVEDKVVDPLPPYLKGLDDLMSGLGA
AGKAVGVAIGAVGGAVASVVEGVATFLKNPFGAFTIILVAIAVVIITY
LIYTRQRRLCTQPLQNLFPYLVSADGTTVTSGSTKDTSLQAPPSYEES
VYNSGRKGPGPPSSDASTAAPPYTNEQAYQMLLALARLDAEQRAQQNG
TDSLDGQTGTQDKGQKPNLLDRLRHRKNGYRHLKDSDEEENV.

In some cases, the TCR present on the surface of a target modified T cell binds a CMV peptide when presented with an HLA complex comprising an MHC class I heavy chain selected from HLA-A*0101, A*0201, A*0301, A*1101, A*2301, A*2402, A*2407, A*3303, and A*3401. In some cases, the TCR present on the surface of a target modified T cell binds a CMV peptide that is restricted to HLA-B*0702, B*0801, B*1502, B*3802, B*4001, B*4601, and/or B*5301. In some cases, the TCR present on the surface of a target modified T cell binds a CMV peptide that is restricted to C*0102, C*0303, C*0304, C*0401, C*0602, C*0701, C*702, C*0801, and/or C*1502. As one example, in some cases, the TCR present on the surface of a target modified T cell binds a peptide epitope having amino acid sequence NLVPMVATV (SEQ ID NO: 172) and having a length of 9 amino acids; where the CMV peptide is presented in a complex comprising: i) an HLA-A*0201 class I heavy chain polypeptide; and ii) a β2M polypeptide.

### HPV Peptides

In some cases, the TCR present on the surface of a target modified T cell binds a peptide of an HPV E6 polypeptide or an HPV E7 polypeptide. The HPV epitope can be an epitope of HPV of any of a variety of genotypes, including, e.g., HPV16, HPV18, HPV31, HPV33, HPV35, HPV39, HPV45, HPV51, HPV52, HPV56, HPV58, HPV59, HPV68, HPV73, or HPV82. In some cases, the epitope is an HPV E6 epitope. In some cases, the epitope is an HPV E7 epitope.

Examples of HPV E6 peptides that can be bound by the TCR present on the surface of a target modified T cell include, but are not limited to, E6 18-26 (KLPQLCTEL; SEQ ID NO: 124); E6 26-34 (LQTTIHDII; SEQ ID NO: 125); E6 49-57 (VYDFAFRDL; SEQ ID NO: 126); E6 52-60 (FAFRDLCIV; SEQ ID NO: 127); E6 75-83 (KFYSKISEY; SEQ ID NO: 128); and E6 80-88 (ISEYRHYCY; SEQ ID NO: 129).

Examples of HPV E7 peptides that can be bound by the TCR present on the surface of a target modified T cell include, but are not limited to, E7 7-15 (TLHEYMLDL; SEQ ID NO:130); E7 11-19 (YMLDLQPET; SEQ ID NO:131); E7 44-52 (QAEPDRAHY; SEQ ID NO: 132); E7 49-57 (RAHYNIVTF (SEQ ID NO: 133); E7 61-69 (CDSTLRLCV; SEQ ID NO: 134); and E7 67-76 (LCVQSTHVDI; SEQ ID NO: 135); E7 82-90 (LLMGTLGIV; SEQ ID NO: 136); E7 86-93 (TLGIVCPI; SEQ ID NO: 137); and E7 92-93 (LLMGTLGIVCPI; SEQ ID NO:138).

In some cases, the TCR present on the surface of a target modified T cell binds an HPV peptide, where the HPV peptide is an HPV E6 peptide that is bound to an MHC complex comprising a β2M polypeptide and an HLA-A24 heavy chain. Non-limiting examples of such HPV E6 peptides include: VYDFAFRDL (SEQ ID NO: 126); CYSLYGTTL (SEQ ID NO: 139); EYRHYCYSL (SEQ ID NO: 140); KLPQLCTEL (SEQ ID NO: 124); DPQERPRKL (SEQ ID NO:141); HYCYSLYGT (SEQ ID NO: 142); DFAFRDLCI (SEQ ID NO: 143); LYGTTLEQQY (SEQ ID NO: 144); HYCYSLYGTT (SEQ ID NO: 145); EVYDFAFRDL (SEQ ID NO: 146); EYRHYCYSLY (SEQ ID NO: 147); VYDFAFRDLC (SEQ ID NO: 148); YCYSIYGTTL (SEQ ID NO: 149); VYCKTVLEL (SEQ ID NO: 150); VYGDTLEKL (SEQ ID NO:151); and LTNTGLYNLL (SEQ ID NO: 152).

In some cases, the TCR present on the surface of a target modified T cell binds an HPV peptide selected from the group consisting of: DLQPETTDL (SEQ ID NO: 153); TLHEYMLDL (SEQ ID NO: 130); TPTLHEYML (SEQ ID NO: 154); RAHYNIVTF (SEQ ID NO: 133); GTLGIVCPI (SEQ ID NO: 155); EPDRAHYNI (SEQ ID NO: 156); QLFLNTLSF (SEQ ID NO: 157); FQQLFLNTL (SEQ ID NO:158); and AFQQLFLNTL (SEQ ID NO: 159).

In some cases, the TCR present on the surface of a target modified T cell binds an HPV peptide that presents an HLA-A*2401-restricted epitope. Non-limiting examples of HPV peptides presenting an HLA-A*2401-restricted epitope are: VYDFAFRDL (SEQ ID NO: 127); RAHYNIVTF (SEQ ID NO: 160); CDSTLRLCV (SEQ ID NO: 134); and LCVQSTHVDI (SEQ ID NO: 135). In some cases, the TCR present on the surface of a target modified T cell binds the peptide VYDFAFRDL (SEQ ID NO: 126). In some cases, the TCR present on the surface of a target modified T cell binds the peptide RAHYNIVTF (SEQ ID NO: 133). In some cases, the TCR present on the surface of a target modified T cell binds the peptide CDSTLRLCV (SEQ ID NO: 134). In some cases, the TCR present on the surface of a target modified T cell binds the peptide LCVQSTHVDI (SEQ ID NO: 135).

### Influenza Virus Peptides

Influenza virus peptides that can be bound by the TCR present on the surface of a target modified T cell include peptides of from 4 amino acids to 25 amino acids in length of an influenza polypeptide, e.g., an influenza polypeptide that is included in a vaccine, or that is present in an influenza virus that infects a human. As one example, the TCR present on the surface of a target modified T cell binds an influenza virus peptide of from 4 amino acids to 25 amino acids in length of an influenza virus nucleoprotein. As another example, the TCR present on the surface of a target modified T cell binds a peptide of from 4 amino acids to 25 amino acids in length of an influenza virus hemagglutinin polypeptide. As another example, the TCR present on the surface of a target modified T cell binds a peptide of from 4 amino acids to 25 amino acids in length of an influenza A virus Matrix protein 1. As another example, the TCR present on the surface of a target modified T cell binds a peptide of from 4 amino acids to 25 amino acids in length of an influenza virus neuraminidase polypeptide. In some cases, the peptide is a peptide that presents an immunodominant influenza virus protein epitope. One non-limiting example of a suitable influenza peptide is a peptide having the sequence GILGFVFTL (SEQ ID NO: 160) and having a length of 9 amino acids.

### Tetanus Peptides

In some cases, the TCR present on the surface of a target modified T cell binds a tetanus peptide (e.g., a peptide of an antigen present in a tetanus vaccine). Tetanus peptides that can be bound by the TCR present on the surface of a target modified T cell include peptides of from 4 amino acids to 25 amino acids in length of a tetanus toxin. Examples of such tetanus peptides include, but are not limited to, QYIKANSKFIGIFE (SEQ ID NO:161); QYIKANSKFIGITE (SEQ ID NO: 162); ILMQYIKANSKFIGI (SEQ ID NO: 163); VNNESSE (SEQ ID NO: 164); PGINGKAIHLVNNESSE (SEQ ID NO: 165); PNRDIL (SEQ ID NO: 166); FIGITEL (SEQ ID NO:167); SYFPSV (SEQ ID NO: 168); NSVDDALINSTKIYSYFPSV (SEQ ID NO: 169); and IDKISDVSTIVPYIGPALNI (SEQ ID NO: 170).

### Methods of Making a Modified CTL

The present disclosure provides a method of making an in vitro composition of cells of the present disclosure. In some cases, the method comprises: a) providing a composition comprising a quantity of T cells, wherein the quantity comprises target T cells having a TCR specific for a preselected antigen; b) at least partially separating target T cells from non-target T cells comprising a TCR that is not specific for the preselected antigen, thereby generating an enriched target T cell population; and c) modifying the target T cells in the enriched target T cell population by introducing into the target T cells one or more nucleic acids comprising nucleotide sequences encoding a CAR, where the CAR comprises an antigen-binding domain specific for a cancer-associated antigen. In some cases, the method comprises: a) at least partially separating target T cells having a TCR specific for a preselected antigen from non-target T cells comprising a TCR that is not specific for the preselected antigen present in a heterogeneous population of T cells, thereby generating an enriched target T cell population; and b) modifying the target T cells in the enriched target T cell population by introducing into the target T cells one or more nucleic acids comprising nucleotide sequences encoding a CAR, where the CAR comprises an antigen-binding domain specific for a cancer-associated antigen.

### Cell Separation

Target T cells (e.g., CTLs) having a TCR specific for a preselected antigen) can be enriched from a quantity of cells (a “starting cell population”; or “heterogeneous cell population”; or “heterogeneous starting cell population”) obtained from an individual, e.g., a patient who has been vaccinated with a particular antigen of interest or who has been infected with a virus. The starting cell population can be whole blood, peripheral blood mononuclear cells (PBMCs), cells obtained by leukapheresis, or other starting cell population that includes leukocytes.

The starting cell population comprises cells having a TCR specific for a preselected antigen (e.g., a viral antigen; a bacterial antigen; as described above). T cells having a TCR specific for a preselected antigen are collectively referred to as “target T cells.” The starting cell population also comprises T cells having a TCR specific for an antigen other than the preselected antigen; such cells are referred to as “non-target T cells.” Thus, the starting cell population comprises both target T cells and non-target T cells.

The proportion of target T cells in the starting T cell population generally will be low, e.g., well less than 1%, but it also can be higher, e.g., if a patient is experiencing an active infection such as the flu that causes a natural increase in target T cells that are specific for a flu antigen. Hence, the proportion can range, e.g., from less than 0.010% to about 10% or higher. For example, the proportion of target T cells in the starting cell population can be from less than 0.01% to 0.01%, from 0.01% to 0.05%, from 0.05% to 0.1%, from 0.1% to 0.5%, from 0.5% to 1%, from 1% to 2%, from 2% to 5%, or from 5% to 10%. The proportion of target T cells in the starting cell population can be more than 10% (e.g., from 10% to about 50%).

The proportion of target T cells in the enriched target T cell population typically will be higher than 10 percent, but can be lower, e.g., less than 1%, about 1%, about 2%, about 3%, about 4%, about 5%, about 6%, about 7%, about 8% about 9%, or 10%. As noted, however, the proportion of target T cells in the enriched target T cell population typically will be higher than 10 percent and often much higher, however, e.g. at least 10%, at least 20%, at least 30%, at least 40%, at least 50%, at least 60%, at least 70%, at least 80%, at least 90%, at least 95%, at least 96%, at least 97%, at least 98%, at least 99%, or 100%. In some cases, therefore, the population of T cells in the composition is a substantially homogeneous population of target modified T cells (e.g., target modified CTLs). The proportion of target T cells in the enriched target T cell population can be from 10% to 15%, from 15% to 20%, from 20% to 25%, from 25% to 30%, from 30% to 35%, from 35% to 40%, from 40% to 45%, from 45% to 50%, from 50% to 55%, from 55% to 60%, from 60% to 65%, from 65% to 70%, from 70% to 75%, from 75% to 80%, from 80% to 85% from 85% to 90%, from 90% to 95%, or from 95% to 100%. The proportion of target T cells in the enriched target T cell population can be from 10% to 25%, from 25% to 50%, from 50% to 75%, from 75% to 95%, or from 95% to 100%, e.g. 96%, 97%, 98% or 99%.

In some cases, the separation step provides for a fold enrichment (an increase in the proportion of total cells in the enriched target T cell population that are target T cells, compared to the proportion of total cells in the starting cell population that are target T cells) of at least 2-fold, at least 2.5-fold, at least 5-fold, at least 10-fold, at least 25-fold, at least 50-fold, at least 100-fold, at least 250-fold, at least 500-fold, at least 1000-fold, at least 1500-fold, or at least 2000-fold.

Sorting or positively selecting antigen-specific T cells (e.g., antigen-specific CTLs) can be carried out using peptide-loaded MHC multimers (e.g., trimers; tetramers; or pentamers); peptide/MHC-coated magnetic beads; or antibody-coated magnetic beads; or some combination of the foregoing. See, e.g., US 2002/0151690; Altman et al. (1996) Science 274:94; Tubb et al. (2018) J. Immunother. Cancer 6:70; Cobbold et al. (2005) J. Exp. Med. 202:379; and Luxembourg et al. (1998) Nat. Biotechnol. 16:281.

Antigen-specific T cells (e.g., antigen-specific CTLs) can additionally be positively selected by contacting a starting population of cells, or a population of cells that has been enriched for specificity for a pre-selected antigen, with antibodies specific for T cell markers (e.g., where the antibody is attached to a bead, such as a magnetic bead). Antibodies that can be used include, but are not limited to, anti-CD3, anti-CD8, anti-CD25, anti-CD54, anti-CD69, anti-CD38, anti-CD45RO, anti-CD49d, anti-CD40L, anti-CD137, and anti-CD134 antibodies. In some cases, a starting population of cells is enriched for CD8⁺ T cells. Thus, in some cases, a method of the present disclosure comprises: a) at least partially separating target T cells from non-target T cells, thereby generating an enriched target T cell population; b) optionally, at least partially separating CD8⁺ T cells from CD8⁻ T cells in the enriched target T cell population, thereby generating a CD8⁺ enriched target T cell population; and c) genetically modifying the enriched target T cells or CD8⁺ enriched target T cells with one or more nucleic acids comprising nucleotide sequences encoding a CAR, where the CAR comprises an antigen-binding domain specific for a cancer-associated antigen. In some cases, a method of the present disclosure comprises: a) at least partially separating CD8⁺ T cells from CD8⁻ T cells in a starting cell population, thereby generating an enriched CD8⁺ cell population; b) at least partially separating target T cells from non-target T cells present in the enriched CD8⁺ cell population, thereby generating a CD8⁺ enriched target T cell population; and c) genetically modifying the CD8⁺ enriched target T cells with one or more nucleic acids comprising nucleotide sequences encoding a CAR, where the CAR comprises an antigen-binding domain specific for a cancer-associated antigen.

MHC multimers, and methods for generating such multimers, are known in the art. For example, an MHC class I heavy chain can be modified to include biotin. The MHC class I heavy chain is allowed to form a complex with a β2M polypeptide. The biotinylated MHC class I heavy chain/β2M complex is multimerized by contacting the complex with avidin or streptavidin. MHC multimers can be modified to include, e.g., phycoerythrin. For example, a biotinylated MHC class I heavy chain/β2M complex can be multimerized by contacting the complex with PE-labeled avidin or streptavidin. See, e.g., Altman et al. (1996) Science 274:94.

As one non-limiting example, a population of PBMCs is obtained from a CMV-seropositive individual. This starting population of PBMCs contains less than 0.1% CMV-specific T cells. CMV has a prevalence of 50% to 90% in human adults. Magnetic beads are coated with HLA-A*0201 heavy chains. Class I MHC complexes are formed by contacting the coated beads with β2M polypeptides, forming magnetic beads coated with an MHC complex comprising HLA-A*0201 heavy chain/β2M heterodimers. The peptide NLMPMVATV (SEQ ID NO:171) is loaded onto the magnetic beads coated with the MHC complexes, forming magnetic beads coated with MHC/peptide. The MHC/peptide-coated magnetic beads are mixed with PBMCs. Application of a magnetic field provides for separation of CMV-specific T cells from the starting population, generating an enriched CMV-specific T cell population. The enriched CMV-specific T cell population can include over 90% CMV-specific T cells.

As another non-limiting example, a population of PBMCs is obtained from a CMV-seropositive individual. This starting population of PBMCs contains less than 0.1% CMV-specific T cells. The starting population of PBMCs is coated with MHC multimers (e.g., trimers; tetramers; pentamers) loaded with the CMV peptide NLMPMVATV (SEQ ID NO:171), generating a population of MHC/peptide-coated PBMCs. The MHC multimers comprises multiple copies of HLA-A*0201 heavy chain/β2M heterodimers loaded with the CMV peptide. Magnetic beads are coated with antibody specific for the MHC heavy chain present in the MHC multimers. The antibody-coated magnetic beads are mixed with the MHC/peptide-coated PBMCs. Application of a magnetic field provides for separation of CMV-specific T cells from the starting population, generating an enriched CMV-specific T cell population. The enriched CMV-specific T cell population can include over 90% CMV-specific T cells.

As another non-limiting example, a population of PBMCs is obtained from a CMV-seropositive individual. This starting population of PBMCs contains less than 0.1% CMV-specific T cells. The starting population of PBMCs is coated with phycoerythrin (PE)-labeled MHC multimers (e.g., trimers; tetramers; pentamers) loaded with the CMV peptide NLMPMVATV (SEQ ID NO:171), generating a population of MHC/peptide-coated PBMCs. The CMV peptide-loaded, PE-labeled MHC multimers comprises multiple copies of HLA-A*0201 heavy chain/β2M heterodimers loaded with the CMV peptide. Magnetic beads are coated with antibody specific for PE. The antibody-coated magnetic beads are mixed with the MHC/peptide-coated PBMCs. Application of a magnetic field provides for separation of CMV-specific T cells from the starting population, generating an enriched CMV-specific T cell population. The enriched CMV-specific T cell population can include over 90% CMV-specific T cells.

Contacting with a TMMP

In the methods described below, the patient's blood optionally may be pre-screened to determine if it contains an appropriate target T cell for modification with a CAR. For example, the patient's blood may be drawn and T cells screened using the methods described above to determine if the patient already has T cells that are specific for a particular antigen, e.g., CMV, EBD, HPV, tetanus, influenza, etc.

In some cases, prior to the step of at least partially separating target T cells from non-target T cells, the composition comprising a quantity of T cells is contacted in vitro or in vivo with a composition comprising a T-cell modulatory polypeptide (e.g., a TMMP, as described herein) that binds to and activates substantially only the T cells comprising a TCR specific for the preselected antigen. A TMMP that binds to and activates substantially only the T cells comprising a TCR specific for the preselected antigen is thus a TMMP that either wholly or largely (but not wholly) binds to and activates only the T cells comprising a TCR specific for the preselected antigen. That is, a TMMP that binds to and activates substantially only the T cells comprising a TCR specific for the preselected antigen may bind to and activate a relatively small percentage of T cells that are non-target T cells. As used herein, the term “in vitro” is intended to connote any process, system, container, apparatus, equipment, etc. that is outside of the patient, i.e., ex vivo, for making, holding and/or delivering to the patient the mCTLs described herein.

In some cases, the starting cell population is obtained from an individual to be treated with a composition of the present disclosure, where the composition comprises a quantity of modified T cells (e.g., modified CTLs), and where, before the starting cell population is obtained from the individual, one or more doses of a TMMP, as described herein, has been administered to the individual. Administration of a TMMP to the individual, before obtaining the starting cell population from the individual, can increase the number of target T cells in the individual and therefore can increase the proportion of target T cells in the starting cell population. For example, a TMMP comprising a peptide epitope is administered to an individual; and, at a time after such administration (e.g., 2-3 weeks), a starting cell population is obtained from the individual, where the starting cell population comprises target T cells comprising a TCR specific for the peptide epitope present in the TMMP. Thus, in some cases, a method of the present disclosure for making an in vitro composition of cells of the present disclosure comprises: a) administering to an individual a TMMP comprising a peptide epitope; b) obtaining from the individual a composition comprising a quantity of T cells, where the quantity comprises target T cells having a TCR specific for the peptide epitope (a peptide epitope present in a preselected antigen); c) at least partially separating target T cells from non-target T cells comprising a TCR that is not specific for the peptide epitope, thereby generating an enriched target T cell population; and d) modifying the target T cells in the enriched target T cell population by introducing into the target T cells one or more nucleic acids comprising nucleotide sequences encoding a CAR, where the CAR comprises an antigen-binding domain specific for a cancer-associated antigen.

In some cases, a method of the present disclosure for making an in vitro composition of cells of the present disclosure comprises: a) administering to an individual a TMMP comprising a peptide epitope; b) at least partially separating target T cells having a TCR specific for the peptide epitope (a peptide epitope present in a preselected antigen) from non-target T cells comprising a TCR that is not specific for the peptide epitope present in a heterogeneous population of T cells obtained from the individual, thereby generating an enriched target T cell population; and c) modifying the target T cells in the enriched target T cell population by introducing into the target T cells one or more nucleic acids comprising nucleotide sequences encoding a CAR, where the CAR comprises an antigen-binding domain specific for a cancer-associated antigen. In some cases, the TMMP is administered to the individual at a period of time of from 1 day to 1 month (e.g., from 1 day to 4 days, from 4 days to 7 days, from 1 week to 2 weeks, from 2 weeks to 3 weeks, or from 3 weeks to 1 month) before the starting cell population is obtained from the individual. In some cases, multiple doses of the TMMP are administered to the individual before the starting cell population is obtained from the individual.

In some cases, prior to step (ii) (i.e., prior to the step of at least partially separating target T cells from non-target T cells), the composition comprising a quantity of T cells is contacted in vitro with a composition comprising a TMMP that binds to and activates substantially only the T cells comprising a TCR specific for the preselected antigen. Suitable TMMPs are described elsewhere herein. Contacting the T cells with a TMMP that binds to and activates substantially only the target T cells comprising a TCR specific for the preselected antigen stimulates proliferation of the target T cells, thereby increasing the number of target T cells.

### Modifying Target T Cells

As noted above, a method of the present disclosure for making an in vitro composition of cells of the present disclosure comprises modifying the target T cells in the enriched target T cell population by introducing into the target T cells one or more nucleic acids comprising nucleotide sequences encoding a CAR, where the CAR comprises an antigen-binding domain specific for a cancer-associated antigen. Any method known to those skilled in the art may be used to introduce into the target T cells one or more nucleic acids comprising nucleotide sequences encoding a CAR. A discussion of some known materials and methods is provided below.

A nucleic acid comprising a nucleotide sequence encoding a CAR can be an expression vector, e.g., recombinant expression vector. In some cases, the recombinant expression vector is a viral construct, e.g., a recombinant adeno-associated virus (AAV) construct, a recombinant adenoviral construct, a recombinant lentiviral construct, a recombinant retroviral construct, etc. In some cases, a nucleic acid comprising a nucleotide sequence encoding a CAR is a recombinant lentivirus vector. In some cases, a nucleic acid comprising a nucleotide sequence encoding a CAR is a recombinant AAV vector.

The nucleotide sequences encoding the CAR can be operably linked to a transcriptional control element such as a promoter. The transcriptional control element (e.g., a promoter) is one that is functional in a T cell. Suitable promoters include constitutive promoters and regulatable (e.g., inducible) promoters.

One example of a suitable promoter is the immediate early cytomegalovirus (CMV) promoter sequence. This promoter sequence is a strong constitutive promoter sequence capable of driving high levels of expression of an operably linked nucleotide sequence. Another example of a suitable promoter is Elongation Growth Factor-1α (EF-1α). However, other constitutive promoter sequences may also be used, including, but not limited to, the simian virus 40 (SV40) early promoter, MND (myeloproliferative sarcoma virus) promoter, mouse mammary tumor virus (MMTV), human immunodeficiency virus (HIV) long terminal repeat (LTR) promoter, Moloney Murine Leukemia Virus (MoMuLV) promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter. Examples of inducible promoters include, but are not limited to, a metallothionine promoter, a glucocorticoid-inducible promoter, a progesterone-inducible promoter, and a tetracycline-inducible promoter.

In some cases, the promoter is a CD8 cell-specific promoter, a CD4 cell-specific promoter, a neutrophil-specific promoter, or an NK-specific promoter. For example, a CD4 gene promoter can be used; see, e.g., Salmon et al. (1993) Proc. Natl. Acad. Sci. USA 90: 7739; and Marodon et al. (2003) Blood 101:3416. As another example, a CD8 gene promoter can be used. NK cell-specific expression can be achieved by use of an Ncr1 (p46) promoter; see, e.g., Eckelhart et al. (2011) Blood 117:1565.

Any method of introducing nucleic acids into cells can be used to introduce nucleic acid(s) encoding a CAR into target T cells. Suitable methods include viral transfection (e.g., where the nucleic acid is a lentiviral vector or other viral vector comprising a nucleotide sequence encoding a CAR), electroporation, diethylaminoethyl (DEAE)-dextran-mediated transfection, lipofection, and the like.

### Use of Allogeneic T Cells

While the methods and compositions described above involve the removal of T cells from a patient and the introduction of target modified T cells back into the same patient, it also is expressly contemplated that allogeneic T cells can be used instead of (or in addition to) T cells removed from the patient. If a heterogenous population of allogeneic T cells is employed as a starting material, then an enriched population optionally can be prepared as described above. The target modified T cells made from allogeneic T cells thus can contain both a TCR specific for a preselected antigen (including such TCRs expressed as a result of gene-editing), as well as one or more nucleic acids comprising nucleotide sequences encoding a CAR, where the CAR comprises an antigen-binding domain specific for a cancer-associated antigen. The target modified T cells prepared from allogeneic T cells then would be employed for treatment in the same manner as described herein for patient-derived target modified T cells.

### Methods of Treating Cancer

The present disclosure provides a method of treating cancer in an individual having a cancer. The method comprises: (a) administering to the individual a composition comprising a quantity of genetically modified CTLs of the present disclosure (e.g., modified CTLs prepared according to a method of the present disclosure), where the genetically modified CTLs express a CAR on their cell surface, where the CAR comprises an antigen-binding domain (e.g., an antibody) specific for a cancer-associated peptide; and (b) administering to the individual a composition comprising a TMMP. The TMMP selectively binds to and activates T cells comprising a TCR specific for the preselected antigen. T cells comprising a TCR specific for the preselected antigen will in many cases be genetically modified CTLs as described herein. By activating genetically modified CTLs, the number and activity of such cells is increased, such that activated, genetically modified CTLs target and kill tumor cells that bear on their surface the cancer-associated epitope for which the CAR is specific.

A method of the present disclosure comprises administering an effective amount of a modified CTL of the present disclosure and an effective amount of a TMMP.

In some cases, “effective amounts” of a modified CTL of the present disclosure and a TMMP are amounts that, when administered in one or more doses to an individual in need thereof, lead to a reduction in the number of cancer cells in the individual. The amount that is an effective amount may depend on whether the patient is receiving additional treatments in combination with the modified CTL, including additional treatments with TMMPs as discussed herein.

For example, in some cases, “effective amounts” of a modified CTL of the present disclosure and a TMMP are amounts that, when administered in one or more doses to an individual in need thereof, leads to a reduction in the number of cancer cells in the individual by at least 10%, at least 15%, at least 20%, at least 25%, at least 30%, at least 40%, at least 50%, at least 60%, at least 70%, at least 80%, at least 90%, or at least 95%, compared to the number of cancer cells in the individual before administration of the modified CTL and the TMMP, or in the absence of administration with the modified CTL and the TMMP. In some cases, “effective amounts” of a modified CTL of the present disclosure and a TMMP are amounts that, when administered in one or more doses to an individual in need thereof, leads to a reduction in the number of cancer cells in the individual to undetectable levels.

In some cases, “effective amounts” of a modified CTL of the present disclosure and a TMMP are amounts that, when administered in one or more doses to an individual in need thereof, leads to a reduction in the tumor mass in the individual. For example, in some cases, “effective amounts” of a modified CTL of the present disclosure and a TMMP are amounts that, when administered in one or more doses to an individual in need thereof (an individual having a tumor), leads to a reduction in the tumor mass in the individual by at least 10%, at least 15%, at least 20%, at least 25%, at least 30%, at least 40%, at least 50%, at least 60%, at least 70%, at least 80%, at least 90%, or at least 95%, compared to the tumor mass in the individual before administration of the modified CTL and the TMMP, or in the absence of administration of the modified CTL and the TMP. In some cases, “effective amounts” of a modified CTL of the present disclosure and a TMMP are amounts that, when administered in one or more doses to an individual in need thereof (an individual having a tumor), leads to a reduction in the tumor volume in the individual. For example, in some cases, “effective amounts” of a modified CTL of the present disclosure and a TMMP are amounts that, when administered in one or more doses to an individual in need thereof (an individual having a tumor), reduces the tumor volume in the individual by at least 10%, at least 15%, at least 20%, at least 25%, at least 30%, at least 40%, at least 50%, at least 60%, at least 70%, at least 80%, at least 90%, or at least 95%, compared to the tumor volume in the individual before administration of the modified CTL and the TMMP, or in the absence of administration with the modified CTL and the TMMP. In some cases, “effective amounts” of a modified CTL of the present disclosure and a TMMP are amounts that, when administered in one or more doses to an individual in need thereof, leads to an increase in survival time of the individual. For example, in some cases, “effective amounts” of a modified CTL of the present disclosure and a TMMP are amounts that, when administered in one or more doses to an individual in need thereof, leads to an increase in survival time of the individual by at least 1 month, at least 2 months, at least 3 months, from 3 months to 6 months, from 6 months to 1 year, from 1 year to 2 years, from 2 years to 5 years, from 5 years to 10 years, or more than 10 years, compared to the expected survival time of the individual in the absence of administration with the modified CTL and the TMMP.

In some cases, the modified CTL and the TMMP are administered at the same time. In some cases, the modified CTL and the TMMP are administered to an individual within about 1 minute to about 24 hours (e.g., within about 1 minute, within about 5 minutes, within about 15 minutes, within about 30 minutes, within about 1 hour, within about 4 hours, within about 8 hours, within about 12 hours, or within about 24 hours) of one another.

In some cases, a method of the present disclosure comprises: a) administering a TMMP; and b) after a period of time, administering a modified CTL. For example, in some cases, a TMMP is administered from 1 week to 4 weeks (e.g., 1 week, 2 weeks, 3 weeks, or 4 weeks) before the modified CTL is administered. In some cases, multiple doses of a TMMP are administered to an individual before a modified CTL is administered to the individual. For example, in some cases, a method of the present disclosure comprises: a) administering multiple doses of a TMMP; and b) after a period of time, administering a modified CTL.

In some cases, a method of the present disclosure comprises: a) administering a modified CTL; and b) after a period of time, administering a TMMP. For example, in some cases, a modified CTL is administered from 1 week to 4 weeks (e.g., 1 week, 2 weeks, 3 weeks, or 4 weeks) before the TMMP is administered. In some cases, multiple doses of a TMMP are administered to an individual after the modified CTL is administered to the individual. For example, in some cases, a method of the present disclosure comprises: a) administering a modified CTL; and b) after a period of time, administering multiple doses of a TMMP to the individual.

In some cases, a method of the present disclosure comprises: a) a) administering a TMMP; b) after a period of time, administering a modified CTL; and c) after a period of time, administering the TMMP. For example, in some cases, a TMMP is administered from 1 week to 4 weeks (e.g., 1 week, 2 weeks, 3 weeks, or 4 weeks) before the modified CTL is administered; and, after the CTL is administered, one or more additional doses of the TMMP is administered to the individual. In some cases, multiple doses of a TMMP are administered to an individual before a modified CTL is administered to the individual; and multiple doses of the TMMP are administered to the individual after the modified CTL is administered.

Cancers that can be treated with a method of the present disclosure include any cancer that can be targeted with a CAR. Cancers that can be treated with a method of the present disclosure include carcinomas, sarcomas, melanoma, leukemias, and lymphomas. Cancers that can be treated with a method of the present disclosure include solid tumors. Cancers that can be treated with a method of the present disclosure include metastatic cancers.

Carcinomas that can treated by a method disclosed herein include, but are not limited to, esophageal carcinoma, hepatocellular carcinoma, basal cell carcinoma (a form of skin cancer), squamous cell carcinoma (various tissues), bladder carcinoma, including transitional cell carcinoma (a malignant neoplasm of the bladder), bronchogenic carcinoma, colon carcinoma, colorectal carcinoma, gastric carcinoma, lung carcinoma, including small cell carcinoma and non-small cell carcinoma of the lung, adrenocortical carcinoma, thyroid carcinoma, pancreatic carcinoma, breast carcinoma, ovarian carcinoma, prostate carcinoma, adenocarcinoma, sweat gland carcinoma, sebaceous gland carcinoma, papillary carcinoma, papillary adenocarcinoma, cystadenocarcinoma, medullary carcinoma, renal cell carcinoma, ductal carcinoma in situ or bile duct carcinoma, choriocarcinoma, seminoma, embryonal carcinoma, Wilm's tumor, cervical carcinoma, uterine carcinoma, testicular carcinoma, osteogenic carcinoma, epithelial carcinoma, and nasopharyngeal carcinoma.

Sarcomas that can be treated by a method disclosed herein include, but are not limited to, fibrosarcoma, myxosarcoma, liposarcoma, chondrosarcoma, chordoma, osteogenic sarcoma, osteosarcoma, angiosarcoma, endotheliosarcoma, lymphangiosarcoma, lymphangioendotheliosarcoma, synovioma, mesothelioma, Ewing's sarcoma, leiomyosarcoma, rhabdomyosarcoma, and other soft tissue sarcomas.

Other solid tumors that can be treated by a method disclosed herein include, but are not limited to, glioma, astrocytoma, medulloblastoma, craniopharyngioma, ependymoma, pinealoma, hemangioblastoma, acoustic neuroma, oligodendroglioma, menangioma, melanoma, neuroblastoma, and retinoblastoma.

Leukemias that can be amenable to therapy by a method disclosed herein include, but are not limited to, a) chronic myeloproliferative syndromes (neoplastic disorders of multipotential hematopoietic stem cells); b) acute myelogenous leukemias (neoplastic transformation of a multipotential hematopoietic stem cell or a hematopoietic cell of restricted lineage potential; c) chronic lymphocytic leukemias (CLL; clonal proliferation of immunologically immature and functionally incompetent small lymphocytes), including B-cell CLL, T-cell CLL prolymphocytic leukemia, and hairy cell leukemia; and d) acute lymphoblastic leukemias (characterized by accumulation of lymphoblasts). Lymphomas that can be treated using a subject method include, but are not limited to, B-cell lymphomas (e.g., Burkitt's lymphoma); Hodgkin's lymphoma; non-Hodgkin's lymphoma, and the like.

Other cancers that can be treated according to the methods disclosed herein include atypical meningioma, islet cell carcinoma, medullary carcinoma of the thyroid, mesenchymoma, hepatocellular carcinoma, hepatoblastoma, clear cell carcinoma of the kidney, and neurofibroma mediastinum.

### Formulations

Suitable formulations comprising a modified CTL, or a TMMP, include a pharmaceutically acceptable excipient. In some cases, a suitable formulation comprises: a) a modified CTL of the present disclosure; and b) a pharmaceutically acceptable excipient. In some cases, a suitable formulation comprises: a) a TMMP; and b) a pharmaceutically acceptable excipient. The composition may comprise a pharmaceutically acceptable excipient, a variety of which are known in the art and need not be discussed in detail herein. Pharmaceutically acceptable excipients have been amply described in a variety of publications, including, for example, “Remington: The Science and Practice of Pharmacy”, 19^{th} Ed. (1995), or latest edition, Mack Publishing Co; A. Gennaro (2000) “Remington: The Science and Practice of Pharmacy”, 20th edition, Lippincott, Williams, & Wilkins; Pharmaceutical Dosage Forms and Drug Delivery Systems (1999) H. C. Ansel et al., eds 7^{th} ed., Lippincott, Williams, & Wilkins; and Handbook of Pharmaceutical Excipients (2000) A. H. Kibbe et al., eds., 3^{rd} ed. Amer. Pharmaceutical Assoc.

A pharmaceutical composition can comprise a modified CTL of the present disclosure, and a pharmaceutically acceptable excipient; or can comprise a TMMP and a pharmaceutically acceptable excipient. In some cases, a subject pharmaceutical composition will be suitable for administration to a subject, e.g., will be sterile. For example, in some cases, a subject pharmaceutical composition will be suitable for administration to a human subject, e.g., where the composition is sterile and is free of detectable pyrogens and/or other toxins.

### Dosages

A suitable dosage of a TMMP or a modified CTL can be determined by an attending physician or other qualified medical personnel, based on various clinical factors. As is well known in the medical arts, dosages for any one patient depend upon many factors, including the patient's size, body surface area, age, the particular TMMP or modified CTL to be administered, sex of the patient, time, and route of administration, general health, and other drugs being administered concurrently.

A TMMP may be administered in amounts between 1 ng/kg body weight and 20 mg/kg body weight per dose, e.g. between 0.1 mg/kg body weight to 10 mg/kg body weight, e.g. between 0.1 mg/kg body weight and 4 mg/kg body weight, between 0.5 mg/kg body weight and 2 mg/kg body weight, or between 0.5 mg/kg body weight and 5 mg/kg body weight; however, doses below or above this exemplary range are envisioned, especially considering the aforementioned factors. If the regimen is a continuous infusion, it can also be in the range of 1 g to 10 mg per kilogram of body weight per minute. A TMMP can be administered in an amount of from about 1 mg/kg body weight to 50 mg/kg body weight, e.g., from about 1 mg/kg body weight to about 5 mg/kg body weight, from about 5 mg/kg body weight to about 10 mg/kg body weight, from about 10 mg/kg body weight to about 15 mg/kg body weight, from about 15 mg/kg body weight to about 20 mg/kg body weight, from about 20 mg/kg body weight to about 25 mg/kg body weight, from about 25 mg/kg body weight to about 30 mg/kg body weight, from about 30 mg/kg body weight to about 35 mg/kg body weight, from about 35 mg/kg body weight to about 40 mg/kg body weight, or from about 40 mg/kg body weight to about 50 mg/kg body weight.

In some cases, a suitable dose of a TMMP is from 0.01 μg to 100 μg per kg of body weight, from 0.1 μg to 10 μg per kg of body weight, from 1 μg to 1 μg per kg of body weight, from 10 μg to 100 mg per kg of body weight, from 100 μg to 10 mg per kg of body weight, or from 100 μg to 1 mg per kg of body weight. Persons of ordinary skill in the art can easily estimate repetition rates for dosing based on measured residence times and concentrations of the administered agent in bodily fluids or tissues. Following successful treatment, it may be desirable to have the patient undergo maintenance therapy to prevent the recurrence of the disease state, wherein a TMMP is administered in maintenance doses, ranging from 0.01 μg to 100 μg per kg of body weight, from 0.1 μg to 10 μg per kg of body weight, from 1 μg to 1 μg per kg of body weight, from 10 μg to 100 mg per kg of body weight, from 100 μg to 10 mg per kg of body weight, or from 100 μg to 1 mg per kg of body weight.

In some cases, a suitable dose of modified CTLs that is equal to or less than a number selected from the group consisting of 10 cells/kg body weight, 10² cells/kg body weight, 10³ cells/kg body weight, 10⁴ cells/kg body weight, 10⁵ cells/kg body weight, 10⁶ cells/kg body weight, 10⁷ cells/kg body weight, 10⁸ cells/kg body weight, and 10⁹ cells/kg body weight. In some cases, a suitable dose of modified CTLs is from about 10 cells/kg body weight to about 10² cells/kg body weight, from about 10² cells/kg body weight to about 10³ cells/kg body weight, from about 10³ cells/kg body weight to about 10⁴ cells/kg body weight, from about 10⁴ cells/kg body weight to about 10⁵ cells/kg body weight, from about 10⁵ cells/kg body weight to about 10⁶ cells/kg body weight, from about 10⁶ cells/kg body weight to about 10⁷ cells/kg body weight, from about 10⁷ cells/kg body weight to about 10⁸ cells/kg body weight, or from about 10⁸ cells/kg body weight to about 10⁹ cells/kg body weight. In some cases, lower doses of modified CTLs can be employed, e.g., less than about 10⁷ cells/kg body weight, less than about 10⁶ cells/kg body weight, less than about 10⁵ cells/kg body weight, less than about 10⁴ cells/kg body weight or less than about 10³ cells/kg body weight.

Those of skill will readily appreciate that dose levels can vary as a function of the specific TMMP or the modified CTL, the severity of the symptoms, and the susceptibility of the subject to side effects. Preferred dosages for a given TMMP or modified CTL are readily determinable by those of skill in the art by a variety of means.

### Routes of Administration

An active agent (a modified CTL; a TMMP) is administered to an individual using any available method and route suitable for drug delivery, including in vivo and ex vivo methods, as well as systemic and localized routes of administration.

Conventional and pharmaceutically acceptable routes of administration include intratumoral, peritumoral, intramuscular, intralymphatic, intratracheal, intracranial, subcutaneous, intradermal, topical application, intravenous, intraarterial, rectal, nasal, oral, and other enteral and parenteral routes of administration. Routes of administration may be combined, if desired, or adjusted depending upon the TMMP, the modified CTL, and/or the desired effect. A modified CTL of the present disclosure can be administered in a single dose or in multiple doses. Similarly, a TMMP can be administered in a single dose or in multiple doses. A modified CTL can be administered via the same route of administration as the TMMP. A modified CTL can be administered via a different route of administration from the TMMP.

In some cases, a modified CTL of the present disclosure is administered intravenously. In some cases, a modified CTL of the present disclosure is administered intralymphatically. In some cases, a modified CTL of the present disclosure is administered locally. In some cases, a modified CTL of the present disclosure is administered intratumorally. In some cases, a modified CTL of the present disclosure is administered peritumorally. In some cases, a modified CTL of the present disclosure is administered intracranially.

In some cases, a TMMP is administered intravenously. In some cases, a TMMP is administered intramuscularly. In some cases, a TMMP is administered intralymphatically. In some cases, a TMMP is administered locally. In some cases, a TMMP is administered intratumorally. In some cases, a TMMP is administered peritumorally. In some cases, a TMMP is administered intracranially. In some cases, a TMMP is administered subcutaneously.

### Combination Therapies

In some cases, a method of the present disclosure for treating cancer in an individual comprises: a) administering a modified CTL of the present disclosure; b) administering a TMMP; and c) administering at least one additional therapeutic agent or therapeutic treatment. Suitable additional therapeutic agents include, but are not limited to, small molecule cancer chemotherapeutic agents, biologic anti-cancer agents, e.g., antibodies, fusion proteins and bi-specific antibodies, and immune checkpoint inhibitors. Suitable additional therapeutic treatments include, e.g., radiation, surgery (e.g., surgical resection of a tumor), and the like.

As an example, a treatment method of the present disclosure can comprise co-administration of: a) a modified CTL of the present disclosure; b) a TMMP; and c) an immune checkpoint inhibitor, such as an antibody specific for an immune checkpoint. In some cases, a modified CTL of the present disclosure and a TMMP are administered to an individual who is undergoing treatment with, or who has undergone treatment with, an antibody or other agent specific for an immune checkpoint.

Exemplary immune checkpoint inhibitors include inhibitors that target immune checkpoint polypeptides such as CD27, CD28, CD40, CD122, CD96, CD73, CD47, OX40, GITR, CSF1R, JAK, PI3K delta, PI3K gamma, TAM, arginase, CD137 (also known as 4-1BB), ICOS, A2AR, B7-H3, B7-H4, BTLA, CTLA-4, LAG3, TIM3, VISTA, CD96, TIGIT, CD122, PD-1, PD-L1, and PD-L2. In some cases, the immune checkpoint polypeptide is a stimulatory checkpoint molecule selected from CD27, CD28, CD40, ICOS, OX40, GITR, CD122 and CD137. In some cases, the immune checkpoint polypeptide is an inhibitory checkpoint molecule selected from A2AR, B7-H3, B7-H4, BTLA, CTLA-4, IDO, KIR, LAG3, PD-1, TIM3, CD96, TIGIT and VISTA.

In some cases, the immune checkpoint inhibitor is an antibody specific for an immune checkpoint. In some cases, the anti-immune checkpoint antibody is a monoclonal antibody. In some cases, the anti-immune checkpoint antibody is humanized, or de-immunized such that the antibody substantially does not elicit an immune response in a human, or elicits an immune response that is acceptable for at least some percentage of patients. In some cases, the anti-immune checkpoint antibody is a humanized monoclonal antibody. In some cases, the anti-immune checkpoint antibody is a de-immunized monoclonal antibody. In some cases, the anti-immune checkpoint antibody is a fully human monoclonal antibody. In some cases, the anti-immune checkpoint antibody inhibits binding of the immune checkpoint polypeptide to a ligand for the immune checkpoint polypeptide. In some cases, the anti-immune checkpoint antibody inhibits binding of the immune checkpoint polypeptide to a receptor for the immune checkpoint polypeptide.

Suitable anti-immune checkpoint antibodies include, but are not limited to, nivolumab (Bristol-Myers Squibb), pembrolizumab (Merck), pidilizumab (Curetech), AMP-224 (GlaxoSmithKline/Amplimmune), MPDL3280A (Roche), MDX-1105 (Medarex, Inc./Bristol Myer Squibb), MEDI-4736 (Medimmune/AstraZeneca), arelumab (Merck Serono), ipilimumab (YERVOY, (Bristol-Myers Squibb), tremelimumab (Pfizer), pidilizumab (CureTech, Ltd.), IMP321 (Immutep S.A.), MGA271 (Macrogenics), BMS-986016 (Bristol-Meyers Squibb), lirilumab (Bristol-Myers Squibb), urelumab (Bristol-Meyers Squibb), PF-05082566 (Pfizer), IPH2101 (Innate Pharma/Bristol-Myers Squibb), MEDI-6469 (MedImmune/AZ), CP-870,893 (Genentech), Mogamulizumab (Kyowa Hakko Kirin), Varlilumab (CellDex Therapeutics), Avelumab (EMD Serono), Galiximab (Biogen Idec), AMP-514 (Amplimmune/AZ), AUNP 12 (Aurigene and Pierre Fabre), Indoximod (NewLink Genetics), NLG-919 (NewLink Genetics), INCB024360 (Incyte); KN035; and combinations thereof. For example, in some cases, the immune checkpoint inhibitor is an anti-PD-1 antibody. Suitable anti-PD-1 antibodies include, e.g., nivolumab, pembrolizumab (also known as MK-3475), pidilizumab, SHR-1210, PDR001, and AMP-224. In some cases, the anti-PD-1 monoclonal antibody is nivolumab, pembrolizumab or PDR001. Suitable anti-PD1 antibodies are described in U.S. Patent Publication No. 2017/0044259. For pidilizumab, see, e.g., Rosenblatt et al. (2011) J. Immunother. 34:409-18. In some cases, the immune checkpoint inhibitor is an anti-CTLA-4 antibody. In some cases, the anti-CTLA-4 antibody is ipilimumab or tremelimumab. For tremelimumab, see, e.g., Ribas et al. (2013) J. Clin. Oncol. 31:616-22. In some cases, the immune checkpoint inhibitor is an anti-PD-L1 antibody. In some cases, the anti-PD-L1 monoclonal antibody is BMS-935559, MEDI4736, MPDL3280A (also known as RG7446), KN035, or MSB0010718C. In some embodiments, the anti-PD-L1 monoclonal antibody is MPDL3280A (atezolizumab) or MEDI4736 (durvalumab). For durvalumab, see, e.g., WO 2011/066389. For atezolizumab, see, e.g., U.S. Pat. No. 8,217,149.

### Subjects Suitable for Treatment

Subjects suitable for treatment with a method of the present disclosure include individuals who have cancer, including individuals who have been diagnosed as having cancer but have not yet undergone treatment, individuals who have been treated for cancer but who failed to respond to the treatment, and individuals who have been treated for cancer and who initially responded but subsequently became refractory to the treatment. In some cases, the individual already will have been treated with an immune checkpoint inhibitor, e.g., nivolumab or pembrolizumab, as described above.

In some cases, the individual being treated according to a method of the present disclosure has not undergone a lymphodepleting regimen prior to administration of a modified CTL of the present disclosure and/or prior to administration of a TMMP. In some cases, the individual being treated according to a method of the present disclosure has undergone a lymphodepleting regimen prior to administration of a modified CTL of the present disclosure and/or prior to administration of a TMMP. In some cases, the lymphodepletion regimen is a non-myeloablative lymphodepletion regimen. Lymphodepletion can be accomplished by administering to the individual: i) cyclophosphamide/fludarabine combination; or ii) cyclophosphamide alone.

### TMMP

A TMMP suitable for use in a method of the present disclosure comprises a heterodimer comprising: a) a first polypeptide comprising: i) a peptide epitope, wherein the peptide epitope is a peptide having a length of at least 4 amino acids (e.g., from 4 amino acids to about 25 amino acids); and ii) a first major histocompatibility complex (MHC) polypeptide; b) a second polypeptide comprising a second MHC polypeptide; and c) at least one immunomodulatory polypeptide, where the first and/or the second polypeptide comprises the at least one immunomodulatory polypeptide. The TMMP optionally also includes an immunoglobulin (Ig) Fc polypeptide or a non-Ig scaffold, where the first and/or the second polypeptide comprises the Ig Fc polypeptide or the non-Ig scaffold.

As used herein, the term “peptide epitope” means a peptide that, when complexed with MHC polypeptides, presents an epitope to a TCR. A peptide epitope has a length of at least 4 amino acids, e.g., from 4 amino acids to about 25 amino acids (e.g., 4 amino acids (aa), 5 aa, 6 aa, 7 aa, 8 aa, 9 aa, 10 aa, 11 aa, 12 aa, 13 aa, 14 aa, 15 aa, 16 aa, 17 aa, 18 aa, 19 aa, 20 aa, 21 aa, 22 aa, 23 aa, 24 aa, or 25 aa, including within a range of from 4 to 20 aa, from 6 to 18 aa, from 8 to 15 aa. from 8 to 12 aa, from 5 to 10 aa, from 10 to 15 aa, from 15 to 20 aa, from 10 to 20 aa, or from 15 to 25 aa. in length). When complexed with MHC polypeptides, a peptide epitope can present one or more epitopes to one or more TCRs. In some cases, the peptide epitope present in a TMMP presents an infectious disease-associated epitope (e.g., a virus-encoded peptide).

As noted above, a TMMP comprises a heterodimeric polypeptide comprising: a) a first polypeptide comprising: i) a peptide epitope; and ii) a first MHC polypeptide; b) a second polypeptide comprising a second MHC polypeptide; c) at least one immunomodulatory polypeptide, where the first and/or the second polypeptide comprises the at least one (i.e., one or more) immunomodulatory polypeptide; and, optionally, d) an Ig Fc polypeptide or a non-Ig scaffold, where the first and/or the second polypeptide comprises the Ig Fc polypeptide or the non-Ig scaffold. In some cases, the at least one immunomodulatory polypeptide is wild-type, i.e., comprises an amino acid sequence of a naturally-occurring immunomodulatory polypeptide.

In some cases, at least one of the one or more immunomodulatory polypeptides is a variant immunomodulatory polypeptide that exhibits reduced affinity to a cognate co-immunomodulatory polypeptide compared to the affinity of a corresponding wild-type immunomodulatory polypeptide for the cognate co-immunomodulatory polypeptide. In some cases, the peptide epitope present in a TMMP of the present disclosure binds to a T-cell receptor (TCR) on a T cell with an affinity of at least 100 μM (e.g., at least 10 μM, at least 1 μM, at least 100 nM, at least 10 nM, or at least 1 nM). In some cases, a TMMP of the present disclosure binds to a first T cell with an affinity that is at least 25% higher than the affinity with which the TMMP binds a second T cell, where the first T cell expresses on its surface the cognate co-immunomodulatory polypeptide and a TCR that binds the epitope with an affinity of at least 100 μM, and where the second T cell expresses on its surface the cognate co-immunomodulatory polypeptide but does not express on its surface a TCR that binds the epitope with an affinity of at least 100 μM (e.g., at least 10 μM, at least 1 μM, at least 100 nM, at least 10 nM, or at least 1 nM).

In some cases, a TMMP is:
A) a heterodimer comprising: a) a first polypeptide comprising a first MHC polypeptide; and b) a second polypeptide comprising a second MHC polypeptide, wherein the first polypeptide or the second polypeptide comprises a peptide epitope, wherein the first polypeptide and/or the second polypeptide comprises one or more immunomodulatory polypeptides that can be the same or different, and wherein at least one of the one or more immunomodulatory polypeptides may be a wild-type immunomodulatory polypeptide or a variant of a wild-type immunomodulatory polypeptide, wherein the variant immunomodulatory polypeptide comprises 1, 2, 3, 4, 5, 6, 7, 8, 9, 10, 11, 12, 13, 14, 15, 16, 17, 18, 19, or 20 amino acid substitutions compared to the amino acid sequence of the corresponding wild-type immunomodulatory polypeptide; wherein the first polypeptide and/or the second polypeptide comprises an Ig Fc polypeptide or a non-Ig scaffold; or
B) a heterodimer comprising: a) a first polypeptide comprising a first MHC polypeptide; and b) a second polypeptide comprising a second MHC polypeptide, wherein the first polypeptide or the second polypeptide comprises an epitope; wherein the first polypeptide and/or the second polypeptide comprises one or more immunomodulatory polypeptides that can be the same or different,
wherein at least one of the one or more immunomodulatory polypeptides is a variant of a wild-type immunomodulatory polypeptide, wherein the variant immunomodulatory polypeptide comprises 1, 2, 3, 4, 5, 6, 7, 8, 9, 10, 11, 12, 13, 14, 15, 16, 17, 18, 19, or 20 amino acid substitutions compared to the amino acid sequence of the corresponding wild-type immunomodulatory polypeptide,
wherein at least one of the one or more immunomodulatory domains is a variant immunomodulatory polypeptide that exhibits reduced affinity to a cognate co-immunomodulatory polypeptide compared to the affinity of a corresponding wild-type immunomodulatory polypeptide for the cognate co-immunomodulatory polypeptide, and wherein the epitope binds to a TCR on a T cell with an affinity of at least 10⁻⁷ M, such that: i) the TMMP polypeptide binds to a first T cell with an affinity that is at least 25% higher than the affinity with which the TMMP binds a second T cell, wherein the first T cell expresses on its surface the cognate co-immunomodulatory polypeptide and a TCR that binds the epitope with an affinity of at least 10⁻⁷ M, and wherein the second T cell expresses on its surface the cognate co-immunomodulatory polypeptide but does not express on its surface a TCR that binds the epitope with an affinity of at least 10⁻⁷ M; and/or ii) the ratio of the binding affinity of a control TMMP, wherein the control comprises a wild-type immunomodulatory polypeptide, to a cognate co-immunomodulatory polypeptide to the binding affinity of the TMMP comprising a variant of the wild-type immunomodulatory polypeptide to the cognate co-immunomodulatory polypeptide, when measured by bio-layer interferometry, is in a range of from 1.5:1 to 10⁶:1; and wherein the variant immunomodulatory polypeptide comprises 1, 2, 3, 4, 5, 6, 7, 8, 9, 10, 11, 12, 13, 14, 15, 16, 17, 18, 19, or 20 amino acid substitutions compared to the amino acid sequence of the corresponding wild-type immunomodulatory polypeptide; and
wherein the first polypeptide and/or the second polypeptide comprises an Ig Fc polypeptide or a non-Ig scaffold; or
C) a heterodimer comprising: a) a first polypeptide comprising, in order from N-terminus to C-terminus: i) an epitope; ii) a first MHC polypeptide; and b) a second polypeptide comprising, in order from N-terminus to C-terminus: i) a second MHC polypeptide; and ii) optionally an immunoglobulin (Ig) Fc polypeptide or a non-Ig scaffold, wherein the TMMP comprises one or more immunomodulatory domains that can be the same or different, wherein at least one of the one or more immunomodulatory domain is: A) at the C-terminus of the first polypeptide; B) at the N-terminus of the second polypeptide; C) at the C-terminus of the second polypeptide; or D) at the C-terminus of the first polypeptide and at the N-terminus of the second polypeptide, and wherein at least one of the one or more immunomodulatory domains may be a wild-type immunomodulatory polypeptide or a variant of a wild-type immunomodulatory polypeptide, wherein the variant immunomodulatory polypeptide comprises 1, 2, 3, 4, 5, 6, 7, 8, 9, 10, 11, 12, 13, 14, 15, 16, 17, 18, 19, or 20 amino acid substitutions compared to the amino acid sequence of the corresponding wild-type immunomodulatory polypeptide; and
optionally wherein at least one of the one or more immunomodulatory domains is a variant immunomodulatory polypeptide that exhibits reduced affinity to a cognate co-immunomodulatory polypeptide compared to the affinity of a corresponding wild-type immunomodulatory polypeptide for the cognate co-immunomodulatory polypeptide, and wherein the epitope binds to a TCR on a T cell with an affinity of at least 10⁻⁷ M, such that: i) the TMMP binds to a first T cell with an affinity that is at least 25% higher than the affinity with which the TMMP binds a second T cell, wherein the first T cell expresses on its surface the cognate co-immunomodulatory polypeptide and a TCR that binds the epitope with an affinity of at least 10⁻⁷ M, and wherein the second T cell expresses on its surface the cognate co-immunomodulatory polypeptide but does not express on its surface a TCR that binds the epitope with an affinity of at least 10⁻⁷ M; and/or ii) the ratio of the binding affinity of a control TMMP, wherein the control comprises a wild-type immunomodulatory polypeptide, to a cognate co-immunomodulatory polypeptide to the binding affinity of the TMMP comprising a variant of the wild-type immunomodulatory polypeptide to the cognate co-immunomodulatory polypeptide, when measured by bio-layer interferometry, is in a range of from 1.5:1 to 10⁶:1; and wherein the variant immunomodulatory polypeptide comprises 1, 2, 3, 4, 5, 6, 7, 8, 9, 10, 11, 12, 13, 14, 15, 16, 17, 18, 19, or 20 amino acid substitutions compared to the amino acid sequence of the corresponding wild-type immunomodulatory polypeptide.

In some cases, the epitope present in a TMMP binds to a TCR on a T cell with an affinity of from about 10⁻⁴ M to about 5×10⁻⁴ M, from about 5×10⁻⁴ M to about 10⁻⁵ M, from about 10⁻⁵ M to 5×10⁻⁵ M, from about 5×10⁻⁵ M to 10⁻⁶ M, from about 10⁻⁶ M to about 5×10⁻⁶ M, from about 5×10⁻⁶ M to about 10⁻⁷ M, from about 10⁻⁷ M to about 5×10⁻⁷ M, from about 5×10⁻⁷ M to about 10⁻⁸ M, or from about 10⁻⁸ M to about 10⁻⁹ M. Expressed another way, in some cases, the epitope present in a TMMP of the present disclosure binds to a TCR on a T cell with an affinity of from about 1 nM to about 5 nM, from about 5 nM to about 10 nM, from about 10 nM to about 50 nM, from about 50 nM to about 100 nM, from about 0.1 μM to about 0.5 μM, from about 0.5 μM to about 1 μM, from about 1 μM to about 5 μM, from about 5 μM to about 10 μM, from about 10 μM to about 25 μM, from about 25 μM to about 50 μM, from about 50 μM to about 75 μM, from about 75 μM to about 100 μM.

In some cases, an immunomodulatory polypeptide present in a TMMP comprises a wild-type (naturally-occurring) amino acid sequence.

In some cases, an immunomodulatory polypeptide present in a TMMP binds to its cognate co-immunomodulatory polypeptide with an affinity that it at least 10% less, at least 15% less, at least 20% less, at least 25% less, at least 30% less, at least 35% less, at least 40% less, at least 45% less, at least 50% less, at least 55% less, at least 60% less, at least 65% less, at least 70% less, at least 75% less, at least 80% less, at least 85% less, at least 90% less, at least 95% less, or more than 95% less, than the affinity of a corresponding wild-type immunomodulatory polypeptide for the cognate co-immunomodulatory polypeptide.

In some cases, a variant immunomodulatory polypeptide present in a TMMP has a binding affinity for a cognate co-immunomodulatory polypeptide that is from 1 nM to 100 nM, or from 100 nM to 100 μM. For example, in some cases, a variant immunomodulatory polypeptide present in a TMMP has a binding affinity for a cognate co-immunomodulatory polypeptide that is from about 100 nM to 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM. In some cases, a variant immunomodulatory polypeptide present in a TMMP of the present disclosure has a binding affinity for a cognate co-immunomodulatory polypeptide that is from about 1 nM to about 5 nM, from about 5 nM to about 10 nM, from about 10 nM to about 50 nM, from about 50 nM to about 100 nM

The combination of the reduced affinity of the immunomodulatory polypeptide for its cognate co-immunomodulatory polypeptide, and the affinity of the epitope for a TCR, provides for enhanced selectivity of a TMMP. For example, in some cases, a TMMP binds selectively to a first T cell that displays both: i) a TCR specific for the epitope present in the TMMP; and ii) a co-immunomodulatory polypeptide that binds to the immunomodulatory polypeptide present in the TMMP, compared to binding to a second T cell that displays: i) a TCR specific for an epitope other than the epitope present in the TMMP; and ii) a co-immunomodulatory polypeptide that binds to the immunomodulatory polypeptide present in the TMMP. For example, in some cases, a TMMP binds to the first T cell with an affinity that is at least 10%, at least 15%, at least 20%, at least 25%, at least 30%, at least 40%, at least 50%, at least 60%, at least 70%, at least 80%, at least 90%, at least 2-fold, at least 2.5-fold, at least 5-fold, at least 10-fold, at least 15-fold, at least 20-fold, at least 25-fold, at least 50-fold, at least 100-fold, or more than 100-fold, higher than the affinity to which it binds the second T cell.

In some cases, a TMMP, when administered to an individual in need thereof, induces both an epitope-specific T cell response and an epitope non-specific T cell response. In other words, in some cases, a TMMP, when administered to an individual in need thereof, induces an epitope-specific T cell response by modulating the activity of a first T cell that displays both: i) a TCR specific for the epitope present in the TMMP; ii) a co-immunomodulatory polypeptide that binds to the immunomodulatory polypeptide present in the TMMP; and induces an epitope non-specific T cell response by modulating the activity of a second T cell that displays: i) a TCR specific for an epitope other than the epitope present in the TMMP; and ii) a co-immunomodulatory polypeptide that binds to the immunomodulatory polypeptide present in the TMMP. The ratio of the epitope-specific T cell response to the epitope-non-specific T cell response is at least 2:1, at least 5:1, at least 10:1, at least 15:1, at least 20:1, at least 25:1, at least 50:1, or at least 100:1. The ratio of the epitope-specific T cell response to the epitope-non-specific T cell response is from about 2:1 to about 5:1, from about 5:1 to about 10:1, from about 10:1 to about 15:1, from about 15:1 to about 20:1, from about 20:1 to about 25:1, from about 25:1 to about 50:1, or from about 50:1 to about 100:1, or more than 100:1. “Modulating the activity” of a T cell can include one or more of: i) activating a cytotoxic (e.g., CD8⁺) T cell; ii) inducing cytotoxic activity of a cytotoxic (e.g., CD8⁺) T cell; iii) inducing production and release of a cytotoxin (e.g., a perforin; a granzyme; a granulysin) by a cytotoxic (e.g., CD8⁺) T cell; iv) inhibiting activity of an autoreactive T cell; and the like.

The combination of the reduced affinity of the immunomodulatory polypeptide for its cognate co-immunomodulatory polypeptide, and the affinity of the epitope for a TCR, provides for enhanced selectivity of a TMMP. Thus, for example, a TMMP binds with higher avidity to a first T cell that displays both: i) a TCR specific for the epitope present in the TMMP; and ii) a co-immunomodulatory polypeptide that binds to the immunomodulatory polypeptide present in the TMMP, compared to the avidity to which it binds to a second T cell that displays: i) a TCR specific for an epitope other than the epitope present in the TMMP; and ii) a co-immunomodulatory polypeptide that binds to the immunomodulatory polypeptide present in the TMMP.

Binding affinity between an immunomodulatory polypeptide and its cognate co-immunomodulatory polypeptide can be determined by bio-layer interferometry (BLI) using purified immunomodulatory polypeptide and purified cognate co-immunomodulatory polypeptide. Binding affinity between a TMMP and its cognate co-immunomodulatory polypeptide can be determined by BLI using purified TMMP and the cognate co-immunomodulatory polypeptide. BLI methods are well known to those skilled in the art. See, e.g., Lad et al. (2015) J. Biomol. Screen. 20(4):498-507; and Shah and Duncan (2014) J. Vis. Exp. 18:e51383.

A BLI assay can be carried out using an Octet RED 96 (Pal FortéBio) instrument, or a similar instrument, as follows. A TMMP (e.g., a TMMP of the present disclosure; a control TMMP (where a control TMMP comprises a wild-type immunomodulatory polypeptide)) is immobilized onto an insoluble support (a “biosensor”). The immobilized TMMP is the “target.” Immobilization can be effected by immobilizing a capture antibody onto the insoluble support, where the capture antibody immobilizes the TMMP. For example, immobilization can be effected by immobilizing anti-Fc (e.g., anti-human IgG Fc) antibodies onto the insoluble support, where the immobilized anti-Fc antibodies bind to and immobilize the TMMP (where the TMMP comprises an IgFc polypeptide). A co-immunomodulatory polypeptide is applied, at several different concentrations, to the immobilized TMMP, and the instrument's response recorded. Assays are conducted in a liquid medium comprising 25 mM HEPES pH 6.8, 5% poly(ethylene glycol) 6000, 50 mM KCl, 0.1% bovine serum albumin, and 0.02% Tween 20 nonionic detergent. Binding of the co-immunomodulatory polypeptide to the immobilized TMMP is conducted at 30° C. As a positive control for binding affinity, an anti-MHC Class I monoclonal antibody can be used. For example, anti-HLA Class I monoclonal antibody W6/32 (American Type Culture Collection No. HB-95; Parham et al. (1979) J. Immunol. 123:342), which has a K_{D} of 7 nM, can be used. A standard curve can be generated using serial dilutions of the anti-MHC Class I monoclonal antibody. The co-immunomodulatory polypeptide, or the anti-MHC Class I mAb, is the “analyte.” BLI analyzes the interference pattern of white light reflected from two surfaces: i) from the immobilized polypeptide (“target”); and ii) an internal reference layer. A change in the number of molecules (“analyte”; e.g., co-immunomodulatory polypeptide; anti-HLA antibody) bound to the biosensor tip causes a shift in the interference pattern; this shift in interference pattern can be measured in real time. The two kinetic terms that describe the affinity of the target/analyte interaction are the association constant (kₐ) and dissociation constant (k_{d}). The ratio of these two terms (k_{d}/ₐ) gives rise to the affinity constant K_{D}.

The BLI assay is carried out in a multi-well plate. To run the assay, the plate layout is defined, the assay steps are defined, and biosensors are assigned in Octet Data Acquisition software. The biosensor assembly is hydrated. The hydrated biosensor assembly and the assay plate are equilibrated for 10 minutes on the Octet instrument. Once the data are acquired, the acquired data are loaded into the Octet Data Analysis software. The data are processed in the Processing window by specifying method for reference subtraction, y-axis alignment, inter-step correction, and Savitzky-Golay filtering. Data are analyzed in the Analysis window by specifying steps to analyze (Association and Dissociation), selecting curve fit model (1:1), fitting method (global), and window of interest (in seconds). The quality of fit is evaluated. K_{D} values for each data trace (analyte concentration) can be averaged if within a 3-fold range. K_{D} error values should be within one order of magnitude of the affinity constant values; R² values should be above 0.95. See, e.g., Abdiche et al. (2008) J. Anal. Biochem. 377:209.

Unless otherwise stated herein, the affinity of a TMMP for a cognate co-immunomodulatory polypeptide, or the affinity of a control TMMP (where a control TMMP comprises a wild-type immunomodulatory polypeptide) for a cognate co-immunomodulatory polypeptide, is determined using BLI, as described above.

In some cases, the ratio of: i) the binding affinity of a control TMMP (where the control comprises a wild-type immunomodulatory polypeptide) to a cognate co-immunomodulatory polypeptide to ii) the binding affinity of a TMMP of the present disclosure comprising a variant of the wild-type immunomodulatory polypeptide to the cognate co-immunomodulatory polypeptide, when measured by BLI (as described above), is at least 1.5:1, at least 2:1, at least 5:1, at least 10:1, at least 15:1, at least 20:1, at least 25:1, at least 50:1, at least 100:1, at least 500:1, at least 10²:1, at least 5×10²:1, at least 10³:1, at least 5×10³:1, at least 10⁴:1, at least 10⁵:1, or at least 10⁶:1. In some cases, the ratio of: i) the binding affinity of a control TMMP (where the control comprises a wild-type immunomodulatory polypeptide) to a cognate co-immunomodulatory polypeptide to ii) the binding affinity of a TMMP comprising a variant of the wild-type immunomodulatory polypeptide to the cognate co-immunomodulatory polypeptide, when measured by BLI, is in a range of from 1.5:1 to 10⁶:1, e.g., from 1.5:1 to 10:1, from 10:1 to 50:1, from 50:1 to 10²:1, from 10²:1 to 10³:1, from 10³:1 to 10⁴:1, from 10⁴:1 to 10⁵:1, or from 10⁵:1 to 10⁶:1.

As an example, where a control TMMP comprises a wild-type IL-2 polypeptide, and where a TMMP comprises a variant IL-2 polypeptide (comprising from 1 to 10 amino acid substitutions relative to the amino acid sequence of the wild-type IL-2 polypeptide) as the immunomodulatory polypeptide, the ratio of: i) the binding affinity of the control TMMP to an IL-2 receptor (i.e., the cognate co-immunomodulatory polypeptide) to ii) the binding affinity of the TMMP of the present disclosure to the IL-2 receptor, when measured by BLI, is at least 1.5:1, at least 2:1, at least 5:1, at least 10:1, at least 15:1, at least 20:1, at least 25:1, at least 50:1, at least 100:1, at least 500:1, at least 10²:1, at least 5×10²:1, at least 10³:1, at least 5×10³:1, at least 10⁴:1, at least 10⁵:1, or at least 10⁶:1. In some cases, where a control TMMP comprises a wild-type IL-2 polypeptide, and where a TMMP comprises a variant IL-2 polypeptide (comprising from 1 to 10 amino acid substitutions relative to the amino acid sequence of the wild-type IL-2 polypeptide) as the immunomodulatory polypeptide, the ratio of: i) the binding affinity of the control TMMP to an IL-2 receptor (i.e., the cognate co-immunomodulatory polypeptide) to ii) the binding affinity of the TMMP to the IL-2 receptor, when measured by BLI, is in a range of from 1.5:1 to 106:1, e.g., from 1.5:1 to 10:1, from 10:1 to 50:1, from 50:1 to 10²:1, from 10²:1 to 10³:1, from 10³:1 to 10⁴:1, from 10⁴:1 to 10⁵:1, or from 10⁵:1 to 10⁶:1.

As another example, where a control TMMP comprises a wild-type CD80 polypeptide, and where a TMMP of the present disclosure comprises a variant CD80 polypeptide (comprising from 1 to 10 amino acid substitutions relative to the amino acid sequence of the wild-type CD80 polypeptide) as the immunomodulatory polypeptide, the ratio of: i) the binding affinity of the control TMMP to a CTLA4 polypeptide (i.e., the cognate co-immunomodulatory polypeptide) to ii) the binding affinity of the TMMP of the present disclosure to the CTLA4 polypeptide, when measured by BLI, is at least 1.5:1, at least 2:1, at least 5:1, at least 10:1, at least 15:1, at least 20:1, at least 25:1, at least 50:1, at least 100:1, at least 500:1, at least 10²:1, at least 5×10²:1, at least 10³:1, at least 5×10³:1, at least 10⁴:1, at least 10⁵:1, or at least 10⁶:1.

As another example, where a control TMMP comprises a wild-type CD80 polypeptide, and where a TMMP of the present disclosure comprises a variant CD80 polypeptide (comprising from 1 to 10 amino acid substitutions relative to the amino acid sequence of the wild-type CD80 polypeptide) as the immunomodulatory polypeptide, the ratio of: i) the binding affinity of the control TMMP to a CD28 polypeptide (i.e., the cognate co-immunomodulatory polypeptide) to ii) the binding affinity of the TMMP of the present disclosure to the CD28 polypeptide, when measured by BLI, is at least 1.5:1, at least 2:1, at least 5:1, at least 10:1, at least 15:1, at least 20:1, at least 25:1, at least 50:1, at least 100:1, at least 500:1, at least 10²:1, at least 5×10²:1, at least 10³:1, at least 5×10³:1, at least 10⁴:1, at least 10⁵:1, or at least 10⁶:1.

As another example, where a control TMMP comprises a wild-type 4-1BBL polypeptide, and where a TMMP of the present disclosure comprises a variant 4-1BBL polypeptide (comprising from 1 to 10 amino acid substitutions relative to the amino acid sequence of the wild-type 4-1BBL polypeptide) as the immunomodulatory polypeptide, the ratio of: i) the binding affinity of the control TMMP to a 4-1BB polypeptide (i.e., the cognate co-immunomodulatory polypeptide) to ii) the binding affinity of the TMMP of the present disclosure to the 4-1BB polypeptide, when measured by BLI, is at least 1.5:1, at least 2:1, at least 5:1, at least 10:1, at least 15:1, at least 20:1, at least 25:1, at least 50:1, at least 100:1, at least 500:1, at least 10²:1, at least 5×10²:1, at least 10³:1, at least 5×10³:1, at least 10⁴:1, at least 10⁵:1, or at least 10⁶:1.

As another example, where a control TMMP comprises a wild-type CD86 polypeptide, and where a TMMP of the present disclosure comprises a variant CD86 polypeptide (comprising from 1 to 10 amino acid substitutions relative to the amino acid sequence of the wild-type CD86 polypeptide) as the immunomodulatory polypeptide, the ratio of: i) the binding affinity of the control TMMP to a CD28 polypeptide (i.e., the cognate co-immunomodulatory polypeptide) to ii) the binding affinity of the TMMP of the present disclosure to the CD28 polypeptide, when measured by BLI, is at least 1.5:1, at least 2:1, at least 5:1, at least 10:1, at least 15:1, at least 20:1, at least 25:1, at least 50:1, at least 100:1, at least 500:1, at least 10²:1, at least 5×10²:1, at least 10³:1, at least 5×10³:1, at least 10⁴:1, at least 10⁵:1, or at least 10⁶:1.

Binding affinity of a TMMP of the present disclosure to a target T cell can be measured in the following manner: A) contacting a TMMP of the present disclosure with a target T-cell expressing on its surface: i) a cognate co-immunomodulatory polypeptide that binds the parental wild-type immunomodulatory polypeptide; and ii) a T-cell receptor that binds to the epitope, where the TMMP comprises an epitope tag, such that the TMMP binds to the target T-cell; B) contacting the target T-cell-bound TMMP with a fluorescently labeled binding agent (e.g., a fluorescently labeled antibody) that binds to the epitope tag, generating a TMMP/target T-cell/binding agent complex; C) measuring the mean fluorescence intensity (MFI) of the TMMP/target T-cell/binding agent complex using flow cytometry. The epitope tag can be, e.g., a FLAG tag, a hemagglutinin tag, a c-myc tag, a poly(histidine) tag, etc. The MFI measured over a range of concentrations of the TMMP library member provides a measure of the affinity. The MFI measured over a range of concentrations of the TMMP library member provides a half maximal effective concentration (EC₅₀) of the TMMP. In some cases, the EC₅₀ of a TMMP of the present disclosure for a target T cell is in the nM range; and the EC₅₀ of the TMMP for a control T cell (where a control T cell expresses on its surface: i) a cognate co-immunomodulatory polypeptide that binds the parental wild-type immunomodulatory polypeptide; and ii) a T-cell receptor that does not bind to the epitope present in the TMMP) is in the M range. In some cases, the ratio of the EC₅₀ of a TMMP of the present disclosure for a control T cell to the EC₅₀ of the TMMP for a target T cell is at least 1.5:1, at least 2:1, at least 5:1, at least 10:1, at least 15:1, at least 20:1, at least 25:1, at least 50:1, at least 100:1, at least 500:1, at least 10²:1, at least 5×10²:1, at least 10³:1, at least 5×10³:1, at least 10⁴:1, at lease 10⁵:1, or at least 10⁶:1. The ratio of the EC₅₀ of a TMMP of the present disclosure for a control T cell to the EC₅₀ of the TMMP for a target T cell is an expression of the selectivity of the TMMP.

In some cases, when measured as described in the preceding paragraph, a TMMP exhibits selective binding to target T-cell, compared to binding of the TMMP library member to a control T cell that comprises: i) the cognate co-immunomodulatory polypeptide that binds the parental wild-type immunomodulatory polypeptide; and ii) a T-cell receptor that binds to an epitope other than the epitope present in the TMMP library member.

### Epitopes

A peptide epitope present in a TMMP can have a length of at least 4 amino acids, e.g., from 4 amino acids to about 25 amino acids in length (e.g., 4 amino acids (aa), 5 aa, 6 aa, 7 aa, 8 aa, 9 aa, 10 aa, 11 aa, 12 aa, 13 aa, 14 aa, 15 aa, 16 aa, 17 aa, 18 aa, 19 aa, 20 aa, 21 aa, 22 aa, 23 aa, 24 aa, or 25 aa, including within a range of from 4 to 20 amino acids, from 6 to 18 amino acids, from 8 to 15 amino acids, from 8 to 12 amino acids, from 5 to 10 amino acids, from 10 to 20 amino acids, and from 15 to 25 amino acids in length).

A TMMP can comprise any of a variety of peptide epitopes. As discussed above, a peptide epitope present in a TMMP is a peptide that, when complexed with MHC polypeptides, presents an epitope to a T-cell receptor (TCR). An epitope-specific T cell binds an epitope having a given amino acid sequence, i.e., a “reference” amino acid sequence, but substantially does not bind an epitope that differs from the reference amino acid sequence, or if it binds at all, binds an epitope that differs from the reference amino acid sequence with only low affinity, e.g., less than 10⁻⁶ M, less than 10⁻⁵ M, or less than 10⁻⁴ M. For example, an epitope-specific T cell binds an epitope that differs from the reference amino acid sequence, if at all, with an affinity that is less than 10⁻⁶ M, less than 10⁻⁵ M, or less than 10⁻⁴ M. An epitope-specific T cell can bind an epitope having a reference amino acid sequence, i.e., for which it is specific, with an affinity of at least 10⁻⁷ M, at least 10⁻⁸ M, at least 10⁻⁹ M, or at least 10⁻¹⁰ M.

In some cases, the epitope peptide present in a TMMP presents an epitope specific to an HLA-A, -B, -C, -E, -F, or -G allele. In an embodiment, the epitope peptide present in a TMMP presents an epitope restricted to HLA-A*0101, A*0201, A*0301, A*1101, A*2301, A*2402, A*2407, A*3303, and/or A*3401. In an embodiment, the epitope peptide present in a TMMP presents an epitope restricted to HLA-B*0702, B*0801, B*1502, B*3802, B*4001, B*4601, and/or B*5301. In an embodiment, the epitope peptide present in a TMMP presents an epitope restricted to C*0102, C*0303, C*0304, C*0401, C*0602, C*0701, C*702, C*0801, and/or C*1502.

In some cases, the peptide epitope is a viral epitope. In some cases, a viral epitope is an epitope present in a viral antigen encoded by a virus that infects a majority of the human population, where such viruses include, e.g., cytomegalovirus (CMV), Epstein-Barr virus (EBV), human papilloma virus, influenza virus, adenovirus, and the like. In some cases, the peptide epitope is a bacterial epitope, e.g., a bacterial epitope that is included in a vaccine and to which a majority of the human population has immunity.

### 1) CMV Peptide Epitopes

In some cases, a TMMP comprises a CMV peptide epitope, i.e., a peptide that when in an MHC/peptide complex (e.g., an HLA/peptide complex), presents a CMV epitope (i.e., an epitope present in a CMV antigen) to a T cell. As with other peptide epitopes of this disclosure, a CMV peptide epitope has a length of at least 4 amino acids, e.g., from 4 amino acids to about 25 amino acids (e.g., 4 amino acids (aa), 5 aa, 6 aa, 7 aa, 8 aa, 9 aa, 10 aa, 11 aa, 12 aa, 13 aa, 14 aa, 15 aa, 16 aa, 17 aa, 18 aa, 19 aa, 20 aa, 21 aa, 22 aa, 23 aa, 24 aa, or 25 aa, including within a range of from 4 to 20 aa, from 6 to 18 aa, from 8 to 15 aa. from 8 to 12 aa, from 5 to 10 aa, from 10 to 15 aa, from 15 to 20 aa, from 10 to 20 aa, or from 15 to 25 aa. in length).

A given CMV epitope-specific T cell binds an epitope having a reference amino acid sequence of a given CMV epitope, but substantially does not bind an epitope that differs from the reference amino acid sequence, or if it binds at all, binds an epitope that differs from the reference amino acid sequence with only low affinity, e.g., less than 10⁻⁶ M, less than 10⁻⁵ M, or less than 10⁻⁴ M. For example, a given CMV epitope-specific T cell binds a CMV epitope having a reference amino acid sequence, and binds an epitope that differs from the reference amino acid sequence, if at all, with an affinity that is less than 10⁻⁶ M, less than 10⁻⁵ M, or less than 10⁻⁴ M. A given CMV epitope-specific T cell can bind an epitope for which it is specific with an affinity of at least 10⁻⁷ M, at least 10⁻⁸ M, at least 10⁻⁹ M, or at least 10⁻¹⁰ M.

In some cases, a CMV peptide epitope present in a TMMP is a peptide from CMV pp65. In some cases, a CMV peptide epitope present in a TMMP is a peptide from CMV gB (glycoprotein B).

For example, in some cases, a CMV peptide epitope present in a TMMP is a peptide of a CMV polypeptide having a length of at least 4 amino acids, e.g., from 4 amino acids to about 25 amino acids (e.g., 4 amino acids (aa), 5 aa, 6 aa, 7 aa, 8 aa, 9 aa, 10 aa, 11 aa, 12 aa, 13 aa, 14 aa, 15 aa, 16 aa, 17 aa, 18 aa, 19 aa, 20 aa, 21 aa, 22 aa, 23 aa, 24 aa, or 25 aa, including within a range of from 4 to 20 aa, from 6 to 18 aa, from 8 to 15 aa. from 8 to 12 aa, from 5 to 10 aa, from 10 to 15 aa, from 15 to 20 aa, from 10 to 20 aa, or from 15 to 25 aa. in length), and comprising an amino acid sequence having at least 80%, at least 85%, at least 90%, at least 95%, at least 98%, at least 99%, or 100%, amino acid sequence identity to the following CMV pp65 amino acid sequence:

(SEQ ID NO: 123)
MESRGRRCPE MISVLGPISG HVLKAVFSRG DTPVLPHETR
LLQTGIHVRV SQPSLILVSQ YTPDSTPCHR GDNQLQVQHT
YFTGSEVENV SVNVHNPTGR SICPSQEPMS IYVYALPLKM
LNIPSINVHH YPSAAERKHR HLPVADAVIH ASGKQMWQAR
LTVSGLAWTR QQNQWKEPDV YYTSAFVFPT KDVALRHVVC
AHELVCSMEN TRATKMQVIG DQYVKVYLES FCEDVPSGKL
FMHVTLGSDV EEDLTMTRNP QPFMRPHERN GFTVLCPKNM
IIKPGKISHI MLDVAFTSHE HFGLLCPKSI PGLSISGNLL
MNGQQIFLEV QAIRETVELR QYDPVAALFF FDIDLLLQRG
PQYSEHPTFT SQYRIQGKLE YRHTWDRHDE GAAQGDDDVW
TSGSDSDEEL VTTERKTPRV TGGGAMAGAS TSAGRKRKSA
SSATACTSGV MTRGRLKAES TVAPEEDTDE DSDNEIHNPA
VFTWPPWQAG ILARNLVPMV ATVQGQNLKY QEFFWDANDI
YRIFAELEGV WQPAAQPKRR RHRQDALPGP CIASTPKKHR G.

As one non-limiting example, a CMV peptide epitope present in a TMMP has the amino acid sequence NLVPMVATV (SEQ ID NO: 172) and has a length of 9 amino acids.

In some cases, a CMV peptide epitope present in a TMMP is a peptide having a length of at least 4 amino acids, e.g., from 4 amino acids to about 25 amino acids (e.g., 4 amino acids (aa), 5 aa, 6 aa, 7 aa, 8 aa, 9 aa, 10 aa, 11 aa, 12 aa, 13 aa, 14 aa, 15 aa, 16 aa, 17 aa, 18 aa, 19 aa, 20 aa, 21 aa, 22 aa, 23 aa, 24 aa, or 25 aa, including within a range of from 4 to 20 aa, from 6 to 18 aa, from 8 to 15 aa. from 8 to 12 aa, from 5 to 10 aa, from 10 to 15 aa, from 15 to 20 aa, from 10 to 20 aa, or from 15 to 25 aa. in length) of a CMV polypeptide comprising an amino acid sequence having at least 80%, at least 85%, at least 90%, at least 95%, at least 98%, at least 99%, or 100%, amino acid sequence identity to the following CMV gB amino acid sequence:

(SEQ ID NO: 173)
MESRIWCLVVCVNLCIVCLGAAVSSSSTSHATSSTHNGSHTSRTTSAQ
TRSVYSQHVTSSEAVSHRANETIYNTTLKYGDVVGVNTTKYPYRVCSM
AQGTDLIRFERNIICTSMKPINEDLDEGIMVVYKRNIVAHTFKVRVYQ
KVLTFRRSYAYIYTTYLLGSNTEYVAPPMWEIHHINKFAQCYSSYSRV
IGGTVFVAYHRDSYENKTMQLIPDDYSNTHSTRYVTVKDQWHSRGSTW
LYRETCNLNCMLTITTARSKYPYHFFATSTGDVVYISPFYNGTNRNAS
YFGENADKFFIFPNYTIVSDFGRPNAAPETHRLVAFLERADSVISWDI
QDEKNVTCQLTFWEASERTIRSEAEDSYHFSSAKMTATFLSKKQEVNM
SDSALDCVRDEAINKLQQIFNTSYNQTYEKYGNVSVFETSGGLVVFWQ
GIKQKSLVELERLANRSSLNITHRTRRSTSDNNTTHLSSMESVHNLVY
AQLQFTYDTLRGYINRALAQIAEAWCVDQRRTLEVFKELSKINPSAIL
SAIYNKPIAARFMGDVLGLASCVTINQTSVKVLRDMNVKESPGRCYSR
PVVIFNFANSSYVQYGQLGEDNEILLGNHRTEECQLPSLKIFIAGNSA
YEYVDYLFKRMIDLSSISTVDSMIALDIDPLENTDFRVLELYSQKELR
SSNVFDLEEIMREFNSYKQRVKYVEDKVVDPLPPYLKGLDDLMSGLGA
AGKAVGVAIGAVGGAVASVVEGVATFLKNPFGAFTIILVAIAVVIITY
LIYTRQRRLCTQPLQNLFPYLVSADGTTVTSGSTKDTSLQAPPSYEES
VYNSGRKGPGPPSSDASTAAPPYTNEQAYQMLLALARLDAEQRAQQNG
TDSLDGQTGTQDKGQKPNLLDRLRHRKNGYRHLKDSDEEENV.

In some cases, the CMV epitope present in a TMMP presents an epitope specific to an HLA-A, -B, -C, -E, -F, or -G allele. In some cases, the epitope peptide present in a TMMP presents an epitope restricted to HLA-A*0101, A*0201, A*0301, A*1101, A*2301, A*2402, A*2407, A*3303, and/or A*3401. In some cases, the CMV epitope present in a TMMP presents an epitope restricted to HLA-B*0702, B*0801, B*1502, B*3802, B*4001, B*4601, and/or B*5301. In some cases, the CMV epitope present in a TMMP presents an epitope restricted to C*0102, C*0303, C*0304, C*0401, C*0602, C*0701, C*702, C*0801, and/or C*1502. As one example, in some cases, a TMMP comprises: a) a CMV peptide epitope having amino acid sequence NLVPMVATV (SEQ ID NO:172) and having a length of 9 amino acids; b) an HLA-A*0201 class I heavy chain polypeptide; and c) a β2M polypeptide.

### 2) HPV Epitopes

An HPV peptide suitable for inclusion in a TMMP can be a peptide of an HPV E6 polypeptide or an HPV E7 polypeptide. The HPV epitope can be an epitope of HPV of any of a variety of genotypes, including, e.g., HPV16, HPV18, HPV31, HPV33, HPV35, HPV39, HPV45, HPV51, HPV52, HPV56, HPV58, HPV59, HPV68, HPV73, or HPV82. In some cases, the epitope is an HPV E6 epitope. In some cases, the epitope is an HPV E7 epitope.

An HPV epitope present in a TMMP is a peptide specifically bound by a T-cell, i.e., the epitope is specifically bound by an HPV epitope-specific T cell. An epitope-specific T cell binds an epitope having a reference amino acid sequence, but substantially does not bind an epitope that differs from the reference amino acid sequence, or if it binds at all, binds an epitope that differs from the reference amino acid sequence with only low affinity, e.g., less than 10⁻⁶ M, less than 10⁻⁵ M, or less than 10⁻⁴ M. For example, an epitope-specific T cell binds an epitope having a reference amino acid sequence, and binds an epitope that differs from the reference amino acid sequence, if at all, with an affinity that is less than 10⁻⁶ M, less than 10⁻⁵ M, or less than 10⁻⁴ M. An epitope-specific T cell can bind an epitope for which it is specific with an affinity of at least 10⁻⁷ M, at least 10⁻⁸ M, at least 10⁻⁹ M, or at least 10⁻¹⁰ M.

Examples of HPV E6 peptides suitable for inclusion in a TMMP include, but are not limited to, E6 18-26 (KLPQLCTEL; SEQ ID NO: 124); E6 26-34 (LQTTIHDII; SEQ ID NO: 125); E6 49-57 (VYDFAFRDL; SEQ ID NO: 126); E6 52-60 (FAFRDLCIV; SEQ ID NO: 127); E6 75-83 (KFYSKISEY; SEQ ID NO: 128); and E6 80-88 (ISEYRHYCY; SEQ ID NO: 129).

Examples of HPV E7 peptides suitable for inclusion in a TMMP include, but are not limited to, E7 7-15 (TLHEYMLDL; SEQ ID NO: 130); E7 11-19 (YMLDLQPET; SEQ ID NO:131); E7 44-52 (QAEPDRAHY; SEQ ID NO: 132); E7 49-57 (RAHYNIVTF (SEQ ID NO: 132); E7 61-69 (CDSTLRLCV; SEQ ID NO: 133); and E7 67-76 (LCVQSTHVDI; SEQ ID NO: 134); E7 82-90 (LLMGTLGIV; SEQ ID NO: 135); E7 86-93 (TLGIVCPI; SEQ ID NO: 136); and E7 92-93 (LLMGTLGIVCPI; SEQ ID NO: 137).

In some cases, a suitable HPV peptide is an HPV E6 peptide that binds HLA-A24 (e.g., is an HLA-A2401-restricted epitope). Non-limiting examples include: VYDFAFRDL (SEQ ID NO: 126); CYSLYGTTL (SEQ ID NO: 139); EYRHYCYSL (SEQ ID NO: 140); KLPQLCTEL (SEQ ID NO: 124); DPQERPRKL (SEQ ID NO:141); HYCYSLYGT (SEQ ID NO: 142); DFAFRDLCI (SEQ ID NO: 143); LYGTTLEQQY (SEQ ID NO: 144); HYCYSLYGTT (SEQ ID NO: 145); EVYDFAFRDL (SEQ ID NO: 146); EYRHYCYSLY (SEQ ID NO: 147); VYDFAFRDLC (SEQ ID NO: 148); YCYSIYGTTL (SEQ ID NO: 149); VYCKTVLEL (SEQ ID NO: 150); VYGDTLEKL (SEQ ID NO:151); and LTNTGLYNLL (SEQ ID NO: 152).

In some cases, a suitable HPV peptide is selected from the group consisting of:

(SEQ ID NO: 153)
DLQPETTDL;
(SEQ ID NO: 130)
TLHEYMLDL;
(SEQ ID NO: 154)
TPTLHEYML;
(SEQ ID NO: 133)
RAHYNIVTF;
(SEQ ID NO: 155)
GTLGIVCPI;
(SEQ ID NO: 156)
EPDRAHYNI;
(SEQ ID NO: 157)
QLFLNTLSF;
(SEQ ID NO: 158)
FQQLFLNTL;
and
(SEQ ID NO: 159)
AFQQLFLNTL.

In some cases, a suitable HPV peptide presents an HLA-A*2401-restricted epitope. Non-limiting examples of HPV peptides presenting an HLA-A*2401-restricted epitope are: VYDFAFRDL (SEQ ID NO: 126); RAHYNIVTF (SEQ ID NO: 133); CDSTLRLCV (SEQ ID NO: 134); and LCVQSTHVDI (SEQ ID NO: 135). In some cases, an HPV peptide suitable for inclusion in a TMMP is VYDFAFRDL (SEQ ID NO: 126). In some cases, an HPV peptide suitable for inclusion in a TMMP is RAHYNIVTF (SEQ ID NO: 133). In some cases, an HPV peptide suitable for inclusion in a TMMP is CDSTLRLCV (SEQ ID NO: 134). In some cases, an HPV peptide suitable for inclusion in a TMMP is LCVQSTHVDI (SEQ ID NO: 135).

### 3) Influenza Virus Epitopes

Influenza virus peptides that are suitable for inclusion as a peptide epitope of a TMMP include peptides of from 4 amino acids to 25 amino acids in length of an influenza polypeptide, e.g., an influenza polypeptide that is included in a vaccine, or that is present in an influenza virus that infects a human. As one example, a peptide suitable for inclusion as a peptide epitope of a TMMP is an influenza virus peptide of from 4 amino acids to 25 amino acids in length of an influenza virus nucleoprotein. As another example, a peptide suitable for inclusion as a peptide epitope of a TMMP is a peptide of from 4 amino acids to 25 amino acids in length of an influenza virus hemagglutinin polypeptide. As another example, a peptide suitable for inclusion as a peptide epitope of a TMMP is a peptide of from 4 amino acids to 25 amino acids in length of an influenza A virus Matrix protein 1. As another example, a peptide suitable for inclusion as a peptide epitope of a TMMP is a peptide of from 4 amino acids to 25 amino acids in length of an influenza virus neuraminidase polypeptide. In some cases, the peptide is a peptide that presents an immunodominant influenza virus protein epitope. One non-limiting example of a suitable influenza peptide is a peptide having the sequence GILGFVFTL (SEQ ID NO:160) and having a length of 9 amino acids.

### 4) Tetanus Epitopes

Tetanus peptides that are suitable for inclusion as a peptide epitope of a TMMP include peptides of from 4 amino acids to 25 amino acids in length of a tetanus toxin. Examples of suitable tetanus peptides include, but are not limited to, QYIKANSKFIGIFE (SEQ ID NO:161); QYIKANSKFIGITE (SEQ ID NO: 162); ILMQYIKANSKFIGI (SEQ ID NO: 163); VNNESSE (SEQ ID NO: 164); PGINGKAIHLVNNESSE (SEQ ID NO: 165); PNRDIL (SEQ ID NO: 166); FIGITEL (SEQ ID NO: 167); SYFPSV (SEQ ID NO: 168); NSVDDALINSTKIYSYFPSV (SEQ ID NO: 169); and IDKISDVSTIVPYIGPALNI (SEQ ID NO: 170).

### MHC Polypeptides

As noted above, a TMMP includes MHC polypeptides. For the purposes of the instant disclosure, the term “major histocompatibility complex (MHC) polypeptides” is meant to include MHC polypeptides of various species, including human MHC (also referred to as human leukocyte antigen (HLA)) polypeptides, rodent (e.g., mouse, rat, etc.) MHC polypeptides, and MHC polypeptides of other mammalian species (e.g., lagomorphs, non-human primates, canines, felines, ungulates (e.g., equines, bovines, ovines, caprines, etc.), and the like. The term “MHC polypeptide” is meant to include Class I MHC polypeptides (e.g., β-2 microglobulin and MHC class I heavy chain).

In some cases, the first MHC polypeptide is an MHC Class I β2M (β2M) polypeptide, and the second MHC polypeptide is an MHC Class I heavy chain (H chain) (“MHC-H”)). In other instances, the first MHC polypeptide is an MHC Class I heavy chain polypeptide; and the second MHC polypeptide is a β2M polypeptide. In some cases, both the β2M and MHC-H chain are of human origin; i.e., the MHC-H chain is an HLA heavy chain, or a variant thereof. Unless expressly stated otherwise, a TMMP of the present disclosure does not include membrane anchoring domains (transmembrane regions) of an MHC Class I heavy chain, or a part of MHC Class I heavy chain sufficient to anchor the resulting TMMP to a cell (e.g., eukaryotic cell such as a mammalian cell) in which it is expressed. In some cases, the MHC Class I heavy chain present in a TMMP of the present disclosure does not include a signal peptide, a transmembrane domain, or an intracellular domain (cytoplasmic tail) associated with a native MHC Class I heavy chain. Thus, e.g., in some cases, the MHC Class I heavy chain present in a TMMP of the present disclosure includes only the α1, α2, and α3 domains of an MHC Class I heavy chain. In some cases, the MHC Class I heavy chain present in a TMMP of the present disclosure has a length of from about 270 amino acids (aa) to about 290 aa. In some cases, the MHC Class I heavy chain present in a TMMP of the present disclosure has a length of 270 aa, 271 aa, 272 aa, 273 aa, 274 aa, 275 aa, 276 aa, 277 aa, 278 aa, 279 aa, 280 aa, 281 aa, 282 aa, 283 aa, 284 aa, 285 aa, 286 aa, 287 aa, 288 aa, 289 aa, or 290 aa.

In some cases, an MHC polypeptide of a TMMP is a human MHC polypeptide, where human MHC polypeptides are also referred to as “human leukocyte antigen” (“HLA”) polypeptides. In some cases, an MHC polypeptide of a TMMP is a Class I HLA polypeptide, e.g., a β2-microglobulin polypeptide, or a Class I HLA heavy chain polypeptide. Class I HLA heavy chain polypeptides include HLA-A heavy chain polypeptides, HLA-B heavy chain polypeptides, HLA-C heavy chain polypeptides, HLA-E heavy chain polypeptides, HLA-F heavy chain polypeptides, and HLA-G heavy chain polypeptides.

### MHC Class I Heavy Chains

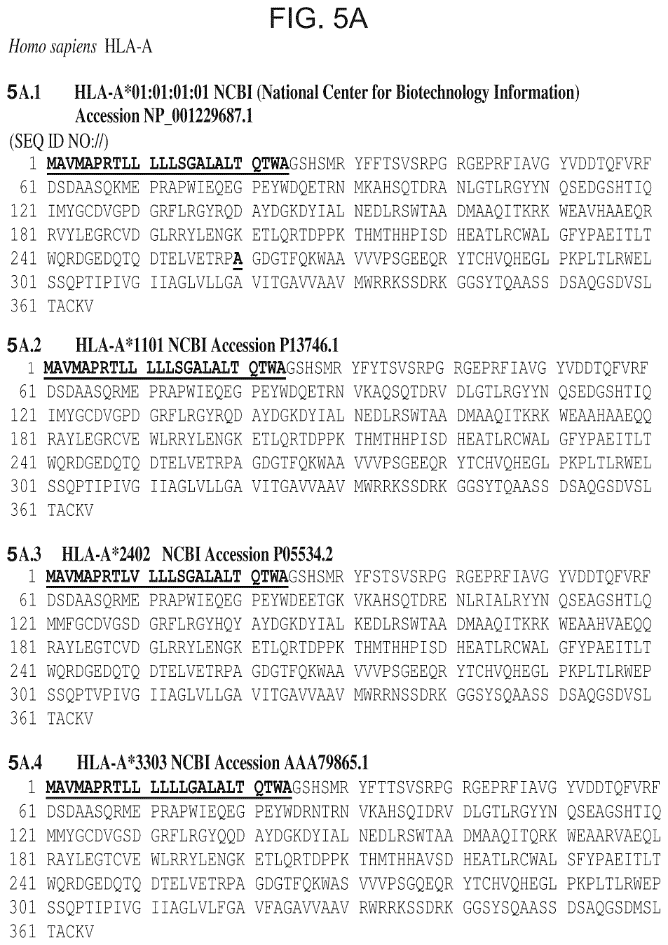

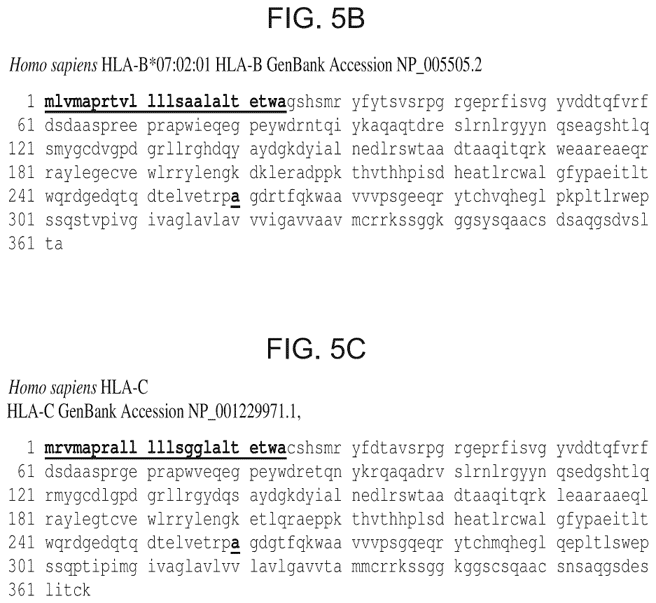

In some cases, an MHC Class I heavy chain polypeptide present in a TMMP of the present disclosure comprises an amino acid sequence having at least 75%, at least 80%, at least 85%, at least 90%, at least 95%, at least 98%, at least 99%, or 100%, amino acid sequence identity to all or part (e.g., 50, 75, 100, 150, 200, or 250 contiguous amino acids) of the amino acid sequence of any of the human HLA heavy chain polypeptides depicted in FIGS. 5-11. In some cases, the MHC Class I heavy chain has a length of 270 aa, 271 aa, 272 aa, 273 aa, 274 aa, 275 aa, 276 aa, 277 aa, 278 aa, 279 aa, 280 aa, 281 aa, 282 aa, 283 aa, 284 aa, 285 aa, 286 aa, 287 aa, 288 aa, 289 aa, or 290 aa. In some cases, an MHC Class I heavy chain polypeptide present in a TMMP of the present disclosure comprises 1-30, 1-5, 5-10, 10-15, 15-20, 20-25 or 25-30 amino acid insertions, deletions, and/or substitutions (in addition to those locations indicated as being variable in the heavy chain consensus sequences) of any one of the amino acid sequences depicted in FIGS. 5-11. In some cases, the MHC Class I heavy chain does not include transmembrane or cytoplasmic domains. As an example, a MHC Class I heavy chain polypeptide of a TMMP of the present disclosure can comprise an amino acid sequence having at least 75%, at least 80%, at least 85%, at least 90%, at least 95%, at least 98%, at least 99%, or 100%, amino acid sequence identity to amino acids 25-300 (lacking all, or substantially all, of the leader, transmembrane and cytoplasmic sequence) or amino acids 25-365 (lacking the leader) of a human HLA-A heavy chain polypeptides depicted in any one of FIGS. 5A, 5B, and 5C.

FIGS. 5A, 5B and 5C provide amino acid sequences of human leukocyte antigen (HLA) Class I heavy chain polypeptides. Signal sequences, amino acids 1-24, are bolded and underlined. FIG. 5A entry: 3A.1 is the HLA-A heavy chain (HLA-A*01:01:01:01 or A*0101) (NCBI accession NP_001229687.1), SEQ ID NO:392; entry 3A.2 is from HLA-A*1101 SEQ ID NO:393; entry 3A.3 is from HLA-A*2402 SEQ ID NO:394 and entry 3A.4 is from HLA-A*3303 SEQ ID NO:395. FIG. 5B provides the sequence HLA-B*07:02:01 (HLA-B*0702) NCBI GenBank Accession NP_005505.2 (see also GenBank Accession AUV50118.1.). FIG. 5C provides the sequence HLA-C*0701 (GenBank Accession NP_001229971.1) (HLA-C*07:01:01:01 or HLA-Cw*070101, HLA-Cw*07 see GenBank Accession CA078194.1).

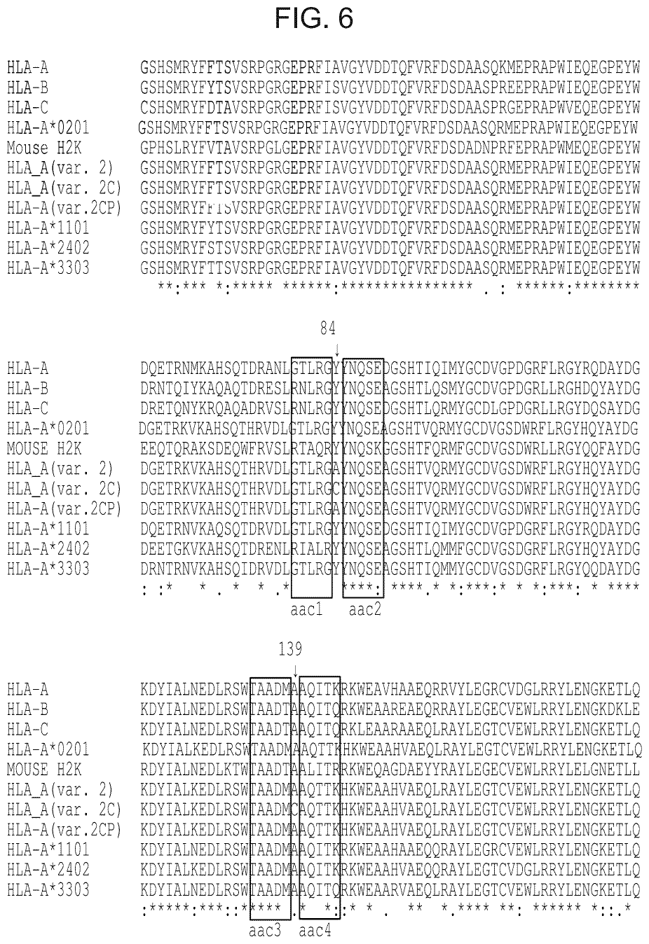

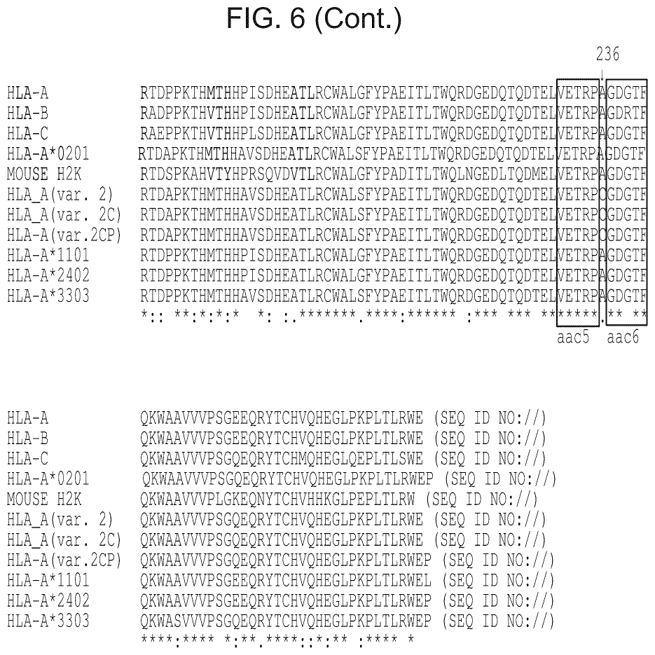

FIG. 6 provides an alignment of eleven mature MHC class I heavy chain amino acid sequences without their leader sequences or transmembrane domains or intracellular domains. The aligned sequences are human HLA-A, HLA-B, and HLA-C, a mouse H2K protein sequence, three variants of HLA-A (var.1, var. 2C, and var.2CP), and 3 human HLA-A variants (HLA-A*1101; HLA-A*2402; and HLA-A*3303). Indicated in the alignment are the locations (84 and 139 of the mature proteins) where cysteine residues may be introduced (e.g., by substitution) for the formation of a disulfide bond to stabilize the MHC H chain-β2M complex. Also shown in the alignment is position 236 (of the mature polypeptide), which may be substituted by a cysteine residue that can form an inter-chain disulfide bond with β2M (e.g., at aa 12). An arrow appears above each of those locations and the residues are bolded. The seventh HLA-A sequence shown in the alignment (var. 2c), shows the sequence of variant 2 substituted with C residues at positions 84, 139 and 236. The boxes flanking residues 84, 139 and 236 show the groups of five amino acids on either sides of those six sets of five residues, denoted aac1 (for “amino acid cluster 1”), aac2 (for “amino acid cluster 2”), aac3 (for “amino acid cluster 3”), aac4 (for “amino acid cluster 4”), aac5 (for “amino acid cluster 5”), and aac6 (for “amino acid cluster 6”), that may be replaced by 1 to 5 amino acids selected independently from (i) any naturally occurring amino acid or (ii) any naturally occurring amino acid except proline or glycine.

With regard to FIG. 6, in some cases: i) aac1 (amino acid cluster 1) may be the amino acid sequence GTLRG (SEQ ID NO: 174) or that sequence with one or two amino acids deleted or substituted with other naturally occurring amino acids (e.g., L replaced by I, V, A or F); ii) aac2 (amino acid cluster 2) may be the amino acid sequence YNQSE (SEQ ID NO: 175) or that sequence with one or two amino acids deleted or substituted with other naturally occurring amino acids (e.g., N replaced by Q, Q replaced by N, and/or E replaced by D); iii) aac3 (amino acid cluster 3) may be the amino acid sequence TAADM (SEQ ID NO: 176) or that sequence with one or two amino acids deleted or substituted with other naturally occurring amino acids (e.g., T replaced by S, A replaced by G, D replaced by E, and/or M replaced by L, V, or I); iv) aac4 (amino acid cluster 4) may be the amino acid sequence AQTTK (SEQ ID NO: 177) or that sequence with one or two amino acids deleted or substituted with other naturally occurring amino acids (e.g., A replaced by G, Q replaced by N, or T replaced by S, and/or K replaced by R or Q); v) aac5 (amino acid cluster 5) may be the amino acid sequence VETRP (SEQ ID NO: 178) or that sequence with one or two amino acids deleted or substituted with other naturally occurring amino acids (e.g., V replaced by I or L, E replaced by D, T replaced by S, and/or R replaced by K); and/or vi) aac6 (amino acid cluster 6) may be the amino acid sequence GDGTF (SEQ ID NO: 179) or that sequence with one or two amino acids deleted or substituted with other naturally occurring amino acids (e.g., D replaced by E, T replaced by S, or F replaced by L, W, or Y).

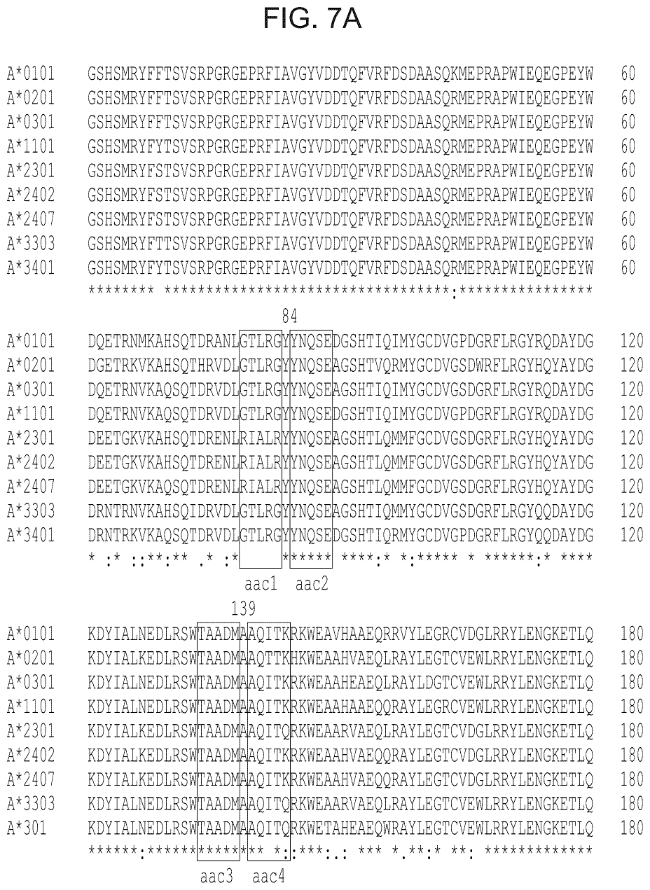

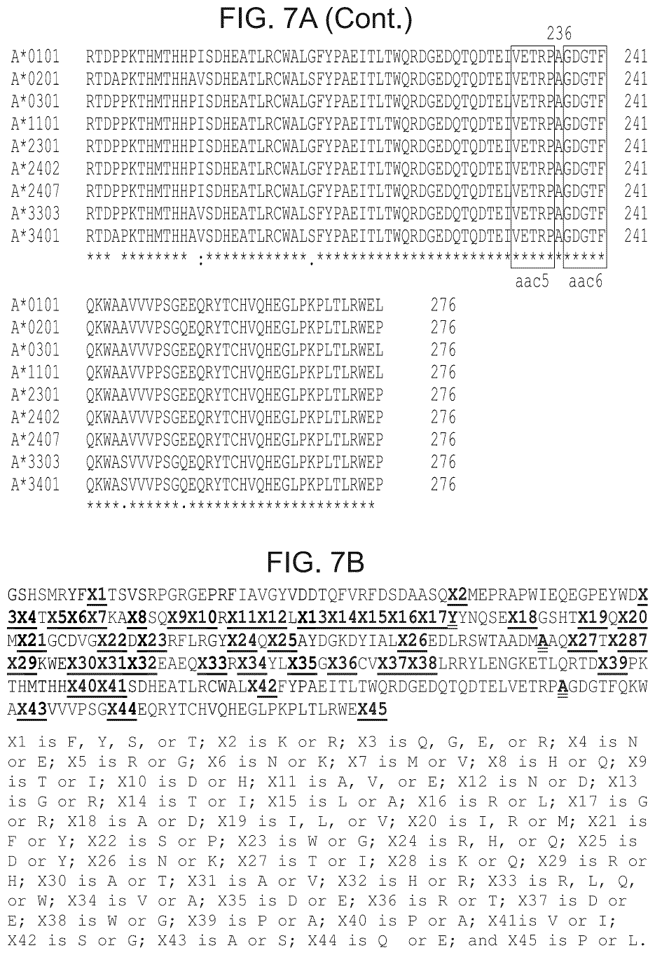

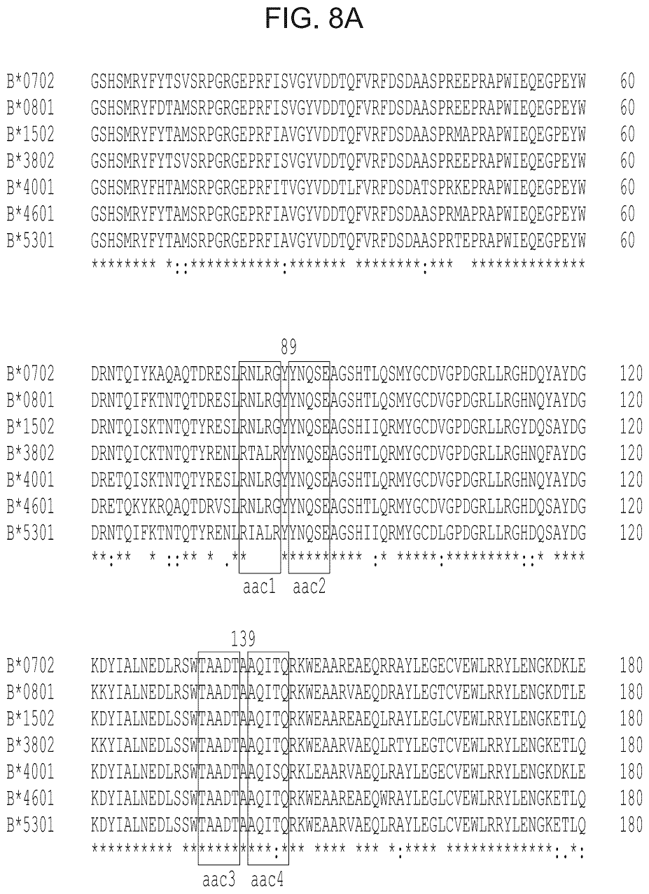

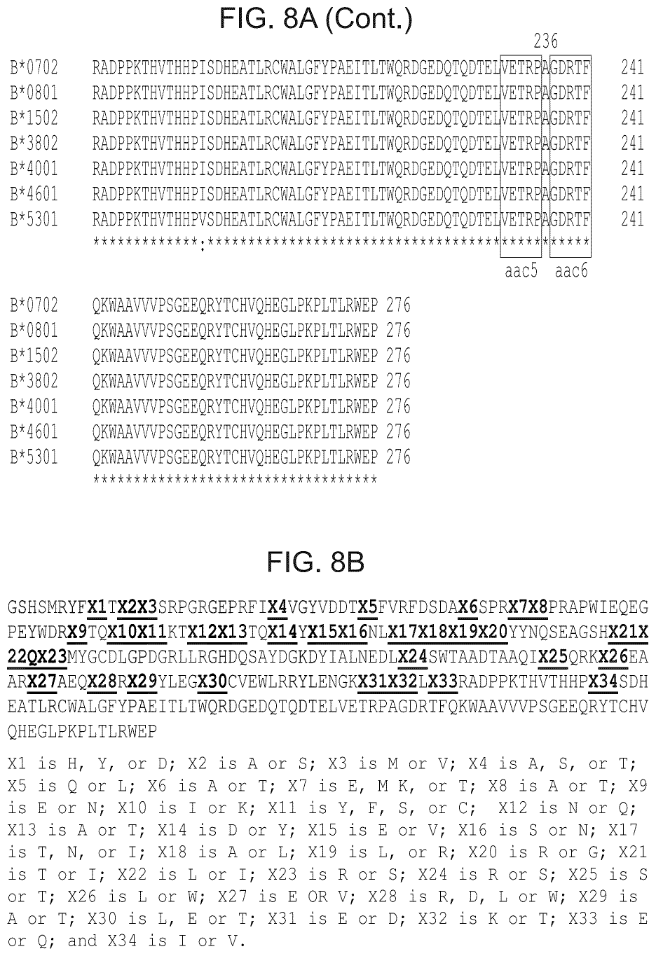

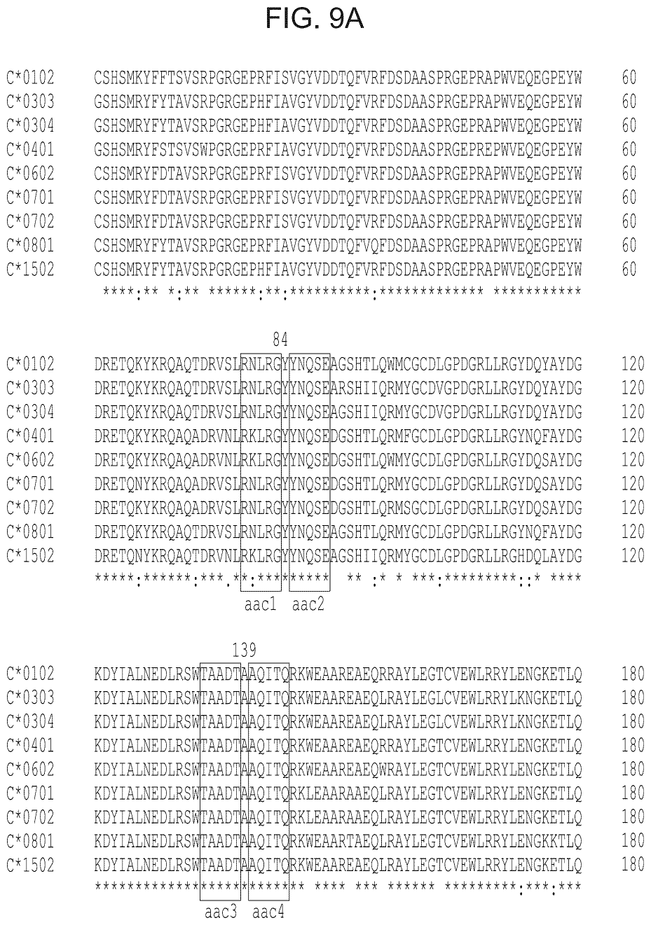

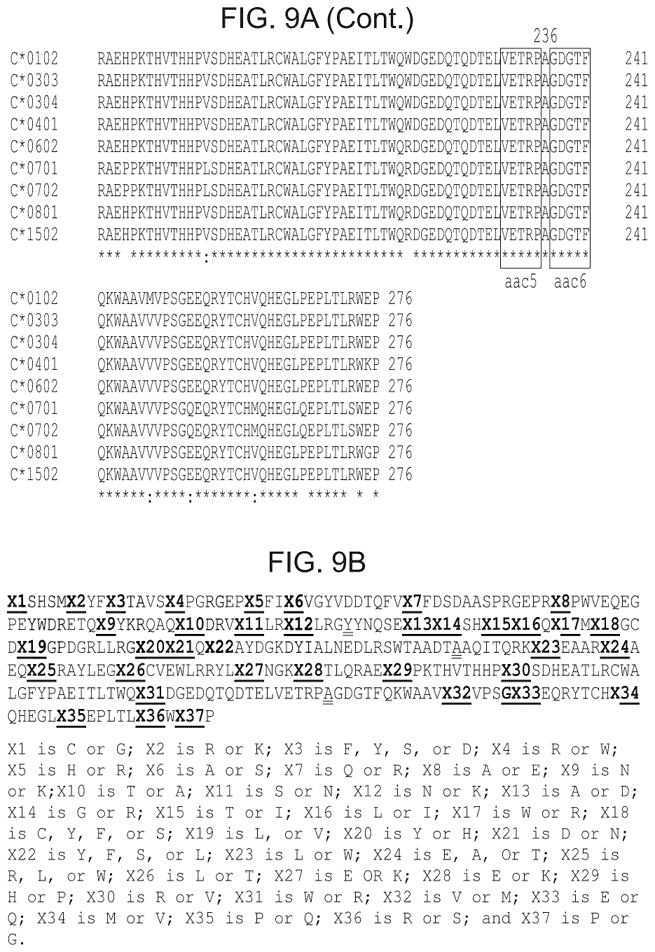

FIGS. 7-9 provide alignments of mature HLA class I heavy chain amino acid sequences (without leader sequences or transmembrane domains or intracellular domains). The aligned amino acid sequences in FIG. 7A are HLA-A class I heavy chains of the following alleles: A*0101, A*0201, A*0301, A*1101, A*2301, A*2402, A*2407, A*3303, and A*3401. The aligned amino acid sequences in FIG. 8A are HLA-B class I heavy chains of the following alleles: B*0702, B*0801, B*1502, B*3802, B*4001, B*4601, and B*5301. The aligned amino acid sequences in FIG. 9A are HLA-C class I heavy chains of the following alleles: C*0102, C*0303, C*0304, C*0401, C*0602, C*0701, C*0801, and C*1502. Indicated in the alignments are the locations (84 and 139 of the mature proteins) where cysteine residues may be introduced (e.g., by substitution) for the formation of a disulfide bond to stabilize the HLA H chain-β2M complex. Also shown in the alignment is position 236 (of the mature polypeptide), which may be substituted by a cysteine residue that can form an inter-chain disulfide bond with β2M (e.g., at aa 12). The boxes flanking residues 84, 139 and 236 show the groups of five amino acids on either sides of those six sets of five residues, denoted aac1 (for “amino acid cluster 1”), aac2 (for “amino acid cluster 2”), aac3 (for “amino acid cluster 3”), aac4 (for “amino acid cluster 4”), aac5 (for “amino acid cluster 5”), and aac6 (for “amino acid cluster 6”), that may be replaced by 1 to 5 amino acids selected independently from (i) any naturally occurring amino acid or (ii) any naturally occurring amino acid except proline or glycine.

FIGS. 7A, 8A, and 9A provide alignments of the amino acid sequences of mature HLA-A, -B, and -C class I heavy chains, respectively. The sequences are provided for the extracellular portion of the mature protein (without leader sequences or transmembrane domains or intracellular domains). As described in FIG. 6, the positions of aa residues 84, 139, and 236 and their flanking residues (aac1 to aac6) that may be replaced by 1 to 5 amino acids selected independently from (i) any naturally occurring amino acid or (ii) any naturally occurring amino acid except proline or glycine ae also shown. FIGS. 7B, 8B, and 9B provide consensus amino acid sequences for the HLA-A, -B, and -C sequences, respectively, provide in FIGS. 7A, 8A, and 9A. The consensus sequences show the variable amino acid positions as “X” residues sequentially numbered and the locations of amino acids 84, 139 and 236 double underlined.

With regard to FIG. 7A, in some cases: i) aac1 (amino acid cluster 1) may be the amino acid sequence GTLRG (SEQ ID NO: 174) or that sequence with one or two amino acids deleted or substituted with other naturally occurring amino acids (e.g., L replaced by I, V, A or F); ii) aac2 (amino acid cluster 2) may be the amino acid sequence YNQSE (SEQ ID NO: 175) or that sequence with one or two amino acids deleted or substituted with other naturally occurring amino acids (e.g., N replaced by Q, Q replaced by N, and/or E replaced by D); iii) aac3 (amino acid cluster 3) may be the amino acid sequence TAADM (SEQ ID NO: 176) or that sequence with one or two amino acids deleted or substituted with other naturally occurring amino acids (e.g., T replaced by S, A replaced by G, D replaced by E, and/or M replaced by L, V, or I); iv) aac4 (amino acid cluster 4) may be the amino acid sequence AQTTK (SEQ ID NO: 177) or that sequence with one or two amino acids deleted or substituted with other naturally occurring amino acids (e.g., A replaced by G, Q replaced by N, or T replaced by S, and or K replaced by R or Q); v) aac5 (amino acid cluster 5) may be the amino acid sequence VETRP (SEQ ID NO: 178) or that sequence with one or two amino acids deleted or substituted with other naturally occurring amino acids (e.g., V replaced by I or L, E replaced by D, T replaced by S, and/or R replaced by K); and/or vi) aac6 (amino acid cluster 6) may be the amino acid sequence GDGTF (SEQ ID NO: 179) or that sequence with one or two amino acids deleted or substituted with other naturally occurring amino acids (e.g., D replaced by E, T replaced by S, or F replaced by L, W, or Y).

With regard to FIG. 8A, in some cases: i) aac1 (amino acid cluster 1) may be the amino acid sequence RNLRG (SEQ ID NO: 180) or that sequence with one or two amino acids deleted or substituted with other naturally occurring amino acids (e.g., N replaced by T or I; and/or L replaced by A; and/or the second R replaced by L; and/or the G replaced by R); ii) aac2 (amino acid cluster 2) may be the amino acid sequence YNQSE (SEQ ID NO: 175) or that sequence with one or two amino acids deleted or substituted with other naturally occurring amino acids (e.g., N replaced by Q, Q replaced by N, and/or E replaced by D); iii) aac3 (amino acid cluster 3) may be the amino acid sequence TAADT (SEQ ID NO:181) or that sequence with one or two amino acids deleted or substituted with other naturally occurring amino acids (e.g., the first T replaced by S; and/or A replaced by G; and/or D replaced by E; and/or the second T replaced by S); iv) aac4 (amino acid cluster 4) may be the amino acid sequence AQITQ (SEQ ID NO: 182) or that sequence with one or two amino acids deleted or substituted with other naturally occurring amino acids (e.g., A replaced by G; and/or the first Q replaced by N; and/or I replaced by L or V; and/or the T replaced by S; and/or the second Q replaced by N); v) aac5 (amino acid cluster 5) may be the amino acid sequence VETRP (SEQ ID NO: 178) or that sequence with one or two amino acids deleted or substituted with other naturally occurring amino acids (e.g., V replaced by I or L, E replaced by D, T replaced by S, and/or R replaced by K); and/or vi) aac6 (amino acid cluster 6) may be the amino acid sequence GDRTF (SEQ ID NO:183) or that sequence with one or two amino acids deleted or substituted with other naturally occurring amino acids (e.g., D replaced by E; and/or T replaced by S; and/or R replaced by K or H; and/or F replaced by L, W, or Y).

With regard to FIG. 9A, in some cases: i) aac1 (amino acid cluster 1) may be the amino acid sequence RNLRG (SEQ ID NO: 180) or that sequence with one or two amino acids deleted or substituted with other naturally occurring amino acids (e.g., N replaced by K; and/or L replaced by A or I; and/or the second R replaced by H; and/or the G replaced by T or S); ii) aac2 (amino acid cluster 2) may be the amino acid sequence YNQSE (SEQ ID NO:175) or that sequence with one or two amino acids deleted or substituted with other naturally occurring amino acids (e.g., N replaced by Q, Q replaced by N, and/or E replaced by D); iii) aac3 (amino acid cluster 3) may be the amino acid sequence TAADT (SEQ ID NO:181) or that sequence with one or two amino acids deleted or substituted with other naturally occurring amino acids (e.g., the first T replaced by S; and/or A replaced by G; and/or D replaced by E; and/or the second T replaced by S); iv) aac4 (amino acid cluster 4) may be the amino acid sequence AQITQ (SEQ ID NO: 182) or that sequence with one or two amino acids deleted or substituted with other naturally occurring amino acids (e.g., A replaced by G; and/or the first Q replaced by N; and/or I replaced by L; and/or the second Q replaced by N or K); v) aac5 (amino acid cluster 5) may be the amino acid sequence VETRP (SEQ ID NO: 178) or that sequence with one or two amino acids deleted or substituted with other naturally occurring amino acids (e.g., V replaced by I or L, E replaced by D, T replaced by S, and/or R replaced by K or H); and/or vi) aac6 (amino acid cluster 6) may be the amino acid sequence GDGTF (SEQ ID NO: 179) or that sequence with one or two amino acids deleted or substituted with other naturally occurring amino acids (e.g., D replaced by E; and/or T replaced by S; and/or F replaced by L, W, or Y).

### 1) HLA-A

In some cases, a TMMP of the present disclosure comprises an HLA-A heavy chain polypeptide. The HLA-A heavy chain peptide sequences, or portions thereof, that may be that may be incorporated into a TMMP of the present disclosure include, but are not limited to, the alleles: A*0101, A*0201, A*0301, A*1101, A*2301, A*2402, A*2407, A*3303, and A*3401, which are aligned without all, or substantially all, of the leader, transmembrane and cytoplasmic sequences in FIG. 7A. Any of those alleles may comprise a mutation at one or more of positions 84, 139 and/or 236 (as shown in FIG. 7A) selected from: a tyrosine to alanine at position 84 (Y84A); a tyrosine to cysteine at position 84 (Y84C); an alanine to cysteine at position 139 (A139C); and an alanine to cysteine substitution at position 236 (A236C). In addition, HLA-A sequence having at least 75% (e.g., at least 80%, at least 85%, at least 90%, at least 95%, at least 98%, at least 99%) or 100% amino acid sequence identity to all Or part (e.g., 50, 75, 100, 150, 200, or 250 contiguous amino acids) of the sequence of those HLA-A alleles may also be employed (e.g., it may comprise 1-25, 1-5, 5-10, 10-15, 15-20, 20-25, or 25-30 amino acid insertions, deletions, and/or substitutions).

In some cases, a TMMP of the present disclosure comprises an HLA-A heavy chain polypeptide comprising the following HLA-A consensus amino acid sequence:

(SEQ ID NO: 184)
GSHSMRYFX1TSVSRPGRGEPRFIAVGYVDDTQF
VRFDSDAASQX2MEPRAPWIEQEGPEYWDX3X4T
X5X6X7KAX8SQX9X10RX11X12LX13X14X15
X16X17YYNQSEX18GSHTX19QX20MX21GCDV
GX22DX23RFLRGYX24QX25AYDGKDYIALX26
EDLRSWTAADMAAQX27TX287X29KWEX30X31
X32EAEQX33RX34YLX35GX36CVX37X38LRR
YLENGKETLQRTDX39PKTHMTHHX40X41SDHE
ATLRCWALX42FYPAEITLTWQRDGEDQTQDTEL
VETRPAGDGTFQKWAX43VVVPSGX44EQRYTCH
VQHEGLPKPLTLRWEX45,
wherein X1 is F, Y, S, or T; X2 is K or
R; X3 is Q, G, E, or R; X4 is N or E;
X5 is R or G; X6 is N or K; X7 is M or
V; X8 is H or Q; X9 is T or I; X10 is D
or H; X11 is A, V, or E; X12 is N or D;
X13 is G or R; X14 is T or I; X15 is L
or A; X16 is R or L; X17 is G or R; X18
is A or D; X19 is I, L, or V; X20 is I,
R or M; X21 is F or Y; X22 is S or P;
X23 is W or G; X24 is R, H, or Q; X25
is D or Y; X26 is N or K; X27 is T or
I; X28 is K or Q; X29 is R or H; X30
is A or T; X31 is A or V; X32 is H or R;
X33 is R, L, Q, or W; X34 is V or A;
X35 is D or E; X36 is R or T; X37 is D
or E; X38 is W or G; X39 is P or A; X40
is P or A; X41 is V or I; X42 is S or
G; X43 is A or S; X44 is Q or E; and
X45 is P or L.

As one example, an MHC Class I heavy chain polypeptide of a TMMP can comprise an amino acid sequence having at least 75%, at least 80%, at least 85%, at least 90%, at least 95%, at least 98%, at least 99%, or 100%, amino acid sequence identity to the following human HLA-A heavy chain amino acid sequence:

(SEQ ID NO: 185)
GSHSMRYFFTSVSRPGRGEPRFIAVGYVDDTQFVRFDSDAASQRMEPR
APWIEQEGPEYWDGETRKVKAHSQTHRVDLGTLRGYYNQSEAGSHTVQ
RMYGCDVGSDWRFLRGYHQYAYDGKDYIALKEDLRSWTAADMAAQTTK
HKWEAAHVAEQLRAYLEGTCVEWLRRYLENGKETLQRTDAPKTHMTHH
AVSDHEATLRCWALSFYPAEITLTWQRDGEDQTQDTELVETRPAGDGT
FQKWAAVVVPSGQEQRYTCHVQHEGLPKPLTLRWEP.

In some cases, an HLA-A heavy chain polypeptide suitable for inclusion in a TMMP of the present disclosure comprises the following amino acid sequence: GSHSMRYFFTSVSRPGRGEPRFIAVGYVDDTQFVRFDSDAASQRMEPRAPWIEQEGPEYWDGET RKVKAHSQTHRVDLGTLRGYYNQSEAGSHTVQRMYGCDVGSDWRFLRGYHQYAYDGKDYIA LKEDLRSWTAADMAAQTTKHKWEAAHVAEQLRAYLEGTCVEWLRRYLENGKETLQRTDAPK THMTHHAVSDHEATLRCWALSFYPAEITLTWQRDGEDQTQDTELVETRPAGDGTFQKWAAVV VPSGQEQRYTCHVQHEGLPKPLTLRWEP (SEQ ID NO: 185). This HLA-A heavy chain polypeptide is also referred to as “HLA-A*0201” or simply “HLA-A02.” In some cases, the C-terminal Pro is not included in a TMMP of the present disclosure. For example, in some cases, an HLA-A02 polypeptide suitable for inclusion in a TMMP of the present disclosure comprises the following amino acid sequence:

(SEQ ID NO: 186)
GSHSMRYFFTSVSRPGRGEPRFIAVGYVDDTQFVRFDSDAASQRMEPR
APWIEQEGPEYWDGETRKVKAHSQTHRVDLGTLRGYYNQSEAGSHTVQ
RMYGCDVGSDWRFLRGYHQYAYDGKDYIALKEDLRSWTAADMAAQTTK
HKWEAAHVAEQLRAYLEGTCVEWLRRYLENGKETLQRTDAPKTHMTHH
AVSDHEATLRCWALSFYPAEITLTWQRDGEDQTQDTELVETRPAGDGT
FQKWAAVVVPSGQEQRYTCHVQHEGLPKPLTLRWE.

### 2) HLA-A (Y84A; A236C)

In some cases, the MHC Class I heavy chain polypeptide comprises Y84A and A236C substitutions. For example, in some cases, the MHC Class I heavy chain polypeptide comprises an amino acid sequence having at least 75%, at least 80%, at least 85%, at least 90%, at least 95%, at least 98%, at least 99%, or 100%, amino acid sequence identity to the following human HLA-A heavy chain (Y84A; A236C) amino acid sequence: GSHSMRYFFTSVSRPGRGEPRFIAVGYVDDTQFVRFDSDAASQRMEPRAPWIEQEGPEYWDGET RKVKAHSQTHRVDLGTLRGAYNQSEAGSHTVQRMYGCDVGSDWRFLRGYHQYAYDGKDYIA LKEDLRSWTAADMAAQTTKHKWEAAHVAEQLRAYLEGTCVEWLRRYLENGKETLQRTDAPK THMTHHAVSDHEATLRCWALSFYPAEITLTWQRDGEDQTQDTELVETRPCGDGTFQKWAAVV VPSGQEQRYTCHVQHEGLPKPLTLRWEP (SEQ ID NO: 187), where amino acid 84 is Ala and amino acid 236 is Cys. In some cases, the Cys-236 forms an interchain disulfide bond with Cys-12 of a variant β2M polypeptide that comprises an R12C substitution.

In some cases, an HLA-A heavy chain polypeptide suitable for inclusion in a TMMP of the present disclosure is an HLA-A02 (Y84A; A236C) polypeptide comprising the following amino acid sequence:

(SEQ ID NO: 187)
GSHSMRYFFTSVSRPGRGEPRFIAVGYVDDTQFVRFDSDAASQRMEPR
APWIEQEGPEYWDGETRKVKAHSQTHRVDLGTLRGAYNQSEAGSHTVQ
RMYGCDVGSDWRFLRGYHQYAYDGKDYIALKEDLRSWTAADMAAQTTK
HKWEAAHVAEQLRAYLEGTCVEWLRRYLENGKETLQRTDAPKTHMTHH
AVSDHEATLRCWALSFYPAEITLTWQRDGEDQTQDTELVETRPCGDGT
FQKWAAVVVPSGQEQRYTCHVQHEGLPKPLTLRWEP.

In some cases, an HLA-A heavy chain polypeptide suitable for inclusion in a TMMP of the present disclosure is an HLA-A02 (Y84A; A236C) polypeptide comprising the following amino acid sequence:

(SEQ ID NO: 188)
GSHSMRYFFTSVSRPGRGEPRFIAVGYVDDTQFVRFDSDAASQRMEPR
APWIEQEGPEYWDGETRKVKAHSQTHRVDLGTLRGAYNQSEAGSHTVQ
RMYGCDVGSDWRFLRGYHQYAYDGKDYIALKEDLRSWTAADMAAQTTK
HKWEAAHVAEQLRAYLEGTCVEWLRRYLENGKETLQRTDAPKTHMTHH
AVSDHEATLRCWALSFYPAEITLTWQRDGEDQTQDTELVETRPCGDGT
FQKWAAVVVPSGQEQRYTCHVQHEGLPKPLTLRWE.

### 3) HLA-A (Y84C; A139C)

In some cases, the MHC Class I heavy chain polypeptide comprises Y84C and A139C substitutions. For example, in some cases, the MHC Class I heavy chain polypeptide comprises an amino acid sequence having at least 75%, at least 80%, at least 85%, at least 90%, at least 95%, at least 98%, at least 99%, or 100%, amino acid sequence identity to the following human HLA-A heavy chain (Y84C; A139C) amino acid sequence: GSHSMRYFFTSVSRPGRGEPRFIAVGYVDDTQFVRFDSDAASQRMEPRAPWIEQEGPEYWDGET RKVKAHSQTHRVDLGTLRGCYNQSEAGSHTVQRMYGCDVGSDWRFLRGYHQYAYDGKDYIA LKEDLRSWTAADMCAQTTKHKWEAAHVAEQLRAYLEGTCVEWLRRYLENGKETLQRTDAPK THMTHHAVSDHEATLRCWALSFYPAEITLTWQRDGEDQTQDTELVETRPAGDGTFQKWAAVV VPSGQEQRYTCHVQHEGLPKPLTLRWEP (SEQ ID NO: 189), where amino acid 84 is Cys and amino acid 139 is Cys. In some cases, Cys-84 forms an intrachain disulfide bond with Cys-139.

### 4) HLA-A11 (HLA-A*1101)

As one non-limiting example, an MHC Class I heavy chain polypeptide of a TMMP can comprise an amino acid sequence having at least 75%, at least 80%, at least 85%, at least 90%, at least 95%, at least 98%, at least 99%, or 100%, amino acid sequence identity to the following human HLA-A11 heavy chain amino acid sequence: GSHSMRYFYTSVSRPGRGEPRFIAVGYVDDTQFVRFDSDAASQRMEPRAPWIEQEGPEYWDQE TRNVKAQSQTDRVDLGTLRGYYNQSEDGSHTIQIMYGCDVGPDGRFLRGYRQDAYDGKDYIA LNEDLRSWTAADMAAQITKRKWEAAHAAEQQRAYLEGTCVEWLRRYLENGKETLQRTDPPKT HMTHHPISDHEATLRCWALGFYPAEITLTWQRDGEDQTQDTELVETRPAGDGTFQKWAAVVV PSGEEQRYTCHVQHEGLPKPLTLRWE (SEQ ID NO: 190). Such an MHC Class I heavy chain may be prominent in Asian populations, including populations of individuals of Asian descent.

### 5) HLA-A11 (Y84A; A236C)

As one non-limiting example, in some cases, the MHC Class I heavy chain polypeptide is an HLA-A11 allele that comprises Y84A and A236C substitutions. For example, in some cases, the MHC Class I heavy chain polypeptide comprises an amino acid sequence having at least 75%, at least 80%, at least 85%, at least 90%, at least 95%, at least 98%, at least 99%, or 100%, amino acid sequence identity to the following human HLA-A A11 heavy chain (Y84A; A236C) amino acid sequence: GSHSMRYFYTSVSRPGRGEPRFIAVGYVDDTQFVRFDSDAASQRMEPRAPWIEQEGPEYWDQE TRNVKAQSQTDRVDLGTLRGAYNQSEDGSHTIQIMYGCDVGPDGRFLRGYRQDAYDGKDYIA LNEDLRSWTAADMAAQITKRKWEAAHAAEQQRAYLEGTCVEWLRRYLENGKETLQRTDPPKT HMTHHPISDHEATLRCWALGFYPAEITLTWQRDGEDQTQDTELVETRPCGDGTFQKWAAVVV PSGEEQRYTCHVQHEGLPKPLTLRWE (SEQ ID NO:191), where amino acid 84 is Ala and amino acid 236 is Cys. In some cases, the Cys-236 forms an interchain disulfide bond with Cys-12 of a variant β2M polypeptide that comprises an R12C substitution.

### 6) HLA-A24 (HLA-A*2402)

As one non-limiting example, an MHC Class I heavy chain polypeptide of a TMMP of the present disclosure can comprise an amino acid sequence having at least 75%, at least 80%, at least 85%, at least 90%, at least 95%, at least 98%, at least 99%, or 100%, amino acid sequence identity to the following human HLA-A24 heavy chain amino acid sequence: GSHSMRYFSTSVSRPGRGEPRFIAVGYVDDTQFVRFDSDAASQRMEPRAPWIEQEGPEYWDEET GKVKAHSQTDRENLRIALRYYNQSEAGSHTLQMMFGCDVGSDGRFLRGYHQYAYDGKDYIAL KEDLRSWTAADMAAQITKRKWEAAHVAEQQRAYLEGTCVDGLRRYLENGKETLQRTDPPKT HMTHHPISDHEATLRCWALGFYPAEITLTWQRDGEDQTQDTELVETRPAGDGTFQKWAAVVV PSGEEQRYTCHVQHEGLPKPLTLRWEPSSQPTVPIVGIIAGLVLLGAVITGAVVAAVMWRRNSS DRKGGSYSQAASSDSAQGSDVSLTACKV (SEQ ID NO: 192). Such an MHC Class I heavy chain may be prominent in Asian populations, including populations of individuals of Asian descent. In some cases, amino acid 84 is an Ala. In some cases, amino acid 84 is a Cys. In some cases, amino acid 236 is a Cys. In some cases, amino acid 84 is an Ala and amino acid 236 is a Cys. In some cases, amino acid 84 is an Cys and amino acid 236 is a Cys.

### 7) HLA-A33 (HLA-A*3303)

As one non-limiting example, an MHC Class I heavy chain polypeptide of a TMMP of the present disclosure can comprise an amino acid sequence having at least 75%, at least 80%, at least 85%, at least 90%, at least 95%, at least 98%, at least 99%, or 100%, amino acid sequence identity to the following human HLA-A33 heavy chain amino acid sequence: GSHSMRYFTTSVSRPGRGEPRFIAVGYVDDTQFVRFDSDAASQRMEPRAPWIEQEGPEYWDRN TRNVKAHSQIDRVDLGTLRGYYNQSEAGSHTIQMMYGCDVGSDGRFLRGYQQDAYDGKDYIA LNEDLRSWTAADMAAQITQRKWEAARVAEQLRAYLEGTCVEWLRRYLENGKETLQRTDPPKT HMTHHAVSDHEATLRCWALSFYPAEITLTWQRDGEDQTQDTELVETRPAGDGTFQKWASVVV PSGQEQRYTCHVQHEGLPKPLTLRWEPSSQPTIPIVGIIAGLVLFGAVFAGAVVAAVRWRRKSSD RKGGSYSQAASSDSAQGSDMSLTACKV (SEQ ID NO: 193). Such an MHC Class I heavy chain may be prominent in Asian populations, including populations of individuals of Asian descent. In some cases, amino acid 84 is an Ala. In some cases, amino acid 84 is a Cys. In some cases, amino acid 236 is a Cys. In some cases, amino acid 84 is an Ala and amino acid 236 is a Cys. In some cases, amino acid 84 is an Cys and amino acid 236 is a Cys.

### 8) HLA-B

In some cases, a TMMP of the present disclosure comprises an HLA-B heavy chain polypeptide. The HLA-B heavy chain peptide sequences, or portions thereof, that may be that may be incorporated into a TMMP of the present disclosure include, but are not limited to, the alleles: B*0702, B*0801, B*1502, B*3802, B*4001, B*4601, and B*5301, which are aligned without all, or substantially all, of the leader, transmembrane and cytoplasmic sequences in FIG. 8A. Any of those alleles may comprise a mutation at one or more of positions 84, 139 and/or 236 (as shown in FIG. 8A) selected from: a tyrosine to alanine at position 84 (Y84A); a tyrosine to cysteine at position 84 (Y84C); an alanine to cysteine at position 139 (A139C); and an alanine to cysteine substitution at position 236 (A236C). In addition, a HLA-B polypeptide comprising an amino acid sequence having at least 75% (e.g., at least 80%, at least 85%, at least 90%, at least 95%, at least 98%, at least 99%) or 100% amino acid sequence identity to all or part (e.g., 50, 75, 100, 150, 200, or 250 contiguous amino acids) of the sequence of those HLA-B alleles may also be employed (e.g., it may comprise 1-25, 1-5, 5-10, 10-15, 15-20, 20-25, or 25-30 amino acid insertions, deletions, and/or substitutions).

In some cases, a TMMP of the present disclosure comprises an HLA-B heavy chain polypeptide comprising the following HLA-B consensus amino acid sequence:

(SEQ ID NO: 194)
GSHSMRYFX1TX2X3SRPGRGEPRF
IX4VGYVDDTX5FVRFDSDAX6SPR
X7X8PRAPWIEQEGPEYWDRX9TQ
X10X11KTX12X13TQX14YX15
X16NLX17X18X19X20YYNQSE
AGSHX21X22QX23MYGCDLGPD
GRLLRGHDQSAYDGKDYIALNEDL
X24SWTAADTAAQIX25QRKX26
EAARX27AEQX28RX29YLEGX30
CVEWLRRYLENGKX31X32LX33
RADPPKTHVTHHPX34SDHEATL
RCWALGFYPAEITLTWQRDGEDQT
QDTELVETRPAGDRTFQKWAAVVV
PSGEEQRYTCHVQHEGLPKPLTLR
WEP,
wherein X1 is H, Y, or D; X2 is A
or S; X3 is M or V; X4 is A, S, or
T; X5 is Q or L; X6 is A or T; X7
is E, M K, or T; X8 is A or T; X9
is E or N; X10 is I or K; X11 is Y,
F, S, or C; X12 is N or Q; X13 is A
or T; X14 is D or Y; X15 is E or V;
X16 is S or N; X17 is T, N, or I;
X18 is A or L; X19 is L, or R; X20
is R or G; X21 is T or I; X22 is L
or I; X23 is R or S; X24 is R or S;
X25 is S or T; X26 is L or W; X27
is E OR V; X28 is R, D, L or W; X29
is A or T; X30 is L, E or T; X31 is
E or D; X32 is K or T; X33 is E or
Q; and X34 is I or V.

As an example, an MHC Class I heavy chain polypeptide of a TMMP of the present disclosure can comprise an amino acid sequence having at least 75%, at least 80%, at least 85%, at least 90%, at least 95%, at least 98%, at least 99%, or 100%, amino acid sequence identity to the following human HLA-B heavy chain amino acid sequence:

(SEQ ID NO: 195)
GSHSMRYFYTSVSRPGRGEPRFISVGYVDDTQFVRFDSDAASPREEP
RAPWIEQEGPEYWDRNTQIYKAQAQTDRESLRNLRGYYNQSEAGSHT
LQSMYGCDVGPDGRLLRGHDQYAYDGKDYIALNEDLRSWTAADTAAQ
ITQRKWEAAREAEQRRAYLEGECVEWLRRYLENGKDKLERADPPKTH
VTHHPISDHEATLRCWALGFYPAEITLTWQRDGEDQTQDTELVETRP
AGDRTFQKWAAVVVPSGEEQRYTCHVQHEGLPKPLTLRWEP.

### 9) HLA-B (Y84A; A236C)

As one non-limiting example, in some cases, the MHC Class I heavy chain polypeptide is an HLA-B polypeptide that comprises Y84A and A236C substitutions. For example, in some cases, the MHC Class I heavy chain polypeptide comprises an amino acid sequence having at least 75%, at least 80%, at least 85%, at least 90%, at least 95%, at least 98%, at least 99%, or 100%, amino acid sequence identity to the following human HLA-B heavy chain (Y84A; A236C) amino acid sequence: GSHSMRYFYTSVSRPGRGEPRFISVGYVDDTQFVRFDSDAASPREEPRAPWIEQEGPEYWDRNT QIYKAQAQTDRESLRNLRGAYNQSEAGSHTLQSMYGCDVGPDGRLLRGHDQYAYDGKDYIAL NEDLRSWTAADTAAQITQRKWEAAREAEQRRAYLEGECVEWLRRYLENGKDKLERADPPKTH VTHHPISDHEATLRCWALGFYPAEITLTWQRDGEDQTQDTELVETRPCGDRTFQKWAAVVVPS GEEQRYTCHVQHEGLPKPLTLRWEP (SEQ ID NO: 196), where amino acid 84 is Ala and amino acid 236 is Cys. In some cases, the Cys-236 forms an interchain disulfide bond with Cys-12 of a variant β2M polypeptide that comprises an R12C substitution.

### 10) HLA-B (Y84C; A139C)

In some cases, the MHC Class I heavy chain polypeptide comprises Y84C and A139C substitutions. For example, in some cases, the MHC Class I heavy chain polypeptide comprises an amino acid sequence having at least 75%, at least 80%, at least 85%, at least 90%, at least 95%, at least 98%, at least 99%, or 100%, amino acid sequence identity to the following human HLA-B heavy chain (Y84C; A139C) amino acid sequence: GSHSMRYFYTSVSRPGRGEPRFISVGYVDDTQFVRFDSDAASPREEPRAPWIEQEGPEYWDRNT QIYKAQAQTDRESLRNLRGCYNQSEAGSHTLQSMYGCDVGPDGRLLRGHDQYAYDGKDYIAL NEDLRSWTAADTCAQITQRKWEAAREAEQRRAYLEGECVEWLRRYLENGKDKLERADPPKTH VTHHPISDHEATLRCWALGFYPAEITLTWQRDGEDQTQDTELVETRPAGDRTFQKWAAVVVPS GEEQRYTCHVQHEGLPKPLTLRWEP (SEQ ID NO: 197), where amino acid 84 is Cys and amino acid 139 is Cys. In some cases, Cys-84 forms an intrachain disulfide bond with Cys-139.

### 11) HLA-B*0702

As an example, in some cases, a MHC Class I heavy chain polypeptide present in a TMMP of the present disclosure comprises an amino acid sequence of HLA-B*0702 (SEQ ID NO: 195) in FIG. 8A, or a sequence having at least 75% (e.g., at least 80%, at least 85%, at least 90%, at least 95%, at least 98%, at least 99%) or 100%, amino acid sequence identity to all or part (e.g., 50, 75, 100, 150, 200, or 250 contiguous amino acids) of that sequence (e.g., it may comprise 1-25, 1-5, 5-10, 10-15, 15-20, 20-25, or 25-30 amino acid insertions, deletions, and/or substitutions). In some cases, where the HLA-B heavy chain polypeptide of TMMP of the present disclosure has less than 100% identity to the sequence labeled HLA-B in FIG. 6, or labeled “B*0702 in FIG. 8A, it may comprise a mutation at one or more of positions 84, 139 and/or 236 selected from: a tyrosine to alanine substitution at position 84 (Y84A); a tyrosine to cysteine substitution at position 84 (Y84C); an alanine to cysteine at position 139 (A139C); and an alanine to cysteine substitution at position 236 (A236C). In some cases, the HLA-B heavy chain polypeptide of TMMP of the present disclosure comprises Y84A and A236C substitutions. In some cases, the HLA-B*0702 heavy chain polypeptide of TMMP of the present disclosure comprises Y84C and A139C substitutions. In some cases, the HLA-B heavy chain polypeptide of TMMP of the present disclosure comprises Y84C, A139C, and A236C substitutions.

### 12) HLA-C

In some cases, a TMMP of the present disclosure comprises an HLA-C heavy chain polypeptide. The HLA-C heavy chain polypeptide, or portions thereof, that may be that may be incorporated into a TMMP of the present disclosure include, but are not limited to, the alleles: C*0102, C*0303, C*0304, C*0401, C*0602, C*0701, C*0801, and C*1502, which are aligned without all, or substantially all, of the leader, transmembrane and cytoplasmic sequences in FIG. 9A. Any of those alleles may comprise a mutation at one or more of positions 84, 139 and/or 236 (as shown in FIG. 9A) selected from: a tyrosine to alanine substitution at position 84 (Y84A); a tyrosine to cysteine substitution at position 84 (Y84C); an alanine to cysteine substitution at position 139 (A139C); and an alanine to cysteine substitution at position 236 (A236C). In addition, an HLA-C polypeptide comprising an amino acid sequence having at least 75% (e.g., at least 80%, at least 85%, at least 90%, at least 95%, at least 98%, at least 99%) or 100% amino acid sequence identity to all or part (e.g., 50, 75, 100, 150, 200, or 250 contiguous amino acids) of the sequence of those HLA-C alleles may also be employed (e.g., it may comprise 1-25, 1-5, 5-10, 10-15, 15-20, 20-25, or 25-30 amino acid insertions, deletions, and/or substitutions).

In some cases, a TMMP of the present disclosure comprises an HLA-C heavy chain polypeptide comprising the following HLA-C consensus amino acid sequence:

(SEQ ID NO: 198)
X1SHSMX2YFX3TAVSX4PGRGEPX5FIX6VGYVDDTQFVX7
FDSDAASPRGEPRX8PWVEQEGPEYWDRETQX9YKRQAQX10
DRVX11LRX12LRGYYNQSEX13X14SHX15X16QX17MX18
GCDX19GPDGRLLRGX20X21QX22AYDGKDYIALNEDLRSW
TAADTAAQITQRKX23EAARX24AEQX25RAYLEGX26CVEW
LRRYLX27NGKX28TLQRAEX29PKTHVTHHPX30SDHEATL
RCWALGFYPAEITLTWQX31DGEDQTQDTELVETRPAGDGTF
QKWAAVX32VPSGX33EQRYTCHX34QHEGLX35EPLTLX36
WX37P,
wherein X1 is C or G; X2 is R or K; X3 is
F, Y, S, or D; X4 is R or W; X5 is H or R;
X6 is A or S; X7 is Q or R; X8 is A or E;
X9 is N or K; X10 is T or A; X11 is S or N;
X12 is N or K; X13 is A or D; X14 is G or
R; X15 is T or I; X16 is L or I; X17 is W
or R; X18 is C, Y, F, or S; X19 is L, or V;
X20 is Y or H; X21 is D or N; X22 is Y, F,
S, or L; X23is L or W; X24 is E, A, Or T;
X25 is R, L, or W; X26 is L or T; X27 is E
OR K; X28 is E or K; X29 is H or P; X30 is
R or V; X31 is W or R; X32 is V or M; X33
is E or Q; X34 is M or V; X35 is P or Q;
X36 is R or S; and X37 is P or G.

As an example, an MHC Class I heavy chain polypeptide of a TMMP of the present disclosure can comprise an amino acid sequence having at least 75%, at least 80%, at least 85%, at least 90%, at least 95%, at least 98%, at least 99%, or 100%, amino acid sequence identity to the following human HLA-C heavy chain amino acid sequence:

(SEQ ID NO: 199)
CSHSMRYFDTAVSRPGRGEPRFISVGYVDDTQFVRFDSDAASPRGEPR
APWVEQEGPEYWDRETQNYKRQAQADRVSLRNLRGYYNQSEDGSHTLQ
RMYGCDLGPDGRLLRGYDQSAYDGKDYIALNEDLRSWTAADTAAQITQ
RKLEAARAAEQLRAYLEGTCVEWLRRYLENGKETLQRAEPPKTHVTHH
PLSDHEATLRCWALGFYPAEITLTWQRDGEDQTQDTELVETRPAGDGT
FQKWAAVVVPSGQEQRYTCHMQHEGLQEPLTLSWEP.

### 13) HLA-C(Y84A; A236C)

As one non-limiting example, in some cases, the MHC Class I heavy chain polypeptide is an HLA-C polypeptide that comprises Y84A and A236C substitutions. For example, in some cases, the MHC Class I heavy chain polypeptide comprises an amino acid sequence having at least 75%, at least 80%, at least 85%, at least 90%, at least 95%, at least 98%, at least 99%, or 100%, amino acid sequence identity to the following human HLA-C heavy chain (Y84A; A236C) amino acid sequence: CSHSMRYFDTAVSRPGRGEPRFISVGYVDDTQFVRFDSDAASPRGEPRAPWVEQEGPEYWDRE TQNYKRQAQADRVSLRNLRGAYNQSEDGSHTLQRMYGCDLGPDGRLLRGYDQSAYDGKDYI ALNEDLRSWTAADTAAQITQRKLEAARAAEQLRAYLEGTCVEWLRRYLENGKETLQRAEPPKT HVTHHPLSDHEATLRCWALGFYPAEITLTWQRDGEDQTQDTELVETRPCGDGTFQKWAAVVV PSGQEQRYTCHMQHEGLQEPLTLSWEP (SEQ ID NO:200), where amino acid 84 is Ala and amino acid 236 is Cys. In some cases, the Cys-236 forms an interchain disulfide bond with Cys-12 of a variant β2M polypeptide that comprises an R12C substitution.

### 14) HLA-C(Y84C; A139C)

In some cases, the MHC Class I heavy chain polypeptide comprises Y84C and A139C substitutions. For example, in some cases, the MHC Class I heavy chain polypeptide comprises an amino acid sequence having at least 75%, at least 80%, at least 85%, at least 90%, at least 95%, at least 98%, at least 99%, or 100%, amino acid sequence identity to the following human HLA-C heavy chain (Y84C; A139C) amino acid sequence: CSHSMRYFDTAVSRPGRGEPRFISVGYVDDTQFVRFDSDAASPRGEPRAPWVEQEGPEYWDRE TQNYKRQAQADRVSLRNLRGCYNQSEDGSHTLQRMYGCDLGPDGRLLRGYDQSAYDGKDYI ALNEDLRSWTAADTCAQITQRKLEAARAAEQLRAYLEGTCVEWLRRYLENGKETLQRAEPPKT HVTHHPLSDHEATLRCWALGFYPAEITLTWQRDGEDQTQDTELVETRPAGDGTFQKWAAVVV PSGQEQRYTCHMQHEGLQEPLTLSWEP (SEQ ID NO:201), where amino acid 84 is Cys and amino acid 139 is Cys. In some cases, Cys-84 forms an intrachain disulfide bond with Cys-139.

### 15) HLA-C*0701

In some cases, a MHC Class I heavy chain polypeptide of a TMMP of the present disclosure comprises an amino acid sequence of HLA-C*0701 of FIG. 9A (labeled HLA-C in FIG. 6), or an amino acid sequence having at least 75% (e.g., at least 80%, at least 85%, at least 90%, at least 95%, at least 98%, at least 99%) or 100% amino acid sequence identity to all or part (e.g., 50, 75, 100, 150, 200, or 250 contiguous amino acids) of that sequence (e.g., it may comprise 1-25, 1-5, 5-10, 10-15, 15-20, 20-25, or 25-30 amino acid insertions, deletions, and/or substitutions). In some cases, where the HLA-C heavy chain polypeptide of a TMMP of the present disclosure has less than 100% identity to the sequence labeled HLA-C*0701 in FIG. 9A, it may comprise a mutation at one or more of positions 84, 139 and/or 236 selected from: a tyrosine to alanine substitution at position 84 (Y84A); a tyrosine to cysteine substitution at position 84 (Y84C); an alanine to cysteine at position 139 (A139C); and an alanine to cysteine substitution at position 236 (A236C). In some cases, the HLA-C heavy chain polypeptide of a TMMP of the present disclosure comprises Y84A and A236C substitutions. In some cases, the HLA-C*0701 heavy chain polypeptide of a T-Cell-MMP or its epitope conjugate comprises Y84C and A139C substitutions. In some cases, the HLA-C heavy chain polypeptide of a TMMP of the present disclosure comprises Y84C, A139C, and A236C substitutions.

### Non-Classical HLA-E, -F, and -G MHC Class I Heavy Chains

In some cases, a TMMP of the present disclosure comprises a non-classical MHC Class I heavy chain polypeptide. Among the non-classical HLA heavy chain polypeptides, or portions thereof, that may be that may be incorporated into a TMMP of the present disclosure include, but are not limited to, those of HLA-E, -F, and -G alleles. Amino acid sequences for HLA-E, -F, and -G heavy chain polypeptides, (and the HLA-A, B and C alleles) may be found on the world wide web hla.alleles.org/nomenclature/index.html, the European Bioinformatics Institute (www(dot)ebi(dot)ac(dot)uk), which is part of the European Molecular Biology Laboratory (EMBL), and at the National Center for Biotechnology Information (www(dot)ncbi(dot)nlm(dot)nih(dot)gov).

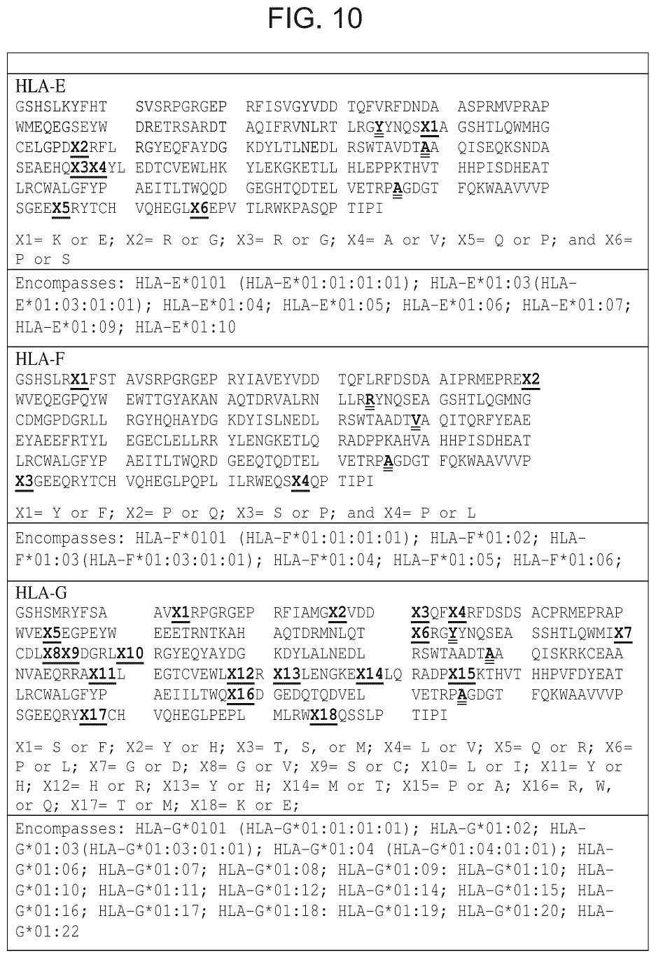

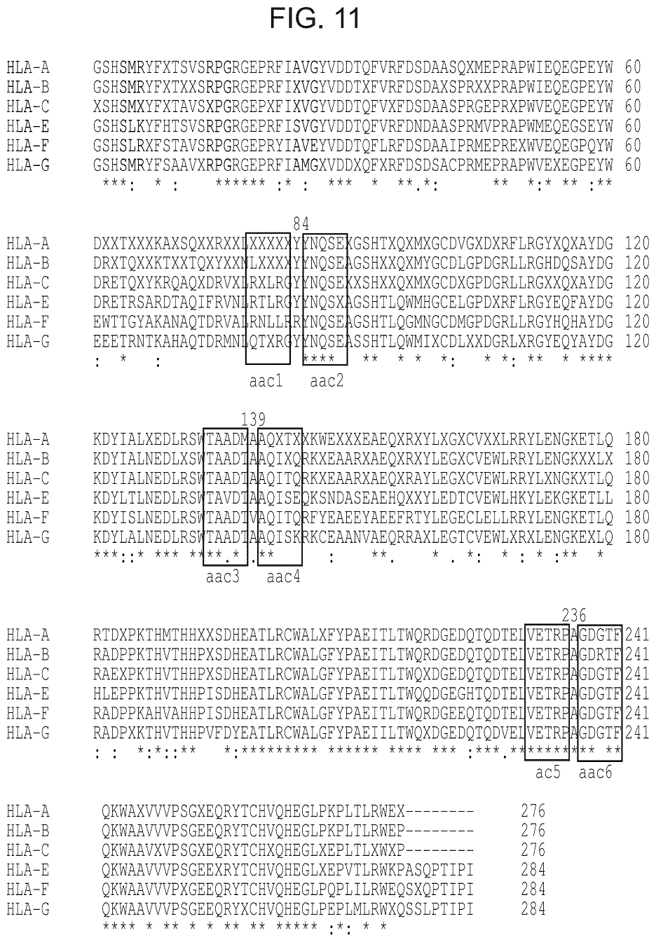

Non-limiting examples of suitable HLA-E alleles include, but are not limited to, HLA-E*0101 (HLA-E*01:01:01:01), HLA-E*01:03 (HLA-E*01:03:01:01), HLA-E*01:04, HLA-E*01:05, HLA-E*01:06, HLA-E*01:07, HLA-E*01:09, and HLA-E*01:10. Non-limiting examples of suitable HLA-F alleles include, but are not limited to, HLA-F*0101 (HLA-F*01:01:01:01), HLA-F*01:02, HLA-F*01:03 (HLA-F*01:03:01:01), HLA-F*01:04, HLA-F*01:05, and HLA-F*01:06. Non-limiting examples of suitable HLA-G alleles include, but are not limited to, HLA-G*0101 (HLA-G*01:01:01:01), HLA-G*01:02, HLA-G*01:03 (HLA-G*01:03:01:01), HLA-G*01:04 (HLA-G*01:04:01:01), HLA-G*01:06, HLA-G*01:07, HLA-G*01:08, HLA-G*01:09: HLA-G*01:10, HLA-G*01:10, HLA-G*01:11, HLA-G*01:12, HLA-G*01:14, HLA-G*01:15, HLA-G*01:16, HLA-G*01:17, HLA-G*01:18: HLA-G*01:19, HLA-G*01:20, and HLA-G*01:22. Consensus sequences for those HLA E, -F and -G alleles without all, or substantially all, of the leader, transmembrane and cytoplasmic sequences are provided in FIG. 10, and aligned with consensus sequences of the above-mentioned HLA-A, -B and -C alleles in FIG. 11.

FIG. 11 provides a consensus sequence for each of HLA-E, -F, and -G with the variable aa positions indicated as “X” residues sequentially numbered and the locations of aas 84, 139 and 236 double underlined.

FIG. 11 provides an alignment of the consensus amino acid sequences for HLA-A, -B, -C, -E, -F, and -G, which are given in FIGS. 7-11. Variable residues in each sequence are listed as “X” with the sequential numbering removed. As indicated in FIG. 6, the locations of aas 84, 139 and 236 are indicated with their flanking five-amino acid clusters that may be replaced by 1 to 5 amino acids selected independently from (i) any naturally occurring amino acid or (ii) any naturally occurring amino acid except proline or glycine are also shown.

Any of the above-mentioned HLA-E, -F, and/or -G alleles may comprise a substitution at one or more of positions 84, 139 and/or 236 as shown in FIG. 11 for the consensus sequences. In some cases, the substitutions may be selected from a: position 84 tyrosine to alanine (Y84A) or cysteine (Y84C), or, in the case of HLA-F, an R84A or R84C substitution; a position 139 alanine to cysteine (A139C), or, in the case of HLA-F, a V139C; and an alanine to cysteine substitution at position 236 (A236C). In addition, an HLA-E, -F and/or -G sequence having at least 75% (e.g., at least 80%, at least 85%, at least 90%, at least 95%, at least 98%, at least 99%) or 100% amino acid sequence identity to all or part (e.g., 50, 75, 100, 150, 200, or 250 contiguous amino acids) of any of the consensus sequences of set forth in FIG. 11 may also be employed (e.g., the sequences may comprise 1-25, 1-5, 5-10, 10-15, 15-20, 20-25, or 25-30 amino acid insertions, deletions, and/or substitutions in addition to changes at variable residues listed therein).

### Mouse H2K

In some cases, a MHC Class I heavy chain polypeptide present in a TMMP of the present disclosure comprises an amino acid sequence of MOUSE H2K (SEQ ID NO:401) (MOUSE H2K in FIG. 6), or a sequence having at least 75%, at least 80%, at least 85%, at least 90%, at least 95%, at least 98%, at least 99%, or 100% amino acid sequence identity to all or part (e.g., 50, 75, 100, 150, 200, or 250 contiguous amino acids) of that sequence (e.g., it may comprise 1-25, 1-5, 5-10, 10-15, 15-20, 20-25, or 25-30 amino acid insertions, deletions, and/or substitutions). In some cases, where the MOUSE H2K heavy chain polypeptide of a TMMP of the present disclosure has less than 100% identity to the sequence labeled MOUSE H2K in FIG. 6, it may comprise a mutation at one or more of positions 84, 139 and/or 236 selected from: a tyrosine to alanine at position 84 (Y84A); a tyrosine to cysteine at position 84 (Y84C); an alanine to cysteine at position 139 (A139C); and an alanine to cysteine substitution at position 236 (A236C). In some cases, the MOUSE H2K heavy chain polypeptide of a TMMP of the present disclosure comprises Y84A and A236C substitutions. In some cases, the MOUSE H2K heavy chain polypeptide of a TMMP of the present disclosure comprises Y84C and A139C substitutions. In some cases, the MOUSE H2K heavy chain polypeptide of a TMMP of the present disclosure comprises Y84C, A139C and A236C substitutions.

### Exemplary Combinations

Table 2, below, presents various combinations of MHC Class I heavy chain sequence modifications that can be incorporated in a TMMP of the present disclosure.

TABLE 2

| | | | Specific Substitutions |
|---|---|---|---|
| | HLA Heavy | | at aa positions |
| Entry | Chain Sequence | Sequence Identity Range [symbol] | 84, 139 and/or 236 |
| 1 | HLA-A | 75%-99.8%, 80%-99.8%, 85%-99.8%, | None; Y84C; Y84A; |
| | Consensus | 90%-99.8%, 95%-99.8%, 98%-99.8%, | A139C; A236C; |
| | (FIG. 7B) | or 99%-99.8%; or 1-25, 1-5, 5-10, 10-15, | (Y84A & A236C); |
| | | 15-20, or 20-25 aa insertions, deletions, | (Y84C & A139C); or |
| | | and/or substitutions (not counting | (Y84C, A139C & A236C) |
| | | variable residues) | |
| 2 | A*0101, A*0201, | 75%-99.8%, 80%-99.8%, 85%-99.8%, | None; Y84C; Y84A; |
| | A*0301, A*1101, | 90%-99.8%, 95%-99.8%, 98%-99.8%, | A139C; A236C; |
| | A*2402, A*2301, | or 99%-99.8%; or 1-25, 1-5, 5-10, 10-15, | (Y84A & A236C); |
| | A*2402, A*2407, | 15-20, or 20-25 aa insertions, deletions, | (Y84C & A139C); or |
| | A*3303, or | and/or substitutions | (Y84C, A139C & A236C) |
| | A*3401 | | |
| | (FIG. 7A) | | |
| 3 | HLA-B | 75%-99.8%, 80%-99.8%, 85%-99.8%, | None; Y84C; Y84A; |
| | Consensus | 90%-99.8%, 95%-99.8%, 98%-99.8%, | A139C; A236C; |
| | (FIG. 8B) | or 99%-99.8%; or 1-25, 1-5, 5-10, 10-15, | (Y84A & A236C); |
| | | 15-20, or 20-25 aa insertions, deletions, | (Y84C & A139C); or |
| | | and/or substitutions (not counting | (Y84C, A139C & A236C) |
| | | variable residues) | |
| 4 | B*0702, B*0801, | 75%-99.8%, 80%-99.8%, 85%-99.8%, | None; Y84C; Y84A; |
| | B*1502, B*3802, | 90%-99.8%, 95%-99.8%, 98%-99.8%, | A139C; A236C; |
| | B*4001, B*4601, | or 99%-99.8%; or 1-25, 1-5, 5-10, 10-15, | (Y84A & A236C); |
| | or B*5301 | 15-20, or 20-25 aa insertions, deletions, | (Y84C & A139C); or |
| | (FIG. 8A) | and/or substitutions | (Y84C, A139C & A236C) |
| 5 | HLA-C | 75%-99.8%, 80%-99.8%, 85%-99.8%, | None; Y84C; Y84A; |
| | Consensus | 90%-99.8%, 95%-99.8%, 98%-99.8%, | A139C; A236C; |
| | (FIG. 9B) | or 99%-99.8%; or 1-25, 1-5, 5-10, 10-15, | (Y84A & A236C); |
| | | 15-20, or 20-25 aa insertions, deletions, | (Y84C & A139C); or |
| | | and/or substitutions (not counting | (Y84C, A139C & A236C) |
| | | variable residues) | |
| 6 | C*0102, C*0303, | 75%-99.8%, 80%-99.8%, 85%-99.8%, | None; Y84C; Y84A; |
| | C*0304, C*0401, | 90%-99.8%, 95%-99.8%, 98%-99.8%, | A139C; A236C; |
| | C*0602, C*0701, | or 99%-99.8%; or 1-25, 1-5, 5-10, 10-15, | (Y84A & A236C); |
| | C*0801, or | 15-20, or 20-25 aa insertions, deletions, | (Y84C & A139C); or |
| | C*1502 | and/or substitutions | (Y84C, A139C & A236C) |
| | (FIG. 9A) | | |
| 7 | HLA-E, F, or G | 75%-99.8%, 80%-99.8%, 85%-99.8%, | None; Y84C; Y84A; |
| | Consensus | 90%-99.8%, 95%-99.8%, 98%-99.8%, | A139C; A236C; |
| | (FIG. 10) | or 99%-99.8%; or 1-25, 1-5, 5-10, 10-15, | (Y84A & A236C); |
| | | 15-20, or 20-25 aa insertions, deletions, | (Y84C & A139C); or |
| | | and/or substitutions (not counting | (Y84C, A139C & A236C) |
| | | variable residues) | |
| 8 | MOUSE H2K | 75%-99.8%, 80%-99.8%, 85%-99.8%, | None; Y84C; Y84A; |
| | (FIG. 6) | 90%-99.8%, 95%-99.8%, 98%-99.8%, | A139C; A236C; |
| | | or 99%-99.8%; or 1-25, 1-5, 5-10, 10-15, | (Y84A & A236C); |
| | | 15-20, or 20-25 aa insertions, deletions, | (Y84C & A139C); or |
| | | and/or substitutions | (Y84C, A139C & A236C) |

[symbol]

The Sequence Identity Range is the permissible range in sequence identity of an MHC-H polypeptide sequence incorporated into a TMMP relative to the corresponding portion of the sequences listed in FIG. 6-11 not counting the variable residues in the consensus sequences.

### Beta-2 Microglobulin

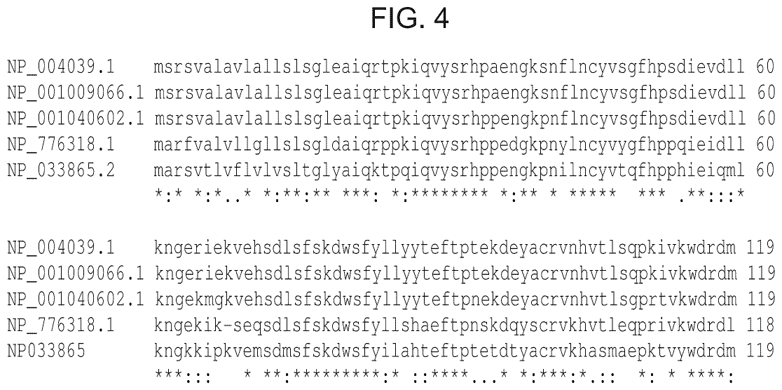

A β2-microglobulin (β2M) polypeptide of a TMMP of the present disclosure can be a human β2M polypeptide, a non-human primate β2M polypeptide, a murine β2M polypeptide, and the like. In some instances, a β2M polypeptide comprises an amino acid sequence having at least 75%, at least 80%, at least 85%, at least 90%, at least 95%, at least 98%, at least 99%, or 100%, amino acid sequence identity to a β2M amino acid sequence depicted in FIG. 4. In some instances, a β2M polypeptide comprises an amino acid sequence having at least 75%, at least 80%, at least 85%, at least 90%, at least 95%, at least 98%, at least 99%, or 100%, amino acid sequence identity to amino acids 21 to 119 of a β2M amino acid sequence depicted in FIG. 4.

In some cases, a suitable β2M polypeptide comprises the following amino acid sequence:

(SEQ ID NO: 202)
IQRTPKIQVY SCHPAENGKS NFLNCYVSGF
HPSDIEVDLLKNGERIEKVE HSDLSFSKDW SFYLLYYTEF
TPTEKDEYAC RVNHVTLSQP KIVKWDRDM;

and the HLA Class I heavy chain polypeptide comprises the following amino acid sequence:

GSHSMRYFFTSVSRPGRGEPRFIAVGYVDDTQFVRFDSDAASQRMEPRAPWIEQ EGPEYWDGETRKVKAHSQTHRVDL(aa1){C}(aa2)AGSHTVQRMYGCDVGSDWRFLRGYHQYA YDGKDYIALKEDLRSW(aa3){C}(aa4))HKWEAAHVAEQLRAYLEGTCVEWLRRYLENGKETLQ RTDAPKTHMTHHAVSDHEATLRCWALSFYPAEITLTWQRDGEDQTQDTEL(aa5)I(aa6)QKWAA VVVPSGQEQRYTCHVQHEGLPKPLTLRWEP (SEQ ID NO:203), where the cysteine residues indicated as {C}form an disulfide bond between the α1 and α2-1 helices and tII residue forms a disulfide bond with the β2M polypeptide cysteine at position 12. In the sequence above, “aa1” is “amino acid cluster 1”; “aa2” is “amino acid cluster 2”; “aa3” is “amino acid cluster 3”; “aa4” is “amino acid cluster 4”; “aa5” is “amino acid cluster 5”; and “aa6” is “amino acid cluster 6”; see, e.g., FIG. 6. Each occurrence of aa1, aa2, aa3, aa4, aa5, and aa6 is and independently selected to be 1-5 amino acid residues, wherein the amino acid residues are i) selected independently from any naturally occurring (e.g., encoded) amino acid or ii) any naturally occurring amino acid except proline or glycine.

In some cases, an MHC polypeptide comprises a single amino acid substitution relative to a reference MHC polypeptide (where a reference MHC polypeptide can be a wild-type MHC polypeptide), where the single amino acid substitution substitutes an amino acid with a cysteine (Cys) residue. Such cysteine residues, when present in an MHC polypeptide of a first polypeptide of a TMMP of the present disclosure, can form a disulfide bond with a cysteine residue present in a second polypeptide chain of a TMMP of the present disclosure.

In some cases, a first MHC polypeptide in a first polypeptide of a TMMP of the present disclosure, and/or the second MHC polypeptide in the second polypeptide of a TMMP of the present disclosure, includes an amino acid substitution to substitute an amino acid with a cysteine, where the substituted cysteine in the first MHC polypeptide forms a disulfide bond with a cysteine in the second MHC polypeptide, where a cysteine in the first MHC polypeptide forms a disulfide bond with the substituted cysteine in the second MHC polypeptide, or where the substituted cysteine in the first MHC polypeptide forms a disulfide bond with the substituted cysteine in the second MHC polypeptide.

For example, in some cases, one of following pairs of residues in an HLA β2-microglobulin and an HLA Class I heavy chain is substituted with cysteines (where residue numbers are those of the mature polypeptide): 1) β2M residue 12, HLA Class I heavy chain residue 236; 2) β2M residue 12, HLA Class I heavy chain residue 237; 3) β2M residue 8, HLA Class I heavy chain residue 234; 4) β2M residue 10, HLA Class I heavy chain residue 235; 5) β2M residue 24, HLA Class I heavy chain residue 236; 6) β2M residue 28, HLA Class I heavy chain residue 232; 7) β2M residue 98, HLA Class I heavy chain residue 192; 8) β2M residue 99, HLA Class I heavy chain residue 234; 9) β2M residue 3, HLA Class I heavy chain residue 120; 10) β2M residue 31, HLA Class I heavy chain residue 96; 11) β2M residue 53, HLA Class I heavy chain residue 35; 12) β2M residue 60, HLA Class I heavy chain residue 96; 13) β2M residue 60, HLA Class I heavy chain residue 122; 14) β2M residue 63, HLA Class I heavy chain residue 27; 15) β2M residue Arg3, HLA Class I heavy chain residue Gly120; 16) β2M residue His31, HLA Class I heavy chain residue Gln96; 17) β2M residue Asp53, HLA Class I heavy chain residue Arg35; 18) β2M residue Trp60, HLA Class I heavy chain residue Gln96; 19) β2M residue Trp60, HLA Class I heavy chain residue Asp122; 20) β2M residue Tyr63, HLA Class I heavy chain residue Tyr27; 21) β2M residue Lys6, HLA Class I heavy chain residue Glu232; 22) β2M residue Gln8, HLA Class I heavy chain residue Arg234; 23) β2M residue Tyr10, HLA Class I heavy chain residue Pro235; 24) β2M residue Ser11, HLA Class I heavy chain residue Gln242; 25) β2M residue Asn24, HLA Class I heavy chain residue Ala236; 26) β2M residue Ser28, HLA Class I heavy chain residue Glu232; 27) β2M residue Asp98, HLA Class I heavy chain residue His192; and 28) β2M residue Met99, HLA Class I heavy chain residue Arg234. The amino acid numbering of the MHC/HLA Class I heavy chain is in reference to the mature MHC/HLA Class I heavy chain, without a signal peptide. For example, in some cases, residue 236 of the mature HLA-A amino acid sequence is substituted with a Cys. In some cases, residue 236 of the mature HLA-B amino acid sequence is substituted with a Cys. In some cases, residue 236 of the mature HLA-C amino acid sequence is substituted with a Cys. In some cases, residue 32 (corresponding to Arg-12 of mature β2M) of an amino acid sequence depicted in FIG. 4 is substituted with a Cys.

In some cases, a β2M polypeptide comprises the amino acid sequence: IQRTPKIQVY SRHPAENGKS NFLNCYVSGF HPSDIEVDLLKNGERIEKVE HSDLSFSKDW SFYLLYYTEF TPTEKDEYAC RVNHVTLSQP KIVKWDRDM (SEQ ID NO:204). In some cases, a β2M polypeptide comprises the amino acid sequence: IQRTPKIQVY SCHPAENGKS NFLNCYVSGF HPSDIEVDLLKNGERIEKVE HSDLSFSKDW SFYLLYYTEF TPTEKDEYAC RVNHVTLSQP KIVKWDRDM (SEQ ID NO:202).

In some cases, an HLA Class I heavy chain polypeptide comprises the amino acid sequence:

(SEQ ID NO: 185)
GSHSMRYFFTSVSRPGRGEPRFIAVGYVDDTQFVRFDSDAASQRMEPR
APWIEQEGPEYWDGETRKVKAHSQTHRVDLGTLRGYYNQSEAGSHTVQ
RMYGCDVGSDWRFLRGYHQYAYDGKDYIALKEDLRSWTAADMAAQTTK
HKWEAAHVAEQLRAYLEGTCVEWLRRYLENGKETLQRTDAPKTHMTHH
AVSDHEATLRCWALSFYPAEITLTWQRDGEDQTQDTELVETRPAGDGT
FQKWAAVVVPSGQEQRYTCHVQHEGLPKPLTLRWEP.

In some cases, an HLA Class I heavy chain polypeptide comprises the amino acid sequence.

(SEQ ID NO: 205)
GSHSMRYFFTSVSRPGRGEPRFIAVGYVDDTQFVRFDSDAASQRMEPR
APWIEQEGPEYWDGETRKVKAHSQTHRVDLGTLRGYYNQSEAGSHTVQ
RMYGCDVGSDWRFLRGYHQYAYDGKDYIALKEDLRSWTAADMAAQTTK
HKWEAAHVAEQLRAYLEGTCVEWLRRYLENGKETLQRTDAPKTHMTHH
AVSDHEATLRCWALSFYPAEITLTWQRDGEDQTQDTELVETRPCGDGT
FQKWAAVVVPSGQEQRYTCHVQHEGLPKPLTLRWEP.

In some cases, an HLA Class I heavy chain polypeptide comprises the amino acid sequence:

(SEQ ID NO: 188)
GSHSMRYFFTSVSRPGRGEPRFIAVGYVDDTQFVRFDSDAASQRMEPR
APWIEQEGPEYWDGETRKVKAHSQTHRVDLGTLRGAYNQSEAGSHTVQ
RMYGCDVGSDWRFLRGYHQYAYDGKDYIALKEDLRSWTAADMAAQTTK
HKWEAAHVAEQLRAYLEGTCVEWLRRYLENGKETLQRTDAPKTHMTHH
AVSDHEATLRCWALSFYPAEITLTWQRDGEDQTQDTELVETRPCGDGT
FQKWAAVVVPSGQEQRYTCHVQHEGLPKPLTLRWE.

In some cases, the β2M polypeptide comprises the following amino acid sequence:

(SEQ ID NO: 202)
IQRTPKIQVY SCHPAENGKS NFLNCYVSGF
HPSDIEVDLLKNGERIEKVE HSDLSFSKDW
SFYLLYYTEF TPTEKDEYAC RVNHVTLSQP KIVKWDRDM;

and the HLA Class I heavy chain polypeptide of a TMMP of the present disclosure comprises the following amino acid sequence:

GSHSMRYFFTSVSRPGRGEPRFIAVGYVDDTQFVRFDSDAASQRMEPRAPWIEQ EGPEYWDGETRKVKAHSQTHRVDLGTLRGYYNQSEAGSHTVQRMYGCDVGSDWRFLRGYHQ YAYDGKDYIALKEDLRSWTAADMAAQTTKHKWEAAHVAEQLRAYLEGTCVEWLRRYLENG KETLQRTDAPKTHMTHHAVSDHEATLRCWALSFYPAEITLTWQRDGEDQTQDTELVETRPCGD GTFQKWAAVVVPSGQEQRYTCHVQHEGLPKPLTLRWEP (SEQ ID NO:205), where the Cys residues that are underlined and in bold form a disulfide bond with one another in the TMMP.

In some cases, the β2M polypeptide comprises the amino acid sequence:

(SEQ ID NO: 202)
IQRTPKIQVYSCHPAENGKSNFLNCYVSGFHPSDIEVDLLKNGERIEK
VEHSDLSFSKDWSFYLLYYTEFTPTEKDEYACRVNHVTLSQPKIVKWD
RDM.

In some cases, the first polypeptide and the second polypeptide of a TMMP of the present disclosure are disulfide linked to one another through: i) a Cys residue present in a linker connecting the peptide epitope and a β2M polypeptide in the first polypeptide chain; and ii) a Cys residue present in an MHC Class I heavy chain in the second polypeptide chain. In some cases, the Cys residue present in the MHC Class I heavy chain is a Cys introduce as a Y84C substitution. In some cases, the linker connecting the peptide epitope and the β2M polypeptide in the first polypeptide chain is GCGGS(G4S)n (SEQ ID NO:206), where n is 1, 2, 3, 4, 5, 6, 7, 8, or 9. For example, in some cases, the linker comprises the amino acid sequence GCGGSGGGGSGGGGSGGGGS (SEQ ID NO:207). As another example, the linker comprises the amino acid sequence GCGGSGGGGSGGGGS (SEQ ID NO:208). Examples of disulfide-linked first and second polypeptides of a TMMP of the present disclosure are depicted schematically in FIG. 1A-1B.

### Immunomodulatory Polypeptides

In some cases, an immunomodulatory polypeptide present in a TMMP is a wild-type immunomodulatory polypeptide. In other cases, an immunomodulatory polypeptide present in a TMMP of the present disclosure is a variant immunomodulatory polypeptide that has reduced affinity for a co-immunomodulatory polypeptide, compared to the affinity of a corresponding wild-type immunomodulatory polypeptide for the co-immunomodulatory polypeptide. Suitable immunomodulatory domains that exhibit reduced affinity for a co-immunomodulatory domain can have from 1 amino acid (aa) to 20 aa differences from a wild-type immunomodulatory domain. For example, in some cases, a variant immunomodulatory polypeptide present in a TMMP of the present disclosure differs in amino acid sequence by 1 aa, 2 aa, 3 aa, 4 aa, 5 aa, 6 aa, 7 aa, 8 aa, 9 aa, or 10 aa, from a corresponding wild-type immunomodulatory polypeptide. As another example, in some cases, a variant immunomodulatory polypeptide present in a TMMP of the present disclosure differs in amino acid sequence by 11 aa, 12 aa, 13 aa, 14 aa, 15 aa, 16 aa, 17 aa, 18 aa, 19 aa, or 20 aa, from a corresponding wild-type immunomodulatory polypeptide. As an example, in some cases, a variant immunomodulatory polypeptide present in a TMMP of the present disclosure includes 1, 2, 3, 4, 5, 6, 7, 8, 9, or 10 amino acid substitutions, compared to a corresponding reference (e.g., wild-type) immunomodulatory polypeptide. In some cases, variant immunomodulatory polypeptide present in a TMMP of the present disclosure includes a single amino acid substitution compared to a corresponding reference (e.g., wild-type) immunomodulatory polypeptide. In some cases, variant immunomodulatory polypeptide present in a TMMP of the present disclosure includes 2 amino acid substitutions (e.g., no more than 2 amino acid substitutions) compared to a corresponding reference (e.g., wild-type) immunomodulatory polypeptide. In some cases, variant immunomodulatory polypeptide present in a TMMP of the present disclosure includes 3 amino acid substitutions (e.g., no more than 3 amino acid substitutions) compared to a corresponding reference (e.g., wild-type) immunomodulatory polypeptide. In some cases, variant immunomodulatory polypeptide present in a TMMP of the present disclosure includes 4 amino acid substitutions (e.g., no more than 4 amino acid substitutions) compared to a corresponding reference (e.g., wild-type) immunomodulatory polypeptide. In some cases, variant immunomodulatory polypeptide present in a TMMP of the present disclosure includes 5 amino acid substitutions (e.g., no more than 5 amino acid substitutions) compared to a corresponding reference (e.g., wild-type) immunomodulatory polypeptide. In some cases, variant immunomodulatory polypeptide present in a TMMP of the present disclosure includes 6 amino acid substitutions (e.g., no more than 6 amino acid substitutions) compared to a corresponding reference (e.g., wild-type) immunomodulatory polypeptide. In some cases, variant immunomodulatory polypeptide present in a TMMP of the present disclosure includes 7 amino acid substitutions (e.g., no more than 7 amino acid substitutions) compared to a corresponding reference (e.g., wild-type) immunomodulatory polypeptide. In some cases, variant immunomodulatory polypeptide present in a TMMP of the present disclosure includes 8 amino acid substitutions (e.g., no more than 8 amino acid substitutions) compared to a corresponding reference (e.g., wild-type) immunomodulatory polypeptide. In some cases, variant immunomodulatory polypeptide present in a TMMP of the present disclosure includes 9 amino acid substitutions (e.g., no more than 9 amino acid substitutions) compared to a corresponding reference (e.g., wild-type) immunomodulatory polypeptide. In some cases, variant immunomodulatory polypeptide present in a TMMP of the present disclosure includes 10 amino acid substitutions (e.g., no more than 10 amino acid substitutions) compared to a corresponding reference (e.g., wild-type) immunomodulatory polypeptide.

In some cases, variant immunomodulatory polypeptide present in a TMMP of the present disclosure includes 11 amino acid substitutions (e.g., no more than 11 amino acid substitutions) compared to a corresponding reference (e.g., wild-type) immunomodulatory polypeptide.

In some cases, variant immunomodulatory polypeptide present in a TMMP of the present disclosure includes 12 amino acid substitutions (e.g., no more than 12 amino acid substitutions) compared to a corresponding reference (e.g., wild-type) immunomodulatory polypeptide.

In some cases, variant immunomodulatory polypeptide present in a TMMP of the present disclosure includes 13 amino acid substitutions (e.g., no more than 13 amino acid substitutions) compared to a corresponding reference (e.g., wild-type) immunomodulatory polypeptide.

In some cases, variant immunomodulatory polypeptide present in a TMMP of the present disclosure includes 14 amino acid substitutions (e.g., no more than 14 amino acid substitutions) compared to a corresponding reference (e.g., wild-type) immunomodulatory polypeptide.

In some cases, variant immunomodulatory polypeptide present in a TMMP of the present disclosure includes 15 amino acid substitutions (e.g., no more than 15 amino acid substitutions) compared to a corresponding reference (e.g., wild-type) immunomodulatory polypeptide.

In some cases, variant immunomodulatory polypeptide present in a TMMP of the present disclosure includes 16 amino acid substitutions (e.g., no more than 16 amino acid substitutions) compared to a corresponding reference (e.g., wild-type) immunomodulatory polypeptide.

In some cases, variant immunomodulatory polypeptide present in a TMMP of the present disclosure includes 17 amino acid substitutions (e.g., no more than 17 amino acid substitutions) compared to a corresponding reference (e.g., wild-type) immunomodulatory polypeptide.

In some cases, variant immunomodulatory polypeptide present in a TMMP of the present disclosure includes 18 amino acid substitutions (e.g., no more than 18 amino acid substitutions) compared to a corresponding reference (e.g., wild-type) immunomodulatory polypeptide.

In some cases, variant immunomodulatory polypeptide present in a TMMP of the present disclosure includes 19 amino acid substitutions (e.g., no more than 19 amino acid substitutions) compared to a corresponding reference (e.g., wild-type) immunomodulatory polypeptide.

In some cases, variant immunomodulatory polypeptide present in a TMMP of the present disclosure includes 20 amino acid substitutions (e.g., no more than 20 amino acid substitutions) compared to a corresponding reference (e.g., wild-type) immunomodulatory polypeptide.

As discussed above, a variant immunomodulatory polypeptide suitable for inclusion in a TMMP of the present disclosure exhibits reduced affinity for a cognate co-immunomodulatory polypeptide, compared to the affinity of a corresponding wild-type immunomodulatory polypeptide for the cognate co-immunomodulatory polypeptide.

Exemplary pairs of immunomodulatory polypeptide and cognate co-immunomodulatory polypeptide include, but are not limited to:
a) 4-1BBL (immunomodulatory polypeptide) and 4-1BB (cognate co-immunomodulatory polypeptide);
b) PD-L1 (immunomodulatory polypeptide) and PD1 (cognate co-immunomodulatory polypeptide);
c) IL-2 (immunomodulatory polypeptide) and IL-2 receptor (cognate co-immunomodulatory polypeptide);
d) CD80 (immunomodulatory polypeptide) and CD86 (cognate co-immunomodulatory polypeptide);
e) CD86 (immunomodulatory polypeptide) and CD28 (cognate co-immunomodulatory polypeptide);
f) OX40L (CD252) (immunomodulatory polypeptide) and OX40 (CD134) (cognate co-immunomodulatory polypeptide);
g) Fas ligand (immunomodulatory polypeptide) and Fas (cognate co-immunomodulatory polypeptide);
h) ICOS-L (immunomodulatory polypeptide) and ICOS (cognate co-immunomodulatory polypeptide);
i) ICAM (immunomodulatory polypeptide) and LFA-1 (cognate co-immunomodulatory polypeptide);
j) CD30L (immunomodulatory polypeptide) and CD30 (cognate co-immunomodulatory polypeptide);
k) CD40 (immunomodulatory polypeptide) and CD40L (cognate co-immunomodulatory polypeptide);
l) CD83 (immunomodulatory polypeptide) and CD83L (cognate co-immunomodulatory polypeptide);
m) HVEM (CD270) (immunomodulatory polypeptide) and CD160 (cognate co-immunomodulatory polypeptide);
n) JAG1 (CD339) (immunomodulatory polypeptide) and Notch (cognate co-immunomodulatory polypeptide);
o) JAG1 (immunomodulatory polypeptide) and CD46 (cognate co-immunomodulatory polypeptide);
p) CD80 (immunomodulatory polypeptide) and CTLA4 (cognate co-immunomodulatory polypeptide);
q) CD86 (immunomodulatory polypeptide) and CTLA4 (cognate co-immunomodulatory polypeptide); and
r) CD70 (immunomodulatory polypeptide) and CD27 (cognate co-immunomodulatory polypeptide).
In some cases, a variant immunomodulatory polypeptide present in a TMMP of the present disclosure has a binding affinity for a cognate co-immunomodulatory polypeptide that is from 100 nM to 100 μM. For example, in some cases, a variant immunomodulatory polypeptide present in a TMMP of the present disclosure has a binding affinity for a cognate co-immunomodulatory polypeptide that is from about 100 nM to 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

A variant immunomodulatory polypeptide present in a TMMP of the present disclosure exhibits reduced affinity for a cognate co-immunomodulatory polypeptide. Similarly, a TMMP of the present disclosure that comprises a variant immunomodulatory polypeptide exhibits reduced affinity for a cognate co-immunomodulatory polypeptide. Thus, for example, a TMMP of the present disclosure that comprises a variant immunomodulatory polypeptide has a binding affinity for a cognate co-immunomodulatory polypeptide that is from 100 nM to 100 μM. For example, in some cases, a TMMP of the present disclosure that comprises a variant immunomodulatory polypeptide has a binding affinity for a cognate co-immunomodulatory polypeptide that is from about 100 nM to 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

### CD80 Variants

In some cases, a variant immunomodulatory polypeptide present in a TMMP of the present disclosure is a variant CD80 polypeptide. Wild-type CD80 binds to CD28. Wild-type CD80 also binds to CD86.

A wild-type amino acid sequence of the ectodomain of human CD80 can be as follows:

(SEQ ID NO: 435)
VIHVTK EVKEVATLSC GHNVSVEELA QTRIYWQKEK
KMVLTMMSGD MNIWPEYKNR TIFDITNNLS IVILALRPSD
EGTYECVVLK YEKDAFKREH LAEVTLSVKA DFPTPSISDF
EIPTSNIRRI ICSTSGGFPE PHLSWLENGE ELNAINTTVS
QDPETELYAV SSKLDFNMTT NHSFMCLIKY GHLRVNQTFN
WNTTKQEHFP DN.

A wild-type CD28 amino acid sequence can be as follows: MLRLLLALNL FPSIQVTGNK ILVKQSPMLV AYDNAVNLSC KYSYNLFSRE FRASLHKGLD SAVEVCVVYG NYSQQLQVYS KTGFNCDGKL GNESVTFYLQ NLYVNQTDIY FCKIEVMYPP PYLDNEKSNG TIIHVKGKHL CPSPLFPGPS KPFWVLVVVG GVLACYSLLV TVAFIIFWVR SKRSRLLHSD YMNMTPRRPG PTRKHYQPYA PPRDFAAYRS (SEQ ID NO:436). In some cases, where a TMMP of the present disclosure comprises a variant CD80 polypeptide, a “cognate co-immunomodulatory polypeptide” is a CD28 polypeptide comprising the amino acid sequence of SEQ ID NO:5.

A wild-type CD28 amino acid sequence can be as follows: MLRLLLALNL FPSIQVTGNK ILVKQSPMLV AYDNAVNLSW KHLCPSPLFP GPSKPFWVLV VVGGVLACYS LLVTVAFIIF WVRSKRSRLL HSDYMNMTPR RPGPTRKHYQ PYAPPRDFAA YRS (SEQ ID NO:437)

A wild-type CD28 amino acid sequence can be as follows: MLRLLLALNL FPSIQVTGKH LCPSPLFPGP SKPFWVLVVV GGVLACYSLL VTVAFIIFWV RSKRSRLLHS DYMNMTPRRP GPTRKHYQPY APPRDFAAYR S (SEQ ID NO:438).

In some cases, a variant CD80 polypeptide exhibits reduced binding affinity to CD28, compared to the binding affinity of a CD80 polypeptide comprising the amino acid sequence set forth in SEQ ID NO:4 for CD28. For example, in some cases, a variant CD80 polypeptide binds CD28 with a binding affinity that is at least 10%, at least 15%, at least 20%, at least 25%, at least 30%, at least 35%, at least 40%, at least 45%, at least 50% less, at least 55% less, at least 60% less, at least 65% less, at least 70% less, at least 75% less, at least 80% less, at least 85% less, at least 90% less, at least 95% less, or more than 95% less, than the binding affinity of a CD80 polypeptide comprising the amino acid sequence set forth in SEQ ID NO:4 for CD28 (e.g., a CD28 polypeptide comprising the amino acid sequence set forth in one of SEQ ID NO:436, 437, or 438).

In some cases, a variant CD80 polypeptide has a binding affinity to CD28 that is from 100 nM to 100 μM. As another example, in some cases, a variant CD80 polypeptide of the present disclosure has a binding affinity for CD28 (e.g., a CD28 polypeptide comprising the amino acid sequence set forth in SEQ ID NO:5, SEQ ID NO:6, or SEQ ID NO:7) that is from about 100 nM to 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

In some cases, a variant CD80 polypeptide has a single amino acid substitution compared to the CD80 amino acid sequence set forth in SEQ ID NO:435. In some cases, a variant CD80 polypeptide has from 2 to 10 amino acid substitutions compared to the CD80 amino acid sequence set forth in SEQ ID NO:435. In some cases, a variant CD80 polypeptide has 2 amino acid substitutions compared to the CD80 amino acid sequence set forth in SEQ ID NO:435. In some cases, a variant CD80 polypeptide has 3 amino acid substitutions compared to the CD80 amino acid sequence set forth in SEQ ID NO:435. In some cases, a variant CD80 polypeptide has 4 amino acid substitutions compared to the CD80 amino acid sequence set forth in SEQ ID NO:435. In some cases, a variant CD80 polypeptide has 5 amino acid substitutions compared to the CD80 amino acid sequence set forth in SEQ ID NO:435. In some cases, a variant CD80 polypeptide has 6 amino acid substitutions compared to the CD80 amino acid sequence set forth in SEQ ID NO:435. In some cases, a variant CD80 polypeptide has 7 amino acid substitutions compared to the CD80 amino acid sequence set forth in SEQ ID NO:435. In some cases, a variant CD80 polypeptide has 8 amino acid substitutions compared to the CD80 amino acid sequence set forth in SEQ ID NO:435. In some cases, a variant CD80 polypeptide has 9 amino acid substitutions compared to the CD80 amino acid sequence set forth in SEQ ID NO:435. In some cases, a variant CD80 polypeptide has 10 amino acid substitutions compared to the CD80 amino acid sequence set forth in SEQ ID NO:435.

Suitable CD80 variants include a polypeptide that comprises an amino acid sequence having at least 90%, at least 95%, at least 98%, at least 99%, or 100%, amino acid sequence identity to any one of the following amino acid sequences:

VIHVTK EVKEVATLSC GHXVSVEELA QTRIYWQKEK KMVLTMMSGD MNIWPEYKNR TIFDITNNLS IVILALRPSD EGTYECVVLK YEKDAFKREH LAEVTLSVKA DFPTPSISDF EIPTSNIRRI ICSTSGGFPE PHLSWLENGE ELNAINTTVS QDPETELYAV SSKLDFNMTT NHSFMCLIKY GHLRVNQTFN WNTTKQEHFP DN (SEQ ID NO:209), where X is any amino acid other than Asn. In some cases, X is Ala;
VIHVTK EVKEVATLSC GHNVSVEELA QTRIYWQKEK KMVLTMMSGD MNIWPEYKNR TIFDITXNLS IVILALRPSD EGTYECVVLK YEKDAFKREH LAEVTLSVKA DFPTPSISDF EIPTSNIRRI ICSTSGGFPE PHLSWLENGE ELNAINTTVS QDPETELYAV SSKLDFNMTT NHSFMCLIKY GHLRVNQTFN WNTTKQEHFP DN (SEQ ID NO:210), where X is any amino acid other than Asn. In some cases, X is Ala;
VIHVTK EVKEVATLSC GHNVSVEELA QTRIYWQKEK KMVLTMMSGD MNIWPEYKNR TIFDITNNLS XVILALRPSD EGTYECVVLK YEKDAFKREH LAEVTLSVKA DFPTPSISDF EIPTSNIRRI ICSTSGGFPE PHLSWLENGE ELNAINTTVS QDPETELYAV SSKLDFNMTT NHSFMCLIKY GHLRVNQTFN WNTTKQEHFP DN (SEQ ID NO:211), where X is any amino acid other than Ile. In some cases, X is Ala;
VIHVTK EVKEVATLSC GHNVSVEELA QTRIYWQKEK KMVLTMMSGD MNIWPEYKNR TIFDITNNLS IVILALRPSD EGTYECVVLX YEKDAFKREH LAEVTLSVKA DFPTPSISDF EIPTSNIRRI ICSTSGGFPE PHLSWLENGE ELNAINTTVS QDPETELYAV SSKLDFNMTT NHSFMCLIKY GHLRVNQTFN WNTTKQEHFP DN (SEQ ID NO:212), where X is any amino acid other than Lys. In some cases, X is Ala;
VIHVTK EVKEVATLSC GHNVSVEELA QTRIYWQKEK KMVLTMMSGD MNIWPEYKNR TIFDITNNLS IVILALRPSD EGTYECVVLK YEKDAFKREH LAEVTLSVKA DFPTPSISDF EIPTSNIRRI ICSTSGGFPE PHLSWLENGE ELNAINTTVS XDPETELYAV SSKLDFNMTT NHSFMCLIKY GHLRVNQTFN WNTTKQEHFP DN (SEQ ID NO:213), where X is any amino acid other than Gln. In some cases, X is Ala;
VIHVTK EVKEVATLSC GHNVSVEELA QTRIYWQKEK KMVLTMMSGD MNIWPEYKNR TIFDITNNLS IVILALRPSD EGTYECVVLK YEKDAFKREH LAEVTLSVKA DFPTPSISDF EIPTSNIRRI ICSTSGGFPE PHLSWLENGE ELNAINTTVS QXPETELYAV SSKLDFNMTT NHSFMCLIKY GHLRVNQTFN WNTTKQEHFP DN (SEQ ID NO:214), where X is any amino acid other than Asp. In some cases, X is Ala;

VIHVTK EVKEVATLSC GHNVSVEEXA QTRJYWQKEK KMVLTMMSGD MNIWPEYKNR TIFDITNNLS IVILALRPSD EGTYECVVLK YEKDAFKREH LAEVTLSVKA DFPTPSISDF EIPTSNIRRI ICSTSGGFPE PHLSWLENGE ELNAINTTVS QDPETELYAV SSKLDFNMTT NHSFMCLIKY GHLRVNQTFN WNTTKQEHFP DN (SEQ ID NO:215), where X is any amino acid other than Leu. In some cases, X is Ala;
VIHVTK EVKEVATLSC GHNVSVEELA QTRIXWQKEK KMVLTMMSGD MNIWPEYKNR TIFDITNNLS IVILALRPSD EGTYECVVLK YEKDAFKREH LAEVTLSVKA DFPTPSISDF EIPTSNIRRI ICSTSGGFPE PHLSWLENGE ELNAINTTVS QDPETELYAV SSKLDFNMTT NHSFMCLIKY GHLRVNQTFN WNTTKQEHFP DN (SEQ ID NO:216), where X is any amino acid other than Tyr. In some cases, X is Ala;
VIHVTK EVKEVATLSC GHNVSVEELA QTRIYWXKEK KMVLTMMSGD MNIWPEYKNR TIFDITNNLS IVILALRPSD EGTYECVVLK YEKDAFKREH LAEVTLSVKA DFPTPSISDF EIPTSNIRRI ICSTSGGFPE PHLSWLENGE ELNAINTTVS QDPETELYAV SSKLDFNMTT NHSFMCLIKY GHLRVNQTFN WNTTKQEHFP DN (SEQ ID NO:217), where X is any amino acid other than Gln. In some cases, X is Ala;
VIHVTK EVKEVATLSC GHNVSVEELA QTRIYWQKEK KXVLTMMSGD MNIWPEYKNR TIFDITNNLS IVILALRPSD EGTYECVVLK YEKDAFKREH LAEVTLSVKA DFPTPSISDF EIPTSNIRRI ICSTSGGFPE PHLSWLENGE ELNAINTTVS QDPETELYAV SSKLDFNMTT NHSFMCLIKY GHLRVNQTFN WNTTKQEHFP DN (SEQ ID NO:218), where X is any amino acid other than Met. In some cases, X is Ala;
VIHVTK EVKEVATLSC GHNVSVEELA QTRIYWQKEK KMXLTMMSGD MNIWPEYKNR TIFDITNNLS IVILALRPSD EGTYECVVLK YEKDAFKREH LAEVTLSVKA DFPTPSISDF EIPTSNIRRI ICSTSGGFPE PHLSWLENGE ELNAINTTVS QDPETELYAV SSKLDFNMTT NHSFMCLIKY GHLRVNQTFN WNTTKQEHFP DN (SEQ ID NO:219), where X is any amino acid other than Val. In some cases, X is Ala;
VIHVTK EVKEVATLSC GHNVSVEELA QTRIYWQKEK KMVLTMMSGD MNXWPEYKNR TIFDITNNLS IVILALRPSD EGTYECVVLK YEKDAFKREH LAEVTLSVKA DFPTPSISDF EIPTSNIRRI ICSTSGGFPE PHLSWLENGE ELNAINTTVS QDPETELYAV SSKLDFNMTT NHSFMCLIKY GHLRVNQTFN WNTTKQEHFP DN (SEQ ID NO:220), where X is any amino acid other than Ile. In some cases, X is Ala;
VIHVTK EVKEVATLSC GHNVSVEELA QTRIYWQKEK KMVLTMMSGD MNIWPEXKNR TIFDITNNLS IVILALRPSD EGTYECVVLK YEKDAFKREH LAEVTLSVKA DFPTPSISDF EIPTSNIRRI ICSTSGGFPE PHLSWLENGE ELNAINTTVS QDPETELYAV SSKLDFNMTT NHSFMCLIKY GHLRVNQTFN WNTTKQEHFP DN (SEQ ID NO:221), where X is any amino acid other than Tyr. In some cases, X is Ala;
VIHVTK EVKEVATLSC GHNVSVEELA QTRIYWQKEK KMVLTMMSGD MNIWPEYKNR TIFXITNNLS IVILALRPSD EGTYECVVLK YEKDAFKREH LAEVTLSVKA DFPTPSISDF EIPTSNIRRI ICSTSGGFPE PHLSWLENGE ELNAINTTVS QDPETELYAV SSKLDFNMTT NHSFMCLIKY GHLRVNQTFN WNTTKQEHFP DN (SEQ ID NO:222), where X is any amino acid other than Asp. In some cases, X is Ala;
VIHVTK EVKEVATLSC GHNVSVEELA QTRIYWQKEK KMVLTMMSGD MNIWPEYKNR TIFDITNNLS IVILALRPSD EGTYECVVLK YEKDAFKREH LAEVTLSVKA DXPTPSISDF EIPTSNIRRI ICSTSGGFPE PHLSWLENGE ELNAINTTVS QDPETELYAV SSKLDFNMTT NHSFMCLIKY GHLRVNQTFN WNTTKQEHFP DN (SEQ ID NO:223), where X is any amino acid other than Phe. In some cases, X is Ala;
VIHVTK EVKEVATLSC GHNVSVEELA QTRIYWQKEK KMVLTMMSGD MNIWPEYKNR TIFDITNNLS IVILALRPSD EGTYECVVLK YEKDAFKREH LAEVTLSVKA DFPTPSISDF EIPTSNIRRI ICSTSGGFPE PHLSWLENGE ELNAINTTVX QDPETELYAV SSKLDFNMTT NHSFMCLIKY GHLRVNQTFN WNTTKQEHFP DN (SEQ ID NO:224), where X is any amino acid other than Ser. In some cases, X is Ala; and

VIHVTK EVKEVATLSC GHNVSVEELA QTRIYWQKEK KMVLTMMSGD MNIWPEYKNR TIFDITNNLS IVILALRPSD EGTYECVVLK YEKDAFKREH LAEVTLSVKA DFPTXSISDF EIPTSNIRRI ICSTSGGFPE PHLSWLENGE ELNAINTTVS QDPETELYAV SSKLDFNMTT NHSFMCLIKY GHLRVNQTFN WNTTKQEHFP DN (SEQ ID NO:225), where X is any amino acid other than Pro. In some cases, X is Ala.

### CD86 Variants

In some cases, a variant immunomodulatory polypeptide present in a TMMP of the present disclosure is a variant CD86 polypeptide. Wild-type CD86 binds to CD28. In some cases, where a TMMP of the present disclosure comprises a variant CD86 polypeptide, a “cognate co-immunomodulatory polypeptide” is a CD28 polypeptide comprising the amino acid sequence of SEQ ID NO:5.

The amino acid sequence of the full ectodomain of a wild-type human CD86 can be as follows:

(SEQ ID NO: 226)
APLKIQAYFNETADLPCQFANSQNQSLSELVVFWQDQENLVLN
EVYLGKEKFDSVHSKYMNRTSFDSDSWTLRLHNLQIKDKGLYQ
CIIHHKKPTGMIRIHQMNSELSVLANFSQPEIVPISNITENVY
INLTCSSIHGYPEPKKMSVLLRTKNSTIEYDGIMQKSQDNVTE
LYDVSISLSVSFPDVTSNMTIFCILETDKTRLLSSPFSIELED
PQPPPDHIP.

The amino acid sequence of the IgV domain of a wild-type human CD86 can be as follows:

(SEQ ID NO: 227)
APLKIQAYFNETADLPCQFANSQNQSLSELVVFWQDQENLVLN
EVYLGKEKFDSVHSKYMNRTSFDSDSWTLRLHNLQIKDKGLYQ
CIIHHKKPTGMIRIHQMNSELSVL.

In some cases, a variant CD86 polypeptide exhibits reduced binding affinity to CD28, compared to the binding affinity of a CD86 polypeptide comprising the amino acid sequence set forth in SEQ ID NO:226 or SEQ ID NO:227 for CD28. For example, in some cases, a variant CD86 polypeptide binds CD28 with a binding affinity that is at least 10%, at least 15%, at least 20%, at least 25%, at least 30%, at least 35%, at least 40%, at least 45%, at least 50% less, at least 55% less, at least 60% less, at least 65% less, at least 70% less, at least 75% less, at least 80% less, at least 85% less, at least 90% less, at least 95% less, or more than 95% less, than the binding affinity of a CD86 polypeptide comprising the amino acid sequence set forth in SEQ ID NO:226 or SEQ ID NO:227 for CD28 (e.g., a CD28 polypeptide comprising the amino acid sequence set forth in one of SEQ ID NO:436, 437, or 438).

In some cases, a variant CD86 polypeptide has a binding affinity to CD28 that is from 100 nM to 100 μM. As another example, in some cases, a variant CD86 polypeptide of the present disclosure has a binding affinity for CD28 (e.g., a CD28 polypeptide comprising the amino acid sequence set forth in one of SEQ ID NOs:5, 6, or 7) that is from about 100 nM to 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 M to about 5 μM, from about 5 μM to about 10 μM, from about 10 μM to about 15 μM, from about 15 M to about 20 μM, from about 20 μM to about 25 μM, from about 25 μM to about 50 μM, from about 50 μM to about 75 μM, or from about 75 μM to about 100 μM.

In some cases, a variant CD86 polypeptide has a single amino acid substitution compared to the CD86 amino acid sequence set forth in SEQ ID NO:226. In some cases, a variant CD86 polypeptide has from 2 to 10 amino acid substitutions compared to the CD86 amino acid sequence set forth in SEQ ID NO:226. In some cases, a variant CD86 polypeptide has 2 amino acid substitutions compared to the CD86 amino acid sequence set forth in SEQ ID NO:226. In some cases, a variant CD86 polypeptide has 3 amino acid substitutions compared to the CD86 amino acid sequence set forth in SEQ ID NO:226. In some cases, a variant CD86 polypeptide has 4 amino acid substitutions compared to the CD86 amino acid sequence set forth in SEQ ID NO:226. In some cases, a variant CD86 polypeptide has 5 amino acid substitutions compared to the CD86 amino acid sequence set forth in SEQ ID NO:226. In some cases, a variant CD86 polypeptide has 6 amino acid substitutions compared to the CD86 amino acid sequence set forth in SEQ ID NO:226. In some cases, a variant CD86 polypeptide has 7 amino acid substitutions compared to the CD86 amino acid sequence set forth in SEQ ID NO:226. In some cases, a variant CD86 polypeptide has 8 amino acid substitutions compared to the CD86 amino acid sequence set forth in SEQ ID NO:226. In some cases, a variant CD86 polypeptide has 9 amino acid substitutions compared to the CD86 amino acid sequence set forth in SEQ ID NO:226. In some cases, a variant CD86 polypeptide has 10 amino acid substitutions compared to the CD86 amino acid sequence set forth in SEQ ID NO:226.

In some cases, a variant CD86 polypeptide has a single amino acid substitution compared to the CD86 amino acid sequence set forth in SEQ ID NO:227. In some cases, a variant CD86 polypeptide has from 2 to 10 amino acid substitutions compared to the CD86 amino acid sequence set forth in SEQ ID NO:227. In some cases, a variant CD86 polypeptide has 2 amino acid substitutions compared to the CD86 amino acid sequence set forth in SEQ ID NO:227. In some cases, a variant CD86 polypeptide has 3 amino acid substitutions compared to the CD86 amino acid sequence set forth in SEQ ID NO:227. In some cases, a variant CD86 polypeptide has 4 amino acid substitutions compared to the CD86 amino acid sequence set forth in SEQ ID NO:227. In some cases, a variant CD86 polypeptide has 5 amino acid substitutions compared to the CD86 amino acid sequence set forth in SEQ ID NO:227. In some cases, a variant CD86 polypeptide has 6 amino acid substitutions compared to the CD86 amino acid sequence set forth in SEQ ID NO:227. In some cases, a variant CD86 polypeptide has 7 amino acid substitutions compared to the CD86 amino acid sequence set forth in SEQ ID NO:227. In some cases, a variant CD86 polypeptide has 8 amino acid substitutions compared to the CD86 amino acid sequence set forth in SEQ ID NO:227. In some cases, a variant CD86 polypeptide has 9 amino acid substitutions compared to the CD86 amino acid sequence set forth in SEQ ID NO:227. In some cases, a variant CD86 polypeptide has 10 amino acid substitutions compared to the CD86 amino acid sequence set forth in SEQ ID NO:227.

Suitable CD86 variants include a polypeptide that comprises an amino acid sequence having at least 90%, at least 95%, at least 98%, at least 99%, or 100%, amino acid sequence identity to any one of the following amino acid sequences:
APLKIQAYFNETADLPCQFANSQNQSLSELVVFWQDQENLVLNEVYLGKEKFDSVHSKY MXRTSFDSDSWTLRLHNLQIKDKGLYQCI IHHKKPTGMIRIHQMNSELSVLANFSQPEIVPISNITENVY INLTCSSIHGYPEPKKMSVLLRTKNSTIEYDGIMQKSQDNVTELYDVSISLSVSFPDVTSNMTI FCILET DKTRLLSSPFSIELEDPQPPPDHIP (SEQ ID NO:228), where X is any amino acid other than Asn. In some cases, X is Ala;
APLKIQAYFNETADLPCQFANSQNQSLSELVVFWQDQENLVLNEVYLGKEKFDSVHSKY MNRTSFXSDSWTLRLHNLQIKDKGLYQCI IHHKKPTGMIRIHQMNSELSVLANFSQPEIVPISNITENVY INLTCSSIHGYPEPKKMSVLLRTKNSTIEYDGIMQKSQDNVTELYDVSISLSVSFPDVTSNMTI FCILET DKTRLLSSPFSIELEDPQPPPDHIP (SEQ ID NO:229), where X is any amino acid other than Asp. In some cases, X is Ala;
APLKIQAYFNETADLPCQFANSQNQSLSELVVFWQDQENLVLNEVYLGKEKFDSVHSKY MNRTSFDSDSXTLRLHNLQIKDKGLYQCIIHHKKPTGMIRIHQMNSELSVLANFSQPEIVPISNITENVY INLTCSSIHGYPEPKKMSVLLRTKNSTIEYDGIMQKSQDNVTELYDVSISLSVSFPDVTSNMTI FCILET DKTRLLSSPFSIELEDPQPPPDHIP (SEQ ID NO:230), where X is any amino acid other than Trp. In some cases, X is Ala;
APLKIQAYFNETADLPCQFANSQNQSLSELVVFWQDQENLVLNEVYLGKEKFDSVHSKY MNRTSFDSDSWTLRLHNLQIKDKGLYQCIIHXKKPTGMIRIHQMNSELSVLANFSQPEIVPISNITENVY INLTCSSIHGYPEPKKMSVLLRTKNSTIEYDGIMQKSQDNVTELYDVSISLSVSFPDVTSNMTI FCILET DKTRLLSSPFSIELEDPQPPPDHIP (SEQ ID NO:231), where X is any amino acid other than His. In some cases, X is Ala;
APLKIQAYFNETADLPCQFANSQNQSLSELVVFWQDQENLVLNEVYLGKEKFDSVHSKY MXRTSFDSDSWTLRLHNLQIKDKGLYQCIIHHKKPTGMIRIHQMNSELSVL (SEQ ID NO:232), where X is any amino acid other than Asn. In some cases, X is Ala;
APLKIQAYFNETADLPCQFANSQNQSLSELVVFWQDQENLVLNEVYLGKEKFDSVHSKY MNRTSFXSDSWTLRLHNLQIKDKGLYQCIIHHKKPTGMIRIHQMNSELSVL (SEQ ID NO:233), where X is any amino acid other than Asp. In some cases, X is Ala;
APLKIQAYFNETADLPCQFANSQNQSLSELVVFWQDQENLVLNEVYLGKEKFDSVHSKY MNRTSFDSDSXTLRLHNLQIKDKGLYQCIIHHKKPTGMIRIHQMNSELSVL (SEQ ID NO:234), where X is any amino acid other than Trp. In some cases, X is Ala;
APLKIQAYFNETADLPCQFANSQNQSLSELVVFWQDQENLVLNEVYLGKEKFDSVHSKY MNRTSFDSDSWTLRLHNLQIKDKGLYQCIIHXKKPTGMIRIHQMNSELSVL (SEQ ID NO:235), where X is any amino acid other than His. In some cases, X is Ala;
APLKIQAYFNETADLPCQFANSQNQSLSELVVFWQDQENLXLNEVYLGKEKFDSVHSKY MNRTSFDSDSWTLRLHNLQIKDKGLYQCIIHHKKPTGMIRIHQMNSELSVLANFSQPEIVPISNITENVY INLTCSSIHGYPEPKKMSVLLRTKNSTIEYDGIMQKSQDNVTELYDVSISLSVSFPDVTSNMTI FCILET DKTRLLSSPFSIELEDPQPPPDHIP (SEQ ID NO:236), where X is any amino acid other than Val. In some cases, X is Ala;
APLKIQAYFNETADLPCQFANSQNQSLSELVVFWQDQENLXLNEVYLGKEKFDSVHSKY MNRTSFDSDSWTLRLHNLQIKDKGLYQCIIHHKKPTGMIRIHQMNSELSVL (SEQ ID NO:237), where X is any amino acid other than Val. In some cases, X is Ala;
APLKIQAYFNETADLPCQFANSQNQSLSELVVFWXDQENLVLNEVYLGKEKFDSVHSKY MNRTSFDSDSWTLRLHNLQIKDKGLYQCIIHHKKPTGMIRIHQMNSELSVLANFSQPEIVPISNITENVY INLTCSSIHGYPEPKKMSVLLRTKNSTIEYDGIMQKSQDNVTELYDVSISLSVSFPDVTSNMTI FCILET DKTRLLSSPFSIELEDPQPPPDHIP (SEQ ID NO:238), where X is any amino acid other than Gln. In some cases, X is Ala;
APLKIQAYFNETADLPCQFANSQNQSLSELVVFWXDQENLVLNEVYLGKEKFDSVHSKY MNRTSFDSDSWTLRLHNLQIKDKGLYQCIIHHKKPTGMIRIHQMNSELSVL (SEQ ID NO:239), where X is any amino acid other than Gln. In some cases, X is Ala;
APLKIQAYFNETADLPCQFANSQNQSLSELVVXWQDQENLVLNEVYLGKEKFDSVHSKY MNRTSFDSDSWTLRLHNLQIKDKGLYQCIIHHKKPTGMIRIHQMNSELSVLANFSQPEIVPISNITENVY INLTCSSIHGYPEPKKMSVLLRTKNSTIEYDGIMQKSQDNVTELYDVSISLSVSFPDVTSNMTI FCILET DKTRLLSSPFSIELEDPQPPPDHIP (SEQ ID NO:240), where X is any amino acid other than Phe. In some cases, X is Ala;
APLKIQAYFNETADLPCQFANSQNQSLSELVVXWQDQENLVLNEVYLGKEKFDSVHSKY MNRTSFDSDSWTLRLHNLQIKDKGLYQCIIHHKKPTGMIRIHQMNSELSVL (SEQ ID NO:241), where X is any amino acid other than Phe. In some cases, X is Ala;
APLKIQAYFNETADLPCQFANSQNQSLSELVVFWQDQENLVLNEVYLGKEKFDSVHSKY MNRTSFDSDSWTXRLHNLQIKDKGLYQCIIHHKKPTGMIRIHQMNSELSVLANFSQPEIVPISNITENVY INLTCSSIHGYPEPKKMSVLLRTKNSTIEYDGIMQKSQDNVTELYDVSISLSVSFPDVTSNMTI FCILET DKTRLLSSPFSIELEDPQPPPDHIP (SEQ ID NO:242), where X is any amino acid other than Leu. In some cases, X is Ala;
APLKIQAYFNETADLPCQFANSQNQSLSELVVFWQDQENLVLNEVYLGKEKFDSVHSKY MNRTSFDSDSWTXRLHNLQIKDKGLYQCIIHHKKPTGMIRIHQMNSELSVL (SEQ ID NO:243), where X is any amino acid other than Leu. In some cases, X is Ala;
APLKIQAYFNETADLPCQFANSQNQSLSELVVFWQDQENLVLNEVYLGKEKFDSVHSKX MNRTSFDSDSWTLRLHNLQIKDKGLYQCIIHHKKPTGMIRIHQMNSELSVLANFSQPEIVPISNITENVY INLTCSSIHGYPEPKKMSVLLRTKNSTIEYDGIMQKSQDNVTELYDVSISLSVSFPDVTSNMTI FCILET DKTRLLSSPFSIELEDPQPPPDHIP (SEQ ID NO:244), where X is any amino acid other than Tyr. In some cases, X is Ala;
APLKIQAYFNETADLPCQFANSQNQSLSELVVFWQDQENLVLNEVYLGKEKFDSVHSKX MNRTSFDSDSWTLRLHNLQIKDKGLYQCIIHHKKPTGMIRIHQMNSELSVL (SEQ ID NO:245), where X is any amino acid other than Tyr. In some cases, X is Ala;
APLKIQAYFNETADLPCQFANSQNQSLSELVVFWQDQENLVLNEVYLGKEKFDSVHSKY MXRTSFDSDSWTLRLHNLQIKDKGLYQCIIHXKKPTGMIRIHQMNSELSVLANFSQPEIVPISNITENVY INLTCSSIHGYPEPKKMSVLLRTKNSTIEYDGIMQKSQDNVTELYDVSISLSVSFPDVTSNMTI FCILET DKTRLLSSPFSIELEDPQPPPDHIP (SEQ ID NO:246), where the first X is any amino acid other than Asn and the second X is any amino acid other than His. In some cases, the first and the second X are both Ala;
APLKIQAYFNETADLPCQFANSQNQSLSELVVFWQDQENLVLNEVYLGKEKFDSVHSKY MXRTSFDSDSWTLRLHNLQIKDKGLYQCIIHXKKPTGMIRIHQMNSELSVL (SEQ ID NO:247), where the first X is any amino acid other than Asn and the second X is any amino acid other than His. In some cases, the first and the second X are both Ala;
APLKIQAYFNETADLPCQFANSQNQSLSELVVFWQDQENLVLNEVYLGKEKFDSVHSKY MNRTSFX₁SDSWTLRLHNLQIKDKGLYQCIIHX₂KKPTGMIRIHQMNSELSVLANFSQPEIVPISNITENV YINLTCSSIHGYPEPKKMSVLLRTKNSTIEYDGIMQKSQDNVTELYDVSISLSVSFPDVTSNMTIFCILE TDKTRLLSSPFSIELEDPQPPPDHIP (SEQ ID NO:248), where X₁ is any amino acid other than Asp, and X₂ is any amino acid other than His. In some cases, X₁ is Ala and X₂ is Ala;
APLKIQAYFNETADLPCQFANSQNQSLSELVVFWQDQENLVLNEVYLGKEKFDSVHSKY MNRTSFX₁SDSWTLRLHNLQIKDKGLYQCIIHX₂KKPTGMIRIHQMNSELSVL (SEQ ID NO:249), where the first X is any amino acid other than Asn and the second X is any amino acid other than His. In some cases, the first and the second X are both Ala;
APLKIQAYFNETADLPCQFANSQNQSLSELVVFWQDQENLVLNEVYLGKEKFDSVHSKY MX₁RTSFX₂SDSWTLRLHNLQIKDKGLYQCI IHX₃KKPTGMIRIHQMNSELSVLANFSQPEIVPISNITEN VYINLTCSSIHGYPEPKKMSVLLRTKNSTIEYDGIMQKSQDNVTELYDVSISLSVSFPDVTSNMTIFCIL ETDKTRLLSSPFSIELEDPQPPPDHIP (SEQ ID NO:250), where X₁ is any amino acid other than Asn, X₂ is any amino acid other than Asp, and X₃ is any amino acid other than His. In some cases, X₁ is Ala, X₂ is Ala, and X₃ is Ala; and
APLKIQAYFNETADLPCQFANSQNQSLSELVVFWQDQENLVLNEVYLGKEKFDSVHSKY MX₁RTSFX₂SDSWTLRLHNLQIKDKGLYQCIIHX₃KKPTGMIRIHQMNSELSVL (SEQ ID NO:251), where X₁ is any amino acid other than Asn, X₂ is any amino acid other than Asp, and X₃ is any amino acid other than His. In some cases, X₁ is Ala, X₂ is Ala, and X₃ is Ala. 4-1BBL variants

In some cases, a variant immunomodulatory polypeptide present in a TMMP of the present disclosure is a variant 4-1BBL polypeptide. Wild-type 4-1BBL binds to 4-1BB (CD137).

A wild-type 4-1BBL amino acid sequence can be as follows: MEYASDASLD PEAPWPPAPR ARACRVLPWA LVAGLLLLLLLAAACAVFLA CPWAVSGARA SPGSAASPRL REGPELSPDD PAGLLDLRQG MFAQLVAQNV LLIDGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:252).

In some cases, a variant 4-1BBL polypeptide is a variant of the tumor necrosis factor (TNF) homology domain (THD) of human 4-1BBL.

A wild-type amino acid sequence of the THD of human 4-1BBL can be, e.g., one of SEQ ID NOs:253-255, as follows:

(SEQ ID NO: 253)
PAGLLDLRQG MFAQLVAQNV LLIDGPLSWY SDPGLAGVSL
TGGLSYKEDT KELVVAKAGV YYVFFQLELR RVVAGEGSGS
VSLALHLQPL RSAAGAAALA LTVDLPPASS EARNSAFGFQ
GRLLHLSAGQ RLGVHLHTEA RARHAWQLTQ GATVLGLFRV
TPEIPAGLPS PRSE.
(SEQ ID NO: 254)
D PAGLLDLRQG MFAQLVAQNV LLIDGPLSWY SDPGLAGVSL
TGGLSYKEDT KELVVAKAGV YYVFFQLELR RVVAGEGSGS
VSLALHLQPL RSAAGAAALA LTVDLPPASS EARNSAFGFQ
GRLLHLSAGQ RLGVHLHTEA RARHAWQLTQ GATVLGLFRV
TPEIPAGLPS PRSE.
(SEQ ID NO: 255)
D PAGLLDLRQG MFAQLVAQNV LLIDGPLSWY SDPGLAGVSL
TGGLSYKEDT KELVVAKAGV YYVFFQLELR RVVAGEGSGS
VSLALHLQPL RSAAGAAALA LTVDLPPASS EARNSAFGFQ
GRLLHLSAGQ RLGVHLHTEA RARHAWQLTQ GATVLGLFRV
TPEIPA.

A wild-type 4-1BB amino acid sequence can be as follows: MGNSCYNIVA TLLLVLNFER TRSLQDPCSN CPAGTFCDNN RNQICSPCPP NSFSSAGGQR TCDICRQCKG VFRTRKECSS TSNAECDCTP GFHCLGAGCS MCEQDCKQGQ ELTKKGCKDC CFGTFNDQKR GICRPWTNCS LDGKSVLVNG TKERDVVCGP SPADLSPGAS SVTPPAPARE PGHSPQIISF FLALTSTALL FLLFFLTLRF SVVKRGRKKL LYIFKQPFMR PVQTTQEEDG CSCRFPEEEE GGCEL (SEQ ID NO:434). In some cases, where a TMMP of the present disclosure comprises a variant 4-1BBL polypeptide, a “cognate co-immunomodulatory polypeptide” is a 4-1BB polypeptide comprising the amino acid sequence of SEQ ID NO:434.

In some cases, a variant 4-1BBL polypeptide exhibits reduced binding affinity to 4-1BB, compared to the binding affinity of a 4-1BBL polypeptide comprising the amino acid sequence set forth in one of SEQ ID NOs:252-255. For example, in some cases, a variant 4-1BBL polypeptide of the present disclosure binds 4-1BB with a binding affinity that is at least 10% less, at least 15% less, at least 20% less, at least 25%, at least 30% less, at least 35% less, at least 40% less, at least 45% less, at least 50% less, at least 55% less, at least 60% less, at least 65% less, at least 70% less, at least 75% less, at least 80% less, at least 85% less, at least 90% less, at least 95% less, or more than 95% less, than the binding affinity of a 4-1BBL polypeptide comprising the amino acid sequence set forth in one of SEQ ID NOs:252-255 for a 4-1BB polypeptide (e.g., a 4-1BB polypeptide comprising the amino acid sequence set forth in SEQ ID NO:434), when assayed under the same conditions.

In some cases, a variant 4-1BBL polypeptide has a binding affinity to 4-1BB that is from 100 nM to 100 μM. As another example, in some cases, a variant 4-1BBL polypeptide has a binding affinity for 4-1BB (e.g., a 4-1BB polypeptide comprising the amino acid sequence set forth in SEQ ID NO: 14) that is from about 100 nM to 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 M, from about 10 μM to about 15 μM, from about 15 μM to about 20 μM, from about 20 μM to about M, from about 25 μM to about 50 μM, from about 50 μM to about 75 μM, or from about 75 μM to about 100 μM.

In some cases, a variant 4-1BBL polypeptide has a single amino acid substitution compared to the 4-1BBL amino acid sequence set forth in one of SEQ ID NOs:252-255. In some cases, a variant 4-1BBL polypeptide has from 2 to 10 amino acid substitutions compared to the 4-1BBL amino acid sequence set forth in one of SEQ ID NOs:252-255. In some cases, a variant 4-1BBL polypeptide has 2 amino acid substitutions compared to the 4-1BBL amino acid sequence set forth in one of SEQ ID NOs:10-13. In some cases, a variant 4-1BBL polypeptide has 3 amino acid substitutions compared to the 4-1BBL amino acid sequence set forth in one of SEQ ID NOs:252-255. In some cases, a variant 4-1BBL polypeptide has 4 amino acid substitutions compared to the 4-1BBL amino acid sequence set forth in one of SEQ ID NOs:252-255. In some cases, a variant 4-1BBL polypeptide has 5 amino acid substitutions compared to the 4-1BBL amino acid sequence set forth in one of SEQ ID NOs:252-255. In some cases, a variant 4-1BBL polypeptide has 6 amino acid substitutions compared to the 4-1BBL amino acid sequence set forth in one of SEQ ID NOs:252-255. In some cases, a variant 4-1BBL polypeptide has 7 amino acid substitutions compared to the 4-1BBL amino acid sequence set forth in one of SEQ ID NOs:252-255. In some cases, a variant 4-1BBL polypeptide has 8 amino acid substitutions compared to the 4-1BBL amino acid sequence set forth in one of SEQ ID NOs:252-255. In some cases, a variant 4-1BBL polypeptide has 9 amino acid substitutions compared to the 4-1BBL amino acid sequence set forth in one of SEQ ID NOs:252-255. In some cases, a variant 4-1BBL polypeptide has 10 amino acid substitutions compared to the 4-1BBL amino acid sequence set forth in one of SEQ ID NOs:252-255.

Suitable 4-1BBL variants include a polypeptide that comprises an amino acid sequence having at least 90%, at least 95%, at least 98%, at least 99%, or 100%, amino acid sequence identity to any one of the following amino acid sequences:
PAGLLDLRQG MFAQLVAQNV LLIDGPLSWY SDPGLAGVSL TGGLSYXEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:256), where X is any amino acid other than Lys. In some cases, X is Ala;
PAGLLDLRQG MFAQLVAQNV LLIDGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWXLTQ GATVLGLFRV TPEIPAGLPS PRSE (SEQ ID NO:257), where X is any amino acid other than Gln. In some cases, X is Ala;
PAGLLDLRQG XFAQLVAQNV LLIDGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:258), where X is any amino acid other than Met. In some cases, X is Ala;
PAGLLDLRQG MXAQLVAQNV LLIDGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:259), where X is any amino acid other than Phe. In some cases, X is Ala;
PAGLLDLRQG MFAXLVAQNV LLIDGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:260), where X is any amino acid other than Gln. In some cases, X is Ala;
PAGLLDLRQG MFAQXVAQNV LLIDGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:261), where X is any amino acid other than Leu. In some cases, X is Ala;
PAGLLDLRQG MFAQLXAQNV LLIDGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:262), where X is any amino acid other than Val. In some cases, X is Ala;
PAGLLDLRQG MFAQLVAXNV LLIDGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:263), where X is any amino acid other than Gln. In some cases, X is Ala;
PAGLLDLRQG MFAQLVAQXV LLIDGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:264), where X is any amino acid other than Asn. In some cases, X is Ala;
PAGLLDLRQG MFAQLVAQNX LLIDGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:265), where X is any amino acid other than Val. In some cases, X is Ala;
PAGLLDLRQG MFAQLVAQNV XLIDGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:266), where X is any amino acid other than Leu. In some cases, X is Ala;
PAGLLDLRQG MFAQLVAQNV LXIDGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:267), where X is any amino acid other than Leu. In some cases, X is Ala;
PAGLLDLRQG MFAQLVAQNV LLXDGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:268), where X is any amino acid other than Ile. In some cases, X is Ala;
PAGLLDLRQG MFAQLVAQNV LLIXGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:269), where X is any amino acid other than Asp. In some cases, X is Ala;
PAGLLDLRQG MFAQLVAQNV LLIDXPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:270), where X is any amino acid other than Gly. In some cases, X is Ala;
PAGLLDLRQG MFAQLVAQNV LLIDGXLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:271), where X is any amino acid other than Pro. In some cases, X is Ala;
PAGLLDLRQG MFAQLVAQNV LLIDGPXSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:272), where X is any amino acid other than Leu. In some cases, X is Ala;
PAGLLDLRQG MFAQLVAQNV LLIDGPLXWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:273), where X is any amino acid other than Ser. In some cases, X is Ala;
PAGLLDLRQG MFAQLVAQNV LLIDGPLSXY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:274), where X is any amino acid other than Trp. In some cases, X is Ala;
PAGLLDLRQG MFAQLVAQNV LLIDGPLSWX SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:275), where X is any amino acid other than Tyr. In some cases, X is Ala;
PAGLLDLRQG MFAQLVAQNV LLIDGPLSWY XDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:276), where X is any amino acid other than Ser. In some cases, X is Ala;
PAGLLDLRQG MFAQLVAQNV LLIDGPLSWY SXPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:277), where X is any amino acid other than Asp. In some cases, X is Ala;
PAGLLDLRQG MFAQLVAQNV LLIDGPLSWY SDXGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:278), where X is any amino acid other than Pro. In some cases, X is Ala;
PAGLLDLRQG MFAQLVAQNV LLIDGPLSWY SDPXLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:279), where X is any amino acid other than Gly. In some cases, X is Ala;
PAGLLDLRQG MFAQLVAQNV LLIDGPLSWY SDPGXAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:280), where X is any amino acid other than Leu. In some cases, X is Ala;
PAGLLDLRQG MFAQLVAQNV LLIDGPLSWY SDPGLAXVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:281), where X is any amino acid other than Gly. In some cases, X is Ala;
PAGLLDLRQG MFAQLVAQNV LLIDGPLSWY SDPGLAGXSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:282), where X is any amino acid other than Val. In some cases, X is Ala;
PAGLLDLRQG MFAQLVAQNV LLIDGPLSWY SDPGLAGVXL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:283), where X is any amino acid other than Ser. In some cases, X is Ala;
PAGLLDLRQG MFAQLVAQNV LLIDGPLSWY SDPGLAGVSX TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:284), where X is any amino acid other than Leu. In some cases, X is Ala;
PAGLLDLRQG MFAQLVAQNV LLIDGPLSWY SDPGLAGVSL XGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:285), where X is any amino acid other than Thr. In some cases, X is Ala;
PAGLLDLRQG MFAQLVAQNV LLIDGPLSWY SDPGLAGVSL TXGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:286), where X is any amino acid other than Gly. In some cases, X is Ala;
PAGLLDLRQG MFAQLVAQNV LLIDGPLSWY SDPGLAGVSL TGXLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:287), where X is any amino acid other than Gly. In some cases, X is Ala;
PAGLLDLRQG MFAQLVAQNV LLIDGPLSWY SDPGLAGVSL TGGXSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:288), where X is any amino acid other than Leu. In some cases, X is Ala;
PAGLLDLRQG MFAQLVAQNV LLIDGPLSWY SDPGLAGVSL TGGLXYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:289), where X is any amino acid other than Ser. In some cases, X is Ala;
PAGLLDLRQG MFAQLVAQNV LLIDGPLSWY SDPGLAGVSL TGGLSXKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:290), where X is any amino acid other than Tyr. In some cases, X is Ala;
PAGLLDLRQG MFAQLVAQNV LLIDGPLSWY SDPGLAGVSL TGGLSYKXDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:291), where X is any amino acid other than Glu. In some cases, X is Ala;
PAGLLDLRQG MFAQLVAQNV LLIDGPLSWY SDPGLAGVSL TGGLSYKEXT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:292), where X is any amino acid other than Asp. In some cases, X is Ala;
PAGLLDLRQG MFAQLVAQNV LLIDGPLSWY SDPGLAGVSL TGGLSYKEDX KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:293), where X is any amino acid other than Thr. In some cases, X is Ala;
PAGLLDLRQG MFAQLVAQNV LLIDGPLSWY SDPGLAGVSL TGGLSYKEDT XELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:294), where X is any amino acid other than Lys. In some cases, X is Ala;
PAGLLDLRQG MFAQLVAQNV LLIDGPLSWY SDPGLAGVSL TGGLSYKEDT KXLVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:295), where X is any amino acid other than Glu. In some cases, X is Ala;
PAGLLDLRQG MFAQLVAQNV LLIDGPLSWY SDPGLAGVSL TGGLSYKEDT KELVVAKAGV YYVXFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:296), where X is any amino acid other than Phe. In some cases, X is Ala;
PAGLLDLRQG MFAQLVAQNV LLIDGPLSWY SDPGLAGVSL TGGLSYKEDT KELVVAKAGV YYVFXQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:297), where X is any amino acid other than Phe. In some cases, X is Ala;
PAGLLDLRQG MFAQLVAQNV LLIDGPLSWY SDPGLAGVSL TGGLSYKEDT KELVVAKAGV YYVFFXLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:298), where X is any amino acid other than Gln. In some cases, X is Ala;
PAGLLDLRQG MFAQLVAQNV LLIDGPLSWY SDPGLAGVSL TGGLSYKEDT KELVVAKAGV YYVFFQXELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:299), where X is any amino acid other than Leu. In some cases, X is Ala;
PAGLLDLRQG MFAQLVAQNV LLIDGPLSWY SDPGLAGVSL TGGLSYKEDT KELVVAKAGV YYVFFQLXLR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:300), where X is any amino acid other than Glu. In some cases, X is Ala;
PAGLLDLRQG MFAQLVAQNV LLIDGPLSWY SDPGLAGVSL TGGLSYKEDT KELVVAKAGV YYVFFQLEXR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:301), where X is any amino acid other than Leu. In some cases, X is Ala;
PAGLLDLRQG MFAQLVAQNV LLIDGPLSWY SDPGLAGVSL TGGLSYKEDT KELVVAKAGV YYVFFQLELX RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:302), where X is any amino acid other than Arg. In some cases, X is Ala;
PAGLLDLRQG MFAQLVAQNV LLIDGPLSWY SDPGLAGVSL TGGLSYKEDT KELVVAKAGV YYVFFQLELR XVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:303), where X is any amino acid other than Arg. In some cases, X is Ala;
PAGLLDLRQG MFAQLVAQNV LLIDGPLSWY SDPGLAGVSL TGGLSYKEDT KELVVAKAGV YYVFFQLELR RXVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:304), where X is any amino acid other than Val. In some cases, X is Ala;
PAGLLDLRQG MFAQLVAQNV LLIDGPLSWY SDPGLAGVSL TGGLSYKEDT KELVVAKAGV YYVFFQLELR RVXAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:305), where X is any amino acid other than Val. In some cases, X is Ala;
PAGLLDLRQG MFAQLVAQNV LLIDGPLSWY SDPGLAGVSL TGGLSYKEDT KELVVAKAGV YYVFFQLELR RVVAXEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:306), where X is any amino acid other than Gly. In some cases, X is Ala;
PAGLLDLRQG MFAQLVAQNV LLIDGPLSWY SDPGLAGVSL TGGLSYKEDT KELVVAKAGV YYVFFQLELR RVVAGXGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:307), where X is any amino acid other than Glu. In some cases, X is Ala;
PAGLLDLRQG MFAQLVAQNV LLIDGPLSWY SDPGLAGVSL TGGLSYKEDT KELVVAKAGV YYVFFQLELR RVVAGEXSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:308), where X is any amino acid other than Gly. In some cases, X is Ala;
PAGLLDLRQG MFAQLVAQNV LLIDGPLSWY SDPGLAGVSL TGGLSYKEDT KELVVAKAGV YYVFFQLELR RVVAGEGXGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:309), where X is any amino acid other than Ser. In some cases, X is Ala;
PAGLLDLRQG MFAQLVAQNV LLIDGPLSWY SDPGLAGVSL TGGLSYKEDT KELVVAKAGV YYVFFQLELR RVVAGEGSGS VSLALHLQPL RSAAGAAALA LTVXLPPASS EARNSAFGFQ GRLLHLSAGQ RLGVHLHTEA RARHAWQLTQ GATVLGLFRV TPEIPAGLPS PRSE (SEQ ID NO:310), where X is any amino acid other than Asp. In some cases, X is Ala;
PAGLLDLRQG MFAQLVAQNV LLIDGPLSWY SDPGLAGVSL TGGLSYKEDT KELVVAKAGV YYVFFQLELR RVVAGEGSGS VSLALHLQPL RSAAGAAALA LTVDXPPASS EARNSAFGFQ GRLLHLSAGQ RLGVHLHTEA RARHAWQLTQ GATVLGLFRV TPEIPAGLPS PRSE (SEQ ID NO:311), where X is any amino acid other than Leu. In some cases, X is Ala;
PAGLLDLRQG MFAQLVAQNV LLIDGPLSWY SDPGLAGVSL TGGLSYKEDT KELVVAKAGV YYVFFQLELR RVVAGEGSGS VSLALHLQPL RSAAGAAALA LTVDLXPASS EARNSAFGFQ GRLLHLSAGQ RLGVHLHTEA RARHAWQLTQ GATVLGLFRV TPEIPAGLPS PRSE (SEQ ID NO:312), where X is any amino acid other than Pro. In some cases, X is Ala;
PAGLLDLRQG MFAQLVAQNV LLIDGPLSWY SDPGLAGVSL TGGLSYKEDT KELVVAKAGV YYVFFQLELR RVVAGEGSGS VSLALHLQPL RSAAGAAALA LTVDLPPAXS EARNSAFGFQ GRLLHLSAGQ RLGVHLHTEA RARHAWQLTQ GATVLGLFRV TPEIPAGLPS PRSE (SEQ ID NO:313), where X is any amino acid other than Ser. In some cases, X is Ala;
PAGLLDLRQG MFAQLVAQNV LLIDGPLSWY SDPGLAGVSL TGGLSYKEDT KELVVAKAGV YYVFFQLELR RVVAGEGSGS VSLALHLQPL RSAAGAAALA LTVDLPPASX EARNSAFGFQ GRLLHLSAGQ RLGVHLHTEA RARHAWQLTQ GATVLGLFRV TPEIPAGLPS PRSE (SEQ ID NO:314), where X is any amino acid other than Ser. In some cases, X is Ala;
PAGLLDLRQG MFAQLVAQNV LLIDGPLSWY SDPGLAGVSL TGGLSYKEDT KELVVAKAGV YYVFFQLELR RVVAGEGSGS VSLALHLQPL RSAAGAAALA LTVDLPPASS XARNSAFGFQ GRLLHLSAGQ RLGVHLHTEA RARHAWQLTQ GATVLGLFRV TPEIPAGLPS PRSE (SEQ ID NO:315), where X is any amino acid other than Glu. In some cases, X is Ala;
PAGLLDLRQG MFAQLVAQNV LLIDGPLSWY SDPGLAGVSL TGGLSYKEDT KELVVAKAGV YYVFFQLELR RVVAGEGSGS VSLALHLQPL RSAAGAAALA LTVDLPPASS EAXNSAFGFQ GRLLHLSAGQ RLGVHLHTEA RARHAWQLTQ GATVLGLFRV TPEIPAGLPS PRSE (SEQ ID NO:316), where X is any amino acid other than Arg. In some cases, X is Ala;
PAGLLDLRQG MFAQLVAQNV LLIDGPLSWY SDPGLAGVSL TGGLSYKEDT KELVVAKAGV YYVFFQLELR RVVAGEGSGS VSLALHLQPL RSAAGAAALA LTVDLPPASS EARXSAFGFQ GRLLHLSAGQ RLGVHLHTEA RARHAWQLTQ GATVLGLFRV TPEIPAGLPS PRSE (SEQ ID NO:317), where X is any amino acid other than Asn. In some cases, X is Ala;
PAGLLDLRQG MFAQLVAQNV LLIDGPLSWY SDPGLAGVSL TGGLSYKEDT KELVVAKAGV YYVFFQLELR RVVAGEGSGS VSLALHLQPL RSAAGAAALA LTVDLPPASS EARNXAFGFQ GRLLHLSAGQ RLGVHLHTEA RARHAWQLTQ GATVLGLFRV TPEIPAGLPS PRSE (SEQ ID NO:318), where X is any amino acid other than Ser. In some cases, X is Ala;
PAGLLDLRQG MFAQLVAQNV LLIDGPLSWY SDPGLAGVSL TGGLSYKEDT KELVVAKAGV YYVFFQLELR RVVAGEGSGS VSLALHLQPL RSAAGAAALA LTVDLPPASS EARNSAXGFQ GRLLHLSAGQ RLGVHLHTEA RARHAWQLTQ GATVLGLFRV TPEIPAGLPS PRSE (SEQ ID NO:319), where X is any amino acid other than Phe. In some cases, X is Ala;
PAGLLDLRQG MFAQLVAQNV LLIDGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGX RLGVHLHTEA RARHAWQLTQ GATVLGLFRV TPEIPAGLPS PRSE (SEQ ID NO:320), where X is any amino acid other than Gln. In some cases, X is Ala;
PAGLLDLRQG MFAQLVAQNV LLIDGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ XLGVHLHTEA RARHAWQLTQ GATVLGLFRV TPEIPAGLPS PRSE (SEQ ID NO:321), where X is any amino acid other than Arg. In some cases, X is Ala;
PAGLLDLRQG MFAQLVAQNV LLIDGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RXGVHLHTEA RARHAWQLTQ GATVLGLFRV TPEIPAGLPS PRSE (SEQ ID NO:322), where X is any amino acid other than Leu. In some cases, X is Ala;
PAGLLDLRQG MFAQLVAQNV LLIDGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLXVHLHTEA RARHAWQLTQ GATVLGLFRV TPEIPAGLPS PRSE (SEQ ID NO:323), where X is any amino acid other than Gly. In some cases, X is Ala;
PAGLLDLRQG MFAQLVAQNV LLIDGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGXHLHTEA RARHAWQLTQ GATVLGLFRV TPEIPAGLPS PRSE (SEQ ID NO:324), where X is any amino acid other than Val. In some cases, X is Ala;
PAGLLDLRQG MFAQLVAQNV LLIDGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVXLHTEA RARHAWQLTQ GATVLGLFRV TPEIPAGLPS PRSE (SEQ ID NO:325), where X is any amino acid other than His. In some cases, X is Ala;
PAGLLDLRQG MFAQLVAQNV LLIDGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHXHTEA RARHAWQLTQ GATVLGLFRV TPEIPAGLPS PRSE (SEQ ID NO:326), where X is any amino acid other than Leu. In some cases, X is Ala;
PAGLLDLRQG MFAQLVAQNV LLIDGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLXTEA RARHAWQLTQ GATVLGLFRV TPEIPAGLPS PRSE (SEQ ID NO:327), where X is any amino acid other than His. In some cases, X is Ala;
PAGLLDLRQG MFAQLVAQNV LLIDGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHXEA RARHAWQLTQ GATVLGLFRV TPEIPAGLPS PRSE (SEQ ID NO:328), where X is any amino acid other than Thr. In some cases, X is Ala;
PAGLLDLRQG MFAQLVAQNV LLIDGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTXA RARHAWQLTQ GATVLGLFRV TPEIPAGLPS PRSE (SEQ ID NO:329), where X is any amino acid other than Glu. In some cases, X is Ala;
PAGLLDLRQG MFAQLVAQNV LLIDGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA XARHAWQLTQ GATVLGLFRV TPEIPAGLPS PRSE (SEQ ID NO:330), where X is any amino acid other than Arg. In some cases, X is Ala;
PAGLLDLRQG MFAQLVAQNV LLIDGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RAXHAWQLTQ GATVLGLFRV TPEIPAGLPS PRSE (SEQ ID NO:331), where X is any amino acid other than Arg. In some cases, X is Ala;
PAGLLDLRQG MFAQLVAQNV LLIDGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARXAWQLTQ GATVLGLFRV TPEIPAGLPS PRSE (SEQ ID NO:332), where X is any amino acid other than His. In some cases, X is Ala;
PAGLLDLRQG MFAQLVAQNV LLIDGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAXQLTQ GATVLGLFRV TPEIPAGLPS PRSE (SEQ ID NO:333), where X is any amino acid other than Trp. In some cases, X is Ala;
PAGLLDLRQG MFAQLVAQNV LLIDGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQXTQ GATVLGLFRV TPEIPAGLPS PRSE (SEQ ID NO:334), where X is any amino acid other than Leu. In some cases, X is Ala;
PAGLLDLRQG MFAQLVAQNV LLIDGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLXQ GATVLGLFRV TPEIPAGLPS PRSE (SEQ ID NO:335), where X is any amino acid other than Thr. In some cases, X is Ala;
PAGLLDLRQG MFAQLVAQNV LLIDGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTX GATVLGLFRV TPEIPAGLPS PRSE (SEQ ID NO:336), where X is any amino acid other than Gln. In some cases, X is Ala;
PAGLLDLRQG MFAQLVAQNV LLIDGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ XATVLGLFRV TPEIPAGLPS PRSE (SEQ ID NO:337), where X is any amino acid other than Gly. In some cases, X is Ala;
PAGLLDLRQG MFAQLVAQNV LLIDGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GAXVLGLFRV TPEIPAGLPS PRSE (SEQ ID NO:338), where X is any amino acid other than Thr. In some cases, X is Ala; and
PAGLLDLRQG MFAQLVAQNV LLIDGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATXLGLFRV TPEIPAGLPS PRSE (SEQ ID NO:339), where X is any amino acid other than Val. In some cases, X is Ala.

### IL-2 Variants

In some cases, a variant immunomodulatory polypeptide present in a TMMP of the present disclosure is a variant IL-2 polypeptide. Wild-type IL-2 binds to IL-2 receptor (IL-2R), i.e., a heterotrimeric polypeptide comprising IL-2Rα, IL-2Rβ, and IL-2Rγ.

A wild-type IL-2 amino acid sequence can be as follows: APTSSSTKKT QLQLEHLLLD LQMILNGINN YKNPKLTRML TFKFYMPKKA TELKHLQCLEEELKPLEEVL NLAQSKNFHL RPRDLISNIN VIVLELKGSE TTFMCEYADE TATIVEFLNRWITFCQSIIS TLT (SEQ ID NO:340).

Wild-type IL2 binds to an IL2 receptor (IL2R) on the surface of a cell. An IL2 receptor is in some cases a heterotrimeric polypeptide comprising an alpha chain (IL-2Rα; also referred to as CD25), a beta chain (IL-2Rβ; also referred to as CD122; and a gamma chain (IL-2Rγ; also referred to as CD132). Amino acid sequences of human IL-2Rα, IL2Rβ, and IL-2Rγ can be as follows.

Human IL-2Rα:
(SEQ ID NO: 341)
ELCDDDPPE IPHATFKAMA YKEGTMLNCE CKRGFRRIKS
GSLYMLCTGN SSHSSWDNQC QCTSSATRNT TKQVTPQPEE
QKERKTTEMQ SPMQPVDQAS LPGHCREPPP WENEATERIY
HFVVGQMVYY QCVQGYRALH RGPAESVCKM THGKTRWTQP
QLICTGEMET SQFPGEEKPQ ASPEGRPESE TSCLVTTTDF
QIQTEMAATM ETSIFTTEYQ VAVAGCVFLL ISVLLLSGLT
WQRRQRKSRR TI.
Human IL-2Rß:
(SEQ ID NO: 342)
VNG TSQFTCFYNS RANISCVWSQ DGALQDTSCQ
VHAWPDRRRW NQTCELLPVS QASWACNLIL GAPDSQKLTT
VDIVTLRVLC REGVRWRVMA IQDFKPFENL RLMAPISLQV
VHVETHRCNI SWEISQASHY FERHLEFEAR TLSPGHTWEE
APLLTLKQKQ EWICLETLTP DTQYEFQVRV KPLQGEFTTW
SPWSQPLAFR TKPAALGKDT IPWLGHLLVG LSGAFGFIIL
VYLLINCRNT GPWLKKVLKC NTPDPSKFFS QLSSEHGGDV
QKWLSSPFPS SSFSPGGLAP EISPLEVLER DKVTQLLLQQ
DKVPEPASLS SNHSLTSCFT NQGYFFFHLP DALEIEACQV
YFTYDPYSEE DPDEGVAGAP TGSSPQPLQP LSGEDDAYCT
FPSRDDLLLF SPSLLGGPSP PSTAPGGSGA GEERMPPSLQ
ERVPRDWDPQ PLGPPTPGVP DLVDFQPPPE LVLREAGEEV
PDAGPREGVS FPWSRPPGQG EFRALNARLP LNTDAYLSLQ
ELQGQDPTHL V.
Human IL-2Rγ:
(SEQ ID NO: 343)
LNTTILTP NGNEDTTADF FLTTMPTDSL SVSTLPLPEV
QCFVFNVEYM NCTWNSSSEP QPTNLTLHYW YKNSDNDKVQ
KCSHYLFSEE ITSGCQLQKK EIHLYQTFVV QLQDPREPRR
QATQMLKLQN LVIPWAPENL TLHKLSESQL ELNWNNRFLN
HCLEHLVQYR TDWDHSWTEQ SVDYRHKFSL PSVDGQKRYT
FRVRSRFNPL CGSAQHWSEW SHPIHWGSNT SKENPFLFAL
EAVVISVGSM GLIISLLCVY FWLERTMPRI PTLKNLEDLV
TEYHGNFSAW SGVSKGLAES LQPDYSERLC LVSEIPPKGG
ALGEGPGASP CNQHSPYWAP PCYTLKPET.

In some cases, where a TMMP of the present disclosure comprises a variant IL-2 polypeptide, a “cognate co-immunomodulatory polypeptide” is an IL-2R comprising polypeptides comprising the amino acid sequences of SEQ ID NO: 16, 17, and 18.

In some cases, a variant IL-2 polypeptide exhibits reduced binding affinity to IL-2R, compared to the binding affinity of a IL-2 polypeptide comprising the amino acid sequence set forth in SEQ ID NO: 15. For example, in some cases, a variant IL-2 polypeptide binds IL-2R with a binding affinity that is at least 10% less, at least 15% less, at least 20% less, at least 25%, at least 30% less, at least 35% less, at least 40% less, at least 45% less, at least 50% less, at least 55% less, at least 60% less, at least 65% less, at least 70% less, at least 75% less, at least 80% less, at least 85% less, at least 90% less, at least 95% less, or more than 95% less, than the binding affinity of an IL-2 polypeptide comprising the amino acid sequence set forth in SEQ ID NO:340 for an IL-2R (e.g., an IL-2R comprising polypeptides comprising the amino acid sequence set forth in SEQ ID NOs:341-343), when assayed under the same conditions.

In some cases, a variant IL-2 polypeptide has a binding affinity to IL-2R that is from 100 nM to 100 μM. As another example, in some cases, a variant IL-2 polypeptide has a binding affinity for IL-2R (e.g., an IL-2R comprising polypeptides comprising the amino acid sequence set forth in SEQ ID NOs:16-18) that is from about 100 nM to 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 M to about 100 μM.

In some cases, a variant IL-2 polypeptide has a single amino acid substitution compared to the IL-2 amino acid sequence set forth in SEQ ID NO:340. In some cases, a variant IL-2 polypeptide has from 2 to 10 amino acid substitutions compared to the IL-2 amino acid sequence set forth in SEQ ID NO:340. In some cases, a variant IL-2 polypeptide has 2 amino acid substitutions compared to the IL-2 amino acid sequence set forth in SEQ ID NO:340. In some cases, a variant IL-2 polypeptide has 3 amino acid substitutions compared to the IL-2 amino acid sequence set forth in SEQ ID NO:340. In some cases, a variant IL-2 polypeptide has 4 amino acid substitutions compared to the IL-2 amino acid sequence set forth in SEQ ID NO:340. In some cases, a variant IL-2 polypeptide has 5 amino acid substitutions compared to the IL-2 amino acid sequence set forth in SEQ ID NO:340. In some cases, a variant IL-2 polypeptide has 6 amino acid substitutions compared to the IL-2 amino acid sequence set forth in SEQ ID NO:340. In some cases, a variant IL-2 polypeptide has 7 amino acid substitutions compared to the IL-2 amino acid sequence set forth in SEQ ID NO:340. In some cases, a variant IL-2 polypeptide has 8 amino acid substitutions compared to the IL-2 amino acid sequence set forth in SEQ ID NO:340. In some cases, a variant IL-2 polypeptide has 9 amino acid substitutions compared to the IL-2 amino acid sequence set forth in SEQ ID NO:340. In some cases, a variant IL-2 polypeptide has 10 amino acid substitutions compared to the IL-2 amino acid sequence set forth in SEQ ID NO:340.

Suitable IL-2 variants include a polypeptide that comprises an amino acid sequence having at least 90%, at least 95%, at least 98%, at least 99%, or 100%, amino acid sequence identity to any one of the following amino acid sequences:
APTSSSTKKT QLQLEHLLLD LQMILNGINN YKNPKLTRML TXKFYMPKKA TELKHLQCLE EELKPLEEVL NLAQSKNFHL RPRDLISNIN VIVLELKGSE TTFMCEYADE TATIVEFLNR WITFCQSIIS TLT (SEQ ID NO:344), where X is any amino acid other than Phe. In some cases, X is Ala. In some cases, X is Met. In some cases, X is Pro. In some cases, X is Ser. In some cases, X is Thr. In some cases, X is Trp. In some cases, X is Tyr. In some cases, X is Val. In some cases, X is His;
APTSSSTKKT QLQLEHLLLX LQMILNGINN YKNPKLTRML TFKFYMPKKA TELKHLQCLE EELKPLEEVL NLAQSKNFHL RPRDLISNIN VIVLELKGSE TTFMCEYADE TATIVEFLNR WITFCQSIIS TLT (SEQ ID NO:345), where X is any amino acid other than Asp. In some cases, X is Ala;
APTSSSTKKT QLQLXHLLLD LQMILNGINN YKNPKLTRML TFKFYMPKKA TELKHLQCLE EELKPLEEVL NLAQSKNFHL RPRDLISNIN VIVLELKGSE TTFMCEYADE TATIVEFLNR WITFCQSIIS TLT (SEQ ID NO:346), where X is any amino acid other than Glu. In some cases, X is Ala.

APTSSSTKKT QLQLEXLLLD LQMILNGINN YKNPKLTRML TFKFYMPKKA TELKHLQCLE EELKPLEEVL NLAQSKNFHL RPRDLISNIN VIVLELKGSE TTFMCEYADE TATIVEFLNR WITFCQSIIS TLT (SEQ ID NO:347), where X is any amino acid other than His. In some cases, X is Ala. In some cases, X is Thr. In some cases, X is Asn. In some cases, X is Cys. In some cases, X is Gln. In some cases, X is Met. In some cases, X is Val. In some cases, X is Trp;
APTSSSTKKT QLQLEXLLLD LQMILNGINN YKNPKLTRML TFKFYMPKKA TELKHLQCLE EELKPLEEVL NLAQSKNFHL RPRDLISNIN VIVLELKGSE TTFMCEYADE TATIVEFLNR WITFCQSIIS TLT (SEQ ID NO:348), where X is any amino acid other than His. In some cases, X is Ala. In some cases, X is Arg. In some cases, X is Asn. In some cases, X is Asp. In some cases, X is Cys. In some cases, X is Glu. In some cases, X is Gln. In some cases, X is Gly. In some cases, X is Ile. In some cases, X is Lys. In some cases, X is Leu. In some cases, X is Met. In some cases, X is Phe. In some cases, X is Pro. In some cases, X is Ser. In some cases, X is Thr. In some cases, X is Tyr. In some cases, X is Trp. In some cases, X is Val;
APTSSSTKKT QLQLEHLLLD LQMILNGINN YKNPKLTRML TFKFXMPKKA TELKHLQCLE EELKPLEEVL NLAQSKNFHL RPRDLISNIN VIVLELKGSE TTFMCEYADE TATIVEFLNR WITFCQSIIS TLT (SEQ ID NO:349), where X is any amino acid other than Tyr. In some cases, X is Ala;

APTSSSTKKT QLQLEHLLLD LQMILNGINN YKNPKLTRML TFKFYMPKKA TELKHLQCLE EELKPLEEVL NLAQSKNFHL RPRDLISNIN VIVLELKGSE TTFMCEYADE TATIVEFLNR WITFCXSIIS TLT (SEQ ID NO:350), where X is any amino acid other than Gln. In some cases, X is Ala;
APTSSSTKKT QLQLEX₁LLLD LQMILNGINN YKNPKLTRML TX₂KFYMPKKA TELKHLQCLE EELKPLEEVL NLAQSKNFHL RPRDLISNIN VIVLELKGSE TTFMCEYADE TATIVEFLNR WITFCQSIIS TLT (SEQ ID NO:351), where X₁ is any amino acid other than His, and where X₂ is any amino acid other than Phe. In some cases, X₁ is Ala. In some cases, X₂ is Ala. In some cases, X₁ is Ala; and X₂ is Ala. In some cases, X₁ is Thr; and X₂ is Ala;
APTSSSTKKT QLQLEHLLLX₁ LQMILNGINN YKNPKLTRML TX₂KFYMPKKA TELKHLQCLE EELKPLEEVL NLAQSKNFHL RPRDLISNIN VIVLELKGSE TTFMCEYADE TATIVEFLNR WITFCQSIIS TLT (SEQ ID NO:352), where X₁ is any amino acid other than Asp; and where X₂ is any amino acid other than Phe. In some cases, X₁ is Ala. In some cases, X₂ is Ala. In some cases, X₁ is Ala; and X₂ is Ala;
APTSSSTKKT QLQLX₁HLLLX₂ LQMILNGINN YKNPKLTRML TX₃KFYMPKKA TELKHLQCLE EELKPLEEVL NLAQSKNFHL RPRDLISNIN VIVLELKGSE TTFMCEYADE TATIVEFLNR WITFCQSIIS TLT (SEQ ID NO:353), where X₁ is any amino acid other than Glu; where X₂ is any amino acid other than Asp; and where X₃ is any amino acid other than Phe. In some cases, X₁ is Ala. In some cases, X₂ is Ala. In some cases, X₃ is Ala. In some cases, X₁ is Ala; X₂ is Ala; and X₃ is Ala;
APTSSSTKKT QLQLEX₁LLLX₂ LQMILNGINN YKNPKLTRML TX₃KFYMPKKA TELKHLQCLE EELKPLEEVL NLAQSKNFHL RPRDLISNIN VIVLELKGSE TTFMCEYADE TATIVEFLNR WITFCQSIIS TLT (SEQ ID NO:354), where X₁ is any amino acid other than His; where X₂ is any amino acid other than Asp; and where X₃ is any amino acid other than Phe. In some cases, X₁ is Ala. In some cases, X₂ is Ala. In some cases, X₃ is Ala. In some cases, X₁ is Ala; X₂ is Ala; and X₃ is Ala;
APTSSSTKKT QLQLEHLLLX₁ LQMILNGINN YKNPKLTRML TX₂KFYMPKKA TELKHLQCLE EELKPLEEVL NLAQSKNFHL RPRDLISNIN VIVLELKGSE TTFMCEYADE TATIVEFLNR WITFCX₃SIIS TLT (SEQ ID NO:355), where X₁ is any amino acid other than Asp; where X₂ is any amino acid other than Phe; and where X₃ is any amino acid other than Gln. In some cases, X₁ is Ala. In some cases, X₂ is Ala. In some cases, X₃ is Ala. In some cases, X₁ is Ala; X₂ is Ala; and X₃ is Ala;
APTSSSTKKT QLQLEHLLLX₁ LQMILNGINN YKNPKLTRML TX₂KFX₃MPKKA TELKHLQCLE EELKPLEEVL NLAQSKNFHL RPRDLISNIN VIVLELKGSE TTFMCEYADE TATIVEFLNR WITFCQSIIS TLT (SEQ ID NO:356), where X₁ is any amino acid other than Asp; where X₂ is any amino acid other than Phe; and where X₃ is any amino acid other than Tyr. In some cases, X₁ is Ala. In some cases, X₂ is Ala. In some cases, X₃ is Ala. In some cases, X₁ is Ala; X₂ is Ala; and X₃ is Ala;
APTSSSTKKT QLQLEX₁LLLX₂ LQMILNGINN YKNPKLTRML TX₃KFX₄MPKKA TELKHLQCLE EELKPLEEVL NLAQSKNFHL RPRDLISNIN VIVLELKGSE TTFMCEYADE TATIVEFLNR WITFCQSIIS TLT (SEQ ID NO:357), where X₁ is any amino acid other than His; where X₂ is any amino acid other than Asp; where X₃ is any amino acid other than Phe; and where X₄ is any amino acid other than Tyr. In some cases, X₁ is Ala. In some cases, X₂ is Ala. In some cases, X₃ is Ala. In some cases, X₄ is Ala. In some cases, X₁ is Ala; X₂ is Ala; X₃ is Ala; and X₄ is Ala;
APTSSSTKKT QLQLEHLLLX₁LQMILNGINN YKNPKLTRML TX₂KFX₃MPKKA TELKHLQCLE EELKPLEEVL NLAQSKNFHL RPRDLISNIN VIVLELKGSE TTFMCEYADE TATIVEFLNR WITFCX₄SIIS TLT (SEQ ID NO:358), where X₁ is any amino acid other than Asp; where X₂ is any amino acid other than Phe; where X₃ is any amino acid other than Tyr; and where X₄ is any amino acid other than Gln. In some cases, X₁ is Ala. In some cases, X₂ is Ala. In some cases, X₃ is Ala. In some cases, X₄ is Ala. In some cases, X₁ is Ala; X₂ is Ala; X₃ is Ala; and X₄ is Ala;
APTSSSTKKT QLQLEX₁LLLX₂ LQMILNGINN YKNPKLTRML TX₃KFX₄MPKKA TELKHLQCLE EELKPLEEVL NLAQSKNFHL RPRDLISNIN VIVLELKGSE TTFMCEYADE TATIVEFLNR WITFCX₅SIIS TLT (SEQ ID NO:359), where X₁ is any amino acid other than His; where X₂ is any amino acid other than Asp; where X₃ is any amino acid other than Phe; where X₄ is any amino acid other than Tyr; and where X₅ is any amino acid other than Gln. In some cases, X₁ is Ala. In some cases, X₂ is Ala. In some cases, X₃ is Ala. In some cases, X₄ is Ala. In some cases, X₅ is Ala. In some cases, X₁ is Ala; X₂ is Ala; X₃ is Ala; X₄ is Ala; X₅ is Ala; and

APTSSSTKKT QLQLEX₁LLLD LQMILNGINN YKNPKLTRML TX₂KFYMPKKA TELKHLQCLE EELKPLEEVL NLAQSKNFHL RPRDLISNIN VIVLELKGSE TTFMCEYADE TATIVEFLNR WITFCX₃SIIS TLT (SEQ ID NO:360), where X₁ is any amino acid other than His; where X₂ is any amino acid other than Phe; and where X₃ is any amino acid other than Gln. In some cases, X₁ is Ala. In some cases, X₂ is Ala. In some cases, X₃ is Ala. In some cases, X₁ is Ala; X₂ is Ala; and X₃ is Ala.

### Scaffold Polypeptides

A TMMP of the present disclosure can comprise an Fc polypeptide, or can comprise another suitable scaffold polypeptide.

Suitable scaffold polypeptides include antibody-based scaffold polypeptides and non-antibody-based scaffolds. Non-antibody-based scaffolds include, e.g., albumin, an XTEN (extended recombinant) polypeptide, transferrin, an Fc receptor polypeptide, an elastin-like polypeptide (see, e.g., Hassouneh et al. (2012)Methods Enzymol. 502:215; e.g., a polypeptide comprising a pentapeptide repeat unit of (Val-Pro-Gly-X-Gly; SEQ ID NO:361), where X is any amino acid other than proline), an albumin-binding polypeptide, a silk-like polypeptide (see, e.g., Valluzzi et al. (2002) Philos Trans R Soc Lond B Biol Sci. 357:165), a silk-elastin-like polypeptide (SELP; see, e.g., Megeed et al. (2002) Adv Drug Deliv Rev. 54:1075), and the like. Suitable XTEN polypeptides include, e.g., those disclosed in WO 2009/023270, WO 2010/091122, WO 2007/103515, US 2010/0189682, and US 2009/0092582; see also Schellenberger et al. (2009) Nat Biotechnol. 27:1186). Suitable albumin polypeptides include, e.g., human serum albumin.

Suitable scaffold polypeptides will in some cases be a half-life extending polypeptides. Thus, in some cases, a suitable scaffold polypeptide increases the in vivo half-life (e.g., the serum half-life) of the TMMP, compared to a control TMMP lacking the scaffold polypeptide. For example, in some cases, a scaffold polypeptide increases the in vivo half-life (e.g., the serum half-life) of the TMMP, compared to a control TMMP lacking the scaffold polypeptide, by at least about 10%, at least about 15%, at least about 20%, at least about 25%, at least about 50%, at least about 2-fold, at least about 2.5-fold, at least about 5-fold, at least about 10-fold, at least about 25-fold, at least about 50-fold, at least about 100-fold, or more than 100-fold. As an example, in some cases, an Fc polypeptide increases the in vivo half-life (e.g., the serum half-life) of the TMMP, compared to a control TMMP lacking the Fc polypeptide, by at least about 10%, at least about 15%, at least about 20%, at least about 25%, at least about 50%, at least about 2-fold, at least about 2.5-fold, at least about 5-fold, at least about 10-fold, at least about 25-fold, at least about 50-fold, at least about 100-fold, or more than 100-fold.

### Fc Polypeptides

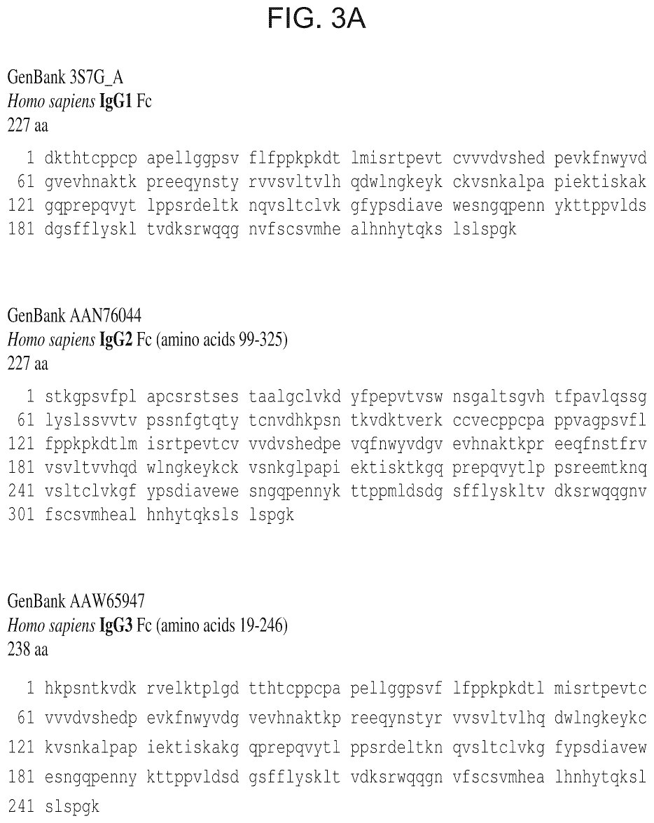

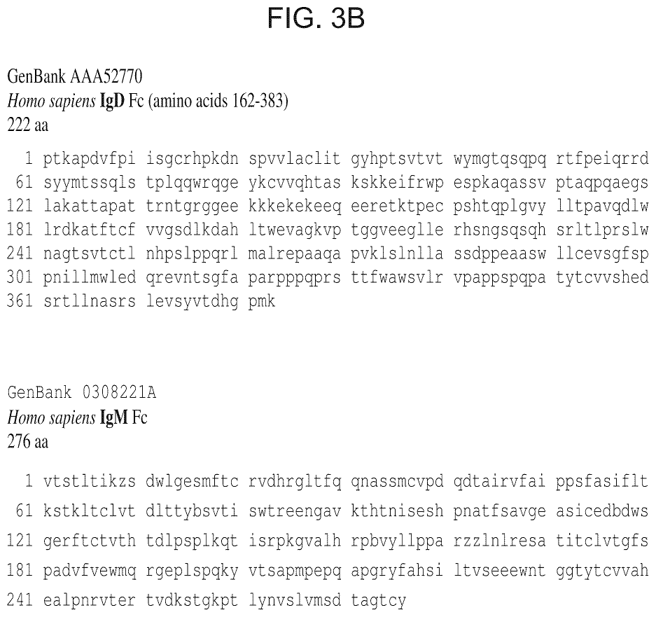

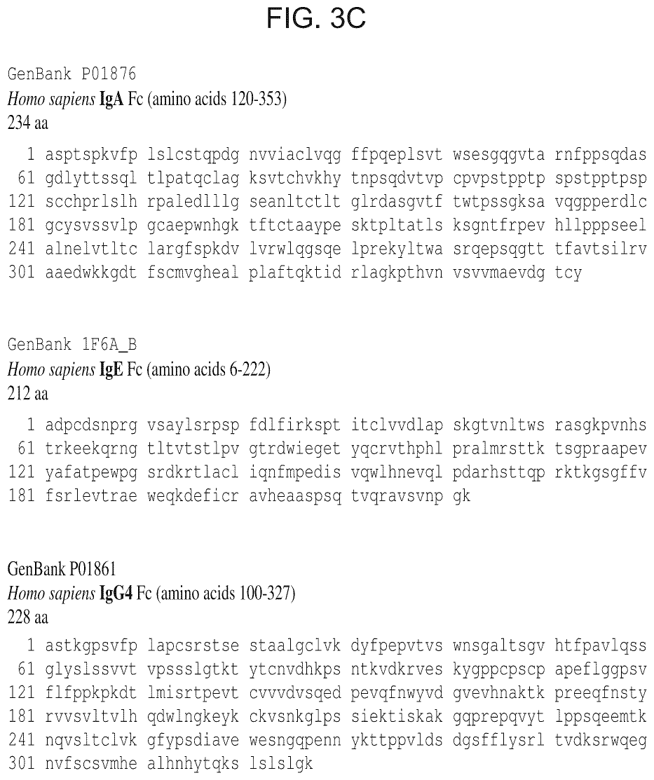

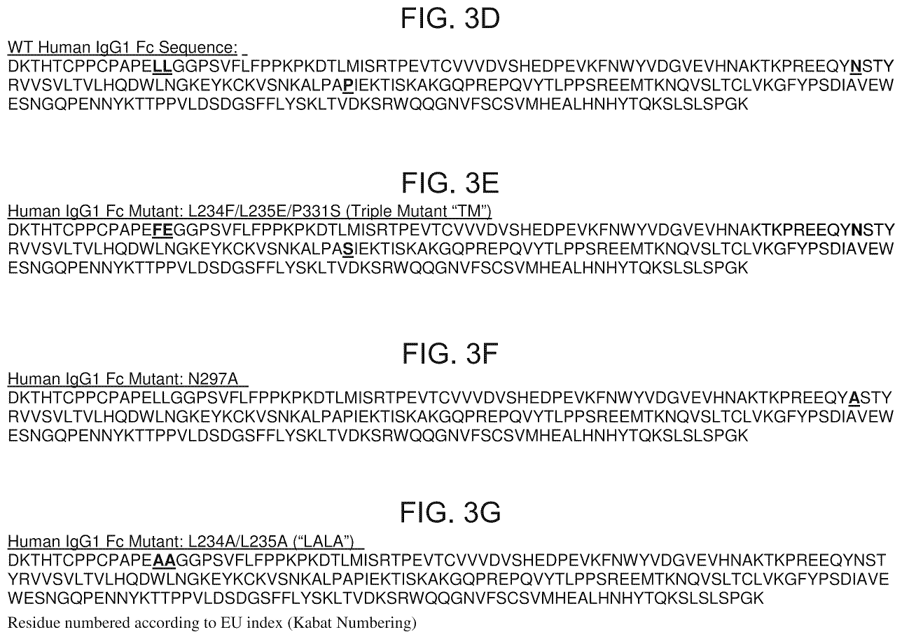

In some cases, the first and/or the second polypeptide chain of a TMMP of the present disclosure comprises an Fc polypeptide. The Fc polypeptide of a TMMP of the present disclosure can be a human IgG1 Fc, a human IgG2 Fc, a human IgG3 Fc, a human IgG4 Fc, etc. In some cases, the Fc polypeptide comprises an amino acid sequence having at least about 70%, at least about 75%, at least about 80%, at least about 85%, at least about 90%, at least about 95%, at least about 98%, at least about 99%, or 100%, amino acid sequence identity to an amino acid sequence of an Fc region depicted in FIG. 3A-3G. In some cases, the Fc region comprises an amino acid sequence having at least about 70%, at least about 75%, at least about 80%, at least about 85%, at least about 90%, at least about 95%, at least about 98%, at least about 99%, or 100%, amino acid sequence identity to the human IgG1 Fc polypeptide depicted in FIG. 3A. In some cases, the Fc region comprises an amino acid sequence having at least about 70%, at least about 75%, at least about 80%, at least about 85%, at least about 90%, at least about 95%, at least about 98%, at least about 99%, or 100%, amino acid sequence identity to the human IgG1 Fc polypeptide depicted in FIG. 3A; and comprises a substitution of N77; e.g., the Fc polypeptide comprises an N77A substitution. In some cases, the Fe polypeptide comprises an amino acid sequence having at least about 70%, at least about 75%, at least about 80%, at least about 85%, at least about 90%, at least about 95%, at least about 98%, at least about 99%, or 100%, amino acid sequence identity to the human IgG2 Fc polypeptide depicted in FIG. 3A; e.g., the Fc polypeptide comprises an amino acid sequence having at least about 70%, at least about 75%, at least about 80%, at least about 85%, at least about 90%, at least about 95%, at least about 98%, at least about 99%, or 100%, amino acid sequence identity to amino acids 99-325 of the human IgG2 Fc polypeptide depicted in FIG. 3A. In some cases, the Fc polypeptide comprises an amino acid sequence having at least about 70%, at least about 75%, at least about 80%, at least about 85%, at least about 90%, at least about 95%, at least about 98%, at least about 99%, or 100%, amino acid sequence identity to the human IgG3 Fc polypeptide depicted in FIG. 3A; e.g., the Fc polypeptide comprises an amino acid sequence having at least about 70%, at least about 75%, at least about 80%, at least about 85%, at least about 90%, at least about 95%, at least about 98%, at least about 99%, or 100%, amino acid sequence identity to amino acids 19-246 of the human IgG3 Fc polypeptide depicted in FIG. 3A. In some cases, the Fc polypeptide comprises an amino acid sequence having at least about 70%, at least about 75%, at least about 80%, at least about 85%, at least about 90%, at least about 95%, at least about 98%, at least about 99%, or 100%, amino acid sequence identity to the human IgM Fc polypeptide depicted in FIG. 3B; e.g., the Fc polypeptide comprises an amino acid sequence having at least about 70%, at least about 75%, at least about 80%, at least about 85%, at least about 90%, at least about 95%, at least about 98%, at least about 99%, or 100%, amino acid sequence identity to amino acids 1-276 to the human IgM Fc polypeptide depicted in FIG. 3B. In some cases, the Fc polypeptide comprises an amino acid sequence having at least about 70%, at least about 75%, at least about 80%, at least about 85%, at least about 90%, at least about 95%, at least about 98%, at least about 99%, or 100%, amino acid sequence identity to the human IgA Fc polypeptide depicted in FIG. 3C; e.g., the Fc polypeptide comprises an amino acid sequence having at least about 70%, at least about 75%, at least about 80%, at least about 85%, at least about 90%, at least about 95%, at least about 98%, at least about 99%, or 100%, amino acid sequence identity to amino acids 1-234 to the human IgA Fc polypeptide depicted in FIG. 3C.

In some cases, the Fc polypeptide comprises an amino acid sequence having at least about 70%, at least about 75%, at least about 80%, at least about 85%, at least about 90%, at least about 95%, at least about 98%, at least about 99%, or 100%, amino acid sequence identity to the human IgG4 Fc polypeptide depicted in FIG. 3C. In some cases, the Fc polypeptide comprises an amino acid sequence having at least about 70%, at least about 75%, at least about 80%, at least about 85%, at least about 90%, at least about 95%, at least about 98%, at least about 99%, or 100%, amino acid sequence identity to amino acids 100 to 327 of the human IgG4 Fc polypeptide depicted in FIG. 3C.

In some cases, the IgG4 Fe polypeptide comprises the following amino acid sequence:

(SEQ ID NO: 362)
PPCPSCPAPEFLGGPSVFLFPPKPKDTLMISRTPEVTCVVVDVS
QEDPEVQFNWYVDGVEVHNAKTKPREEQFNSTYRVVSVLTVLHQ
DWLNGKEYKCKVSNKGLPSSIEKTISKAKGQPREPQVYTLPPSQ
EEMTKNQVSLTCLVKGFYPSDIAVEWESNGQPENNYKTTPPVLD
SDGSFFLYSRLTVDKSRWQEGNVFSCSVMHEALHNHYTQKSLSL
SPG.

In some cases, the Fc polypeptide present in a TMMP comprises the amino acid sequence depicted in FIG. 3A (human IgG1 Fc). In some cases, the Fc polypeptide present in a TMMP comprises the amino acid sequence depicted in FIG. 3A (human IgG1 Fc), except for a substitution of N297 (N77 of the amino acid sequence depicted in FIG. 3A) with an amino acid other than asparagine. In some cases, the Fc polypeptide present in a TMMP comprises the amino acid sequence depicted in FIG. 3C (human IgG1 Fc comprising an N297A substitution, which is N77 of the amino acid sequence depicted in FIG. 3A). In some cases, the Fc polypeptide present in a TMMP comprises the amino acid sequence depicted in FIG. 3A (human IgG1 Fc), except for a substitution of L234 (L14 of the amino acid sequence depicted in FIG. 3A) with an amino acid other than leucine. In some cases, the Fc polypeptide present in a TMMP comprises the amino acid sequence depicted in FIG. 3A (human IgG1 Fc), except for a substitution of L235 (L15 of the amino acid sequence depicted in FIG. 3A) with an amino acid other than leucine.

In some cases, the Fc polypeptide present in a TMMP comprises the amino acid sequence depicted in FIG. 3E. In some cases, the Fc polypeptide present in a TMMP comprises the amino acid sequence depicted in FIG. 3F. In some cases, the Fc polypeptide present in a TMMP comprises the amino acid sequence depicted in FIG. 5G (human IgG1 Fc comprising an L234A substitution and an L235A substitution, corresponding to positions 14 and 15 of the amino acid sequence depicted in FIG. 3G). In some cases, the Fc polypeptide present in a TMMP comprises the amino acid sequence depicted in FIG. 3A (human IgG1 Fc), except for a substitution of P331 (P111 of the amino acid sequence depicted in FIG. 3A) with an amino acid other than proline; in some cases, the substitution is a P331S substitution. In some cases, the Fc polypeptide present in a TMMP comprises the amino acid sequence depicted in FIG. 3A (human IgG1 Fc), except for substitutions at L234 and L235 (L14 and L15 of the amino acid sequence depicted in FIG. 3A) with amino acids other than leucine. In some cases, the Fc polypeptide present in a TMMP comprises the amino acid sequence depicted in FIG. 3A (human IgG1 Fc), except for substitutions at L234 and L235 (L14 and L15 of the amino acid sequence depicted in FIG. 3A) with amino acids other than leucine, and a substitution of P331 (P111 of the amino acid sequence depicted in FIG. 3A) with an amino acid other than proline. In some cases, the Fc polypeptide present in a TMMP comprises the amino acid sequence depicted in FIG. 3E (human IgG1 Fc comprising L234F, L235E, and P331S substitutions (corresponding to amino acid positions 14, 15, and 111 of the amino acid sequence depicted in FIG. 3E). In some cases, the Fc polypeptide present in a TMMP is an IgG1 Fc polypeptide that comprises L234A and L235A substitutions (substitutions of L14 and L15 of the amino acid sequence depicted in FIG. 3A with Ala), as depicted in FIG. 3G.

### Linkers

A TMMP of the present disclosure can include one or more linkers, where the one or more linkers are between one or more of: i) an MHC Class I polypeptide and an Ig Fc polypeptide, where such a linker is referred to herein as “L1”; ii) an immunomodulatory polypeptide and an MHC Class I polypeptide, where such a linker is referred to herein as “L2”; iii) a first immunomodulatory polypeptide and a second immunomodulatory polypeptide, where such a linker is referred to herein as “L3”; iv) a peptide antigen (“epitope”) and an MHC Class I polypeptide; v) an MHC Class I polypeptide and a dimerization polypeptide (e.g., a first or a second member of a dimerizing pair); and vi) a dimerization polypeptide (e.g., a first or a second member of a dimerizing pair) and an IgFc polypeptide.

Suitable linkers (also referred to as “spacers”) can be readily selected and can be of any of a number of suitable lengths, such as from 1 amino acid to 25 amino acids, from 3 amino acids to 20 amino acids, from 2 amino acids to 15 amino acids, from 3 amino acids to 12 amino acids, including 4 amino acids to 10 amino acids, 5 amino acids to 9 amino acids, 6 amino acids to 8 amino acids, or 7 amino acids to 8 amino acids. A suitable linker can be 1, 2, 3, 4, 5, 6, 7, 8, 9, 10, 11, 12, 13, 14, 15, 16, 17, 18, 19, 20, 21, 22, 23, 24, or 25 amino acids in length. In some cases, a linker has a length of from 25 amino acids to 50 amino acids, e.g., from 25 to 30, from 30 to 35, from 35 to 40, from 40 to 45, or from 45 to 50 amino acids in length.

Exemplary linkers include glycine polymers (G)ₙ, glycine-serine polymers (including, for example, (GS)ₙ, (GSGGS)ₙ (SEQ ID NO:363) and (GGGS)ₙ(SEQ ID NO:364), where n is an integer of at least one), glycine-alanine polymers, alanine-serine polymers, and other flexible linkers known in the art. Glycine and glycine-serine polymers can be used; both Gly and Ser are relatively unstructured, and therefore can serve as a neutral tether between components. Glycine polymers can be used; glycine accesses significantly more phi-psi space than even alanine, and is much less restricted than residues with longer side chains (see Scheraga, Rev. Computational Chem. 11173-142 (1992)). Exemplary linkers can comprise amino acid sequences including, but not limited to, GGSG (SEQ ID NO:365), GGSGG (SEQ ID NO:366), GSGSG (SEQ ID NO:367), GSGGG (SEQ ID NO:368), GGGSG (SEQ ID NO:369), GSSSG (SEQ ID NO:370), and the like. Exemplary linkers can include, e.g., Gly(Ser₄)n (SEQ ID NO:371), where n is 1, 2, 3, 4, 5, 6, 7, 8, 9, or 10. In some cases, a linker comprises the amino acid sequence (GSSSS)n (SEQ ID NO:371), where n is 4. In some cases, a linker comprises the amino acid sequence (GSSSS)n (SEQ ID NO:371), where n is 5. In some cases, a linker comprises the amino acid sequence (GGGGS)n (SEQ ID NO:5), where n is 1. In some cases, a linker comprises the amino acid sequence (GGGGS)n (SEQ ID NO:5), where n is 2. In some cases, a linker comprises the amino acid sequence (GGGGS)n (SEQ ID NO:5), where n is 3. In some cases, a linker comprises the amino acid sequence (GGGGS)n (SEQ ID NO:5), where n is 4. In some cases, a linker comprises the amino acid sequence (GGGGS)n (SEQ ID NO:5), where n is 5. In some cases, a linker comprises the amino acid sequence (GGGGS)n (SEQ ID NO:5), where n is 6. In some cases, a linker comprises the amino acid sequence (GGGGS)n (SEQ ID NO:5), where n is 7, In some cases, a linker comprises the amino acid sequence (GGGGS)n (SEQ ID NO:5), where n is 8, In some cases, a linker comprises the amino acid sequence (GGGGS)n (SEQ ID NO:5), where n is 9, In some cases, a linker comprises the amino acid sequence (GGGGS)n (SEQ ID NO:5), where n is 10. In some cases, a linker comprises the amino acid sequence AAAGG (SEQ ID NO:372).

In some cases, a linker polypeptide, present in a first polypeptide of a TMMP of the present disclosure, includes a cysteine residue that can form a disulfide bond with a cysteine residue present in a second polypeptide of a TMMP of the present disclosure. In some cases, for example, a suitable linker comprises the amino acid sequence GCGGSGGGGSGGGGS (SEQ ID NO:208). As another example, a suitable linker can comprise the amino acid sequence GCGGS(G4S)n (SEQ ID NO:206), where n is 1, 2, 3, 4, 5, 6, 7, 8, or 9. For example, in some cases, the linker comprises the amino acid sequence GCGGSGGGGSGGGGSGGGGS (SEQ ID NO:207). As another example, the linker comprises the amino acid sequence GCGGSGGGGSGGGGS (SEQ ID NO:208).

### Multiple Disulfide Bonded TMMPs

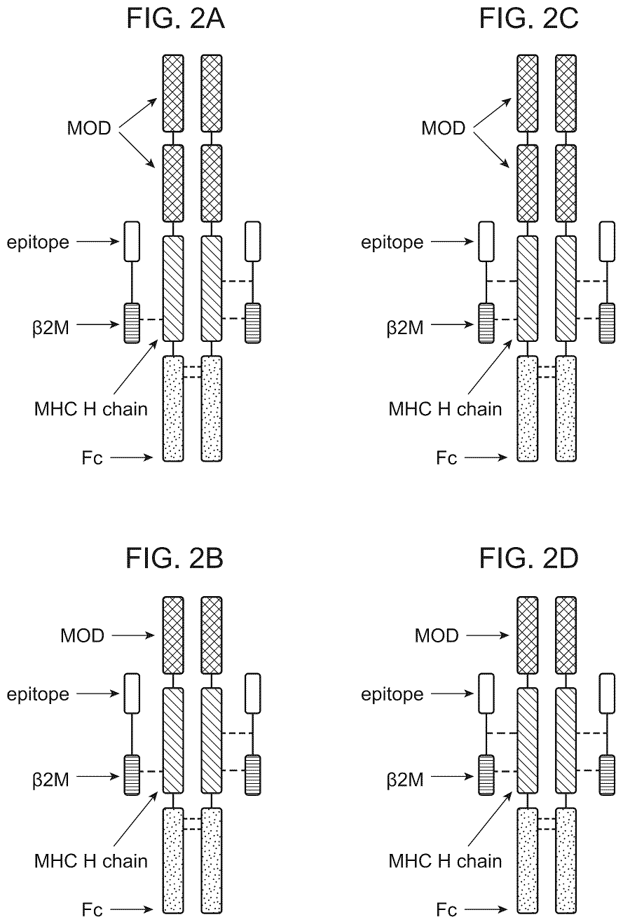

In some cases, the first polypeptide and the second polypeptide of a TMMP of the present disclosure are linked to one another by at least two disulfide bonds (i.e., two interchain disulfide bonds). Examples of such multiple disulfide-linked TMMP are depicted schematically in FIGS. 2A and 2B. In addition, where a TMMP of the present disclosure comprises an IgFc polypeptide, a heterodimeric TMMP can be dimerized, such that disulfide bonds link the IgFc polypeptides in the two heterodimeric TMMPs. Such an arrangement is depicted schematically in FIGS. 2A and 2B, where disulfide bonds are represented by dashed lines. Unless otherwise stated, the at least two disulfide bonds described in the multiple disulfide-linked TMMPPs in this section are not referring to disulfide bonds linking IgFc polypeptides in dimerized TMMPs.

As noted above, in some cases, the first polypeptide and the second polypeptide of a TMMP of the present disclosure are linked to one another by at least two disulfide bonds (i.e., two interchain disulfide bonds). For example, in some instances, the first polypeptide and the second polypeptide of a TMMP of the present disclosure are linked to one another by 2 interchain disulfide bonds. As another example, in some instances, the first polypeptide and the second polypeptide of a TMMP of the present disclosure are linked to one another by 3 interchain disulfide bonds. As another example, in some instances, the first polypeptide and the second polypeptide of a TMMP of the present disclosure are linked to one another by 4 interchain disulfide bonds.

In some cases where a peptide epitope in a first polypeptide of a TMMP of the present disclosure is linked to a β2M polypeptide by a linker comprising a Cys, at least one of the at least two disulfide bonds links a Cys in the linker to a Cys in an MHC Class I heavy chain in the second polypeptide. In some cases, where a peptide epitope in a first polypeptide of a TMMP of the present disclosure is linked to an MHC Class I heavy chain polypeptide by a linker, at least one of the at least two disulfide bonds links a Cys in the linker to a Cys in a β2M polypeptide present in the second polypeptide.

A multiple disulfide-linked TMMP of the present disclosure (e.g., a double disulfide-linked TMMP) can comprise, for example: a) a first polypeptide comprising: i) a peptide epitope (e.g., a peptide of from 4 amino acids to about 25 amino acids in length, that is bound by a TCR when the peptide is complexed with MHC polypeptides); and ii) a first MHC polypeptide, where the first polypeptide comprises a peptide linker between the KRAS peptide and the first MHC polypeptide, where the peptide linker comprises a Cys residue, and where the first MHC polypeptide is a β2M polypeptide that comprises an amino acid substitution that introduces a Cys residue; b) and a second polypeptide comprising a second MHC polypeptide, where the second MHC polypeptide is a Class I heavy chain comprising a Y84C substitution and an A236C substitution, based on the amino acid numbering of HLA-A*0201 (depicted in FIG. 7A), or at corresponding positions in another Class I heavy chain allele, where the TMMP comprises a disulfide bond between the Cys residue in the peptide linker and the Cys residue at amino acid position 84 of the Class I heavy chain or corresponding position of another Class I heavy chain allele, and where the TMMP comprises a disulfide bond between the introduced Cys residue in the β2M polypeptide and the Cys at amino acid position 236 of the Class I heavy chain or corresponding position of another Class I heavy chain allele; and c) at least one immunomodulatory polypeptide, where the first and/or the second polypeptide comprises the at least one immunomodulatory polypeptide.

In some cases, the peptide linker comprises the amino acid sequence GCGGS (SEQ ID NO:373). In some cases, the peptide linker comprises the amino acid sequence GCGGS(GGGGS)n (SEQ ID NO:206), where n is an integer from 1 to 10. In some cases, the peptide linker comprises the amino acid sequence GCGGS(GGGGS)n (SEQ ID NO:206), where n is 1. In some cases, the peptide linker comprises the amino acid sequence GCGGS(GGGGS)n (SEQ ID NO:206), where n is 2. In some cases, the peptide linker comprises the amino acid sequence GCGGS(GGGGS)n (SEQ ID NO:206), where n is 3. In some cases, the peptide linker comprises the amino acid sequence GCGGS(GGGGS)n (SEQ ID NO:206), where n is 4. In some cases, the peptide linker comprises the amino acid sequence GCGGS(GGGGS)n (SEQ ID NO:206), where n is 5. In some cases, the peptide linker comprises the amino acid sequence GCGGS(GGGGS)n (SEQ ID NO:206), where n is 6. In some cases, the peptide linker comprises the amino acid sequence GCGGS(GGGGS)n (SEQ ID NO:206), where n is 7. In some cases, the peptide linker comprises the amino acid sequence GCGGS(GGGGS)n (SEQ ID NO:206), where n is 8. In some cases, the peptide linker comprises the amino acid sequence GCGGS(GGGGS)n (SEQ ID NO:206), where n is 9. In some cases, the peptide linker comprises the amino acid sequence GCGGS(GGGGS)n (SEQ ID NO:206), where n is 10.

In some cases, the peptide linker comprises the amino acid sequence CGGGS (SEQ ID NO:374). In some cases, the peptide linker comprises the amino acid sequence CGGGS(GGGGS)n (SEQ ID NO:375), where n is an integer from 1 to 10. In some cases, the peptide linker comprises the amino acid sequence CGGGS(GGGGS)n (SEQ ID NO:375), where n is 1. In some cases, the peptide linker comprises the amino acid sequence CGGGS(GGGGS)n (SEQ ID NO:375), where n is 2. In some cases, the peptide linker comprises the amino acid sequence CGGGS(GGGGS)n (SEQ ID NO:375), where n is 3. In some cases, the peptide linker comprises the amino acid sequence CGGGS(GGGGS)n (SEQ ID NO:375), where n is 4. In some cases, the peptide linker comprises the amino acid sequence CGGGS(GGGGS)n (SEQ ID NO:375), where n is 5. In some cases, the peptide linker comprises the amino acid sequence CGGGS(GGGGS)n (SEQ ID NO:375), where n is 6. In some cases, the peptide linker comprises the amino acid sequence CGGGS(GGGGS)n (SEQ ID NO:375), where n is 7. In some cases, the peptide linker comprises the amino acid sequence CGGGS(GGGGS)n (SEQ ID NO:375), where n is 8. In some cases, the peptide linker comprises the amino acid sequence CGGGS(GGGGS)n (SEQ ID NO:375), where n is 9. In some cases, the peptide linker comprises the amino acid sequence CGGGS(GGGGS)n (SEQ ID NO:375), where n is 10.

### Dimerized TMMPs

A TMMP can be dimerized. For example, a dimeric TMMP can comprise two heterodimeric TMMPs. A dimeric TMMP can comprise: A) a first heterodimer comprising: a) a first polypeptide comprising: i) a peptide epitope; and ii) a first major histocompatibility complex (MHC) polypeptide; and b) a second polypeptide comprising: i) a second MHC polypeptide, wherein the first heterodimer comprises one or more immunomodulatory polypeptides; and B) a second heterodimer comprising: a) a first polypeptide comprising: i) a peptide epitope; and ii) a first MHC polypeptide; and b) a second polypeptide comprising: i) a second MHC polypeptide, wherein the second heterodimer comprises one or more immunomodulatory polypeptides, and wherein the first heterodimer and the second heterodimer are covalently linked to one another.

In some cases, the two TMMPs are identical to one another in amino acid sequence. In some cases, the first heterodimer and the second heterodimer are covalently linked to one another via a C-terminal region of the second polypeptide of the first heterodimer and a C-terminal region of the second polypeptide of the second heterodimer. In some cases, first heterodimer and the second heterodimer are covalently linked to one another via the C-terminal amino acid of the second polypeptide of the first heterodimer and the C-terminal region of the second polypeptide of the second heterodimer; for example, in some cases, the C-terminal amino acid of the second polypeptide of the first heterodimer and the C-terminal region of the second polypeptide of the second heterodimer are linked to one another, either directly or via a linker. The linker can be a peptide linker. The peptide linker can have a length of from 1 amino acid to 200 amino acids (e.g., from 1 amino acid (aa) to 5 aa, from 5 aa to 10 aa, from 10 aa to 25 aa, from 25 aa to 50 aa, from 50 aa to 100 aa, from 100 aa to 150 aa, or from 150 aa to 200 aa). In some cases, the peptide epitope of the first heterodimer and the peptide epitope of the second heterodimer comprise the same amino acid sequence. In some cases, the first MHC polypeptide of the first and the second heterodimer is an MHC Class I β2-microglobulin, and wherein the second MHC polypeptide of the first and the second heterodimer is an MHC Class I heavy chain. In some cases, the immunomodulatory polypeptide of the first heterodimer and the immunomodulatory polypeptide of the second heterodimer comprise the same amino acid sequence. In some cases, the immunomodulatory polypeptide of the first heterodimer and the immunomodulatory polypeptide of the second heterodimer are variant immunomodulatory polypeptides that comprise from 1 to 10 amino acid substitutions compared to a corresponding parental wild-type immunomodulatory polypeptide, and wherein the from 1 to 10 amino acid substitutions result in reduced affinity binding of the variant immunomodulatory polypeptide to a cognate co-immunomodulatory polypeptide. In some cases, the immunomodulatory polypeptide of the first heterodimer and the immunomodulatory polypeptide of the second heterodimer are each independently selected from the group consisting of IL-2, 4-1BBL, PD-L1, CD80, CD86, ICOS-L, OX-40L, FasL, JAG1 (CD339), TGFβ, CD70, and ICAM. Examples, of suitable MHC polypeptides, immunomodulatory polypeptides, and peptide epitopes are described below. The first and/or the second polypeptide comprises: i) an Ig Fc polypeptide or a non-Ig scaffold; and ii) a tumor-targeting polypeptide.

### EXAMPLES OF NON-LIMITING ASPECTS OF THE DISCLOSURE

Aspects, including embodiments, of the present subject matter described above may be beneficial alone or in combination, with one or more other aspects or embodiments. Without limiting the foregoing description, certain non-limiting aspects of the disclosure numbered 1-90 are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:

Aspect 1. An in vitro composition comprising a quantity of modified cytotoxic T cells (“mCTLs”), wherein the quantity comprises target mCTLs that comprise: a) a T-cell receptor (TCR) specific for a preselected antigen present in a human; and b) one or more nucleic acids comprising nucleotide sequences encoding a chimeric antigen receptor (CAR), wherein the CAR comprises an antigen-binding domain specific for a cancer-associated antigen, and wherein the percentage of target mCTLs cells in the composition exceeds at least 1% of the total number of T cells in the composition.

Aspect 2. A composition according to aspect 1, wherein the preselected antigen is an antigen encoded by a virus or a bacterium.

Aspect 3. A composition according to aspect 2, wherein the preselected antigen is an antigen encoded by a virus or bacteria selected from the group consisting of cytomegalovirus (CMV), Epstein-Barr virus (EBV), human papilloma virus (HPV), adenovirus, influenza virus, and Clostridium tetani.

Aspect 4. A composition according to aspect 3, wherein the preselected antigen is a CMV polypeptide.

Aspect 5. A composition according to aspect 4, wherein the CMV antigen is a CMV pp65 polypeptide.

Aspect 6. A composition according to any one of aspects 1-5, wherein the CAR comprises: a) an extracellular domain comprising the antigen-binding domain; b) a transmembrane region; and c) a cytoplasmic domain comprising an intracellular signaling domain.

Aspect 7. A composition according to aspect 6, wherein the cytoplasmic domain comprises one or more costimulatory polypeptides.

Aspect 8. A composition according to aspect 7, wherein the costimulatory polypeptide is selected from CD28, 4-1BB, and OX-40.

Aspect 9. A composition according to any one of aspects 6-8, wherein the intracellular signaling domain comprises a signaling domain from the zeta chain of human CD3.

Aspect 10. A composition according to any one of aspects 1-9, wherein the antigen-binding domain is a single-chain Fv polypeptide or a nanobody.

Aspect 11. A composition according to any one of aspects 1-10, wherein the CAR is a single polypeptide chain CAR.

Aspect 12. A composition according to any one of aspects 1-10, wherein the CAR comprises at least two polypeptide chains.

Aspect 13. A composition according to any one of aspects 1-12, wherein the cancer-associated antigen is selected from AFP, BCMA, CD10, CD117, CD123, CD133, CD128, CD171, CD19, CD20, CD22, CD30, CD33, CD34, CD38, CD5, CD56, CD7, CD70, CD80, CD86, CEA, CLD18, CLL-1, cMet, EGFR, EGFRvIII, EpCAM, EphA2, GD-2, glypican-3, GPC3, HER-2, kappa immunoglobulin, LeY, LMP1, mesothelin, MG7, MUC1, NKG2D ligand, PD-L1, PSCA, PSMA, ROR1, ROR1R, TACI, and VEGFR2.

Aspect 14. A composition according to any one of aspects 1-13, wherein the percentage of total number of T cells in the composition that are target mCTLs is selected from the group consisting of at least 10%, at least 20%, at least 30%, at least 40%, at least 50%, at least 60%, at least 70%, at least 80%, at least 90%, at least 95%, at least 96%, at least 97%, at least 98%, at least 99%, and 100%.

Aspect 15. A composition according to any one of aspects 1-14, wherein the target mCTLs are CD8⁺ T cells.

Aspect 16. A pharmaceutical composition comprising a composition according to any one of aspects 1-15.

Aspect 17. A pharmaceutical composition according to aspect 16 comprising a pharmaceutically acceptable carrier.

Aspect 18. A pharmaceutical composition according to aspect 17, wherein the pharmaceutically acceptable carrier comprises saline.

Aspect 19. A pharmaceutical composition according to any one of aspects 16-18, wherein the target mCTLs are present in the composition in a concentration of from about 10⁶ cells/mL to about 10⁹ cells/mL.

Aspect 20. A method of making an in vitro composition according to any one of aspects 1-15, comprising the steps of:
(i) providing a composition comprising a quantity of T cells, wherein the quantity comprises target T cells having a T-cell receptor (TCR) specific for the preselected antigen;
(ii) at least partially separating target T cells from non-target T cells comprising a T-cell receptor (TCR) that is not specific for the preselected antigen, thereby generating a quantity of at least partially separated target T cells; and
(iii) modifying the at least partially separated target T cells by introducing into the at least partially separated target T cells one or more nucleic acids comprising nucleotide sequences encoding a chimeric antigen receptor (CAR) that comprises an antigen-binding domain specific for a cancer-associated antigen.

Aspect 21. A method according to aspect 20, wherein step of at least partially separating target T cells comprises binding the target T cells to a polypeptide that binds to the TCR of the target T cells.

Aspect 22. A method according to aspect 21, wherein said polypeptide that binds to the TCR of the target T cells is on a surface.

Aspect 23. A method according to aspect 22, wherein the polypeptide that binds to the TCR of the target T cells is present on the surface of a bead.

Aspect 24. A method according to aspect 23, wherein the polypeptide that binds to the TCR is a peptide-loaded MHC multimer.

Aspect 25. A method according to any of aspects 20-24, wherein the preselected antigen present in a human is an antigen encoded by a virus or a bacterium.

Aspect 26. A method according to any of aspects 20-24, wherein the preselected antigen is an antigen encoded by a virus or bacterium selected from the group consisting of cytomegalovirus (CMV), Epstein-Barr virus (EBV), human papilloma virus (HPV), adenovirus, influenza virus, and Clostridium tetani.

Aspect 27. A method according to aspect 26, wherein the preselected antigen is a CMV polypeptide.

Aspect 28. A method according to aspect 27, wherein the CMV polypeptide is a CMV pp65 polypeptide.

Aspect 29. A method according to any of aspects 20-28, wherein the CAR comprises: a) an extracellular domain comprising the antigen-binding domain; b) a transmembrane region; and c) a cytoplasmic domain comprising an intracellular signaling domain.

Aspect 30. A method according to aspect 29, wherein the intracellular signaling domain comprises a signaling domain from the zeta chain of human CD3.

Aspect 31. A method according to aspect 29 or 30, wherein the cytoplasmic domain comprises one or more costimulatory polypeptides.

Aspect 32. A method according to aspect 31, wherein the costimulatory polypeptide is selected from CD28, 4-1BB, and OX-40.

Aspect 33. A method according to any one of aspects 20-32, wherein the antigen-binding domain is a single-chain Fv polypeptide or a nanobody.

Aspect 34. A method according to any one of aspects 20-33, wherein the CAR is a single polypeptide chain CAR.

Aspect 35. A method according to any one of aspects 20-33, wherein the CAR comprises two polypeptide chains.

Aspect 36. A method according to any one of aspects 20-35, wherein the cancer-associated antigen is selected from AFP, BCMA, CD10, CD117, CD123, CD133, CD128, CD171, CD19, CD20, CD22, CD30, CD33, CD34, CD38, CD5, CD56, CD7, CD70, CD80, CD86, CEA, CLD18, CLL-1, cMet, EGFR, EGFRvIII, EpCAM, EphA2, GD-2, glypican-3, GPC3, HER-2, kappa immunoglobulin, LeY, LMP1, mesothelin, MG7, MUC1, NKG2D ligand, PD-L1, PSCA, PSMA, ROR1, ROR1R, TACI, and VEGFR2.

Aspect 37. A method according to any one of aspects 20-36, wherein the percentage of total number of T cells in the composition that are target CTLs is selected from the group consisting of at least 10%, at least 20%, at least 30%, at least 40%, at least 50%, at least 60%, at least 70%, at least 80%, at least 90%, at least 95%, at least 96%, at least 97%, at least 98%, at least 99%, and 100%.

Aspect 38. A method according to any one of aspects 20-37, wherein prior to step (ii), the composition comprising a quantity of T cells is contacted in vitro or in vivo with a composition comprising a T-cell modulatory polypeptide that binds to and activates substantially only the T cells comprising a T-cell receptor (TCR) specific for the preselected antigen.

Aspect 39. A method according to aspect 38, wherein the T-cell modulatory polypeptide is a T cell multimeric polypeptide (TMMP) that comprises at least one heterodimer, said heterodimer comprising:
(i) a first polypeptide comprising a peptide epitope and a first major histocompatibility complex (MHC) polypeptide, wherein the peptide epitope is a peptide having a length of from 4 amino acids to about 25 amino acids, and wherein the peptide epitope is an epitope of the preselected antigen;
(ii) a second polypeptide comprising a second MHC polypeptide; and
(iii) at least one immunomodulatory polypeptide that activates T cells comprising a T-cell receptor (TCR) specific for the preselected antigen,
wherein the first and/or the second polypeptide comprises the immunomodulatory polypeptide, and
optionally, wherein the multimeric polypeptide comprises an immunoglobulin (Ig) Fc polypeptide or a non-Ig scaffold.

Aspect 40. A method according to aspect 39, wherein the TMMP comprises two heterodimers, wherein both heterodimers comprise an Ig Fc polypeptide, and wherein the heterodimers are covalently bound by one or more disulfide bonds between the Ig Fc polypeptides of the first and second heterodimers.

Aspect 41. A method according to aspect 39, wherein the TMMP comprises:
a1) a first polypeptide comprising, in order from N-terminus to C-terminus:
   i) a peptide epitope; and
   ii) a first MHC polypeptide; and
b1) a second polypeptide comprising, in order from N-terminus to C-terminus:
   i) at least one immunomodulatory polypeptide;
   ii) a second MHC polypeptide; and
   iii) an immunoglobulin (Ig) Fc polypeptide; or
a2) a first polypeptide comprising, in order from N-terminus to C-terminus:
   i) a peptide epitope;
   ii) a first MHC polypeptide; and
   iii) at least one immunomodulatory polypeptide; and
b2) a second polypeptide comprising, in order from N-terminus to C-terminus:
   i) a second MHC polypeptide; and
   ii) an Ig Fc polypeptide; or
a3) a first polypeptide comprising, in order from N-terminus to C-terminus:
   i) a peptide epitope; and
   ii) a first MHC polypeptide; and
b3) a second polypeptide comprising, in order from N-terminus to C-terminus:
   i) a second MHC polypeptide; and
   ii) an Ig Fc polypeptide; and
   iii) at least one immunomodulatory polypeptide; or
a4) a first polypeptide comprising, in order from N-terminus to C-terminus:
   i) a peptide epitope; and
   ii) a first MHC polypeptide; and
b4) a second polypeptide comprising, in order from N-terminus to C-terminus:
   i) a second MHC polypeptide; and
   ii) at least one immunomodulatory polypeptide; or
a5) a first polypeptide comprising, in order from N-terminus to C-terminus:
   i) a peptide epitope; and
   ii) a first MHC polypeptide; and
b5) a second polypeptide comprising, in order from N-terminus to C-terminus:
   i) at least one immunomodulatory polypeptide; and
   ii) a second MHC polypeptide; or
a6) a first polypeptide comprising, in order from N-terminus to C-terminus:
   i) a peptide epitope;
   ii) a first MHC polypeptide; and
   iii) at least one immunomodulatory polypeptide; and
b6) a second polypeptide comprising:
   i) a second MHC polypeptide.

Aspect 42. A method according to any one of aspects 39-41, wherein the at least one immunomodulatory polypeptide is a naturally occurring polypeptide, a variant of a naturally occurring polypeptide, or a fragment of a naturally occurring or variant polypeptide, and wherein the polypeptide is selected from the group consisting of a 4-1BBL polypeptide, a B7-1 polypeptide; a B7-2 polypeptide, an ICOS-L polypeptide, an OX-40L polypeptide, a CD80 polypeptide, a CD86 polypeptide, a PD-L1 polypeptide, a FasL polypeptide, a cytokine, a PD-L2 polypeptide, and combinations thereof.

Aspect 43. A method according to aspect 42, wherein the at least one immunomodulatory polypeptide is a naturally occurring cytokine, a variant of a naturally occurring cytokine, or a fragment of a naturally occurring cytokine.

Aspect 44. A method according to aspect 42, wherein the cytokine is IL-2.

Aspect 45. A method of any of aspects 39-44, wherein the at least one immunomodulatory polypeptide is a naturally occurring polypeptide, a variant of a naturally occurring polypeptide, or a fragment of a naturally occurring or variant polypeptide, wherein the at least one immunomodulatory polypeptide is selected from a 4-1BBL polypeptide and a CD80 polypeptide.

Aspect 46. A method according to any one of aspects 39-45, wherein the TMMP comprises 2 or more immunomodulatory polypeptides.

Aspect 47. A method according to aspect 46, wherein the 2 or more immunomodulatory polypeptides are in tandem.

Aspect 48. A method according to any one of aspects 45-47, wherein the multimeric polypeptide comprises:
a1) a first polypeptide comprising, in order from N-terminus to C-terminus:
   i) a peptide epitope; and
   ii) a first MHC polypeptide; and
b1) a second polypeptide comprising, in order from N-terminus to C-terminus:
   i) at least one immunomodulatory polypeptide;
   ii) a second MHC polypeptide; and
   iii) an immunoglobulin (Ig) Fc polypeptide.

Aspect 49. A method according to any one of aspects 45-47, wherein the multimeric polypeptide comprises:
a3) a first polypeptide comprising, in order from N-terminus to C-terminus:
   i) a peptide epitope; and
   ii) a first MHC polypeptide; and
b3) a second polypeptide comprising, in order from N-terminus to C-terminus:
   i) a second MHC polypeptide; and
   ii) an Ig Fc polypeptide; and
   iii) at least one immunomodulatory polypeptide.

Aspect 50. A method according to any one of aspects 45-47, wherein the multimeric polypeptide comprises:
a first polypeptide comprising, in order from N-terminus to C-terminus:
   i) a peptide epitope; and
   ii) a first MHC polypeptide; and
b4) a second polypeptide comprising, in order from N-terminus to C-terminus:
   i) a second MHC polypeptide; and
   ii) at least one immunomodulatory polypeptide.

Aspect 51. A method according to any one of aspects 45-50, wherein the first polypeptide and the second polypeptide are linked to one another by a disulfide bond.

Aspect 52. A method according to any of aspects 45-51, wherein the first MHC polypeptide is a β2M polypeptide, and the second MHC polypeptide is a class I MHC heavy chain.

Aspect 53. A method according to aspect 52, wherein the β2M polypeptide and the MHC heavy chain polypeptide are joined by a disulfide bond that joins a Cys residue in the β2M polypeptide and a Cys residue in the MHC heavy chain polypeptide.

Aspect 54. A method according to aspect 53, wherein a Cys at amino acid residue 12 of the β2M polypeptide is disulfide bonded to a Cys at amino acid residue 236 of the MHC heavy chain polypeptide.

Aspect 55. A method according to any of aspects 51-54, wherein the first polypeptide chain comprises a linker between the peptide epitope and the β2M polypeptide.

Aspect 56. A method according to any of aspects 51-54, wherein the first polypeptide chain comprises a linker between the peptide epitope and the β2M polypeptide, and wherein the disulfide bond links a Cys substituted for Gly2 in the linker with a Cys substituted for Tyr84 of the MHC heavy chain polypeptide.

Aspect 57. A method according to any one of aspects 39-56, wherein the immunomodulatory polypeptide is an activating polypeptide.

Aspect 58. A method according to any one of aspects 39-57, wherein at least 50% of the target T cells are CD8⁺ T cells.

Aspect 59. A method according to any one of aspects 20-57, comprising, between steps (i) and (ii), enriching the T cells for CD8⁺ T cells.

Aspect 60. A method according to any one of aspects 20-57, comprising, between steps (ii) and (iii), enriching the T cells for CD8⁺ T cells.

Aspect 61. A method of treating a cancer in an individual, the method comprising introducing into the individual: i) a composition comprising a quantity of modified cytotoxic T cells according to any one of aspects 1-15; ii) a pharmaceutical composition according to any one of aspects 16-19; or iii) a composition prepared according to the method of any one of aspects 20-60.

Aspect 62. A method of treating cancer according to aspect 61, further comprising administering to the individual a composition comprising a T-cell modulatory polypeptide, wherein the T-cell modulatory polypeptide largely binds to and activates only the T cells comprising a T-cell receptor (TCR) specific for the preselected antigen.

Aspect 63. A method according to aspect 61 or aspect 62, wherein said administering a composition comprising a quantity of genetically modified cytotoxic T cells comprises administering a quantity of genetically modified cytotoxic T cells that is equal to or less than a number selected from the group consisting of 10 cells/kg body weight, 10² cells/kg body weight, 10³ cells/kg body weight, 10⁴ cells/kg body weight, 10⁵ cells/kg body weight, 10⁶ cells/kg body weight, 10⁷ cells/kg body weight, 10⁸ cells/kg body weight and 10⁹ cells/kg body weight.

Aspect 64. A method according to any of aspects 61-63, wherein said administering a composition comprising a quantity of genetically modified cytotoxic T cells comprises administering a quantity of genetically modified cytotoxic T cells that is equal to or less than 10⁷ cells/kg body weight.

Aspect 65. A method according to any of aspects 61-64, wherein the individual does not undergo a lymphodepleting regimen prior to the introducing step.

Aspect 66. A method according to any of aspects 62-65, wherein the T-cell modulatory polypeptide is a T cell multimeric polypeptide (TMMP) that comprises at least one heterodimer, said heterodimer comprising:
(i) a first polypeptide comprising a peptide epitope and a first major histocompatibility complex (MHC) polypeptide, wherein the peptide epitope is a peptide having a length of from 4 amino acids to about 25 amino acids, wherein the peptide epitope is an epitope of the preselected antigen;
(ii) a second polypeptide comprising a second MHC polypeptide; and
(iii) at least one immunomodulatory polypeptide,
wherein the first and/or the second polypeptide comprises the immunomodulatory polypeptide, and
optionally, wherein the multimeric polypeptide comprises an immunoglobulin (Ig) Fc polypeptide or a non-Ig scaffold.

Aspect 67. A method according to aspect 66, wherein the TMMP comprises:
a1) a first polypeptide comprising, in order from N-terminus to C-terminus:
   i) a peptide epitope; and
   ii) a first MHC polypeptide; and
b1) a second polypeptide comprising, in order from N-terminus to C-terminus:
   i) at least one immunomodulatory polypeptide;
   ii) a second MHC polypeptide; and
   iii) an immunoglobulin (Ig) Fc polypeptide; or
a2) a first polypeptide comprising, in order from N-terminus to C-terminus:
   i) a peptide epitope;
   ii) a first MHC polypeptide; and
   iii) at least one immunomodulatory polypeptide; and
b2) a second polypeptide comprising, in order from N-terminus to C-terminus:
   i) a second MHC polypeptide; and
   ii) an Ig Fc polypeptide; or
a3) a first polypeptide comprising, in order from N-terminus to C-terminus:
   i) a peptide epitope; and
   ii) a first MHC polypeptide; and
b3) a second polypeptide comprising, in order from N-terminus to C-terminus:
   i) a second MHC polypeptide; and
   ii) an Ig Fc polypeptide; and
   iii) at least one immunomodulatory polypeptide; or
a4) a first polypeptide comprising, in order from N-terminus to C-terminus:
   i) a peptide epitope; and
   ii) a first MHC polypeptide; and
b4) a second polypeptide comprising, in order from N-terminus to C-terminus:
   i) a second MHC polypeptide; and
   ii) at least one immunomodulatory polypeptide; or
a5) a first polypeptide comprising, in order from N-terminus to C-terminus:
   i) a peptide epitope; and
   ii) a first MHC polypeptide; and
b5) a second polypeptide comprising, in order from N-terminus to C-terminus:
   i) at least one immunomodulatory polypeptide; and
   ii) a second MHC polypeptide; or
a6) a first polypeptide comprising, in order from N-terminus to C-terminus:
   i) a peptide epitope;
   ii) a first MHC polypeptide; and
   iii) at least one immunomodulatory polypeptide; and
b6) a second polypeptide comprising, in order from N-terminus to C-terminus:
   i) a second MHC polypeptide.

Aspect 68. A method according to aspect 66 or aspect 67, wherein the at least one immunomodulatory polypeptide is a naturally occurring polypeptide, a variant of a naturally occurring polypeptide, or a fragment of a naturally occurring or variant polypeptide, and wherein the polypeptide is selected from the group consisting of a 4-1BBL polypeptide, a B7-1 polypeptide; a B7-2 polypeptide, an ICOS-L polypeptide, an OX-40L polypeptide, a CD80 polypeptide, a CD86 polypeptide, a PD-L1 polypeptide, a FasL polypeptide, a cytokine, a PD-L2 polypeptide, and combinations thereof.

Aspect 69. A method according to aspect 68, wherein the at least one immunomodulatory polypeptide is a naturally occurring cytokine, a variant of a naturally occurring cytokine, or a fragment of a naturally occurring cytokine.

Aspect 70. A method according to aspect 68, wherein the cytokine is IL-2.

Aspect 71. A method of aspect 66 or aspect 67, wherein the at least one immunomodulatory polypeptide is a naturally occurring polypeptide, a variant of a naturally occurring polypeptide, or a fragment of a naturally occurring or variant polypeptide, optionally wherein the at least one immunomodulatory polypeptide is selected from a 4-1BBL polypeptide and a CD80 polypeptide.

Aspect 72. A method according to any one of aspects 66-71, wherein the TMMP comprises 2 or more immunomodulatory polypeptides.

Aspect 73. A method according to aspect 72, wherein the 2 or more immunomodulatory polypeptides are in tandem.

Aspect 74. A method according to any one of aspects 66-73, wherein the multimeric polypeptide comprises:
a) a first polypeptide comprising, in order from N-terminus to C-terminus:
   i) a peptide epitope; and
   ii) a first MHC polypeptide; and
b) a second polypeptide comprising, in order from N-terminus to C-terminus:
   i) at least one immunomodulatory polypeptide;
   ii) a second MHC polypeptide; and
   iii) an immunoglobulin (Ig) Fc polypeptide.

Aspect 75. A method according to any one of aspects 66-73, wherein the multimeric polypeptide comprises:
a) a first polypeptide comprising, in order from N-terminus to C-terminus:
   i) a peptide epitope; and
   ii) a first MHC polypeptide; and
b) a second polypeptide comprising, in order from N-terminus to C-terminus:
   i) a second MHC polypeptide; and
   ii) an Ig Fc polypeptide; and
   iii) at least one immunomodulatory polypeptide.

Aspect 76. A method according to any one of aspects 66-73, wherein the multimeric polypeptide comprises:
a) a first polypeptide comprising, in order from N-terminus to C-terminus:
   i) a peptide epitope; and
   ii) a first MHC polypeptide; and
b) a second polypeptide comprising, in order from N-terminus to C-terminus:
   i) a second MHC polypeptide; and
   ii) at least one immunomodulatory polypeptide.

Aspect 77. A method according to any one of aspects 66-76, wherein the first polypeptide and the second polypeptide are linked to one another by a disulfide bond.

Aspect 78. A method according to any of aspects 66-77, wherein the first MHC polypeptide is a β2M polypeptide, and the second MHC polypeptide is a class I MHC heavy chain.

Aspect 79. A method according to aspect 78, wherein the β2M polypeptide and the MHC heavy chain polypeptide are joined by a disulfide bond that joins a Cys residue in the β2M polypeptide and a Cys residue in the MHC heavy chain polypeptide.

Aspect 80. A method according to aspect 79, wherein a Cys at amino acid residue 12 of the β2M polypeptide is disulfide bonded to a Cys at amino acid residue 236 of the MHC heavy chain polypeptide.

Aspect 81. A method according to any of aspects 66-80, wherein the first polypeptide chain comprises a linker between the peptide epitope and the β2M polypeptide.

Aspect 82. A method according to any of aspects 66-81, wherein the first polypeptide chain comprises a linker between the peptide epitope and the β2M polypeptide, and wherein the disulfide bond links a Cys substituted for Gly2 in the linker with a Cys substituted for Tyr84 of the MHC heavy chain polypeptide.

Aspect 83. A method according to any one of aspects 66-82, wherein the immunomodulatory polypeptide is an activating polypeptide.

Aspect 84. A method according to any one of aspects 61-83, wherein said administering is intramuscular, intravenous, peritumoral, or intratumoral.

Aspect 85. A method according to any one of aspects 61-84, further comprising administering one or more checkpoint inhibitors to the individual.

Aspect 86. A method according to aspect 85, wherein the checkpoint inhibitor is an antibody that binds to a polypeptide selected from the group consisting of CD27, CD28, CD40, CD122, CD96, CD73, CD47, OX40, GITR, CSF1R, JAK, PI3K delta, PI3K gamma, TAM, arginase, CD137, ICOS, A2AR, B7-H3, B7-H4, BTLA, CTLA-4, LAG3, TIM3, VISTA, CD96, TIGIT, CD122, PD-1, PD-L1, and PD-L2.

Aspect 87. A method according to aspect 85, wherein the checkpoint inhibitor is an antibody specific for PD-1, PD-L1, or CTLA4.

Aspect 88. A method according to aspect 85, wherein the one or more checkpoint inhibitors is selected from the group consisting of nivolumab, pembrolizumab, pidilizumab, AMP-224, MPDL3280A, MDX-1105, MEDI-4736, arelumab, ipilimumab, tremelimumab, pidilizumab, IMP321, MGA271, BMS-986016, lirilumab, urelumab, PF-05082566, IPH2101, MEDI-6469, CP-870,893, Mogamulizumab, Varlilumab, Avelumab, Galiximab, AMP-514, AUNP 12, Indoximod, NLG-919, INCB024360, KN035, and combinations thereof.

Aspect 89. A composition according to any of Aspects 1-19 or a method according to any of Aspects 20-88, wherein the T cell used to make the mCTL is an allogeneic T cell.

Aspect 90. A composition or method according to Aspect 89, wherein the allogeneic T cell has been modified to present a TCR that is specific for a preselected antigen.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

1. (canceled)

2. A method of modulating a modified cytotoxic T cell (“mCTL”) that comprises a chimeric antigen receptor (CAR), wherein the CAR comprises an antigen-binding domain specific for a cancer-associated antigen,
wherein the method comprises contacting the mCTL with a dimeric T cell modulatory polypeptide (TMMP),
wherein the dimeric TMMP comprises two heterodimeric TMMPs, wherein each heterodimer comprises:
(a) a first polypeptide comprising a β-2 microglobulin (β2M) polypeptide, and a viral antigen peptide epitope; and
(b) a second polypeptide comprising a class I major histocompatibility complex (MHC) heavy chain polypeptide, one or more variant IL-2 polypeptides, and an immunoglobulin (Ig) Fc polypeptide;
wherein the β2M polypeptide, the MHC heavy chain polypeptide, and the peptide epitope and together form a peptide-MHC (pMHC) complex that specifically binds to a T cell receptor (TCR) on the mCTL,
wherein the one or more variant IL-2 polypeptides comprises the amino acid sequence set forth in SEQ ID NO:351, wherein amino acid 16 is Ala, and amino acid 42 is Ala, and
wherein the heterodimeric TMMPs are covalently linked together by one or more disulfide bonds between the Ig Fc polypeptides of each heterodimer.

3. The method of claim 2, wherein the peptide epitope is a human papilloma virus 16 (HPV16) E7 epitope.

4. The method of claim 3, wherein each heterodimeric TMMP comprises two variant IL-2 polypeptides in tandem.

5. The method of claim 4, wherein the class I MHC heavy chain polypeptide of each heterodimeric TMMP comprises an HLA-A heavy chain polypeptide, and the Ig Fc polypeptide comprises an IgG1 Fc polypeptide.

6. The method of claim 5, wherein:
a) the first polypeptide of each heterodimeric TMMP comprises:
i) the HPV16 E7 peptide epitope; and
ii) the β2M polypeptide,
b) the second polypeptide of each heterodimeric TMMP comprises:
i) the first variant IL-2 polypeptide;
ii) the second variant IL-2 variant polypeptide;
iii) the HLA-A heavy chain polypeptide; and
vii) the Ig Fc polypeptide, wherein the Ig Fc polypeptide is an IgG1 Fc polypeptide that comprises an amino acid sequence having at least about 95% percent amino acid sequence identity to SEQ ID NO:376,
wherein the β2M polypeptide comprises an amino acid sequence having at least 95% percent amino acid sequence identity to amino acids 21 to 119 of SEQ ID NO:388,
wherein the HLA-A heavy chain polypeptide comprises an amino acid sequence having at least 95% amino acid sequence identity to the amino acid sequence set forth in SEQ ID NO:185,
wherein the TMMP may include one or more independently selected peptide linkers, and
wherein the percent sequence identity is determinable by a sequence alignment performed using BLAST.

7. The method of claim 6, wherein the β2M polypeptide comprises a Cys at position 12, and the HLA-A heavy chain polypeptide comprises an Ala at position 84 and a Cys at position 236, and
wherein the heterodimeric TMMP comprises a disulfide bond joining the Cys at position 12 of the β2M polypeptide, based on the amino acid numbering of SEQ ID NO:202, to the Cys at position 236 of the HLA-A heavy chain polypeptide.

8. The method of claim 7, wherein the IgG1 Fc comprises an L234A substitution and an L235A substitution, which substitutions correspond to positions 14 and 15 of the amino acid sequence set forth in SEQ ID NO:387.

9. A composition comprising a quantity of modified cytotoxic T cells (“mCTLs”), wherein the mCTLs comprise: a) a T-cell receptor (TCR) that is specific for a preselected antigen that is an epitope presented by a peptide-MHC (pMHC) complex, wherein the pMHC complex comprises a β2M polypeptide, an MHC heavy chain polypeptide, and a peptide epitope; and b) a chimeric antigen receptor (CAR), wherein the CAR comprises an antigen-binding domain specific for a cancer-associated antigen, and wherein the percentage of mCTLs cells in the composition exceeds at least 1% of the total number of T cells in the composition.

10. The composition of claim 9, wherein the peptide epitope is an epitope of an antigen encoded by a virus or a bacterium.

11. The composition of claim 10, wherein the peptide epitope is an epitope of an antigen encoded by a virus or bacteria selected from the group consisting of cytomegalovirus (CMV), Epstein-Barr virus (EBV), human papilloma virus (HPV), adenovirus, influenza virus, and Clostridium tetani.

12. The composition of claim 11, wherein the CAR comprises: a) an extracellular domain comprising the antigen-binding domain; b) a transmembrane region; and c) a cytoplasmic domain comprising an intracellular signaling domain.

13. A method of treating a cancer in an individual, the method comprising the steps of:
(i) administering to the individual the composition of claim 9, and
(ii) administering to the individual a composition comprising a dimeric T-cell modulatory polypeptide (TMMP), wherein the TMMP comprises a peptide-major histocompatibility complex (pMHC) complex that presents an epitope of the preselected antigen,
wherein the dimeric TMMP comprises two heterodimeric TMMPs, wherein each heterodimer comprises:
(a) a first polypeptide comprising a β-2 microglobulin (β2M) polypeptide, and a viral antigen peptide epitope; and
(b) a second polypeptide comprising a class I major histocompatibility complex (MHC) heavy chain polypeptide, one or more variant IL-2 polypeptides, and an immunoglobulin (Ig) Fc polypeptide;
wherein the β2M polypeptide, the MHC heavy chain polypeptide, and the peptide epitope and together form a peptide-MHC (pMHC) complex that specifically binds to a T cell receptor (TCR) on the mCTL,
wherein the one or more variant IL-2 polypeptides comprises the amino acid sequence set forth in SEQ ID NO:351, wherein amino acid 16 is Ala, and amino acid 42 is Ala, and
wherein the heterodimeric TMMPs are covalently linked together by one or more disulfide bonds between the Ig Fc polypeptides of each heterodimer.

14. The method of claim 13, wherein the individual does not undergo a lymphodepleting regimen prior to administering step (i).

15. The method of claim 13, wherein the composition of claim 9 is administered before the composition comprising the TMMP, after the composition comprising the TMMP, or at the same time as the composition comprising the TMMP.

16. The method of claim 15, wherein the peptide epitope is an epitope of an antigen encoded by a virus or bacteria selected from the group consisting of cytomegalovirus (CMV), Epstein-Barr virus (EBV), human papilloma virus (HPV), adenovirus, influenza virus, and Clostridium tetani.

17. A method of making an in vitro composition comprising a quantity of modified cytotoxic T cells, wherein the mCTLs comprise: a) a T-cell receptor (TCR) that is specific for a preselected antigen that is an epitope presented by a peptide-MHC (pMHC) complex, wherein the pMHC complex comprises a β2M polypeptide, an MHC heavy chain polypeptide, and a peptide epitope; and b) a chimeric antigen receptor (CAR), wherein the CAR comprises an antigen-binding domain specific for a cancer-associated antigen, and wherein the percentage of mCTLs cells in the composition exceeds at least 1% of the total number of T cells in the composition (“mCTLs”), wherein the method comprises the steps of:
(i) providing a composition comprising a quantity of T cells having a T-cell receptor (TCR) specific for the pNMC complex; and
(ii) modifying the composition in step (i) by introducing into the at least partially separated target T cells one or more nucleic acids comprising nucleotide sequences encoding a chimeric antigen receptor (CAR) that comprises an antigen-binding domain specific for a cancer-associated antigen.

18. The method of claim 17, wherein the CAR comprises: a) an extracellular domain comprising the antigen-binding domain; b) a transmembrane region; and c) a cytoplasmic domain comprising an intracellular signaling domain.

19. The method of claim 18, wherein the intracellular signaling domain comprises a signaling domain from the zeta chain of human CD3.

20. The method of claim 19, wherein the cytoplasmic domain comprises one or more costimulatory polypeptides.

21. The method of claim 20, wherein the costimulatory polypeptide is selected from CD28, 4-1BB, and OX-40.
